(19) United States
(12) Patent Application Publication
(10) Pub. No.: US 2024/0025862 A1
(43) Pub. Date: Jan. 25, 2024

(54) SUBSTITUTED N-PHENYLURACILS AND SALTS THEREOF, AND USE THEREOF AS HERBICIDAL ACTIVE SUBSTANCES

(71) Applicant: Bayer Aktiengesellschaft, Leverkusen (DE)

(72) Inventors: Ines HEINEMANN, Hofheim (DE); Harald JAKOBI, Frankfurt (DE); Hendrik HELMKE, Liederbach (DE); Jens FRACKENPOHL, Frankfurt (DE); Christopher Hugh ROSINGER, Hofheim (DE); Elmar GATZWEILER, Bad Nauheim (DE); Elisabeth ASMUS, Hoesbach (DE); Birgit BOLLENBACH-WAHL, Weiler/Bingen (DE)

(21) Appl. No.: 18/042,466

(22) PCT Filed: Aug. 20, 2021
(86) PCT No.: PCT/EP2021/073129

§ 371 Date: Feb. 22, 2023

(30) Foreign Application Priority Data
Aug. 24, 2020 (EP) 20192356.2

(51) Int. Cl. C07D 239/10; C07D 405/12; C07D 409/12; C07D 403/12; C07D 401/12; C07F 7/08; C07F 5/02; C07D 417/12

(52) U.S. Cl. CPC C07D 239/10; C07D 405/12; C07D 409/12; C07D 417/12; C07D 401/12; C07F 7/0812; C07F 5/025; C07D 403/12

(57) ABSTRACT

Substituted N-phenyluracils and salts thereof and use thereof as herbicidal agents.

The present invention relates to substituted N-phenyluracils of the general formula (I) or salts thereof,

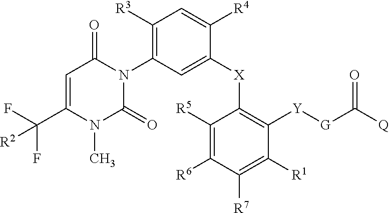

where the radicals in the general formula (I) conform to the definitions given in the description, and to the use thereof as herbicides, especially for control of broad-leaved weeds and/or weed grasses in crops of useful plants and/or as plant growth regulators for influencing the growth of crops of useful plants.

The invention relates to the technical field of crop protection agents, in particular that of herbicides for the selective control of broad-leaved weeds and weed grasses in crops of useful plants.

Specifically, the present invention relates to substituted N-phenyluracils and salts thereof, to processes for preparation thereof and to the use thereof as herbicides, especially for control of broad-leaved weeds and/or weed grasses in crops of useful plants and/or as plant growth regulators for influencing the growth of crops of useful plants.

In their application, crop protection agents known to date for the selective control of harmful plants in crops of useful plants or active ingredients for controlling unwanted vegetation sometimes have disadvantages, whether (a) that they have no or else insufficient herbicidal activity against particular harmful plants, (b) that the spectrum of harmful plants which can be controlled with an active ingredient is not wide enough, (c) that their selectivity in crops of useful plants is too low and/or (d) that they have a toxicologically unfavorable profile. Furthermore, some active ingredients which can be used as plant growth regulators for a number of useful plants cause undesirably reduced harvest yields in other useful plants or are compatible with the crop plant only within a narrow application rate range, if at all. Some of the known active ingredients cannot be produced economically on an industrial scale owing to precursors and reagents which are difficult to obtain, or they have only insufficient chemical stabilities. In the case of other active ingredients, the activity is too highly dependent on environmental conditions, such as weather and soil conditions.

The herbicidal action of these known compounds, especially at low application rates, and/or the compatibility thereof with crop plants is still in need of improvement.

It is known from various documents that particular substituted N-bonded aryluracils can be used as active herbicidal ingredients (cf. EP1106607, EP408382, EP473551, EP648749, U.S. Pat. Nos. 4,943,309, 5,084,084, 5,127,935, 6,537,948, JP2001/348376, JP2001/354661, JP2002/003480, JP2002/363010, JP2002/363170, WO91/00278, WO95/29168, WO95/30661, WO96/35679, WO97/01541, WO98/25909, WO 2001/034575, WO2001/39597, WO2001/85907, WO2002/098227, WO2002/098228, WO2003/028462, WO2003/028463, WO2003/0284647, WO2016/095768). However, the known aryluracils have a number of gaps in activity, especially with respect to monocotyledonous weeds. A number of combinations of active herbicidal ingredients based on N-bonded aryluracils have likewise become known (cf. DE4437197, EP714602, WO96/07323, WO96/08151, JP11189506, JP2003/104808, JP2003/104809, JP2003/104810, JP2003/160415 and JP2003/160416). However, the properties of these active ingredient combinations were not entirely satisfactory.

Also known are substituted uracils having an N-bonded and further-substituted diaryl ether group or a corresponding heteroaryl aryl ether radical (cf. U.S. Pat. Nos. 6,333,296, 6,121,201, WO2001/85907, EP1122244, EP1397958, EP1422227, WO 2002/098227). There have additionally been descriptions of highly substituted N-phenyluracils having a carbonylalkyloxy group with specific substitution (cf. WO2011/137088). WO2018/019842 describes the use of specifically substituted N-phenyluracils for controlling particular dicotyledonous weeds having specific resistance to established herbicides. Substituted 3-phenyl-5-alkyl-6-(trifluoromethyl)pyrimidine-2,4(1H,3H)-diones (cf. WO2019/101551) have likewise become known.

It has now been found that, surprisingly, selected N-phenyluracils having a substituted alkyl ester side chain or salts thereof are very suitable as herbicides and can be used particularly advantageously as active ingredients for controlling monocotyledonous and dicotyledonous weeds in crops of useful plants.

The present invention thus provides substituted N-phenyluracils of the general formula (I) or salts thereof

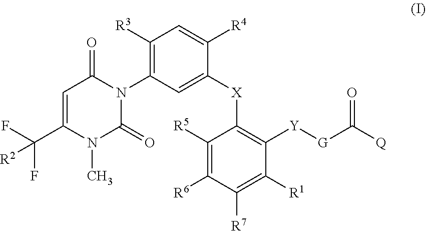

in which
R¹ is hydrogen, halogen, cyano, (C₁-C₈)-alkyl, (C₁-C₈)-haloalkyl, (C₁-C₈)-alkoxy, (C₁-C₈)-haloalkoxy,
R² is hydrogen, fluorine, chlorine, bromine, trifluoromethyl, (C₁-C₈)-alkoxy,
R³ is hydrogen, halogen, (C₁-C₈)-alkoxy,
R⁴ is halogen, cyano, NO₂, C(O)NH₂, C(S)NH₂, (C₁-C₈)-haloalkyl, (C₂-C₈)-alkynyl,
R⁵, R⁶ and R⁷ are independently hydrogen, halogen, cyano, (C₁-C₈)-alkyl, (C₁-C₈)-haloalkyl, (C₁-C₈)-alkoxy, (C₁-C₈)-haloalkoxy,
G is unbranched or branched (C₁-C₈)-alkylene,
Q is a radical of the formula

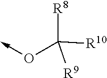

R⁸ is hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, (C₁-C₈)-alkoxy-(C₁-C₈)-alkyl,
R⁹ is hydrogen or (C₁-C₈)-alkyl,
R¹⁰ is cyano, NO₂, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris[(C₁-C₈)-alkyl]silyl-(C₁-C₈)-alkyl, bis[(C₁-C₈)-alkyl](aryl)silyl(C₁-C₈)-alkyl, [(C₁-C₈)-alkyl]bis(aryl)silyl-(C₁-C₈)-alkyl, tris[(C₁-C₈)-alkyl]silyl, bishydroxyboryl-(C₁-C₈)-alkyl, bis[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)R¹⁴, bis(C₁-C₈)-alkoxymethyl, bis(C₁-C₈)-alkoxymethyl-(C₁-C₈)-alkyl, or
R⁸ and R¹⁰ together with the carbon atom to which they are bonded form a fully saturated or partly saturated 3- to 10-membered monocyclic or bicyclic heterocyclyl optionally having further substitution,
R¹¹ and R¹² are the same or different and are independently hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis[(C₁-C₈)-alkyl]aminocarbonyl-(C₁-C₈)-alkyl, (C₁-C₈)-alkylaminocarbonyl-(C₁-C₈)-alkyl, aryl-(C₁-C₈)-alkylaminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis[(C₁-C₈)-alkyl]aminocarbonyl-(C₁-C₈)-alkyl, (C₁-C₈)-alkylaminocarbonyl-(C₁-C₈)-alkyl, aryl-(C₁-C₈)-alkylaminocarbonyl-(C₁-C₈)-alkyl, bis[(C₁-C₈)-alkyl]amino-(C₂-C₆)-alkyl, (C₁-C₈)-alkylamino-(C₂-C₆)-alkyl, aryl-(C₁-C₈)-alkylamino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris[(C₁-C₈)-alkyl]silyl-(C₁-C₈)-alkyl, bis[(C₁-C₈)-alkyl](aryl)silyl(C₁-C₈)-alkyl, [(C₁-C₈)-alkyl]bis(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, (C₁-C₈)-alkoxycarbonyl,
R¹⁴ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino; N-azetidinyl, N-pyrrolidinyl, N-piperidinyl, N-morpholinyl,
and
X and Y are independently O (oxygen) or S (sulfur).

The invention particularly preferably provides compounds of the general formula (I), in which
R¹ is hydrogen, halogen, cyano, (C₁-C₆)-alkyl, (C₁-C₆)-haloalkyl, (C₁-C₆)-alkoxy, (C₁-C₆)-haloalkoxy,
R² is hydrogen, fluorine, chlorine, bromine, trifluoromethyl, (C₁-C₆)-alkoxy,
R³ is hydrogen, halogen, (C₁-C₆)-alkoxy,
R⁴ is halogen, cyano, NO₂, C(O)NH₂, C(S)NH₂, (C₁-C₆)-haloalkyl, (C₂-C₆)-alkynyl,
R⁵, R⁶ and R⁷ are independently hydrogen, halogen, cyano, (C₁-C₆)-alkyl, (C₁-C₆)-haloalkyl, (C₁-C₆)-alkoxy, (C₁-C₆)-haloalkoxy,
G is unbranched or branched (C₁-C₆)-alkylene,
Q is a radical of the formula

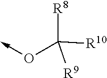

R⁸ is hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ is hydrogen or (C₁-C₄)-alkyl,
R¹⁰ is cyano, NO₂, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris[(C₁-C₆)-alkyl]silyl-(C₁-C₆)-alkyl, bis[(C₁-C₆)-alkyl](aryl)silyl(C₁-C₆)-alkyl, [(C₁-C₆)-alkyl]bis(aryl)silyl-(C₁-C₆)-alkyl, tris[(C₁-C₆)-alkyl]silyl, bishydroxyboryl-(C₁-C₆)-alkyl, bis[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)R¹³, bis(C₁-C₆)-alkoxymethyl, bis(C₁-C₆)-alkoxymethyl-(C₁-C₆)-alkyl,
R⁸ and R¹⁰ together with the carbon atom to which they are bonded form fully saturated or partly saturated 3- to 10-membered monocyclic or bicyclic heterocyclyl optionally having further substitution,
R¹¹ and R¹² are the same or different and are independently hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis[(C₁-C₆)-alkyl]aminocarbonyl-(C₁-C₆)-alkyl, (C₁-C₆)-alkylaminocarbonyl-(C₁-C₆)-alkyl, aryl-(C₁-C₆)-alkylaminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis[(C₁-C₆)-alkyl]aminocarbonyl-(C₁-C₆)-alkyl, (C₁-C₆)-alkylaminocarbonyl-(C₁-C₆)-alkyl, aryl-(C₁-C₆)-alkylaminocarbonyl-(C₁-C₆)-alkyl, bis[(C₁-C₆)-alkyl]amino-(C₂-C₄)-alkyl, (C₁-C₆)-alkylamino-(C₂-C₄)-alkyl, aryl-(C₁-C₆)-alkylamino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris[(C₁-C₆)-alkyl]silyl-(C₁-C₆)-alkyl, bis[(C₁-C₆)-alkyl](aryl)silyl(C₁-C₆)-alkyl, [(C₁-C₆)-alkyl]bis(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁₀)-cycloalkenyl-(C₁-C₆)-alkyl, bis[(C₁-C₆)-alkyl]amino, (C₁-C₆)-alkylamino, aryl-(C₁-C₆)-amino, aryl-(C₁-C₂)-alkylamino, aryl-[(C₁-C₆)-alkyl]amino; (C₃-C₆)-cycloalkylamino, (C₃-C₆)-cycloalkyl-[(C₁-C₆)-alkyl]amino; N-azetidinyl, N-pyrrolidinyl, N-piperidinyl, N-morpholinyl,
and
X and Y are independently O (oxygen) or S (sulfur).

The invention very particularly preferably provides compounds of the general formula (I) in which
R¹ is hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
R² is hydrogen, fluorine, chlorine, bromine, trifluoromethyl, methoxy, ethoxy, prop-1-yloxy, but-1-yloxy,
R³ is hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ is fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ are independently hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
X and Y are independently O (oxygen) or S (sulfur)
and
Q is one of the moieties Q-1 to Q-406 specified below, where the arrow in the structural formulae in the table below represents a bond of the respective Q group to the carbonyl group in the general formula (I):

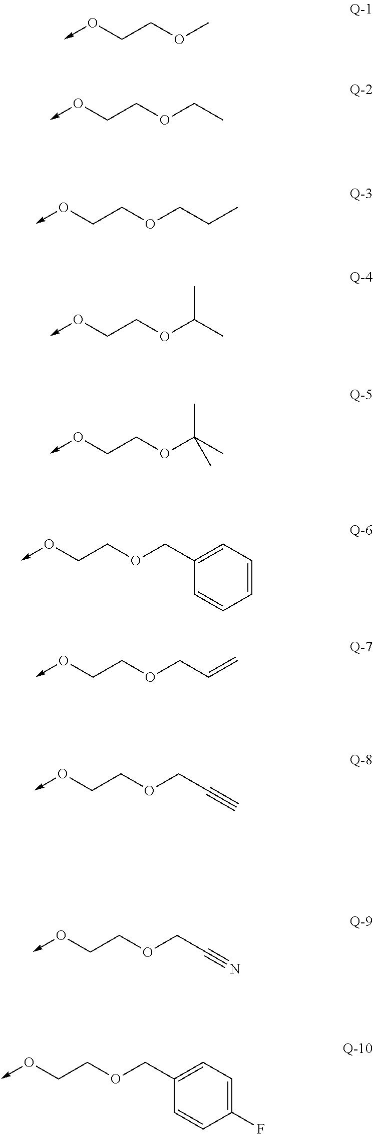

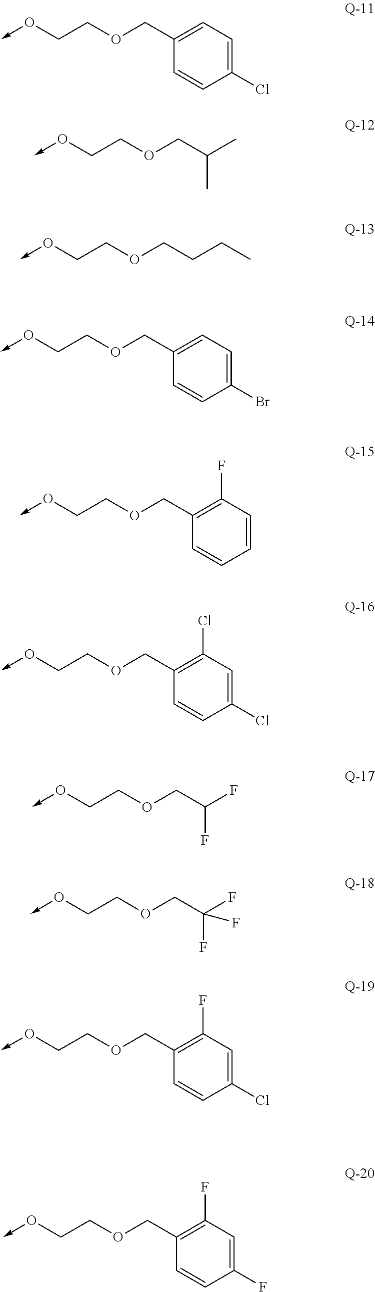

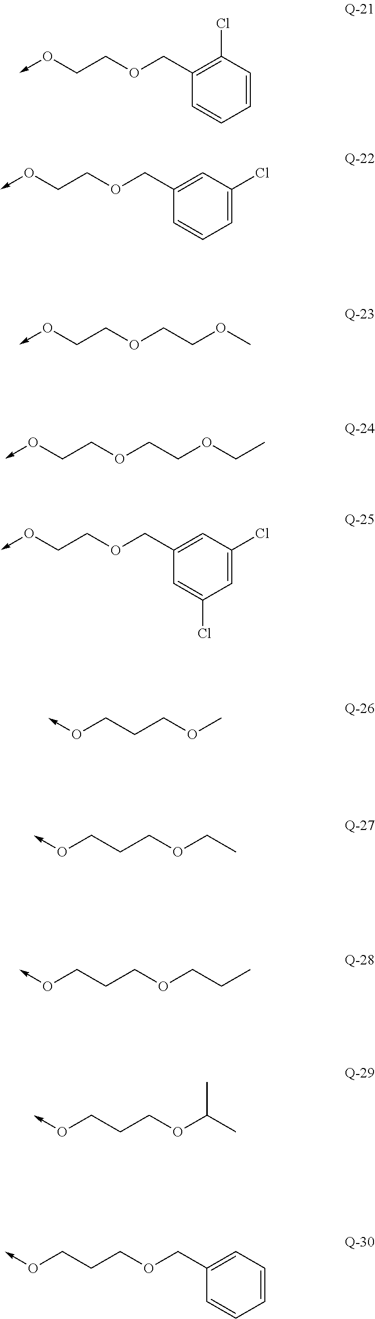

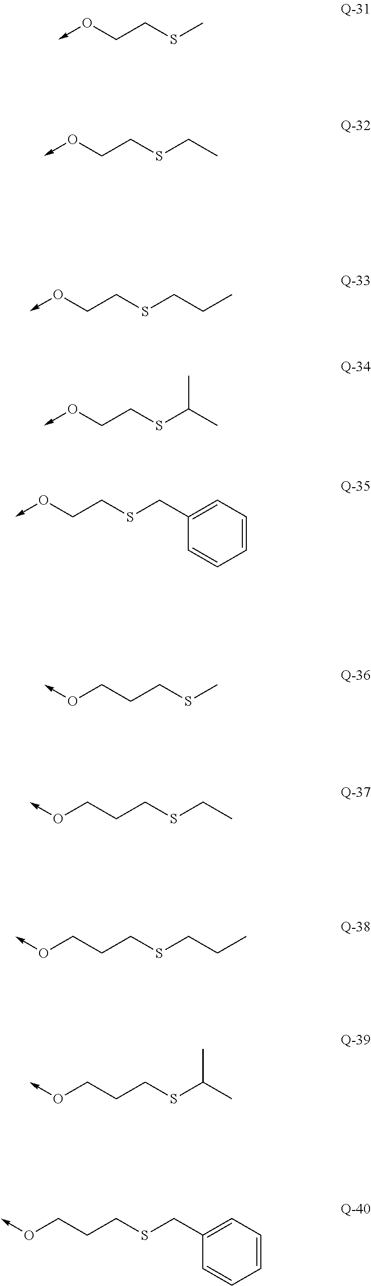

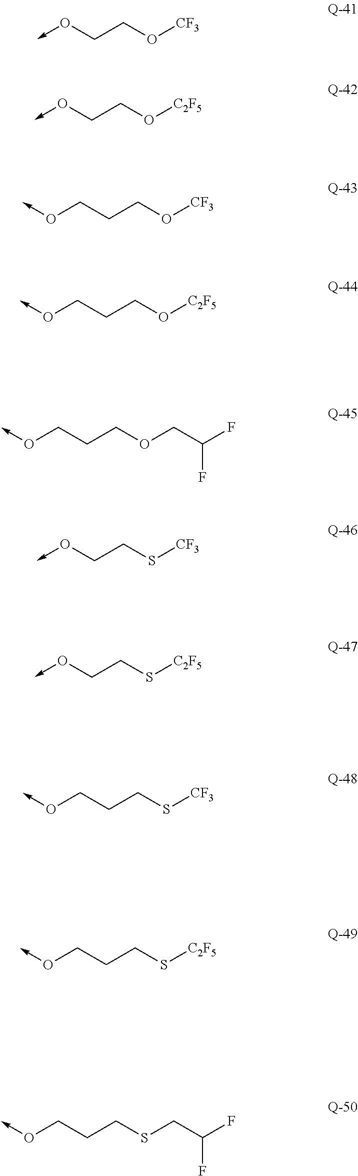

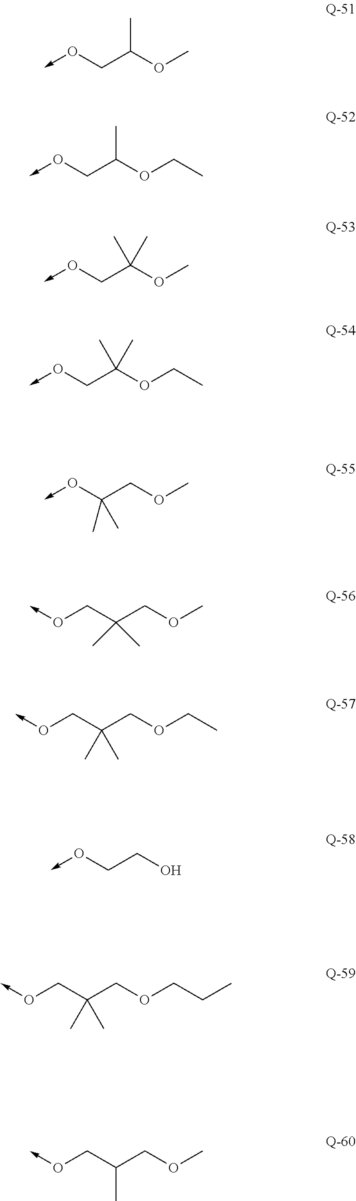

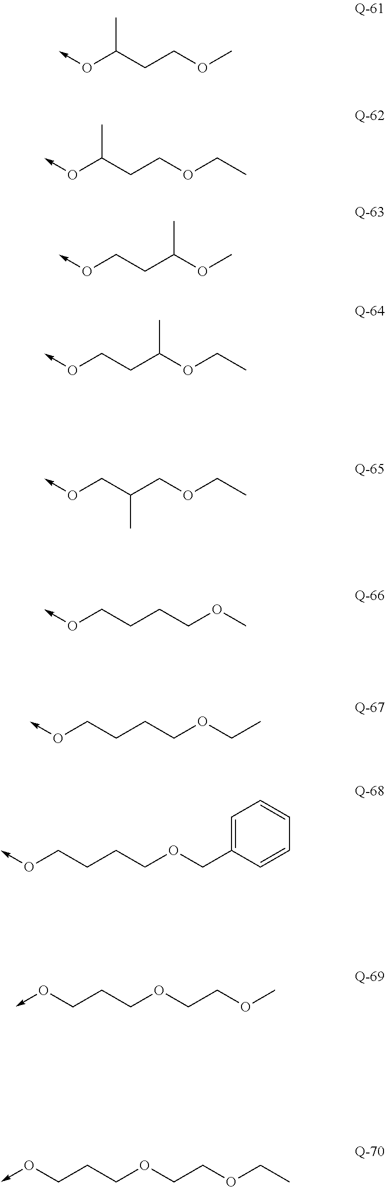

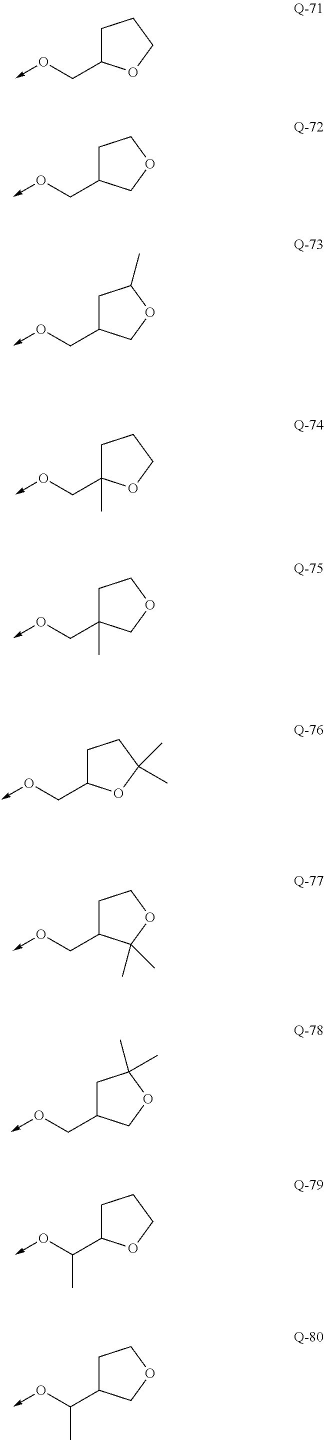

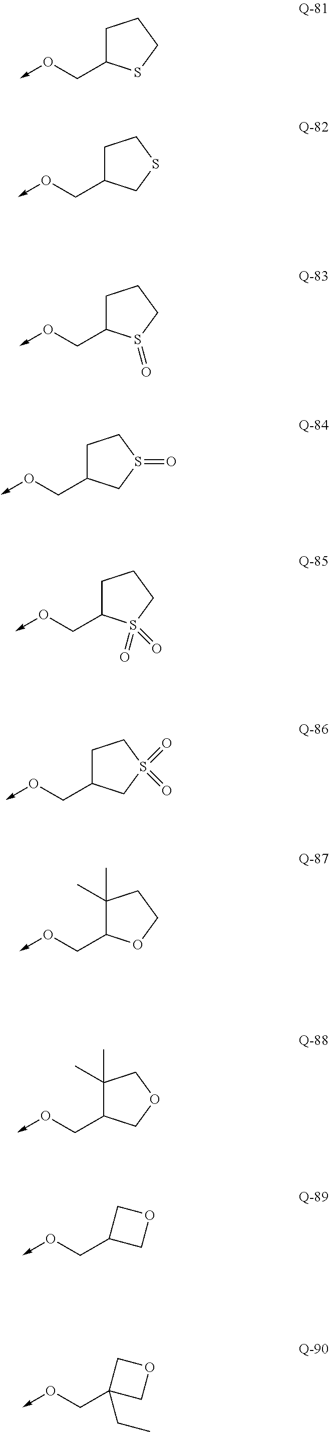

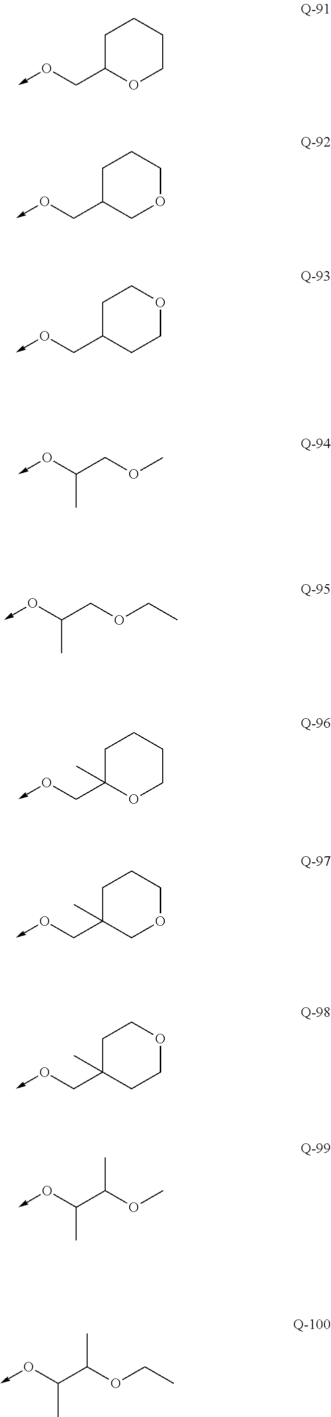

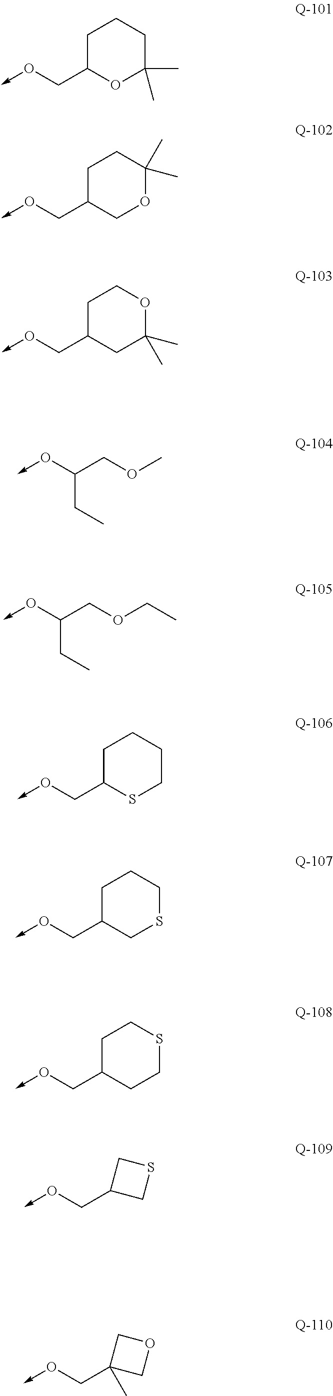

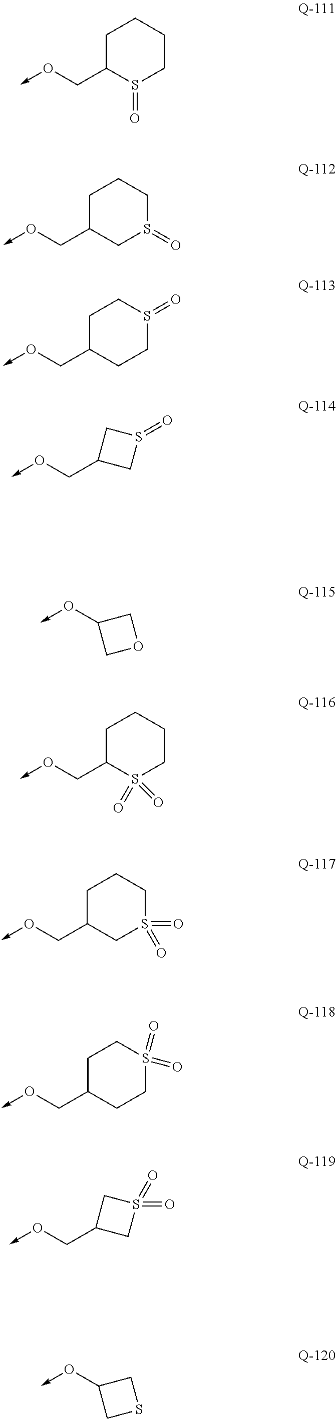

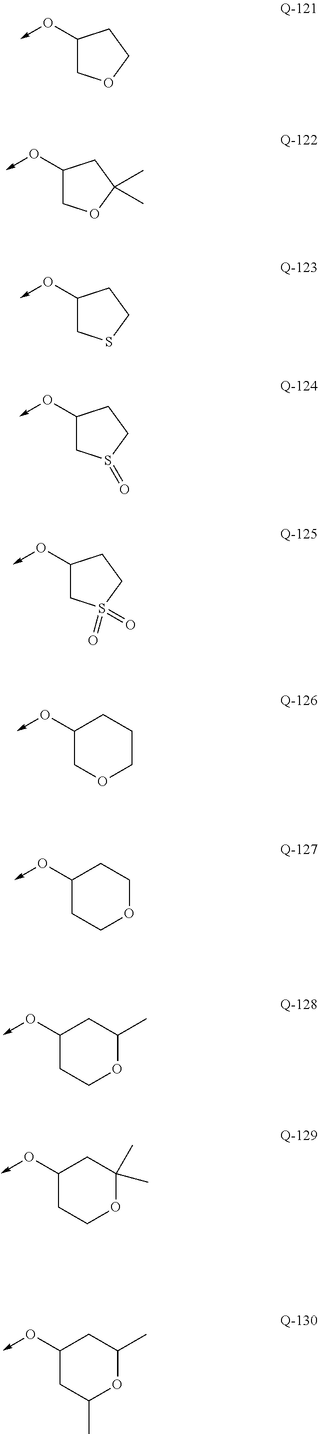

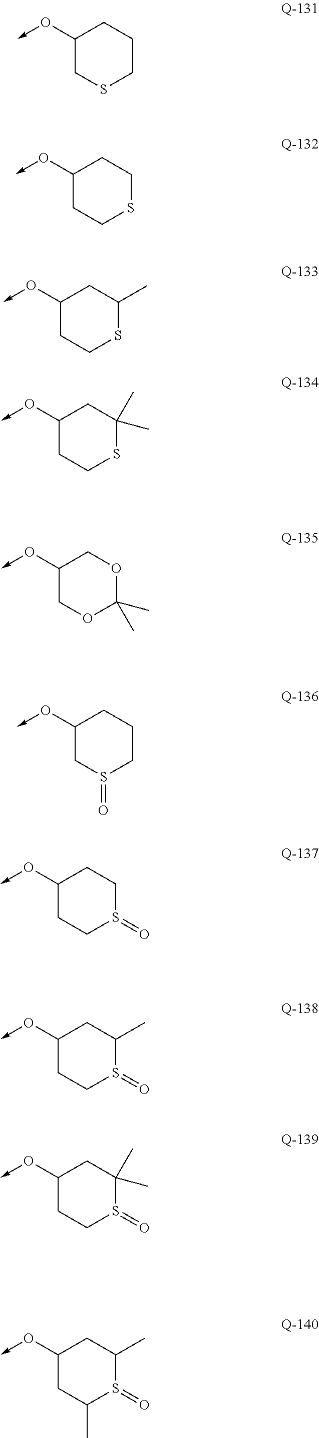

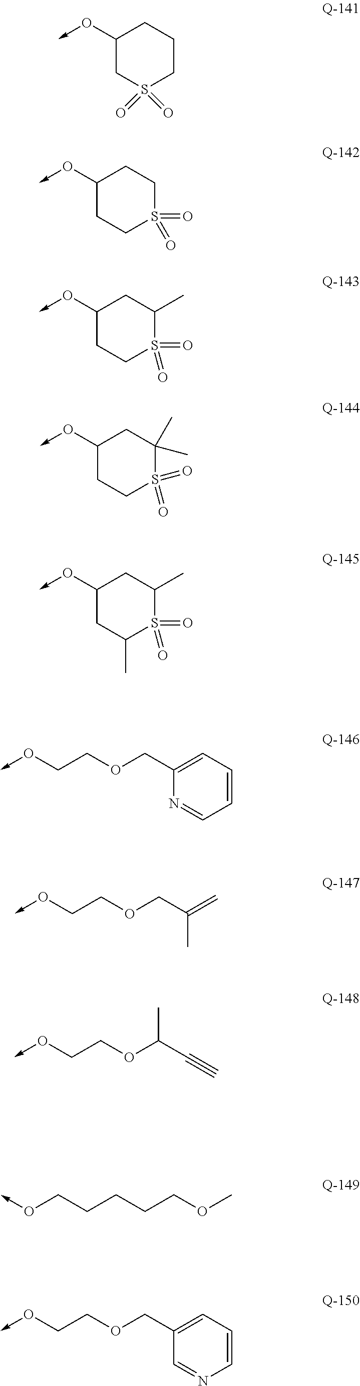

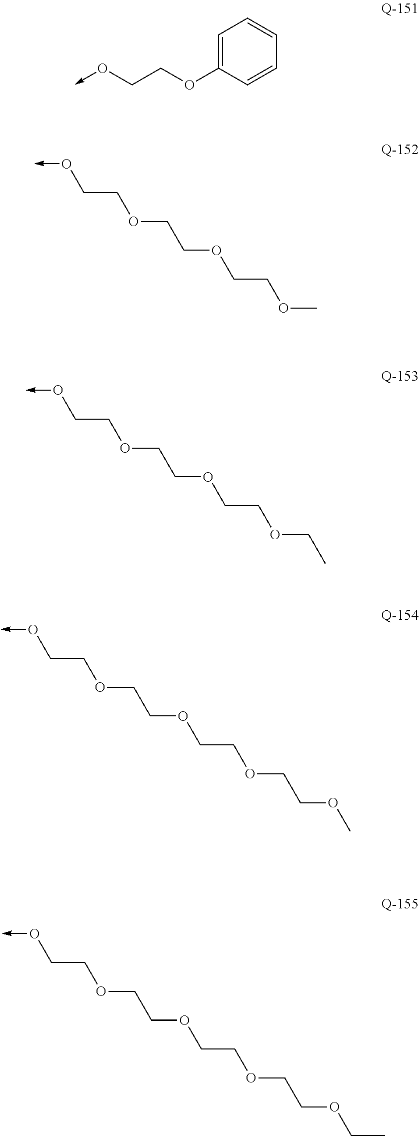

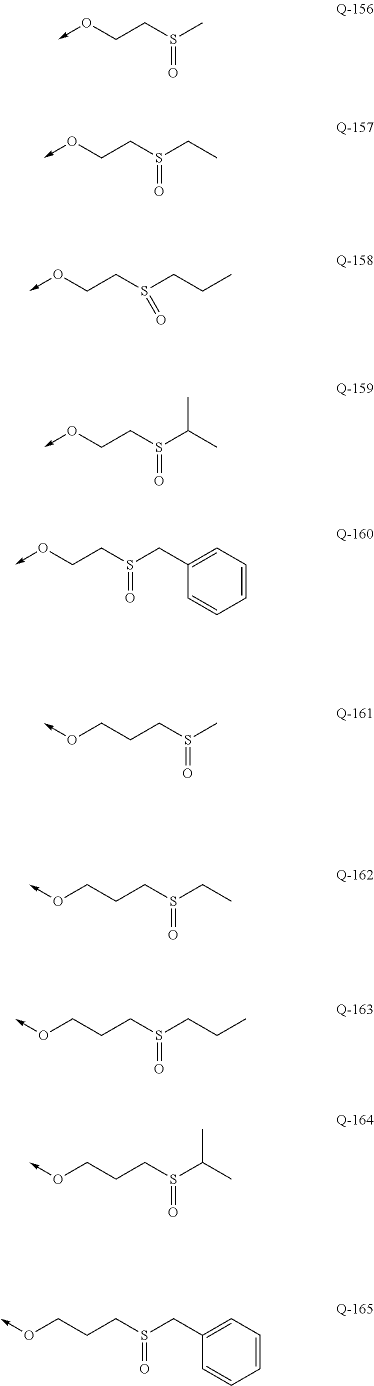

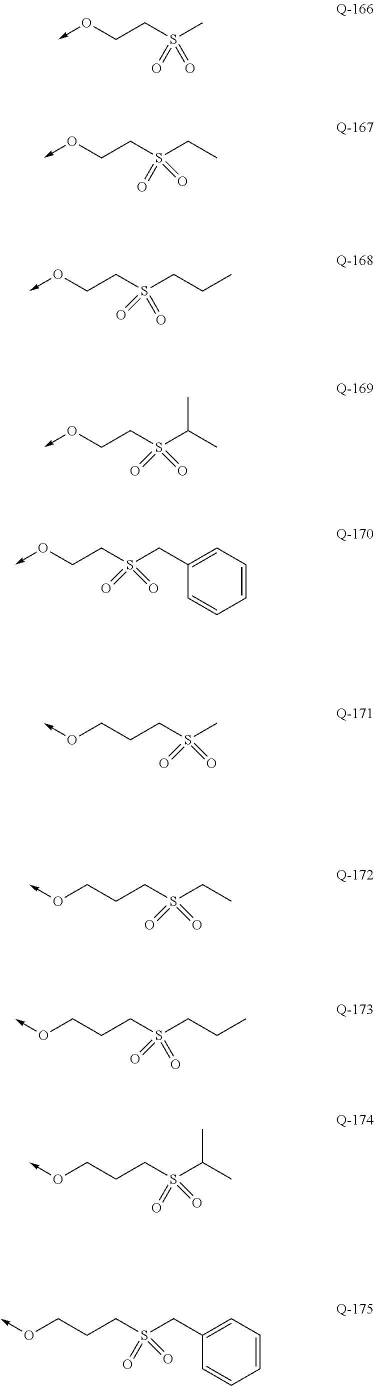

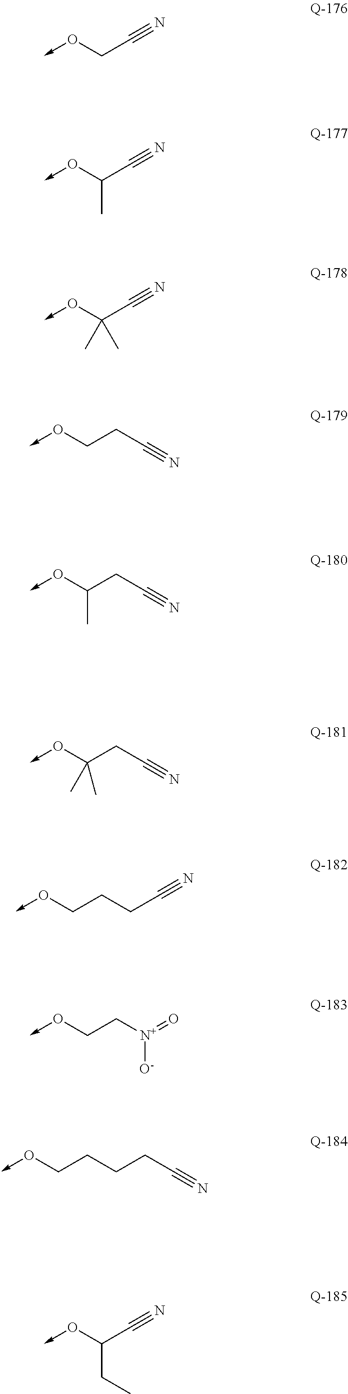

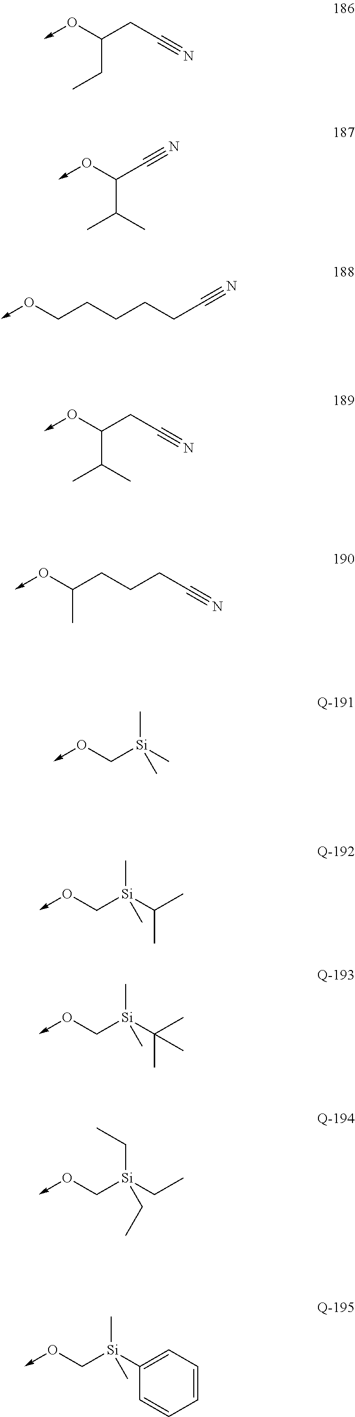

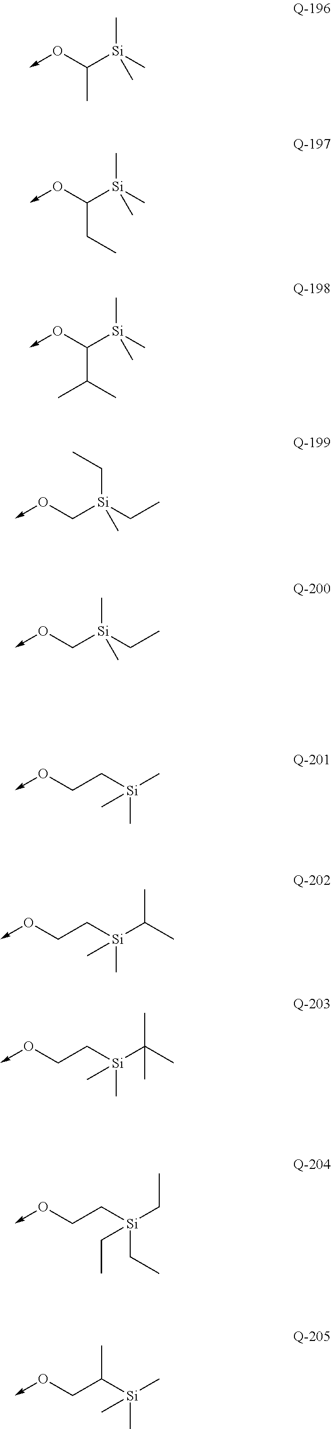

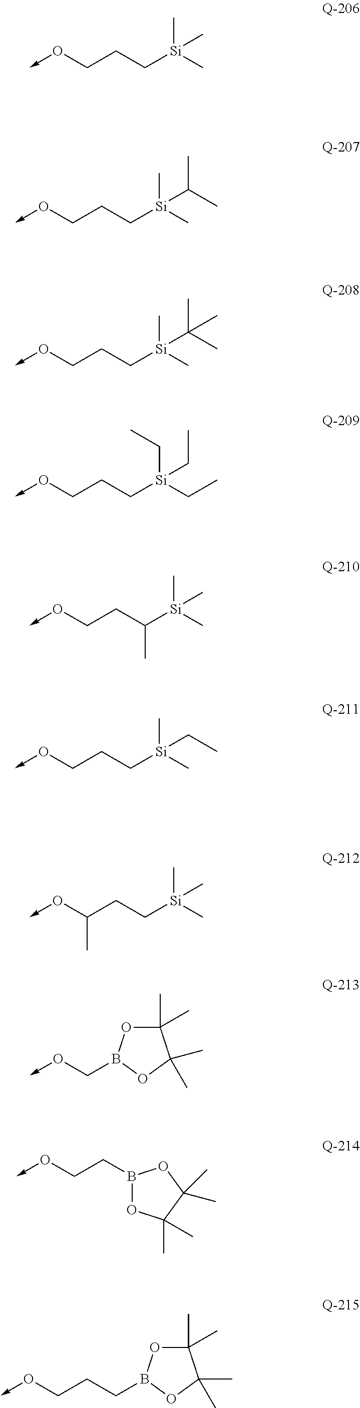

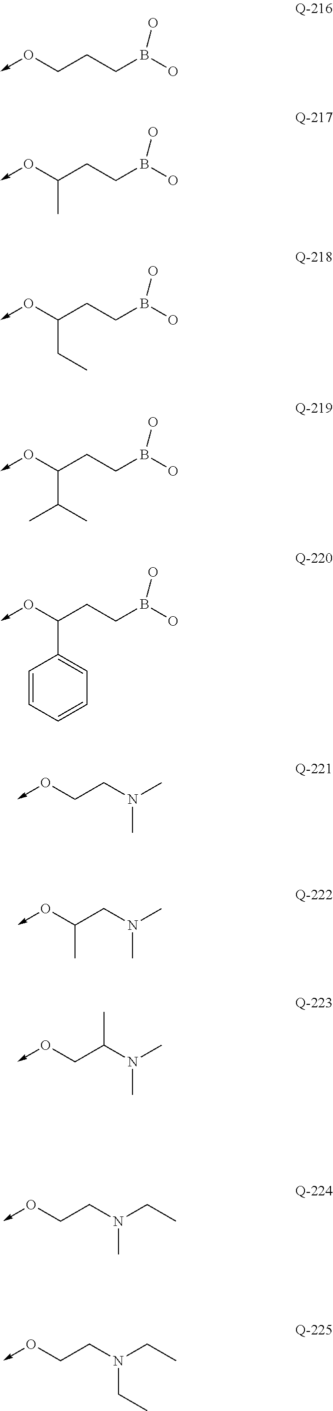

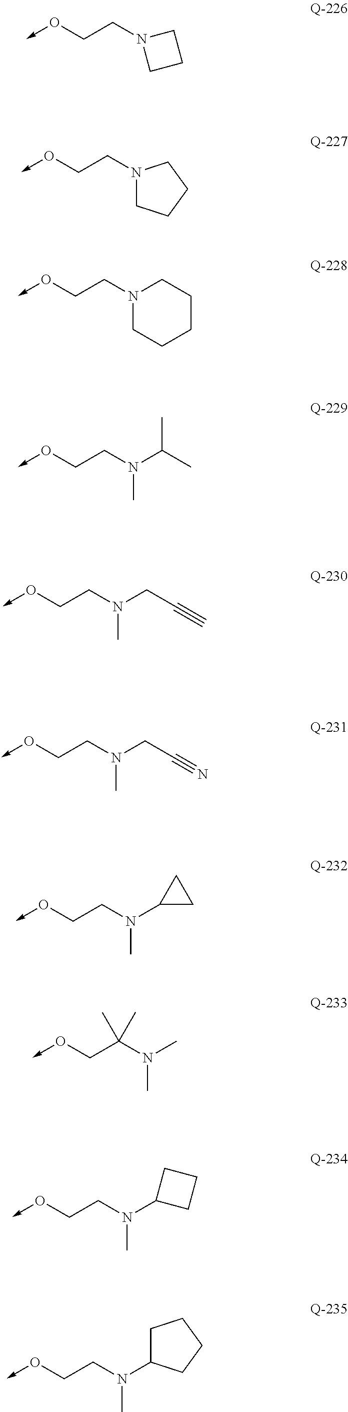

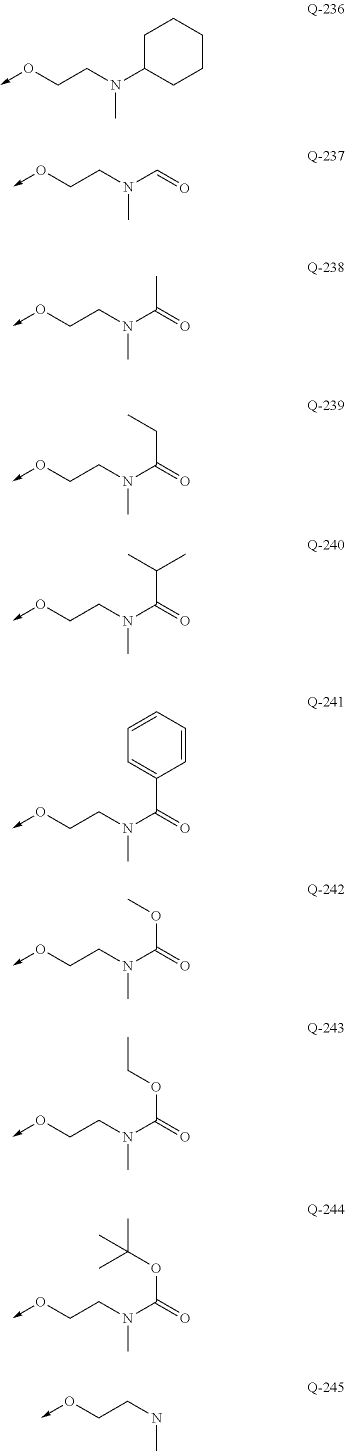

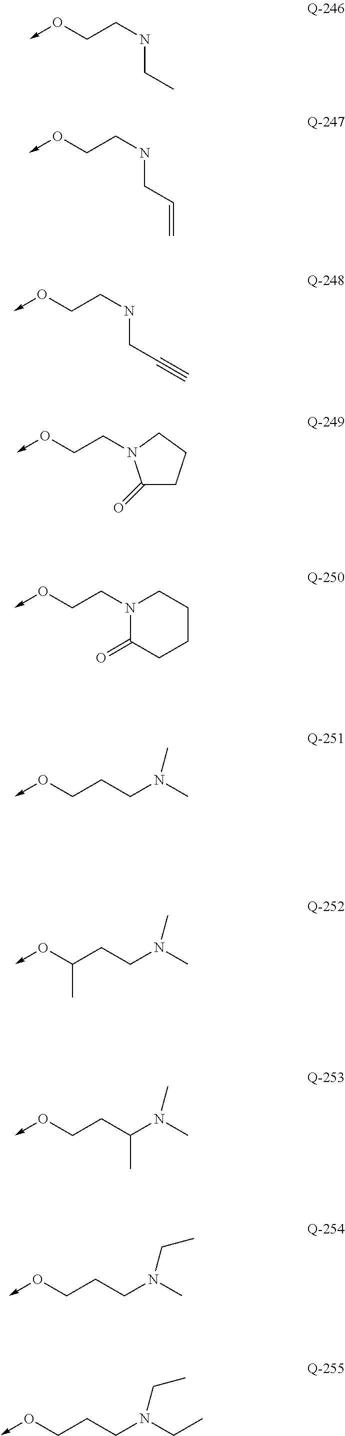

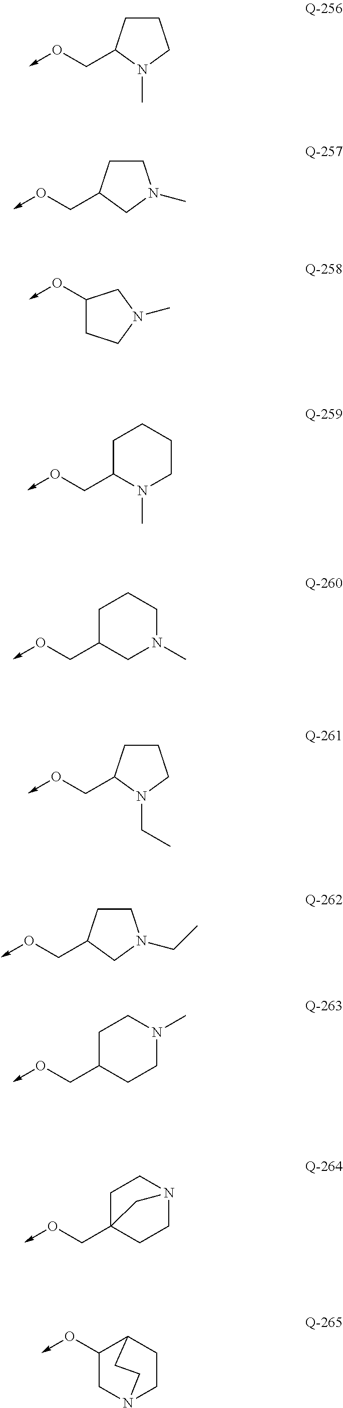

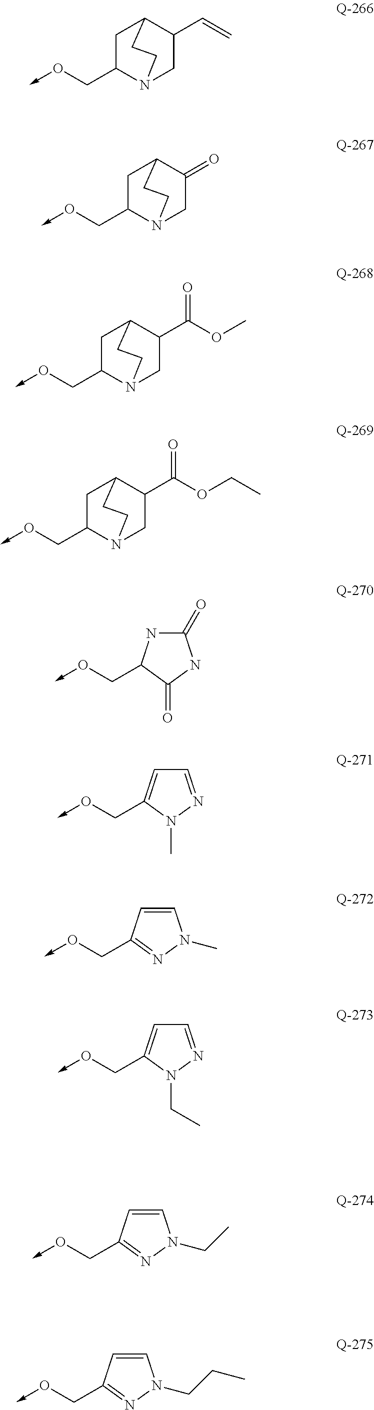

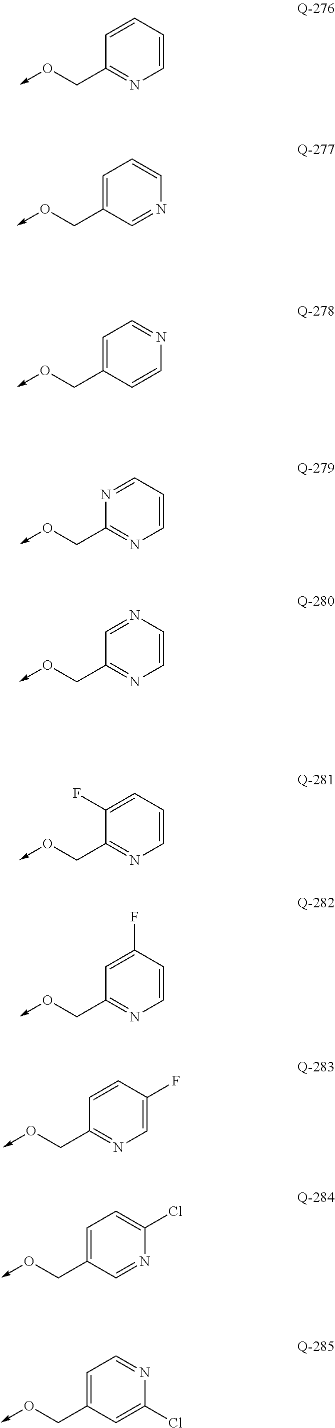

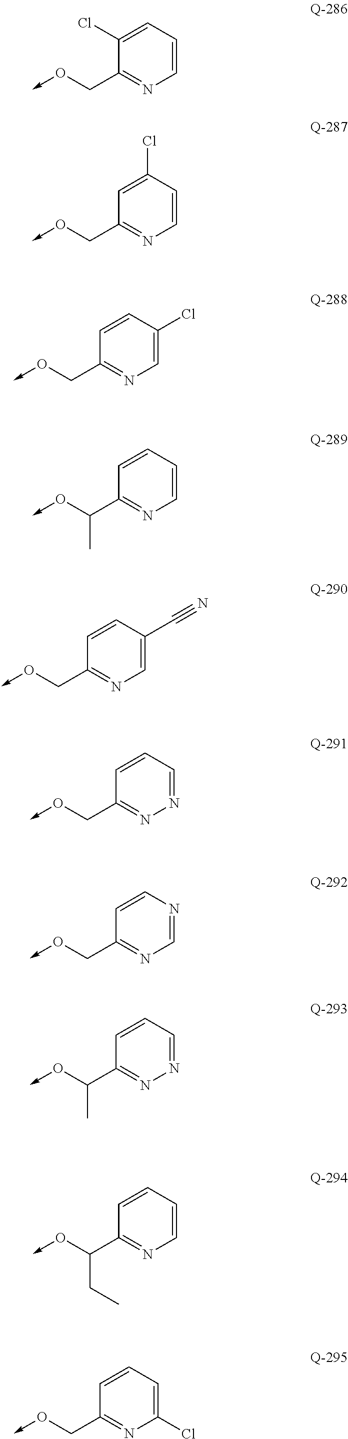

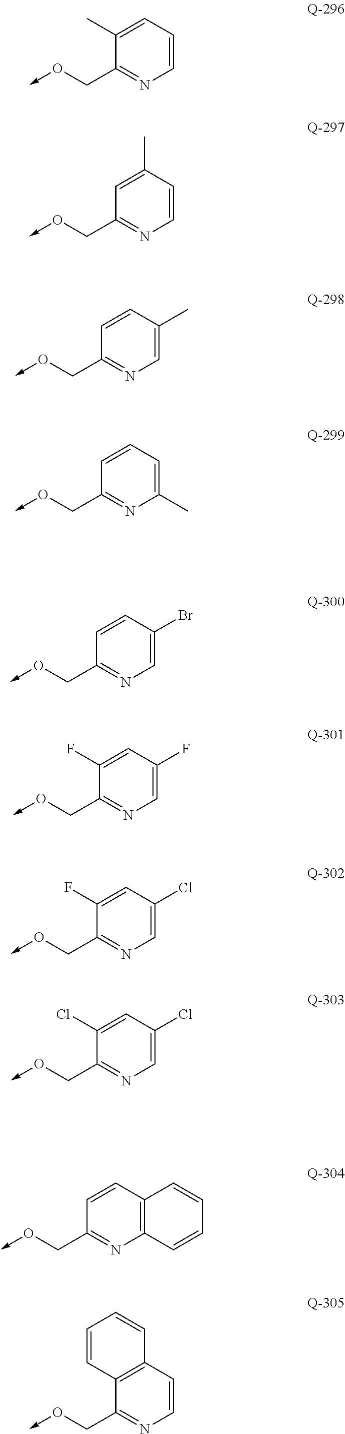

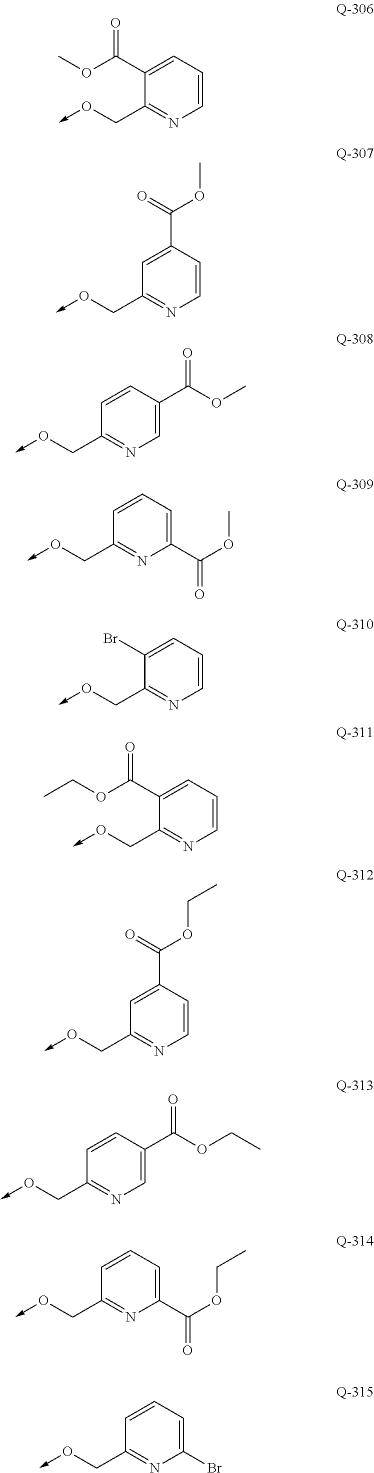

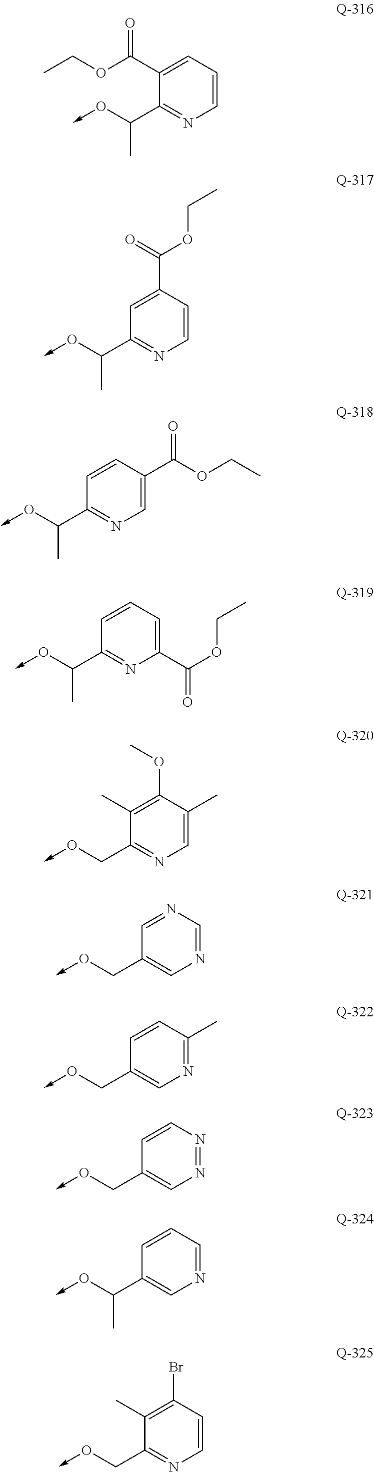

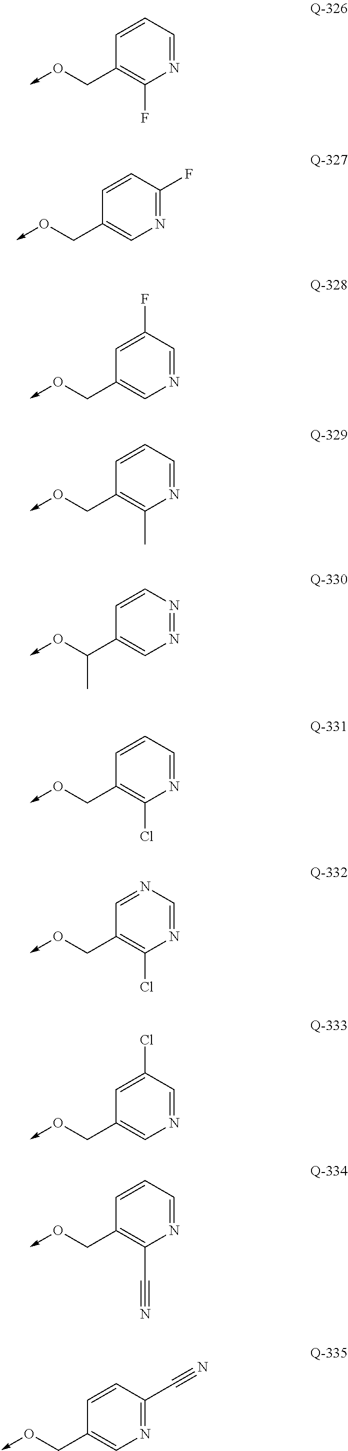

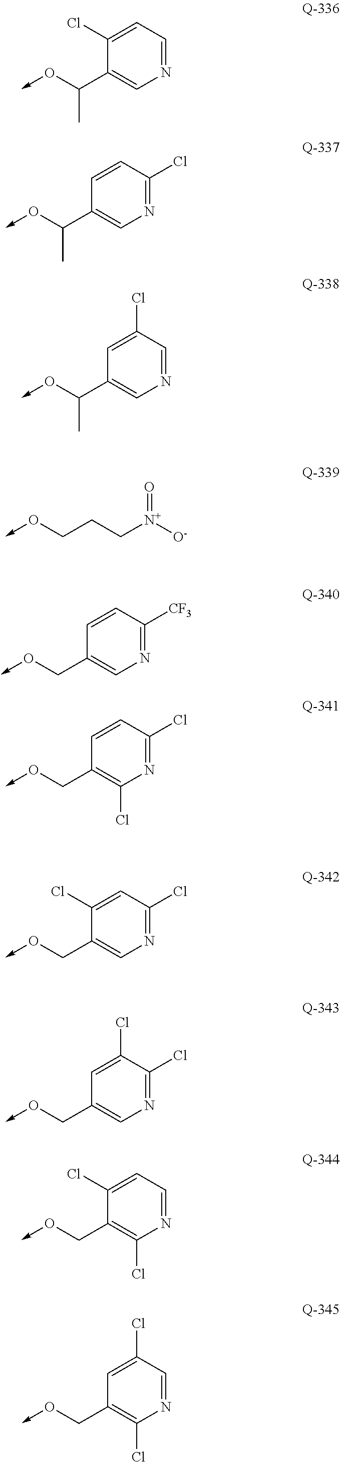

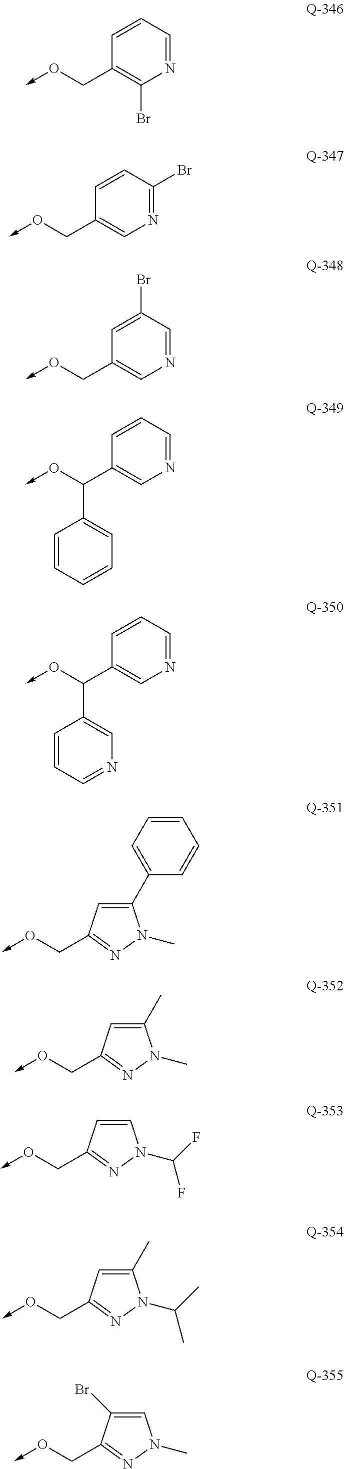

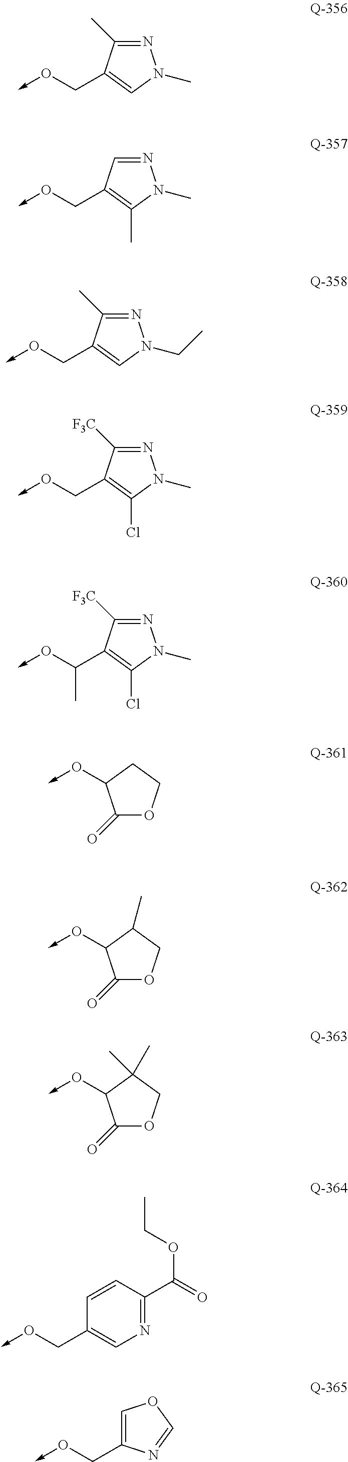

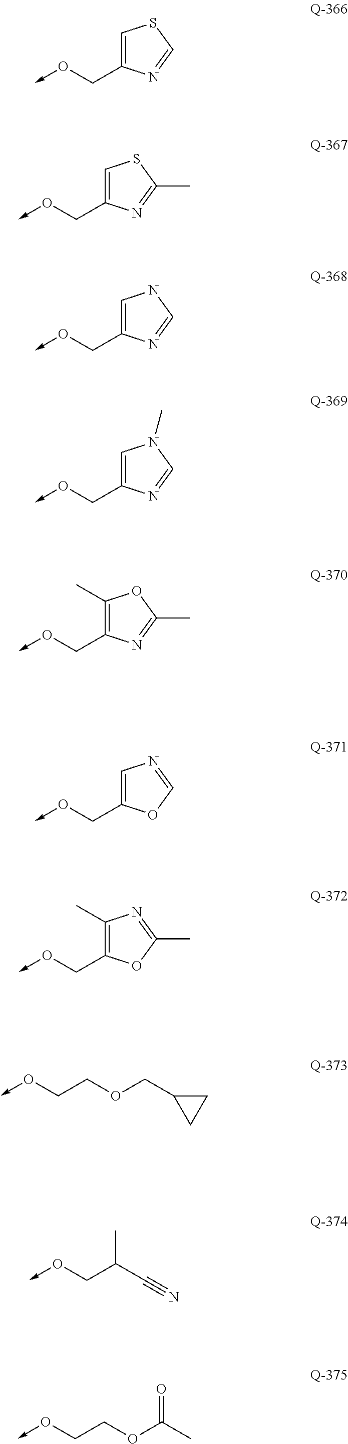

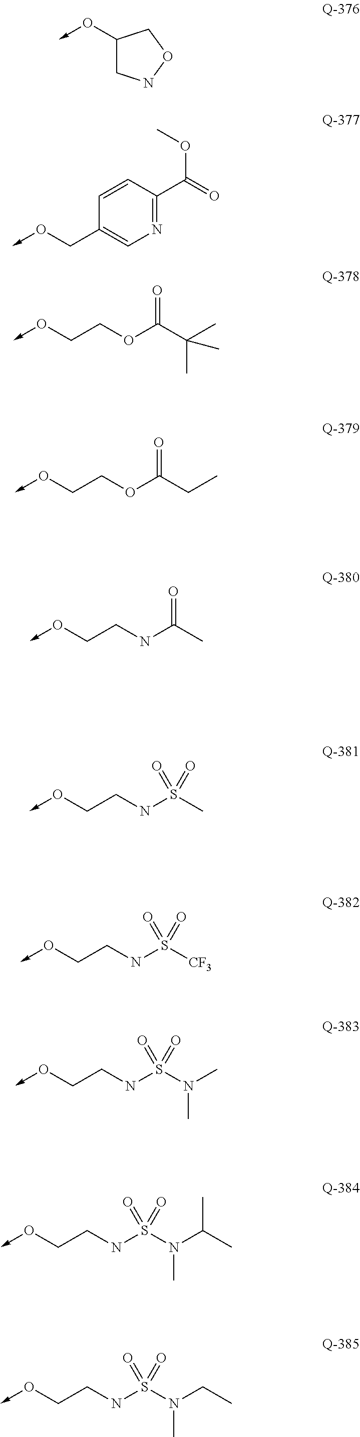

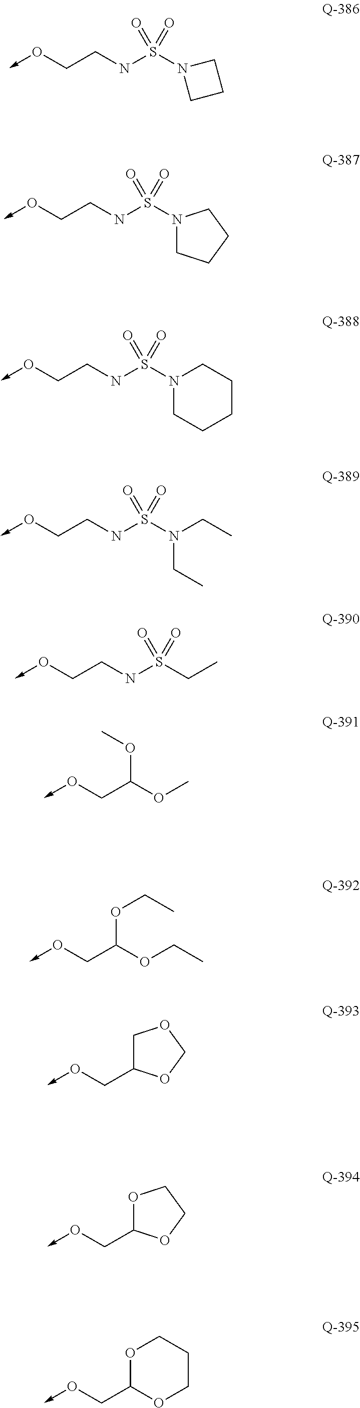

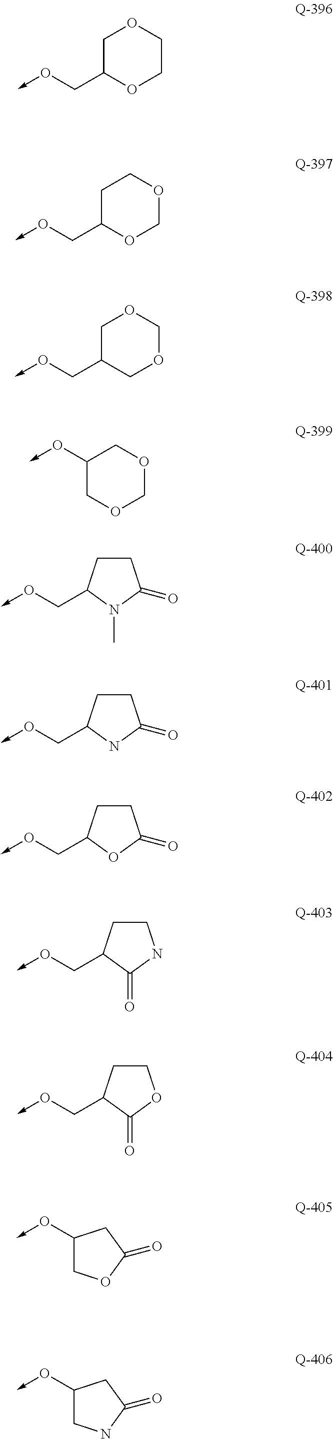

The invention especially preferably provides compounds of the general formula (I) in which
R¹ is hydrogen, fluorine, chlorine, bromine, iodine, cyano, methyl, ethyl, trifluoromethyl, difluoromethyl, methoxy, ethoxy, difluoromethoxy, trifluoromethoxy,
R² is fluorine,
R³ is hydrogen, fluorine, chlorine, bromine, methoxy,
R⁴ is fluorine, chlorine, bromine, cyano, NO₂, C(O)NH₂, C(S)NH₂, trifluoromethyl, ethynyl, propyn-1-yl,
R⁵, R⁶, R⁷ are independently hydrogen, fluorine, chlorine, bromine, iodine, cyano, methyl, ethyl, trifluoromethyl, difluoromethyl, methoxy, ethoxy, difluoromethoxy, trifluoromethoxy,
G is methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene,
X and Y are independently O (oxygen) or S (sulfur)
and
Q is one of the moieties Q-1 to Q-406 specifically mentioned above.

The invention very especially preferably provides compounds of the general formula (I) in which
R¹ is hydrogen, fluorine, chlorine, bromine, cyano, methyl, trifluoromethyl, methoxy, trifluoromethoxy,
R² is fluorine,
R³ is fluorine,
R⁴ is chlorine, bromine, cyano, NO₂, C(O)NH₂, C(S)NH₂,
R⁵, R⁶ and R⁷ are independently hydrogen, fluorine, chlorine, bromine, cyano, methyl, trifluoromethyl, methoxy, trifluoromethoxy,
G is methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
X and Y are independently O (oxygen) or S (sulfur)
and
Q is one of the moieties Q-1 to Q-406 specifically mentioned above.

The invention particularly especially preferably provides compounds of the general formula (I) in which
R¹ is hydrogen, fluorine, chlorine, bromine,
R² is fluorine,
R³ is fluorine,
R⁴ is chlorine, bromine, cyano, NO₂,
R⁵ is hydrogen, fluorine, chlorine, bromine,
R⁶ is hydrogen, fluorine, chlorine, bromine, cyano,
R⁷ is hydrogen, fluorine, chlorine, bromine,
G is methylene, (methyl)methylene, (ethyl)methylene,
X and Y are independently O (oxygen) or S (sulfur)
and
Q is one of the moieties Q-1 to Q-406 specifically mentioned above.

The invention very particularly especially preferably provides compounds of the general formula (I) in which
R¹ is hydrogen,
R² is fluorine,
R³ is fluorine,
R⁴ is chlorine, bromine, cyano, NO₂,
R⁵ is hydrogen, fluorine, chlorine, bromine,
R⁶ is hydrogen, fluorine, chlorine, bromine, cyano,
R⁷ is hydrogen,
G is methylene, (methyl)methylene,
X is O (oxygen) or S (sulfur),
Y is O (oxygen)
and
Q is one of the moieties Q-1 to Q-406 specifically mentioned above.

The invention very especially preferably provides compounds of the general formula (I) in which
R¹ is hydrogen,
R² is fluorine,
R³ is fluorine,
R⁴ is chlorine, bromine, cyano, NO₂,
R⁵ is hydrogen, fluorine,
R⁶ is hydrogen, fluorine, bromine, cyano,
R⁷ is hydrogen,
G is methylene, (methyl)methylene,
X is O (oxygen) or S (sulfur),
Y is O (oxygen)
and
Q is one of the moieties Q-1 to Q-35, Q-41 to Q-45, Q-58, Q-71 to Q-80, Q-89, Q-94, Q-95, Q-115, Q-120 to Q-123, Q-152 to Q-155, Q-166 to Q-170, Q-176 to Q-190, Q-261 to Q-348, Q-352 to Q-372, Q-377, Q-391-Q-399 specifically mentioned above.

The invention extremely especially preferably provides compounds of the general formula (I) in which
R¹ is hydrogen,
R² is fluorine,
R³ is fluorine,
R⁴ is chlorine, bromine, cyano, NO₂,
R⁵ is hydrogen, fluorine,
R⁶ is hydrogen, fluorine, bromine, cyano,
R⁷ is hydrogen,
G is methylene, (methyl)methylene,
X is O (oxygen) or S (sulfur),
Y is O (oxygen)
and
Q is one of the moieties Q-1, Q-2, Q-3, Q-4, Q-7, Q-8, Q-9, Q-17, Q-18, Q-23, Q-24, Q-26, Q-27, Q-41, Q-42, Q-43, Q-58, Q-71, Q-72, Q-89, Q-94, Q-115, Q-121, Q-176, Q-177, Q-179, Q-183, Q-272, Q-274, Q-275, Q-276, Q-277, Q-278, Q-281, Q-282, Q-283, Q-284, Q-286, Q-288, Q-291, Q-296, Q-301, Q-302, Q-303, Q-308, Q-309, Q-321, Q-327, Q-328, Q-329, Q-331, Q-335, Q-339, Q-356, Q-365, Q-366, Q-367, Q-371, Q-394 specified above.

The abovementioned general or preferred radical definitions apply both to the end products of the general formula (I) and, correspondingly, to the starting materials or the intermediates required in each case for the preparation. These radical definitions can be combined with one another as desired, i.e. including combinations between the given preferred ranges.

Of particular interest, primarily for reasons of higher herbicidal activity, better selectivity and/or better preparability, are inventive compounds of the general formula (I) given or salts thereof or the inventive use thereof in which individual radicals have one of the preferred meanings already specified or specified below, or in particular those in which one or more of the preferred meanings already specified or specified below occur in combination.

If the compounds can form, through a hydrogen shift, tautomers whose structure would not formally be covered by the general formula (I), these tautomers are nevertheless encompassed by the definition of the inventive compounds of the general formula (I), unless a particular tautomer is under consideration. For example, many carbonyl compounds may be present both in the keto form and in the enol form, both forms being encompassed by the definition of the compound of the general formula (I).

Depending on the nature of the substituents and the manner in which they are attached, the compounds of the general formula (I) may be present as stereoisomers. The possible stereoisomers defined by the specific three-dimensional form thereof, such as enantiomers, diastereomers, Z and E isomers, are all encompassed by the general formula (I). If, for example, one or more alkenyl groups are present, diastereomers (Z and E isomers) may occur. If, for example, one or more asymmetric carbon atoms are present, enantiomers and diastereomers may occur. Stereoisomers can be obtained from the mixtures obtained in the preparation by customary separation methods. The chromatographic separation can be effected either on the analytical scale to find the enantiomeric excess or the diastereomeric excess, or else on the preparative scale to produce test specimens for biological testing. It is likewise possible to selectively prepare stereoisomers by using stereoselective reactions with use of optically active starting materials and/or auxiliaries. The invention thus also relates to all stereoisomers which are embraced by the general formula (I) but are not shown in their specific stereometric form, and to mixtures thereof.

If the compounds are obtained as solids, the purification can also be carried out by recrystallization or digestion. If individual compounds (I) cannot be obtained in a satisfactory manner by the routes described below, they can be prepared by derivatization of other compounds (I).

Suitable isolation methods, purification methods and methods for separating stereoisomers of compounds of the general formula (I) are methods generally known to the person skilled in the art from analogous cases, for example by physical processes such as crystallization, chromatographic methods, in particular column chromatography and HPLC (high pressure liquid chromatography), distillation, optionally under reduced pressure, extraction and other methods, any mixtures that remain can generally be separated by chromatographic separation, for example on chiral solid phases. Suitable for preparative amounts or on an industrial scale are processes such as crystallization, for example of diastereomeric salts which can be obtained from the diastereomer mixtures using optically active acids and, if appropriate, provided that acidic groups are present, using optically active bases.

With regard to the compounds of the invention, the terms used above and further down will be elucidated. These are familiar to the person skilled in the art and especially have the definitions elucidated hereinafter:

Unless defined differently, names of chemical groups are generally to be understood such that attachment to the skeleton or the remainder of the molecule is via the structural element of the relevant chemical group mentioned last, i.e. for example in the case of (C₂-C₈)-alkenyloxy via the oxygen atom and in the case of heterocyclyl-(C₁-C₈)-alkyl or (C₁-C₆)-alkoxy-(C₁-C₆)-alkoxy-(C₁-C₆)-alkyl in each case via the carbon atom of the alkyl group.

According to the invention, “alkylthio”—alone or as part of a chemical group—refers to straight-chain or branched S-alkyl, preferably having 1 to 8 or 1 to 6 carbon atoms, such as (C₁-C₁₀)-, (C₁-C₆)- or (C₁-C₄)-alkylthio, for example (but not limited to) (C₁-C₆)-alkylthio such as methylthio, ethylthio, propylthio, 1-methylethylthio, butylthio, 1-methylpropylthio, 2-methylpropylthio, 1,1-dimethylethylthio, pentylthio, 1-methylbutylthio, 2-methylbutylthio, 3-methylbutylthio, 1,1-dimethylpropylthio, 1,2-dimethylpropylthio, 2,2-dimethylpropylthio, 1-ethylpropylthio, hexylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio and 1-ethyl-2-methylpropylthio.

“Alkoxy” denotes an alkyl radical bonded via an oxygen atom, for example (but not limited to) (C₁-C₆)-alkoxy such as methoxy, ethoxy, propoxy, 1-methylethoxy, butoxy, 1-methylpropoxy, 2-methylpropoxy, 1,1-dimethylethoxy, pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy and 1-ethyl-2-methylpropoxy. Alkenyloxy denotes an alkenyl radical bonded via an oxygen atom, and alkynyloxy denotes an alkynyl radical bonded via an oxygen atom, such as (C₂-C₁₀)-, (C₂-C₆)- or (C₂-C₄)-alkenoxy and (C₃-C₁₀)-, (C₃-C₆)- or (C₃-C₄)-alkynoxy.

According to the invention, “alkylcarbonyl” (alkyl-C(═O)—), unless defined differently elsewhere, denotes alkyl radicals bonded to the skeleton via —C(═O)—, such as (C₁-C₁₀)-, (C₁-C₆)- or (C₁-C₄)-alkylcarbonyl. The number of the carbon atoms here relates to the alkyl radical in the alkylcarbonyl group.

“Alkoxycarbonyl (alkyl-O—C(═O)—)”, unless defined differently elsewhere: alkyl radicals bonded to the skeleton via —O—C(═O)—, such as (C₁-C₁₀)-, (C₁-C₆)- or (C₁-C₄)-alkoxycarbonyl. The number of the carbon atoms here relates to the alkyl radical in the alkoxycarbonyl group. Analogously, “alkenyloxycarbonyl” and “alkynyloxycarbonyl”, unless defined differently elsewhere, in accordance with the invention, are respectively alkenyl and alkynyl radicals bonded to the skeleton via —O—C(═O)—, such as (C₂-C₁₀)-, (C₂-C₆)- or (C₂-C₄)-alkenyloxycarbonyl and (C₃-C₁₀)-, (C₃-C₆)- or (C₃-C₄)-alkynyloxycarbonyl respectively. The number of the carbon atoms here refers to the alkenyl or alkynyl radical in the alkenyloxycarbonyl or alkynyloxycarbonyl group respectively.

According to the invention, the term “alkylcarbonyloxy” (alkyl-C(═O)—O—), unless defined differently elsewhere, represents alkyl radicals bonded to the skeleton via the oxygen of a carbonyloxy group (—C(═O)—O—), such as (C₁-C₁₀)-, (C₁-C₆)- or (C₁-C₄)-alkylcarbonyloxy. The number of the carbon atoms here relates to the alkyl radical in the alkylcarbonyloxy group.

In short forms such as C(O)R¹³, C(O)OR¹³, OC(O)NR¹¹R¹² or C(O)NR¹¹R¹², for example, the short form O shown in brackets is an oxygen atom bonded to the adjacent carbon atom via a double bond.

In short forms such as OC(S)OR¹³, OC(S)SR¹⁴, OC(S)NR¹¹R¹², the short form S shown in brackets is a sulfur atom bonded to the adjacent carbon atom via a double bond.

The term “aryl” denotes an optionally substituted mono-, bi- or polycyclic aromatic system having preferably 6 to 14, especially 6 to 10, ring carbon atoms, for example phenyl, naphthyl, anthryl, phenanthrenyl and the like, preferably phenyl.

The term “optionally substituted aryl” also embraces polycyclic systems, such as tetrahydronaphthyl, indenyl, indanyl, fluorenyl, biphenylyl, where the bonding site is on the aromatic system. In systematic terms, “aryl” is generally also encompassed by the term “optionally substituted phenyl”. Preferred aryl substituents here are, for example, hydrogen, halogen, alkyl, cycloalkyl, cycloalkylalkyl, cycloalkenyl, halocycloalkyl, alkenyl, alkynyl, aryl, arylalkyl, arylalkenyl, heteroaryl, heteroarylalkyl, heterocyclyl, heterocyclylalkyl, alkoxyalkyl, alkylthio, haloalkylthio, haloalkyl, alkoxy, haloalkoxy, cycloalkoxy, cycloalkylalkoxy, aryloxy, heteroraryloxy, alkoxyalkoxy, alkynylalkoxy, alkenyloxy, bisalkylaminoalkoxy, tris[alkyl]silyl, bis[alkyl]arylsilyl, bis[alkyl]alkylsilyl, tris[alkyl]silylalkynyl, arylalkynyl, heteroarylalkynyl, alkylalkynyl, cycloalkylalkynyl, haloalkylalkynyl, heterocyclyl-N-alkoxy, nitro, cyano, amino, alkylamino, bisalkylamino, alkylcarbonylamino, cycloalkylcarbonylamino, arylcarbonylamino, alkoxycarbonylamino, alkoxycarbonylalkylamino, arylalkoxycarbonylalkylamino, hydroxycarbonyl, alkoxycarbonyl, aminocarbonyl, alkylaminocarbonyl, cycloalkylaminocarbonyl, bisalkylaminocarbonyl, heteroarylalkoxy, arylalkoxy.

A heterocyclic radical (heterocyclyl) contains at least one heterocyclic ring (=carbocyclic ring in which at least one carbon atom has been replaced by a heteroatom, preferably by a heteroatom from the group of N, O, S, P) which is saturated, unsaturated, partly saturated or heteroaromatic and may be unsubstituted or substituted, in which case the bonding site is localized on a ring atom. If the heterocyclyl radical or the heterocyclic ring is optionally substituted, it may be fused to other carbocyclic or heterocyclic rings. In the case of optionally substituted heterocyclyl, polycyclic systems are also included, for example 8-azabicyclo[3.2.1]octanyl, 8-azabicyclo[2.2.2]octanyl or 1-azabicyclo[2.2.1]heptyl. Optionally substituted heterocyclyl also includes spirocyclic systems, for example 1-oxa-5-azaspiro[2.3]hexyl. Unless defined differently, the heterocyclic ring preferably contains 3 to 9 ring atoms, especially 3 to 6 ring atoms, and one or more, preferably 1 to 4, especially 1, 2 or 3, heteroatoms in the heterocyclic ring, preferably from the group of N, O and S, but no two oxygen atoms should be directly adjacent, for example with one heteroatom from the group of N, O and S: 1- or 2- or 3-pyrrolidinyl, 3,4-dihydro-2H-pyrrol-2- or -3-yl, 2,3-dihydro-1H-pyrrol-1- or -2- or -3- or -4- or -5-yl; 2,5-dihydro-1H-pyrrol-1- or -2- or -3-yl, 1- or 2- or 3- or 4-piperidinyl; 2,3,4,5-tetrahydropyridin-2- or -3- or -4- or -5-yl or -6-yl; 1,2,3,6-tetrahydropyridin-1- or -2- or -3- or -4- or -5- or -6-yl; 1,2,3,4-tetrahydropyridin-1- or -2- or -3- or -4- or -5- or -6-yl; 1,4-dihydropyridin-1- or -2- or -3- or -4-yl; 2,3-dihydropyridin-2- or -3- or -4- or -5- or -6-yl; 2,5-dihydropyridin-2- or -3- or -4- or -5- or -6-yl, 1- or 2- or 3- or 4-azepanyl; 2,3,4,5-tetrahydro-1H-azepin-1- or -2- or -3- or -4- or -5- or -6- or -7-yl; 2,3,4,7-tetrahydro-1H-azepin-1- or -2- or -3- or -4- or -5- or -6- or -7-yl; 2,3,6,7-tetrahydro-1H-azepin-1- or -2- or -3- or -4-yl; 3,4,5,6-tetrahydro-2H-azepin-2- or -3- or -4- or -5- or -6- or -7-yl; 4,5-dihydro-1H-azepin-1- or -2- or -3- or -4-yl; 2,5-dihydro-1H-azepin-1- or -2- or -3- or -4- or -5- or -6- or -7-yl; 2,7-dihydro-1H-azepin-1- or -2- or -3- or -4-yl; 2,3-dihydro-1H-azepin-1- or -2- or -3- or -4- or -5- or -6- or -7-yl; 3,4-dihydro-2H-azepin-2- or -3- or -4- or -5- or -6- or -7-yl; 3,6-dihydro-2H-azepin-2- or -3- or -4- or -5- or -6- or -7-yl; 5,6-dihydro-2H-azepin-2- or -3- or -4- or -5- or -6- or -7-yl; 4,5-dihydro-3H-azepin-2- or -3- or -4- or -5- or -6- or -7-yl; 1H-azepin-1- or -2- or -3- or -4- or -5- or -6- or -7-yl; 2H-azepin-2- or -3- or -4- or -5- or -6- or -7-yl; 3H-azepin-2- or -3- or -4- or -5- or -6- or -7-yl; 4H-azepin-2- or -3- or -4- or -5- or -6- or -7-yl, 2- or 3-oxolanyl (=2- or 3-tetrahydrofuranyl); 2,3-dihydrofuran-2- or -3- or -4- or -5-yl; 2,5-dihydrofuran-2- or -3-yl, 2- or 3- or 4-oxanyl (=2- or 3- or 4-tetrahydropyranyl); 3,4-dihydro-2H-pyran-2- or -3- or -4- or -5- or -6-yl; 3,6-dihydro-2H-pyran-2- or -3- or -4- or -5- or -6-yl; 2H-pyran-2- or -3- or -4- or -5- or -6-yl; 4H-pyran-2- or -3- or -4-yl, 2- or 3- or 4-oxepanyl; 2,3,4,5-tetrahydrooxepin-2- or -3- or -4- or -5- or -6- or -7-yl; 2,3,4,7-tetrahydrooxepin-2- or -3- or -4- or -5- or -6- or -7-yl; 2,3,6,7-tetrahydrooxepin-2- or -3- or -4-yl; 2,3-dihydrooxepin-2- or -3- or -4- or -5- or -6- or -7-yl; 4,5-dihydrooxepin-2- or -3- or -4-yl; 2,5-dihydrooxepin-2- or -3- or -4- or -5- or -6- or -7-yl; oxepin-2- or -3- or -4- or -5- or -6- or -7-yl; 2- or 3-tetrahydrothiophenyl; 2,3-dihydrothiophen-2- or -3- or -4- or -5-yl; 2,5-dihydrothiophen-2- or -3-yl; tetrahydro-2H-thiopyran-2- or -3- or -4-yl; 3,4-dihydro-2H-thiopyran-2- or -3- or -4- or -5- or -6-yl; 3,6-dihydro-2H-thiopyran-2- or -3- or -4- or -5- or -6-yl; 2H-thiopyran-2- or -3- or -4- or -5- or -6-yl; 4H-thiopyran-2- or -3- or -4-yl. Preferred 3-membered and 4-membered heterocycles are, for example, 1- or 2-aziridinyl, oxiranyl, thiiranyl, 1- or 2- or 3-azetidinyl, 2- or 3-oxetanyl, 2- or 3-thietanyl, 1,3-dioxetan-2-yl. Further examples of “heterocyclyl” are a partly or fully hydrogenated heterocyclic radical having two heteroatoms from the group of N, O and S, for example 1- or 2- or 3- or 4-pyrazolidinyl; 4,5-dihydro-3H-pyrazol-3- or 4- or 5-yl; 4,5-dihydro-1H-pyrazol-1- or 3- or 4- or 5-yl; 2,3-dihydro-1H-pyrazol-1- or 2- or 3- or 4- or 5-yl; 1- or 2- or 3- or 4-imidazolidinyl; 2,3-dihydro-1H-imidazol-1- or 2- or 3- or 4-yl; 2,5-dihydro-1H-imidazol-1- or 2- or 4- or 5-yl; 4,5-dihydro-1H-imidazol-1- or 2- or 4- or 5-yl; hexahydropyridazin-1- or 2- or 3- or 4-yl; 1,2,3,4-tetrahydropyridazin-1- or 2- or 3- or 4- or 5- or 6-yl; 1,2,3,6-tetrahydropyridazin-1- or 2- or 3- or 4- or 5- or 6-yl; 1,4,5,6-tetrahydropyridazin-1- or 3- or 4- or 5- or 6-yl; 3,4,5,6-tetrahydropyridazin-3- or 4- or 5-yl; 4,5-dihydropyridazin-3- or 4-yl; 3,4-dihydropyridazin-3- or 4- or 5- or 6-yl; 3,6-dihydropyridazin-3- or 4-yl; 1,6-dihydropyridazin-1- or 3- or 4- or 5- or 6-yl; hexahydropyrimidin-1- or 2- or 3- or 4-yl; 1,4,5,6-tetrahydropyrimidin-1- or 2- or 4- or 5- or 6-yl; 1,2,5,6-tetrahydropyrimidin-1- or 2- or 4- or 5- or 6-yl; 1,2,3,4-tetrahydropyrimidin-1- or 2- or 3- or 4- or 5- or 6-yl; 1,6-dihydropyrimidin-1- or 2- or 4- or 5- or 6-yl; 1,2-dihydropyrimidin-1- or 2- or 4- or 5- or 6-yl; 2,5-dihydropyrimidin-2- or 4- or 5-yl; 4,5-dihydropyrimidin-4- or 5- or 6-yl; 1,4-dihydropyrimidin-1- or 2- or 4- or 5- or 6-yl; 1- or 2- or 3-piperazinyl; 1,2,3,6-tetrahydropyrazin-1- or 2- or 3- or 5- or 6-yl; 1,2,3,4-tetrahydropyrazin-1- or 2- or 3- or 4- or 5- or 6-yl; 1,2-dihydropyrazin-1- or 2- or 3- or 5- or 6-yl; 1,4-dihydropyrazin-1- or 2- or 3-yl; 2,3-dihydropyrazin-2- or 3- or 5- or 6-yl; 2,5-dihydropyrazin-2- or 3-yl; 1,3-dioxolan-2- or 4- or 5-yl; 1,3-dioxol-2- or 4-yl; 1,3-dioxan-2- or 4- or 5-yl; 4H-1,3-dioxin-2- or 4- or 5- or 6-yl; 1,4-dioxan-2- or 3- or 5- or 6-yl; 2,3-dihydro-1,4-dioxin-2- or 3- or 5- or 6-yl; 1,4-dioxin-2- or 3-yl; 1,2-dithiolan-3- or 4-yl; 3H-1,2-dithiol-3- or 4- or 5-yl; 1,3-dithiolan-2- or 4-yl; 1,3-dithiol-2- or 4-yl; 1,2-dithian-3- or 4-yl; 3,4-dihydro-1,2-dithiin-3- or 4- or 5- or 6-yl; 3,6-dihydro-1,2-dithiin-3- or 4-yl; 1,2-dithiin-3- or 4-yl; 1,3-dithian-2- or 4- or 5-yl; 4H-1,3-dithiin-2- or 4- or 5- or 6-yl; isoxazolidin-2- or 3- or 4- or 5-yl; 2,3-dihydroisoxazol-2- or 3- or 4- or 5-yl; 2,5-dihydroisoxazol-2- or 3- or 4- or 5-yl; 4,5-dihydroisoxazol-3- or 4- or 5-yl; 1,3-oxazolidin-2- or 3- or 4- or 5-yl; 2,3-dihydro-1,3-oxazol-2- or 3- or 4- or 5-yl; 2,5-dihydro-1,3-oxazol-2- or 4- or 5-yl; 4,5-dihydro-1,3-oxazol-2- or 4- or 5-yl; 1,2-oxazinan-2- or 3- or 4- or 5- or 6-yl; 3,4-dihydro-2H-1,2-oxazin-2- or 3- or 4- or 5- or 6-yl; 3,6-dihydro-2H-1,2-oxazin-2- or 3- or 4- or 5- or 6-yl; 5,6-dihydro-2H-1,2-oxazin-2- or 3- or 4- or 5- or 6-yl; 5,6-dihydro-4H-1,2-oxazin-3- or 4- or 5- or 6-yl; 2H-1,2-oxazin-2- or 3- or 4- or 5- or 6-yl; 6H-1,2-oxazin-3- or 4- or 5- or 6-yl; 4H-1,2-oxazin-3- or 4- or 5- or 6-yl; 1,3-oxazinan-2- or 3- or 4- or 5- or 6-yl; 3,4-dihydro-2H-1,3-oxazin-2- or 3- or 4- or 5- or 6-yl; 3,6-dihydro-2H-1,3-oxazin-2- or 3- or 4- or 5- or 6-yl; 5,6-dihydro-2H-1,3-oxazin-2- or 4- or 5- or 6-yl; 5,6-dihydro-4H-1,3-oxazin-2- or 4- or 5- or 6-yl; 2H-1,3-oxazin-2- or 4- or 5- or 6-yl; 6H-1,3-oxazin-2- or 4- or 5- or 6-yl; 4H-1,3-oxazin-2- or 4- or 5- or 6-yl; morpholin-2- or 3- or 4-yl; 3,4-dihydro-2H-1,4-oxazin-2- or 3- or 4- or 5- or 6-yl; 3,6-dihydro-2H-1,4-oxazin-2- or 3- or 5- or 6-yl; 2H-1,4-oxazin-2- or 3- or 5- or 6-yl; 4H-1,4-oxazin-2- or 3-yl; 1,2-oxazepan-2- or 3- or 4- or 5- or 6- or 7-yl; 2,3,4,5-tetrahydro-1,2-oxazepin-2- or 3- or 4- or 5- or 6- or 7-yl; 2,3,4,7-tetrahydro-1,2-oxazepin-2- or 3- or 4- or 5- or 6- or 7-yl; 2,3,6,7-tetrahydro-1,2-oxazepin-2- or 3- or 4- or 5- or 6- or 7-yl; 2,5,6,7-tetrahydro-1,2-oxazepin-2- or 3- or 4- or 5- or 6- or 7-yl; 4,5,6,7-tetrahydro-1,2-oxazepin-3- or 4- or 5- or 6- or 7-yl; 2,3-dihydro-1,2-oxazepin-2- or 3- or 4- or 5- or 6- or 7-yl; 2,5-dihydro-1,2-oxazepin-2- or 3- or 4- or 5- or 6- or 7-yl; 2,7-dihydro-1,2-oxazepin-2- or 3- or 4- or 5- or 6- or 7-yl; 4,5-dihydro-1,2-oxazepin-3- or 4- or 5- or 6- or 7-yl; 4,7-dihydro-1,2-oxazepin-3- or 4- or 5- or 6- or 7-yl; 6,7-dihydro-1,2-oxazepin-3- or 4- or 5- or 6- or 7-yl; 1,2-oxazepin-3- or 4- or 5- or 6- or 7-yl; 1,3-oxazepan-2- or 3- or 4- or 5- or 6- or 7-yl; 2,3,4,5-tetrahydro-1,3-oxazepin-2- or 3- or 4- or 5- or 6- or 7-yl; 2,3,4,7-tetrahydro-1,3-oxazepin-2- or 3- or 4- or 5- or 6- or 7-yl; 2,3,6,7-tetrahydro-1,3-oxazepin-2- or 3- or 4- or 5- or 6- or 7-yl; 2,5,6,7-tetrahydro-1,3-oxazepin-2- or 4- or 5- or 6- or 7-yl; 4,5,6,7-tetrahydro-1,3-oxazepin-2- or 4- or 5- or 6- or 7-yl; 2,3-dihydro-1,3-oxazepin-2- or 3- or 4- or 5- or 6- or 7-yl; 2,5-dihydro-1,3-oxazepin-2- or 4- or 5- or 6- or 7-yl; 2,7-dihydro-1,3-oxazepin-2- or 4- or 5- or 6- or 7-yl; 4,5-dihydro-1,3-oxazepin-2- or 4- or 5- or 6- or 7-yl; 4,7-dihydro-1,3-oxazepin-2- or 4- or 5- or 6- or 7-yl; 6,7-dihydro-1,3-oxazepin-2- or 4- or 5- or 6- or 7-yl; 1,3-oxazepin-2- or 4- or 5- or 6- or 7-yl; 1,4-oxazepan-2- or 3- or 5- or 6- or 7-yl; 2,3,4,5-tetrahydro-1,4-oxazepin-2- or 3- or 4- or 5- or 6- or 7-yl; 2,3,4,7-tetrahydro-1,4-oxazepin-2- or 3- or 4- or 5- or 6- or 7-yl; 2,3,6,7-tetrahydro-1,4-oxazepin-2- or 3- or 5- or 6- or 7-yl; 2,5,6,7-tetrahydro-1,4-oxazepin-2- or 3- or 5- or 6- or 7-yl; 4,5,6,7-tetrahydro-1,4-oxazepin-2- or 3- or 4- or 5- or 6- or 7-yl; 2,3-dihydro-1,4-oxazepin-2- or 3- or 5- or 6- or 7-yl; 2,5-dihydro-1,4-oxazepin-2- or 3- or 5- or 6- or 7-yl; 2,7-dihydro-1,4-oxazepin-2- or 3- or 5- or 6- or 7-yl; 4,5-dihydro-1,4-oxazepin-2- or 3- or 4- or 5- or 6- or 7-yl; 4,7-dihydro-1,4-oxazepin-2- or 3- or 4- or 5- or 6- or 7-yl; 6,7-dihydro-1,4-oxazepin-2- or 3- or 5- or 6- or 7-yl; 1,4-oxazepin-2- or 3- or 5- or 6- or 7-yl; isothiazolidin-2- or 3- or 4- or 5-yl; 2,3-dihydroisothiazol-2- or 3- or 4- or 5-yl; 2,5-dihydroisothiazol-2- or 3- or 4- or 5-yl; 4,5-dihydroisothiazol-3- or 4- or 5-yl; 1,3-thiazolidin-2- or 3- or 4- or 5-yl; 2,3-dihydro-1,3-thiazol-2- or 3- or 4- or 5-yl; 2,5-dihydro-1,3-thiazol-2- or 4- or 5-yl; 4,5-dihydro-1,3-thiazol-2- or 4- or 5-yl; 1,3-thiazinan-2- or 3- or 4- or 5- or 6-yl; 3,4-dihydro-2H-1,3-thiazin-2- or 3- or 4- or 5- or 6-yl; 3,6-dihydro-2H-1,3-thiazin-2- or 3- or 4- or 5- or 6-yl; 5,6-dihydro-2H-1,3-thiazin-2- or 4- or 5- or 6-yl; 5,6-dihydro-4H-1,3-thiazin-2- or 4- or 5- or 6-yl; 2H-1,3-thiazin-2- or 4- or 5- or 6-yl; 6H-1,3-thiazin-2- or 4- or 5- or 6-yl; 4H-1,3-thiazin-2- or 4- or 5- or 6-yl. Further examples of “heterocyclyl” are a partly or fully hydrogenated heterocyclic radical having 3 heteroatoms from the group of N, O and S, for example 1,4,2-dioxazolidin-2- or 3- or 5-yl; 1,4,2-dioxazol-3- or 5-yl; 1,4,2-dioxazinan-2- or -3- or 5- or 6-yl; 5,6-dihydro-1,4,2-dioxazin-3- or 5- or 6-yl; 1,4,2-dioxazin-3- or 5- or 6-yl; 1,4,2-dioxazepan-2- or 3- or 5- or 6- or 7-yl; 6,7-dihydro-5H-1,4,2-dioxazepin-3- or 5- or 6- or 7-yl; 2,3-dihydro-7H-1,4,2-dioxazepin-2- or 3- or 5- or 6- or 7-yl; 2,3-dihydro-5H-1,4,2-dioxazepin-2- or 3- or 5- or 6- or 7-yl; 5H-1,4,2-dioxazepin-3- or 5- or 6- or 7-yl; 7H-1,4,2-dioxazepin-3- or 5- or 6- or 7-yl. Structural examples of heterocycles which are optionally substituted further are also listed below:

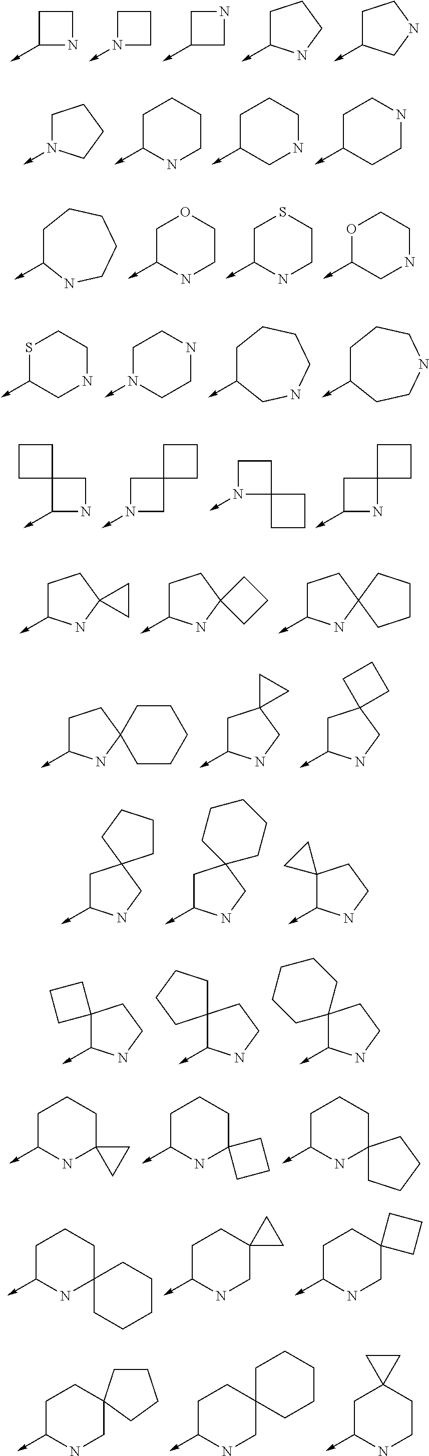

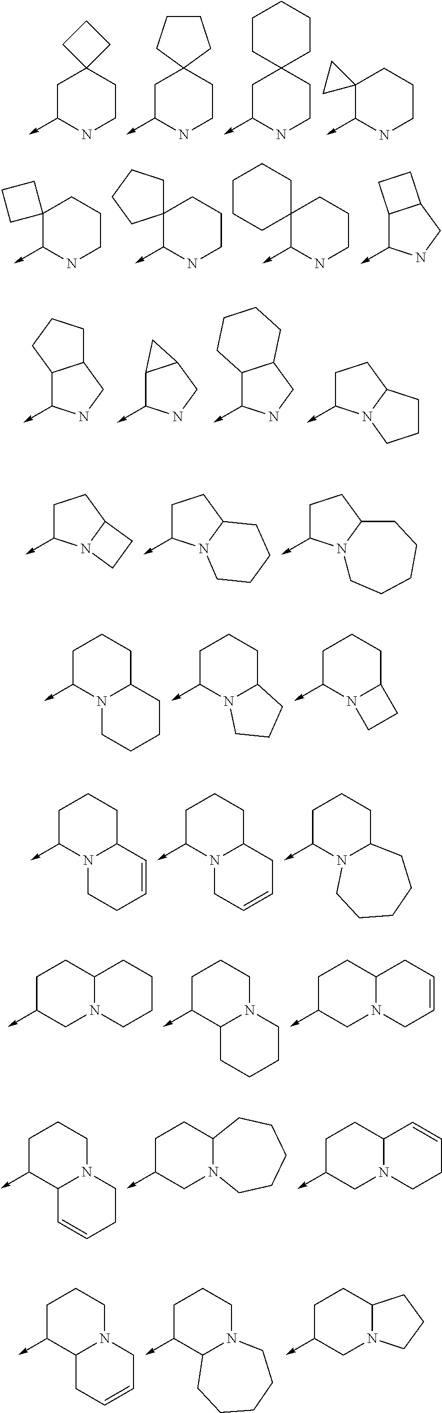

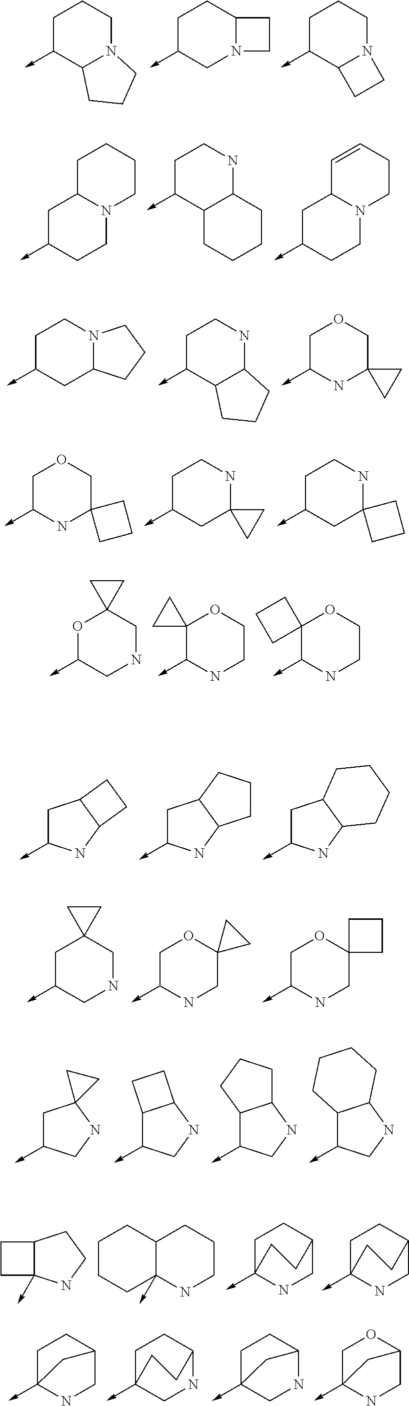

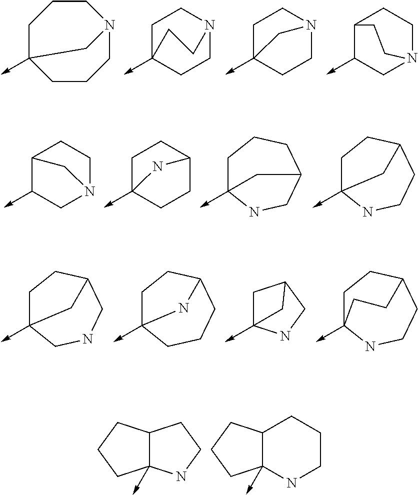

The heterocycles listed above are preferably substituted, for example, by hydrogen, halogen, alkyl, haloalkyl, hydroxyl, alkoxy, cycloalkoxy, aryloxy, alkoxyalkyl, alkoxyalkoxy, cycloalkyl, halocycloalkyl, aryl, arylalkyl, heteroaryl, heterocyclyl, alkenyl, alkylcarbonyl, cycloalkylcarbonyl, arylcarbonyl, heteroarylcarbonyl, alkoxycarbonyl, hydroxycarbonyl, cycloalkoxycarbonyl, cycloalkylalkoxycarbonyl, alkoxycarbonylalkyl, arylalkoxycarbonyl, arylalkoxycarbonylalkyl, alkynyl, alkynylalkyl, alkylalkynyl, trisalkylsilylalkynyl, nitro, amino, cyano, haloalkoxy, haloalkylthio, alkylthio, hydrothio, hydroxyalkyl, oxo, heteroarylalkoxy, arylalkoxy, heterocyclylalkoxy, heterocyclylalkylthio, heterocyclyloxy, heterocyclylthio, heteroaryloxy, bisalkylamino, alkylamino, cycloalkylamino, hydroxycarbonylalkylamino, alkoxycarbonylalkylamino, arylalkoxycarbonylalkylamino, alkoxycarbonylalkyl(alkyl)amino, aminocarbonyl, alkylaminocarbonyl, bisalkylaminocarbonyl, cycloalkylaminocarbonyl, hydroxycarbonylalkylaminocarbonyl, alkoxycarbonylalkylaminocarbonyl, arylalkoxycarbonylalkylaminocarbonyl.

When a base structure is substituted “by one or more radicals” from a list of radicals (=group) or a generically defined group of radicals, this in each case includes simultaneous substitution by a plurality of identical and/or structurally different radicals.

In the case of a partly or fully saturated nitrogen heterocycle, this may be joined to the remainder of the molecule either via carbon or via the nitrogen.

Suitable substituents for a substituted heterocyclic radical are the substituents specified further down, and additionally also oxo and thioxo. The oxo group as a substituent on a ring carbon atom is then, for example, a carbonyl group in the heterocyclic ring. As a result, lactones and lactams are preferably also included. The oxo group may also occur on the ring heteroatoms, which may exist in different oxidation states, for example in the case of N and S, and in that case form, for example, the divalent N(O), S(O) (also SO for short) and S(O)₂ (also SO₂ for short) groups in the heterocyclic ring. In the case of —N(O)— and —S(O)— groups, both enantiomers in each case are included.

According to the invention, the expression “heteroaryl” represents heteroaromatic compounds, i.e. fully unsaturated aromatic heterocyclic compounds, preferably 5- to 7-membered rings having 1 to 4, preferably 1 or 2, identical or different heteroatoms, preferably O, S or N. Inventive heteroaryls are, for example, 1H-pyrrol-1-yl; 1H-pyrrol-2-yl; 1H-pyrrol-3-yl; furan-2-yl; furan-3-yl; thien-2-yl; thien-3-yl, 1H-imidazol-1-yl; 1H-imidazol-2-yl; 1H-imidazol-4-yl; 1H-imidazol-5-yl; 1H-pyrazol-1-yl; 1H-pyrazol-3-yl; 1H-pyrazol-4-yl; 1H-pyrazol-5-yl, 1H-1,2,3-triazol-1-yl, 1H-1,2,3-triazol-4-yl, 1H-1,2,3-triazol-5-yl, 2H-1,2,3-triazol-2-yl, 2H-1,2,3-triazol-4-yl, 1H-1,2,4-triazol-1-yl, 1H-1,2,4-triazol-3-yl, 4H-1,2,4-triazol-4-yl, 1,2,4-oxadiazol-3-yl, 1,2,4-oxadiazol-5-yl, 1,3,4-oxadiazol-2-yl, 1,2,3-oxadiazol-4-yl, 1,2,3-oxadiazol-5-yl, 1,2,5-oxadiazol-3-yl, azepinyl, pyridin-2-yl, pyridin-3-yl, pyridin-4-yl, pyrazin-2-yl, pyrazin-3-yl, pyrimidin-2-yl, pyrimidin-4-yl, pyrimidin-5-yl, pyridazin-3-yl, pyridazin-4-yl, 1,3,5-triazin-2-yl, 1,2,4-triazin-3-yl, 1,2,4-triazin-5-yl, 1,2,4-triazin-6-yl, 1,2,3-triazin-4-yl, 1,2,3-triazin-5-yl, 1,2,4-, 1,3,2-, 1,3,6- and 1,2,6-oxazinyl, isoxazol-3-yl, isoxazol-4-yl, isoxazol-5-yl, 1,3-oxazol-2-yl, 1,3-oxazol-4-yl, 1,3-oxazol-5-yl, isothiazol-3-yl, isothiazol-4-yl, isothiazol-5-yl, 1,3-thiazol-2-yl, 1,3-thiazol-4-yl, 1,3-thiazol-5-yl, oxepinyl, thiepinyl, 1,2,4-triazolonyl and 1,2,4-diazepinyl, 2H-1,2,3,4-tetrazol-5-yl, 1H-1,2,3,4-tetrazol-5-yl, 1,2,3,4-oxatriazol-5-yl, 1,2,3,4-thiatriazol-5-yl, 1,2,3,5-oxatriazol-4-yl, 1,2,3,5-thiatriazol-4-yl. The heteroaryl groups of the invention may also be substituted by one or more identical or different radicals. If two adjacent carbon atoms are part of a further aromatic ring, the systems are fused heteroaromatic systems, such as benzofused or polyannelated heteroaromatics. Preferred examples are quinolines (e.g. quinolin-2-yl, quinolin-3-yl, quinolin-4-yl, quinolin-5-yl, quinolin-6-yl, quinolin-7-yl, quinolin-8-yl); isoquinolines (e.g. isoquinolin-1-yl, isoquinolin-3-yl, isoquinolin-4-yl, isoquinolin-5-yl, isoquinolin-6-yl, isoquinolin-7-yl, isoquinolin-8-yl); quinoxaline; quinazoline; cinnoline; 1,5-naphthyridine; 1,6-naphthyridine; 1,7-naphthyridine; 1,8-naphthyridine; 2,6-naphthyridine; 2,7-naphthyridine; phthalazine; pyridopyrazines; pyridopyrimidines; pyridopyridazines; pteridines; pyrimidopyrimidines. Examples of heteroaryl are also 5- or 6-membered benzofused rings from the group of 1H-indol-1-yl, 1H-indol-2-yl, 1H-indol-3-yl, 1H-indol-4-yl, 1H-indol-5-yl, 1H-indol-6-yl, 1H-indol-7-yl, 1-benzofuran-2-yl, 1-benzofuran-3-yl, 1-benzofuran-4-yl, 1-benzofuran-5-yl, 1-benzofuran-6-yl, 1-benzofuran-7-yl, 1-benzothiophen-2-yl, 1-benzothiophen-3-yl, 1-benzothiophen-4-yl, 1-benzothiophen-5-yl, 1-benzothiophen-6-yl, 1-benzothiophen-7-yl, 1H-indazol-1-yl, 1H-indazol-3-yl, 1H-indazol-4-yl, 1H-indazol-5-yl, 1H-indazol-6-yl, 1H-indazol-7-yl, 2H-indazol-2-yl, 2H-indazol-3-yl, 2H-indazol-4-yl, 2H-indazol-5-yl, 2H-indazol-6-yl, 2H-indazol-7-yl, 2H-isoindol-2-yl, 2H-isoindol-1-yl, 2H-isoindol-3-yl, 2H-isoindol-4-yl, 2H-isoindol-5-yl, 2H-isoindol-6-yl; 2H-isoindol-7-yl, 1H-benzimidazol-1-yl, 1H-benzimidazol-2-yl, 1H-benzimidazol-4-yl, 1H-benzimidazol-5-yl, 1H-benzimidazol-6-yl, 1H-benzimidazol-7-yl, 1,3-benzoxazol-2-yl, 1,3-benzoxazol-4-yl, 1,3-benzoxazol-5-yl, 1,3-benzoxazol-6-yl, 1,3-benzoxazol-7-yl, 1,3-benzothiazol-2-yl, 1,3-benzothiazol-4-yl, 1,3-benzothiazol-5-yl, 1,3-benzothiazol-6-yl, 1,3-benzothiazol-7-yl, 1,2-benzisoxazol-3-yl, 1,2-benzisoxazol-4-yl, 1,2-benzisoxazol-5-yl, 1,2-benzisoxazol-6-yl, 1,2-benzisoxazol-7-yl, 1,2-benzisothiazol-3-yl, 1,2-benzisothiazol-4-yl, 1,2-benzisothiazol-5-yl, 1,2-benzisothiazol-6-yl, 1,2-benzisothiazol-7-yl.

The term “halogen” denotes, for example, fluorine, chlorine, bromine or iodine. If the term is used for a radical, “halogen” denotes, for example, a fluorine, chlorine, bromine or iodine atom.

According to the invention, “alkyl” denotes a straight-chain or branched open-chain, saturated hydrocarbon radical which is optionally mono- or polysubstituted, and in the latter case is referred to as “substituted alkyl”. Preferred substituents are halogen atoms, alkoxy, haloalkoxy, cyano, alkylthio, haloalkylthio, amino or nitro groups, particular preference being given to methoxy, methyl, fluoroalkyl, cyano, nitro, fluorine, chlorine, bromine or iodine. The prefix “bis” also includes the combination of different alkyl radicals, e.g. methyl(ethyl) or ethyl(methyl).

“Haloalkyl”, “-alkenyl” and “-alkynyl” respectively denote alkyl, alkenyl and alkynyl partly or fully substituted by identical or different halogen atoms, e.g. monohaloalkyl, for example CH₂CH₂Cl, CH₂CH₂Br, CHClCH₃, CH₂Cl, CH₂F; perhaloalkyl, for example CCl₃, CClF₂, CFCl₂, CF₂CClF₂, CF₂CClFCF₃; polyhaloalkyl, for example CH₂CHFCl, CF₂CClFH, CF₂CBrFH, CH₂CF₃; the term “perhaloalkyl” also includes the term “perfluoroalkyl”.

“Haloalkoxy” is, for example, OCF₃, OCHF₂, OCH₂F, OCF₂CF₃, OCH₂CF₃ and OCH₂CH₂Cl; this applies correspondingly to haloalkenyl and other halogen-substituted radicals.

The expression “(C₁-C₄)-alkyl” mentioned here by way of example is a brief notation for straight-chain or branched alkyl having one to 4 carbon atoms according to the range stated for carbon atoms, i.e. encompasses the methyl, ethyl, 1-propyl, 2-propyl, 1-butyl, 2-butyl, 2-methylpropyl or tert-butyl radicals. General alkyl radicals with a larger specified range of carbon atoms, e.g. “(C₁-C₆)-alkyl”, correspondingly also encompass straight-chain or branched alkyl radicals with a greater number of carbon atoms, i.e. according to the example also the alkyl radicals having 5 and 6 carbon atoms.

Unless stated specifically, preference is given to the lower carbon skeletons, for example having from 1 to 6 carbon atoms, or having from 2 to 6 carbon atoms in the case of unsaturated groups, in the case of the hydrocarbon radicals such as alkyl, alkenyl and alkynyl radicals, including in composite radicals. Alkyl radicals, including in composite radicals such as alkoxy, haloalkyl, etc., are, for example, methyl, ethyl, n-propyl or i-propyl, n-, i-, t- or 2-butyl, pentyls, hexyls such as n-hexyl, i-hexyl and 1,3-dimethylbutyl, heptyls such as n-heptyl, 1-methylhexyl and 1,4-dimethylpentyl; alkenyl and alkynyl radicals are defined as the possible unsaturated radicals corresponding to the alkyl radicals, where at least one double bond or triple bond is present. Preference is given to radicals having one double bond or triple bond.

The term “alkenyl” also includes, in particular, straight-chain or branched open-chain hydrocarbon radicals having more than one double bond, such as 1,3-butadienyl and 1,4-pentadienyl, but also allenyl or cumulenyl radicals having one or more cumulated double bonds, for example allenyl (1,2-propadienyl), 1,2-butadienyl and 1,2,3-pentatrienyl. Alkenyl denotes, for example, vinyl which may optionally be substituted by further alkyl radicals, for example (but not limited thereto) (C₂-C₆)-alkenyl such as ethenyl, 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl and 1-ethyl-2-methyl-2-propenyl.

The term “alkynyl” also includes, in particular, straight-chain or branched open-chain hydrocarbon radicals having more than one triple bond, or else having one or more triple bonds and one or more double bonds, for example 1,3-butatrienyl or 3-penten-1-yn-1-yl. (C₂-C₆)-Alkynyl denotes, for example, ethynyl, 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl and 1-ethyl-1-methyl-2-propynyl.

The term “cycloalkyl” means a carbocyclic saturated ring system having preferably 3-8 ring carbon atoms, for example cyclopropyl, cyclobutyl, cyclopentyl or cyclohexyl, which optionally has further substitution, preferably by hydrogen, alkyl, alkoxy, cyano, nitro, alkylthio, haloalkylthio, halogen, alkenyl, alkynyl, haloalkyl, amino, alkylamino, bisalkylamino, alkoxycarbonyl, hydroxycarbonyl, arylalkoxycarbonyl, aminocarbonyl, alkylaminocarbonyl, cycloalkylaminocarbonyl. In the case of optionally substituted cycloalkyl, cyclic systems with substituents are included, also including substituents with a double bond on the cycloalkyl radical, for example an alkylidene group such as methylidene. In the case of optionally substituted cycloalkyl, polycyclic aliphatic systems are also included, for example bicyclo[1.1.0]butan-1-yl, bicyclo[1.1.0]butan-2-yl, bicyclo[2.1.0]pentan-1-yl, bicyclo[1.1.1]pentan-1-yl, bicyclo[2.1.0]pentan-2-yl, bicyclo[2.1.0]pentan-5-yl, bicyclo[2.1.1]hexyl, bicyclo[2.2.1]hept-2-yl, bicyclo[2.2.2]octan-2-yl, bicyclo[3.2.1]octan-2-yl, bicyclo[3.2.2]nonan-2-yl, adamantan-1-yl and adamantan-2-yl, but also systems such as 1,1′-bi(cyclopropyl)-1-yl, 1,1′-bi(cyclopropyl)-2-yl, for example. The term “(C₃-C₇)-cycloalkyl” is a brief notation for cycloalkyl having three to seven carbon atoms, corresponding to the range specified for carbon atoms.

In the case of substituted cycloalkyl, spirocyclic aliphatic systems are also included, for example spiro[2.2]pent-1-yl, spiro[2.3]hex-1-yl, spiro[2.3]hex-4-yl, 3-spiro[2.3]hex-5-yl, spiro[3.3]hept-1-yl, spiro[3.3]hept-2-yl.

“Cycloalkenyl” denotes a carbocyclic, nonaromatic, partly unsaturated ring system having preferably 4-8 carbon atoms, e.g. 1-cyclobutenyl, 2-cyclobutenyl, 1-cyclopentenyl, 2-cyclopentenyl, 3-cyclopentenyl, or 1-cyclohexenyl, 2-cyclohexenyl, 3-cyclohexenyl, 1,3-cyclohexadienyl or 1,4-cyclohexadienyl, also including substituents with a double bond on the cycloalkenyl radical, for example an alkylidene group such as methylidene. In the case of optionally substituted cycloalkenyl, the elucidations for substituted cycloalkyl apply correspondingly.

The term “alkylidene”, also, for example, in the form (C₁-C₁₀)-alkylidene, means the radical of a straight-chain or branched open-chain hydrocarbon radical which is attached via a double bond. Possible bonding sites for alkylidene are naturally only positions on the base structure where two hydrogen atoms can be replaced by the double bond; radicals are, for example, ═CH₂, ═CH—CH₃, ═C(CH₃)—CH₃, ═C(CH₃)—C₂H₅ or ═C(C₂H₅)—C₂H₅. Cycloalkylidene denotes a carbocyclic radical bonded via a double bond.

The term “alkylene”, also, for example, in the form (C₁-C₈)-alkylene, denotes the radical of a straight-chain or branched open-chain hydrocarbon radical which is attached at two positions to further groups.

“Alkoxyalkyl” is an alkoxy radical bonded via an alkyl group, and “alkoxyalkoxy” denotes an alkoxyalkyl radical bonded via an oxygen atom, for example (but not limited to) methoxymethoxy, methoxyethoxy, ethoxyethoxy, methoxy-n-propyloxy.

“Arylalkyl” is an aryl radical bonded via an alkyl group, “heteroarylalkyl” denotes a heteroaryl radical bonded via an alkyl group, and “heterocyclylalkyl” denotes a heterocyclyl radical bonded via an alkyl group.

“Cycloalkylalkyl” is a cycloalkyl radical bonded via an alkyl group, for example (but not limited to) cyclopropylmethyl, cyclobutylmethyl, cyclopentylmethyl, cyclohexylmethyl, 1-cyclopropyleth-1-yl, 2-cyclopropyleth-1-yl, 1-cyclopropylprop-1-yl, 3-cyclopropylprop-1-yl.

“Arylalkenyl” is an aryl radical bonded via an alkenyl group, “heteroarylalkenyl” denotes a heteroaryl radical bonded via an alkenyl group, and “heterocyclylalkenyl” denotes a heterocyclyl radical bonded via an alkenyl group.

“Arylalkynyl” is an aryl radical bonded via an alkynyl group, “heteroarylalkynyl” denotes a heteroaryl radical bonded via an alkynyl group, and “heterocyclylalkynyl” denotes a heterocyclyl radical bonded via an alkynyl group.

According to the invention, “haloalkylthio”—on its own or as constituent part of a chemical group—is straight-chain or branched S-haloalkyl, preferably having 1 to 8, or having 1 to 6 carbon atoms, such as (C₁-C₈)-, (C₁-C₆)- or (C₁-C₄)-haloalkylthio, for example (but not limited to) trifluoromethylthio, pentafluoroethylthio, difluoromethyl, 2,2-difluoroeth-1-ylthio, 2,2,2-difluoroeth-1-ylthio, 3,3,3-prop-1-ylthio.

“Halocycloalkyl” and “halocycloalkenyl” respectively denote cycloalkyl or cycloalkenyl that are partly or fully substituted by identical or different halogen atoms, such as F, Cl and Br, or by haloalkyl, such as trifluoromethyl or difluoromethyl, for example 1-fluorocycloprop-1-yl, 2-fluorocycloprop-1-yl, 2,2-difluorocycloprop-1-yl, 1-fluorocyclobut-1-yl, 1-trifluoromethylcycloprop-1-yl, 2-trifluoromethylcycloprop-1-yl, 1-chlorocycloprop-1-yl, 2-chlorocycloprop-1-yl, 2,2-dichlorocycloprop-1-yl, 3,3-difluorocyclobutyl.

Synthesis of substituted N-phenyluracils of the general formula (I).

The substituted N-phenyluracils of the general formula (I) according to the invention can be prepared using known processes. The synthesis routes used and examined proceed from commercially available or easily preparable heteroaromatic amines and from correspondingly substituted hydroxy esters. In the schemes which follow, the moieties G, Q, R¹, R², R³, R⁴, R⁵, R⁶, R⁷, X and Y of the general formula (I) have the meanings defined above, unless illustrative but non-limiting definitions are given. As a first key intermediate for the synthesis of the inventive compounds of the general formula (Ia) in which X is sulfur (S) and Y is oxygen (O), a mercaptophenyl-1H-pyrimidine-2,4-dione, which is optionally substituted further, is prepared. By way of example but without limitation, this is shown by the synthesis of 3-(4-chloro-2-fluoro-5-mercaptophenyl)-1-methyl-6-trifluoromethyl-1H-pyrimidine-2,4-dione (IIa) (Scheme 1). To this end, a suitable substituted aniline, by way of example but without limitation, 2-fluoro-4-chloroaniline, is converted with a suitable reagent (e.g. triphosgene) in a suitable polar aprotic solvent (e.g. dichloromethane) to the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) to the corresponding pyrimidine-2,4-dione optionally having further substitution, by way of example but without limitation 3-(4-chloro-2-fluorophenyl)-1-methyl-6-trifluoromethyl-1H-pyrimidine-2,4-dione (Scheme 1). By subsequent sulfochlorination with a suitable reagent (e.g. chlorosulfonic acid) followed by reduction with a suitable reducing agent (e.g. Zn in EtOH and HCl, tin(II) chloride hydrate or triphenylphosphine), it is possible to prepare the desired mercaptophenyl-1H-pyrimidine-2,4-dione, by way of example but without limitation 3-(4-chloro-2-fluoro-5-mercaptophenyl)-1-methyl-6-trifluoromethyl-1H-pyrimidine-2,4-dione (IIa) (cf. KR1345394; EP1122244; EP408382; WO 2003/029226; WO2010/038953; US2011/0224083; KR2011/110420). In Scheme 1 below, R² and R³, by way of example but without limitation, are fluorine, R⁴, by way of example but without limitation, is chlorine, and X, by way of example but without limitation, is sulfur.

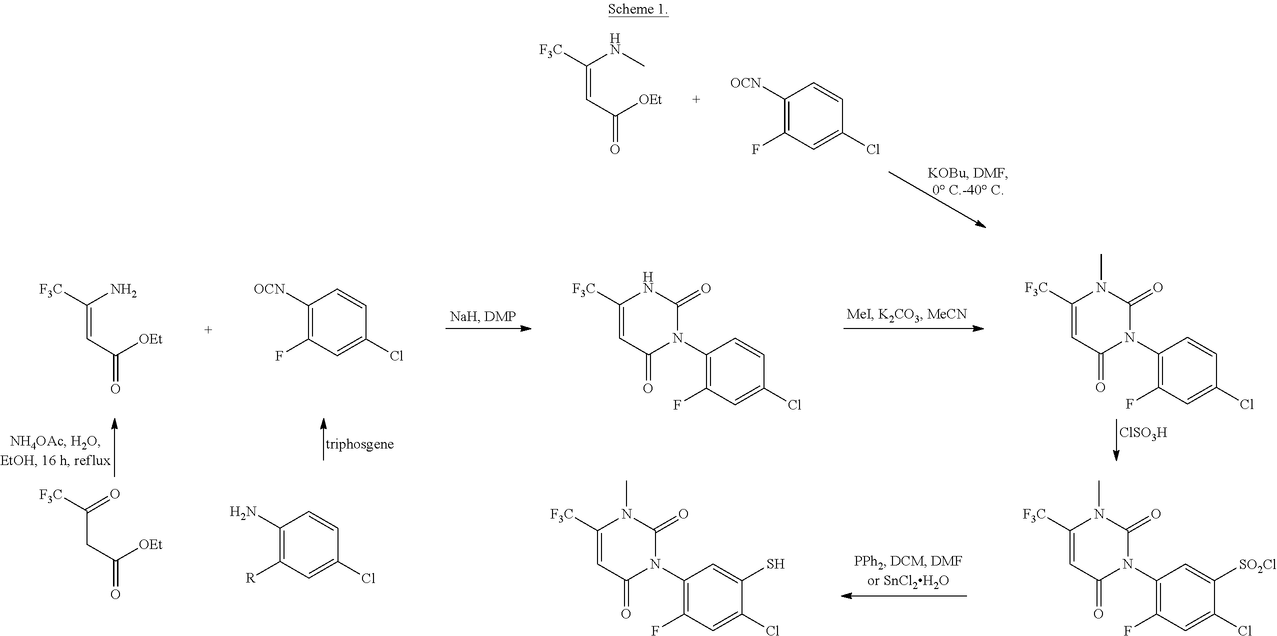

The synthesis of the key intermediate (IIa) described in Scheme 1 can also be applied to the preparation of similar intermediates. The respective further-substituted N-methyl-5-mercaptophenyl-1H-pyrimidine-2,4-dione intermediates (II) can then be converted by various routes to the desired inventive compounds of the general formula (Ia) in which X is sulfur (S) and Y is oxygen (O) (Scheme 2), after the compounds (II) have been converted to intermediates (III) in a first step with the aid of a suitable optionally further-substituted iodophenol using a suitable base or using a suitable transition metal catalyst (e.g. tris(dibenzylideneacetone)dipalladium(0)) having a suitable ligand (e.g. 4,5-bis(diphenylphosphino)-9,9-dimethylxanthene) and a suitable base (e.g. diispropyl(ethyl)amine) in a suitable polar aprotic solvent (e.g. dioxane). In Scheme 2 below, Q, R², R³, R³, and R⁴ have the inventive meanings above. In addition, R¹, R⁵, R⁶, R⁷, by way of example but without limitation, are hydrogen, X, by way of example but without limitation, is sulfur, Y, by way of example but without limitation, is oxygen and G, by way of example but without limitation, is CH₂. The corresponding intermediate (III) described by way of example but without limitation in Scheme 2 can be converted by reaction with a suitable optionally further-substituted iodoalkanoic ester (in Scheme 3, by way of example but without limitation, an iodoacetic ester) using a suitable base (e.g. silver(I) carbonate) in a suitable polar aprotic solvent (e.g. n-hexane or cyclohexane) at elevated temperature (e.g. under microwave conditions) to a corresponding oxyalkanoic ester intermediate (IVa, IVb) or the desired target compounds of the general formula (Ia) (cf. Synthesis 2009, 2725). The corresponding iodoalkanoic esters can be prepared by routes known from the literature (cf. Eur. J. Org. Chem., 2006, 71, 8459; WO2012037573; Organometallics, 2009, 28, 132).

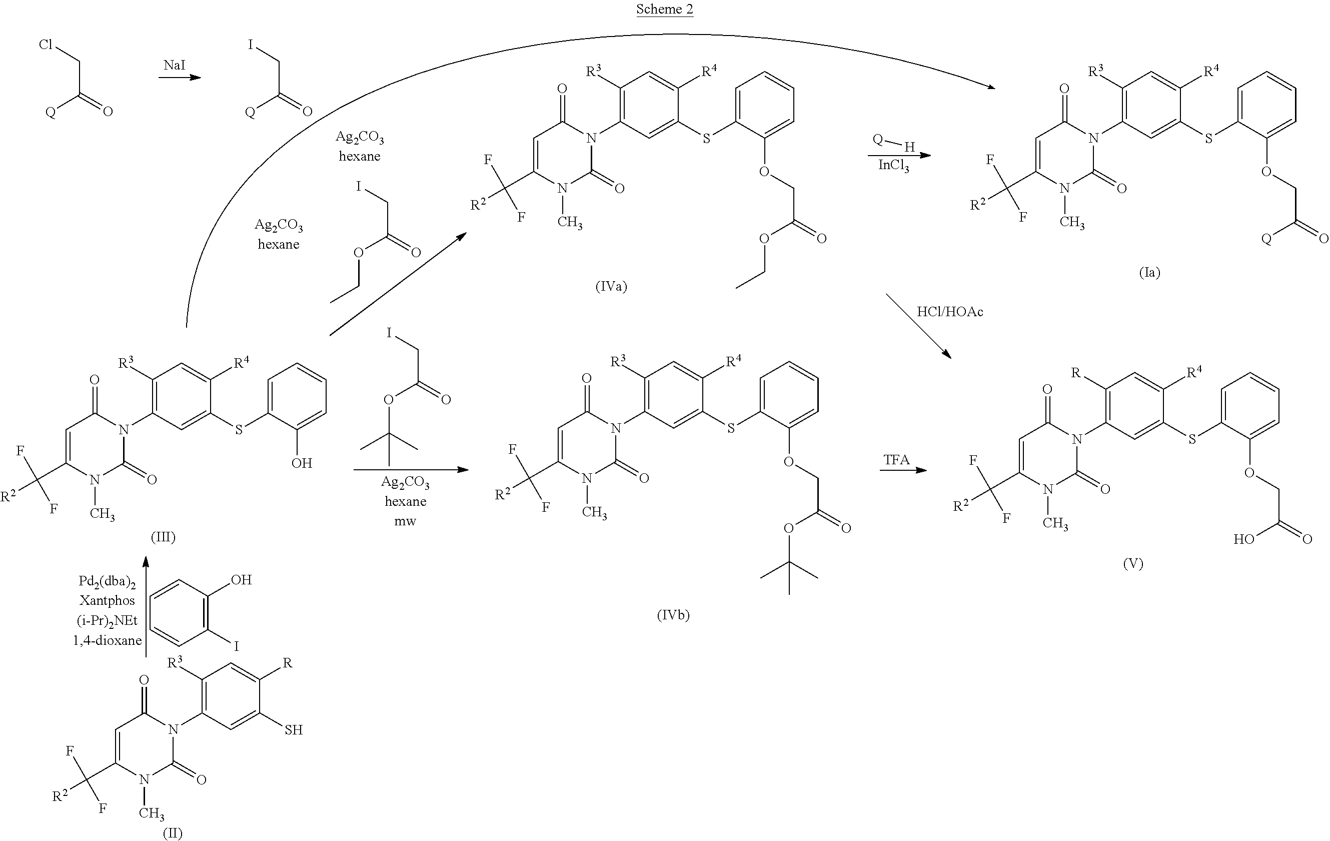

The ethyl ester (IVa) and tert-butyl ester (IVb) intermediates can then be converted under suitable reaction conditions [use of a suitable acid such as hydrochloric acid or acetic acid in the case of (IVa) or trifluoroacetic acid (TFA) in the case of (IVb)] to the corresponding free acid (V). By reaction of the corresponding acid intermediate (V) with a suitable compound Q-H with mediation by suitable coupling reagents (e.g. HOBt=1-hydroxybenzotriazole, EDC=1-ethyl-3-(3-dimethylaminopropyl)carbodiimide, HATU=O-(7-azabenzotriazol-1-yl)-N,N,N′,N′-tetramethyluronium hexafluorophosphate, T3P=2,4,6-tripropyl-1,3,5,2,4,6-trioxatriphosphorinane 2,4,6-trioxide) and suitable bases (e.g. diisopropylethylamine, triethylamine) in a suitable polar aprotic solvent (e.g. dichloromethane, chloroform), it is possible to prepare the desired substituted N-phenyluracils of the general formula (Ia). Alternatively, the ethyl ester (IVa) can be converted by coupling with a suitable compound Q-H with mediation by a suitable Lewis acid (e.g. indium(III) chloride) to the corresponding desired substituted N-phenyluracil of the general formula (Ia) (cf. WO2011/1307088).

The preparation of the compounds of the general formula (I) in which X and Y, by way of example but without limitation, are oxygen (O) proceeds via the synthesis of key intermediates (VI) having a fluorine substituent at position 5, such as 3-(2,5-difluoro-4-nitro)-1-methyl-6-trifluoromethyl-1H-pyrimidine-2,4-dione (VIa). To this end, a suitable substituted aniline, by way of example but without limitation 2,5-difluoroaniline, is converted with a suitable reagent (e.g. triphosgene) in a suitable polar aprotic solvent (e.g. dichloromethane) to the corresponding isocyanate which, in the next step, is converted by reaction with a suitable aminoacrylic ester using a suitable base (e.g. sodium hydride or potassium tert-butoxide) in a suitable polar aprotic solvent (e.g. N,N-dimethylformamide) to the corresponding pyrimidine-2,4-dione optionally having further substitution, here by way of example but without limitation 3-(2,5-difluorophenyl)-6-trifluoromethyl-1H-pyrimidine-2,4-dione (Scheme 3). Nitration with a suitable nitration reagent and subsequent N-methylation with a suitable methylating reagent affords the desired intermediate, here by way of example but without limitation 3-(2,5-difluoro-4-nitro)-1-methyl-6-trifluoromethyl-1H-pyrimidine-2,4-dione (VIa). In Scheme 3 below, R² and R³, by way of example but without limitation, are fluorine, and R⁴, by way of example but without limitation, is nitro.

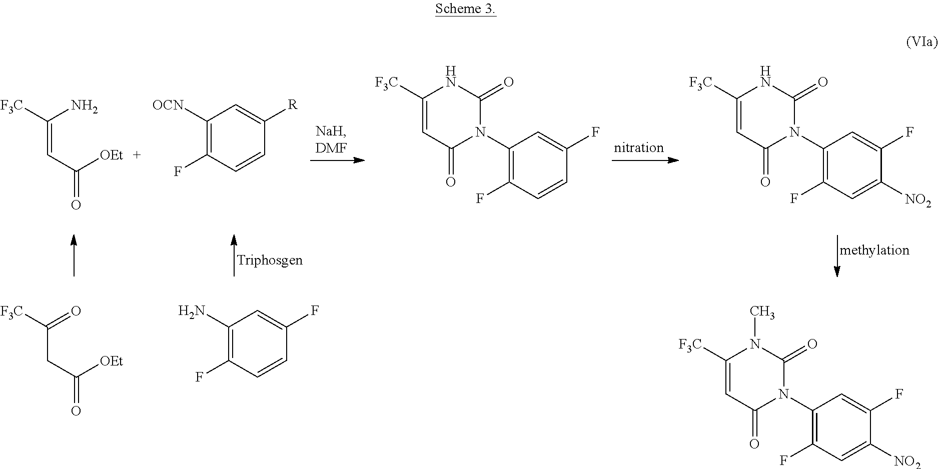

The intermediate (VI) obtained in the manner described above, e.g. compound (VIa), can then be converted with a suitable substituted 2-carbonylalkyloxy-1-hydroxybenzene (VII) using a suitable base (e.g. potassium carbonate) in a suitable polar aprotic solvent (e.g. N,N-dimethylformamide (DMF)) to a desired substituted N-phenyluracil (Ib, R⁴=nitro). The intermediate (VII) used for this purpose can be obtained by a multistage synthesis proceeding from commercially available 1-chloro-2-nitrobenzene, via (i) base-mediated coupling (e.g. with sodium hydride) with a suitable substituted hydroxyalkylcarbonyl reagent in a suitable polar aprotic solvent (e.g. tetrahydrofuran or dioxane) or alternatively by reaction of 2-nitrophenol with a suitable substituted chloromethylcarbonyl reagent, (ii) reduction of the nitro group with a suitable reducing agent (e.g. hydrogen, palladium on carbon in a suitable polar protic solvent), (iii) diazotization (with a suitable diazotizing reagent, e.g. tert-butyl nitrite (t-BuONO), boron trifluoride etherate (BF₃—OEt₂) in suitable polar aprotic solvents (e.g. dichloromethane (DCM), dimethoxyethane), (iv) reaction with acetic anhydride and (v) release of the hydroxy group by removal of the acetyl protective group (e.g. base-mediated with potassium carbonate in a polar protic solvent). The nitro group in compound (Ib) can then be converted by reduction and subsequent Sandmeyer reaction to a halogen substituent (e.g. chlorine, bromine), such that the desired substituted N-phenyluracil (Ic) can be obtained in this manner. In Scheme 4 below, Q and R² have the above inventive meanings. In addition, R³, by way of example but without limitation, is fluorine, R⁴, by way of example but without limitation, is chlorine or nitro, R¹, R⁵, R⁶, R⁷, by way of example but without limitation, are hydrogen, X and Y, by way of example but without limitation, are oxygen, and G, by way of example but without limitation, is CH₂.

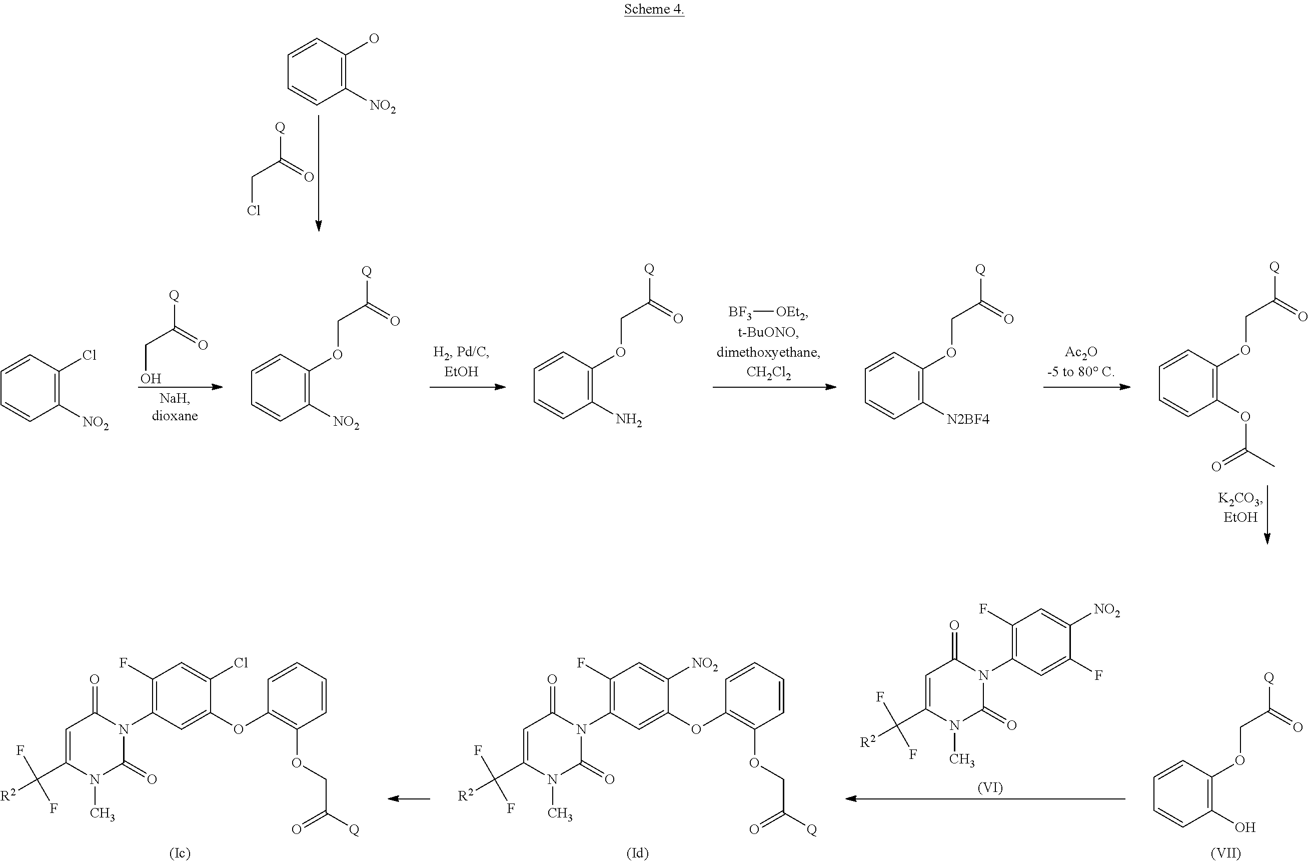

Accordingly, the intermediate (VI) obtained in the manner described above can be converted with a suitable substituted 2-carbonylalkylthio-1-hydroxybenzene (VIII) using a suitable base (e.g. potassium carbonate) in a suitable polar aprotic solvent (e.g. N,N-dimethylformamide (DMF)) to a desired substituted N-phenyluracil (Id, R⁴=nitro) where X=O (oxygen) and Y=S (sulfur). The intermediate (VIII) used for this purpose can be prepared by a multi-step synthesis analogously to the synthesis of intermediate (VII) described in Scheme 4 starting with commercially available 1-chloro-2-nitrobenzene or 2-nitrothiophenol. The nitro group in compound (Id) can then be converted by reduction and subsequent Sandmeyer reaction to a halogen substituent (e.g. chlorine, bromine), such that the desired substituted N-phenyluracil (Ie) can be obtained in this manner. In Scheme 5 below, Q and R² have the above inventive meanings. In addition, R³, by way of example but without limitation, is fluorine, R⁴, by way of example but without limitation, is chlorine or nitro, R¹, R⁵, R⁶, R⁷ by way of example but without limitation, are hydrogen, X, by way of example but without limitation, is oxygen, Y, by way of example but without limitation, is sulfur and G, by way of example but without limitation, is CH₂.

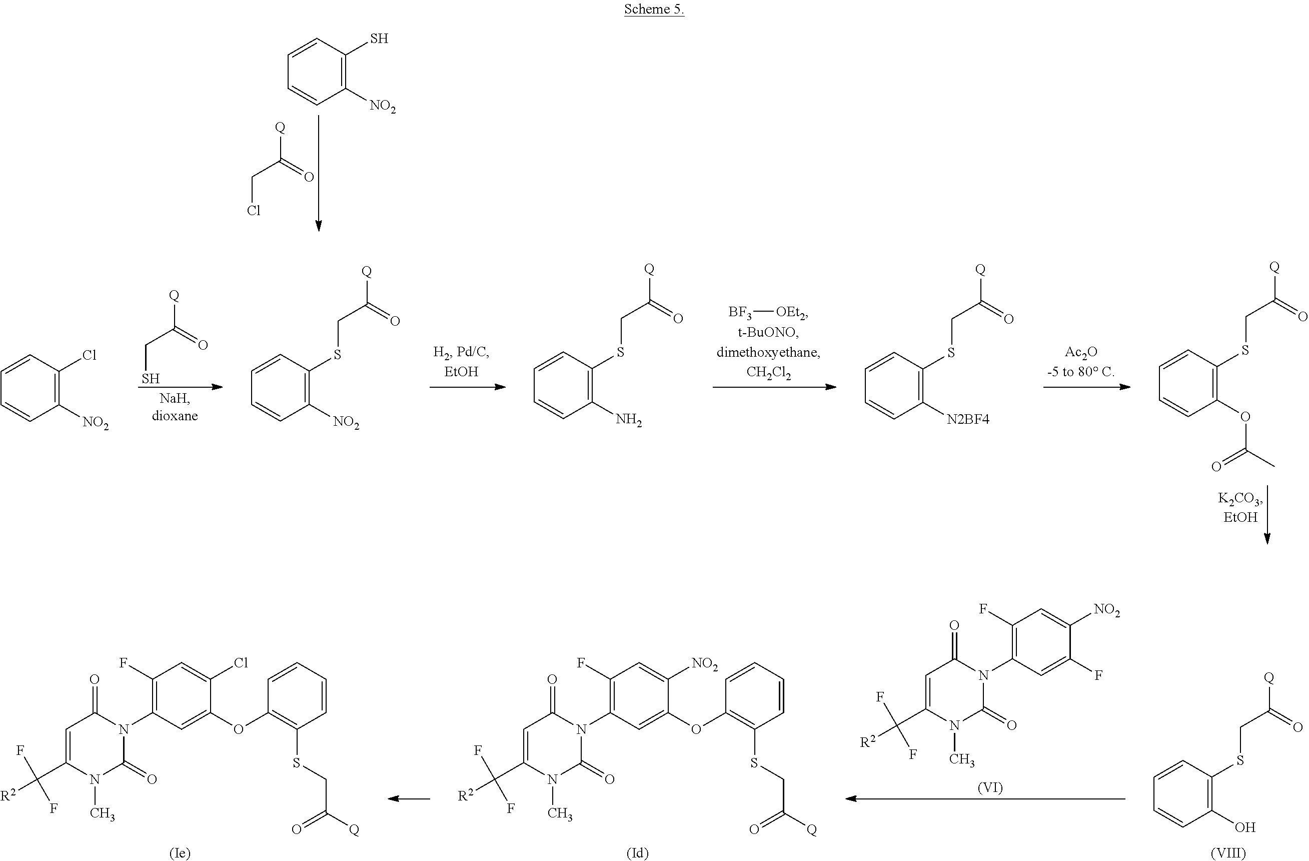

The further-substituted N-methyl-5-mercaptophenyl-1H-pyrimidine-2,4-dione intermediates (II) can also be converted to the desired inventive compounds of the general formula (If) in which X and Y are sulfur (S) (Scheme 6), after the compounds (III) have been converted to intermediates of the (IX) type, in a first step with the aid of a suitable optionally further-substituted iodothiophenol using a suitable base or using a suitable transition metal catalyst (e.g. tris(dibenzylideneacetone)dipalladium(0)) having a suitable ligand (e.g. 4,5-bis(diphenylphosphino)-9,9-dimethylxanthene) and a suitable base (e.g. diispropyl(ethyl)amine) in a suitable polar aprotic solvent (e.g. dioxane). As the case may be, intermediate protection of the thiol group using a suitable protecting group may be required. The intermediates (IX) can then be reacted with haloalkanecarboxylic acids having various substitutions using suitable bases, to afford the desired compounds of the general formula (If). In Scheme 6 below, Q, R², R³, and R⁴ have the inventive meanings above. In addition, R¹, R⁵, R⁶, R⁷, by way of example but without limitation, are hydrogen, X and Y, by way of example but without limitation, are sulfur and G, by way of example but without limitation, is CH₂. In addition, the reaction pathways are described in Scheme 6 below for better clarity, by way of example but without limitation using iodoacetic esters.

Also suitable for coupling with intermediate (IX) are comparable haloalkanecarboxylic acids (halogen=bromine or chlorine).

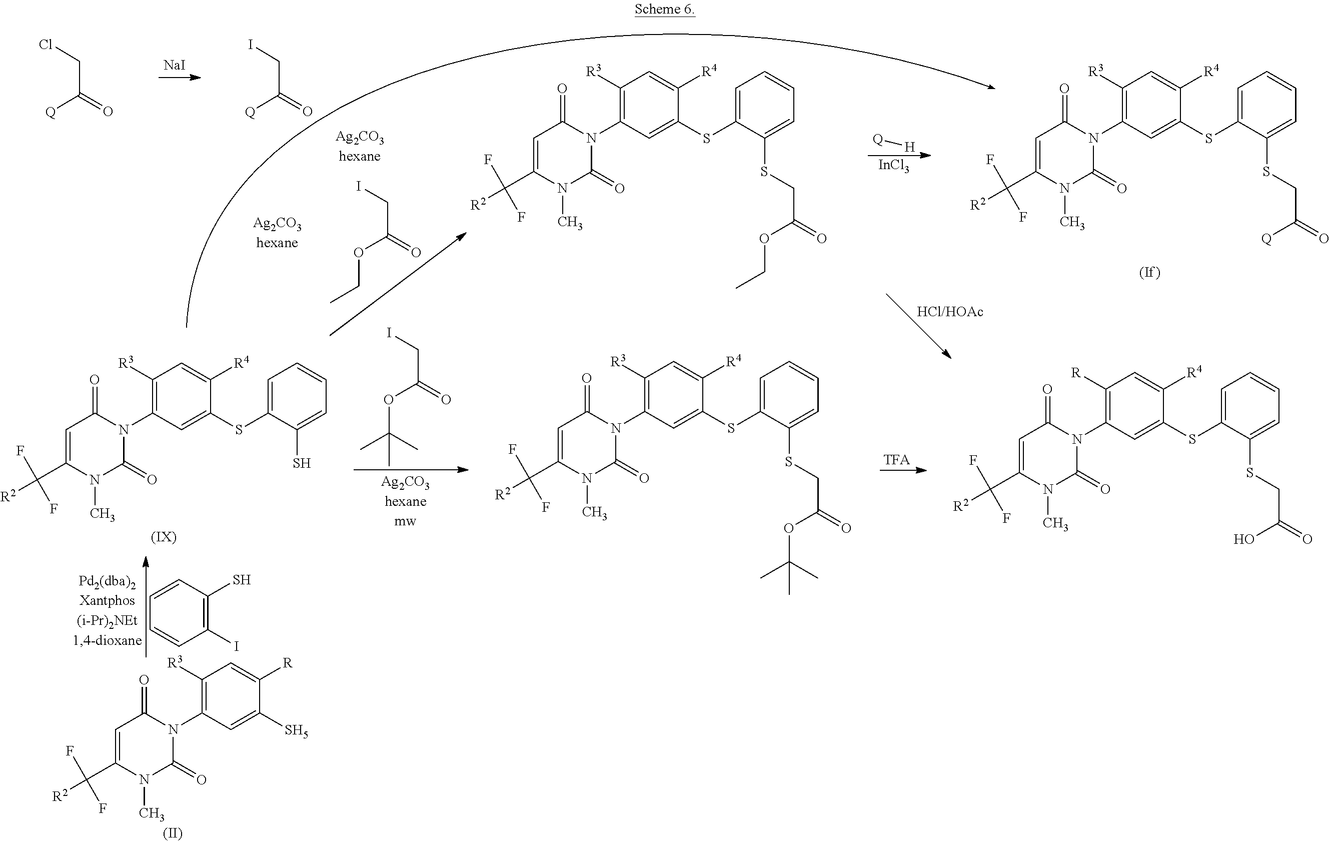

Selected detailed synthesis examples for the inventive compounds of the general formula (I) are adduced below. The example numbers mentioned correspond to the numbering scheme in Tables I.1 to I.33 below. The ¹H NMR, ¹³C-NMR and ¹⁹F-NMR spectroscopy data reported for the chemical examples described in the sections which follow (400 MHz for ¹H NMR and 150 MHz for ¹³C-NMR and 375 MHz for ¹⁹F-NMR, solvent CDCl₃, CD₃OD or d₆-DMSO, internal standard: tetramethylsilane δ=0.00 ppm) were obtained on a Bruker instrument, and the signals listed have the meanings given below: br=broad; s=singlet, d=doublet, t=triplet, dd=doublet of doublets, ddd=doublet of a doublet of doublets, m=multiplet, q=quartet, quint=quintet, sext=sextet, sept=septet, dq=doublet of quartets, dt=doublet of triplets. In the case of diastereomer mixtures, either the significant signals for each of the two diastereomers are reported or the characteristic signal of the main diastereomer is reported. The abbreviations used for chemical groups have, for example, the following meanings: Me=CH₃, Et=CH₂CH₃,

t-Hex=C(CH₃)₂CH(CH₃)₂, t-Bu=C(CH₃)₃, n-Bu=unbranched butyl, n-Pr=unbranched propyl, i-Pr=branched propyl, c-Pr=cyclopropyl, c-Hex=cyclohexyl.

### SYNTHESIS EXAMPLES

### Ex. I.1-1: 2-Methoxyethyl [2-({2-chloro-4-fluoro-5-[3-methyl-2,6-dioxo-4-(trifluoromethyl)-3,6-dihydropyrimidin-1(2H)-yl]phenyl}sulfanyl)phenoxy]acetate

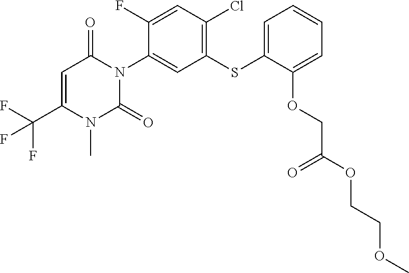 [SMILES: COCCOC(=O)COc1ccccc1Sc1cc(-n2c(=O)cc(C(F)(F)F)n(C)c2=O)c(F)cc1Cl]

To a solution of methylglycol (0.012 g, 0.155 mmol) in 4 ml of dichloromethane were added [2-({2-chloro-4-fluoro-5-[3-methyl-2,6-dioxo-4-(trifluoromethyl)-3,6-dihydropyrimidin-1(2H)-yl]phenyl}sulfanyl)phenoxy]acetic acid (0.060 g, 0.119 mmol) and then, in succession, 1-hydroxy-1H-benzotriazole hydrate (0.024 g, 0.155 mmol), 1-(3-dimethylaminopropyl)-3-ethylcarbodiimide hydrochloride (0.030 g, 0.155 mmol) and 4-dimethylaminopyridine (10 mol %), and the mixture was stirred at room temperature overnight. The reaction mixture was purified by column chromatography on silica gel with an n-heptane/ethyl acetate gradient. This gave 0.044 g (64% of theory) of 2-methoxyethyl [2-({2-chloro-4-fluoro-5-[3-methyl-2,6-dioxo-4-(trifluoromethyl)-3,6-dihydropyrimidin-1(2H)-yl]phenyl}sulfanyl)phenoxy]acetate. ¹H-NMR (CDCl₃ δ, ppm) 7.44-7.41 (m, 1H), 7.34-7.30 (m, 2H), 7.08 (d, 1H), 7.01-6.97 (m, 1H), 6.78-6.76 (m, 1H), 6.27 (s, 1H), 4.70 (s, 2H), 4.29-4.26 (m, 2H), 3.58-3.56 (m, 2H), 3.50-3.49 (m, 3H), 3.35 (s, 3H).

### Ex. I.2-1: 2-Methoxyethyl (2RS)-2-[2-({2-chloro-4-fluoro-5-[3-methyl-2,6-dioxo-4-(trifluoromethyl)-3,6-dihydropyrimidin-1(2H)-yl]phenyl}sulfanyl)phenoxy]propanoate

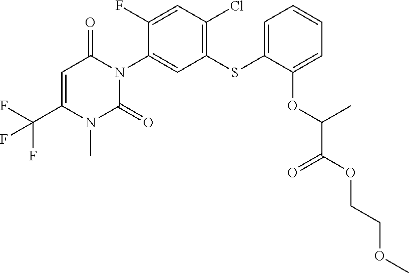 [SMILES: COCCOC(=O)C(C)Oc1ccccc1Sc1cc(-n2c(=O)cc(C(F)(F)F)n(C)c2=O)c(F)cc1Cl]

To a solution of methylglycol (0.011 g, 0.150 mmol) in 4 ml of dichloromethane were added (2RS)-2-[2-({2-chloro-4-fluoro-5-[3-methyl-2,6-dioxo-4-(trifluoromethyl)-3,6-dihydropyrimidin-1(2H)-yl]phenyl}sulfanyl)phenoxy]propanoic acid (0.060 g, 0.116 mmol) and then, in succession, 1-hydroxy-1H-benzotriazole hydrate (0.023 g, 0.150 mmol), 1-(3-dimethylaminopropyl)-3-ethylcarbodiimide hydrochloride (0.029 g, 0.150 mmol) and 4-dimethylaminopyridine (10 mol %), and the mixture was stirred at room temperature overnight. The reaction mixture was purified by column chromatography on silica gel with an n-heptane/ethyl acetate gradient. This gave 0.056 g (80% of theory) of 2-methoxyethyl (2RS)-2-[2-({2-chloro-4-fluoro-5-[3-methyl-2,6-dioxo-4-(trifluoromethyl)-3,6-dihydropyrimidin-1(2H)-yl]phenyl}sulfanyl)phenoxy]propanoate. ¹H-NMR (CDCl₃ δ, ppm) 7.43-7.40 (m, 1H), 7.32-7.28 (m, 2H), 7.10-6.95 (m, 2H), 6.76-6.72 (m, 1H), 6.27-6.26 (m, 1H), 4.79-4.73 (m, 1H), 4.32-4.22 (m, 2H), 3.56-3.53 (m, 2H), 3.49 (m, 3H), 3.33 (s, 3H), 1.51-1.50 (m, 3H).

### Ex. I.7-176: Cyanomethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

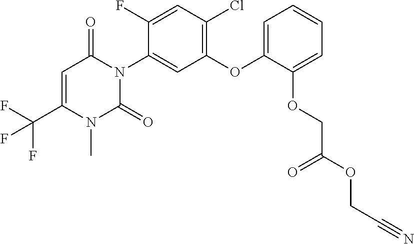 [SMILES: Cn1c(C(F)(F)F)cc(=O)n(-c2cc(Oc3ccccc3OCC(=O)OCC#N)c(Cl)cc2F)c1=O]

To a solution of glycolonitrile (0.027 g, 0.286 mmol) in 10 ml of dichloromethane were added (2-{2-chloro-4-fluoro-5-[3-methyl-2,6-dioxo-4-(trifluoromethyl)-3,6-dihydropyrimidin-1(2H)-yl]phenoxy}phenoxy)acetic acid (0.100 g, 0.205 mmol) and then, in succession, 1-hydroxy-1H-benzotriazole hydrate (0.041 g, 0.266 mmol), 1-(3-dimethylaminopropyl)-3-ethylcarbodiimide hydrochloride (0.051 g, 0.266 mmol) and 4-dimethylaminopyridine (10 mol %), and the mixture was stirred at room temperature for 2 h. Water and dichloromethane were added to the reaction mixture, the aqueous phase was extracted repeatedly with dichloromethane, the combined organic phases were dried over sodium sulfate, and the solvent was removed under reduced pressure. After purification by column chromatography on silica gel with an n-heptane/ethyl acetate gradient, 0.095 g (79% of theory) of a colorless solid was obtained. ¹H-NMR (CDCl₃ δ, ppm) 7.39 (d, 1H), 7.18-7.12 (m, 1H), 7.11-7.03 (m, 2H), 6.94 (d, 1H), 6.68 (d, 1H), 6.29 (s, 1H), 4.76 (s, 2H), 4.74 (s, 2H), 3.50 (s, 3H).

### Ex. I.7-71: Tetrahydrofuran-2-ylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

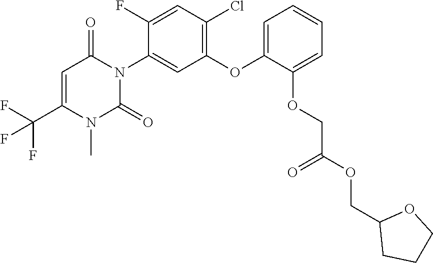 [SMILES: Cn1c(C(F)(F)F)cc(=O)n(-c2cc(Oc3ccccc3OCC(=O)OCC3CCCO3)c(Cl)cc2F)c1=O]

To a solution of tetrahydrofuran-2-ylmethanol (0.042 g, 0.412 mmol) in 15 ml of dichloromethane were added (2-{2-chloro-4-fluoro-5-[3-methyl-2,6-dioxo-4-(trifluoromethyl)-3,6-dihydropyrimidin-1(2H)-yl]phenoxy}phenoxy)acetic acid (0.160 g, 0.327 mmol) and then, in succession, 1-hydroxy-1H-benzotriazole hydrate (0.059 g, 0.383 mmol), 1-(3-dimethylaminopropyl)-3-ethylcarbodiimide hydrochloride (0.073 g, 0.383 mmol) and 4-dimethylaminopyridine (10 mol %), and the mixture was stirred at room temperature for 2 h. Water and dichloromethane were added to the reaction mixture, the aqueous phase was extracted repeatedly with dichloromethane, the combined organic phases were dried over sodium sulfate, and the solvent was removed under reduced pressure. After purification by column chromatography on silica gel with an n-heptane/ethyl acetate gradient, 0.129 g (73% of theory) of a colorless solid was obtained. ¹H-NMR (CDCl₃ δ, ppm) 7.36 (d, 1H), 7.14-7.10 (m, 1H), 7.07-7.04 (m, 1H), 7.01-6.97 (m, 1H), 6.92 (d, 1H), 6.77 (d, 1H), 6.28 (s, 1H), 4.70 (s, 2H), 4.21-4.18 (m, 1H), 4.13-4.05 (m, 2H), 3.83-3.73 (m, 2H), 3.50 (s, 3H), 2.04-1.83 (m, 3H), 1.59-1.52 (m, 1H).

### Ex. I.7-276: Pyridin-2-ylmethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

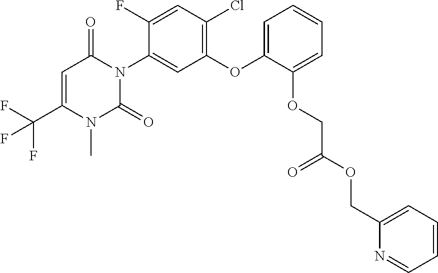 [SMILES: Cn1c(C(F)(F)F)cc(=O)n(-c2cc(Oc3ccccc3OCC(=O)OCc3ccccn3)c(Cl)cc2F)c1=O]

To a solution of pyridin-2-ylmethanol (0.028 g, 0.258 mmol) in 10 ml of dichloromethane were added (2-{2-chloro-4-fluoro-5-[3-methyl-2,6-dioxo-4-(trifluoromethyl)-3,6-dihydropyrimidin-1(2H)-yl]phenoxy}phenoxy)acetic acid (0.100 g, 0.205 mmol) and then, in succession, 1-hydroxy-1H-benzotriazole hydrate (0.037 g, 0.239 mmol), 1-(3-dimethylaminopropyl)-3-ethylcarbodiimide hydrochloride (0.046 g, 0.239 mmol) and 4-dimethylaminopyridine (10 mol %), and the mixture was stirred at room temperature for 2 h. Water and dichloromethane were added to the reaction mixture, the aqueous phase was extracted repeatedly with dichloromethane, the combined organic phases were dried over sodium sulfate, and the solvent was removed under reduced pressure. After purification by column chromatography on silica gel with an n-heptane/ethyl acetate gradient, 0.096 g (81% of theory) of a colorless solid was obtained. ¹H-NMR (CDCl₃ δ, ppm) 8.57-8.55 (m, 1H), 7.66 (t, 1H), 7.32 (d, 1H), 7.26-7.21 (m, 2H), 7.14-7.08 (m, 2H), 7.02-6.94 (m, 2H), 6.75 (d, 1H), 6.24 (s, 1H), 5.28 (s, 2H), 4.77 (s, 2H), 3.48 (s, 3H).

### Ex. I.7-183: 2-Nitroethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

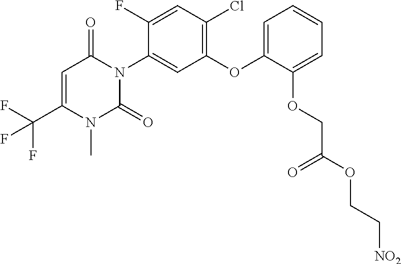 [SMILES: Cn1c(C(F)(F)F)cc(=O)n(-c2cc(Oc3ccccc3OCC(=O)OCC[N+](=O)[O-])c(Cl)cc2F)c1=O]

To a solution of (2-{2-chloro-4-fluoro-5-[3-methyl-2,6-dioxo-4-(trifluoromethyl)-3,6-dihydropyrimidin-1(2H)-yl]phenoxy}phenoxy)acetic acid (0.150 g, 0.306 mmol) in 1.0 ml of toluene were successively added 2-nitroethanol (0.139 g, 1.531 mmol), concentrated acetic acid (0.092 g, 1.531 mmol), concentrated sulfuric acid (0.015 g, 0.153 mmol), and the mixture was stirred under reflux for 5 h. The reaction mixture was left to stand overnight and stirred under reflux for a further 5 h. Water and dichloromethane were added to the reaction mixture, the aqueous phase was extracted repeatedly with dichloromethane, the combined organic phases were dried over sodium sulfate, and the solvent was removed under reduced pressure. After purification by column chromatography on silica gel with an n-heptane/acetone gradient followed by a further purification by column chromatography on silica gel with an n-heptane/ethyl acetate gradient, 0.036 g (20% of theory) of 2-nitroethyl (2-{2-chloro-4-fluoro-5-[3-methyl-2,6-dioxo-4-(trifluoromethyl)-3,6-dihydropyrimidin-1(2H)-yl]phenoxy}phenoxy)acetate was obtained. ¹H-NMR (CDCl₃ δ, ppm) 7.37 (d, 1H), 7.16-7.12 (m, 1H), 7.09-7.07 (m, 1H), 7.04-7.00 (m, 1H), 6.90 (d, 1H), 6.67 (d, 1H), 6.28 (s, 1H), 4.67 (s, 2H), 4.66-4.64 (m, 2H), 4.59-4.56 (m, 2H), 3.50 (s, 3H).

### Ex. I.7-177: 1-Cyanoethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

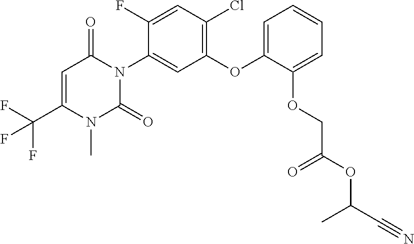 [SMILES: CC(C#N)OC(=O)COc1ccccc1Oc1cc(-n2c(=O)cc(C(F)(F)F)n(C)c2=O)c(F)cc1Cl]

To a solution of (2-{2-chloro-4-fluoro-5-[3-methyl-2,6-dioxo-4-(trifluoromethyl)-3,6-dihydropyrimidin-1(2H)-yl]phenoxy}phenoxy)acetic acid (0.160 g, 0.327 mmol) in 4.0 ml of acetonitrile were successively added 4-dimethylaminopyridine (0.005 g, 0.039 mmol), potassium carbonate (0.056 g, 0.404 mmol), 2-bromopropanenitrile (0.052 g, 0.389 mmol), and the mixture was stirred at room temperature for 2 h and then under reflux for 1 h. Water and dichloromethane were added to the reaction mixture, the aqueous phase was extracted repeatedly with dichloromethane, the combined organic phases were dried over sodium sulfate, and the solvent was removed under reduced pressure. After purification by column chromatography with an n-heptane/ethyl acetate gradient and subsequent drying of the product obtained under reduced pressure at 40° C., 0.160 g (95% of theory) of 1-cyanoethyl (2-{2-chloro-4-fluoro-5-[3-methyl-2,6-dioxo-4-(trifluoromethyl)-3,6-dihydropyrimidin-1(2H)-yl]phenoxy}phenoxy)acetate was obtained. ¹H-NMR (CDCl₃ δ, ppm) 7.38 (d, 1H), 7.17-7.13 (m, 1H), 7.10-7.02 (m, 2H), 6.95-6.93 (m, 1H), 6.72-6.68 (m, 1H), 6.29 (d, 1H), 5.45-5.42 (m, 1H), 4.72 (s, 2H), 3.50 (s, 3H), 1.64-1.61 (m, 3H).

### Ex. I.7-1: 2-Methoxyethyl (2-{2-chloro-4-fluoro-5-[3-methyl-2,6-dioxo-4-(trifluoromethyl)-3,6-dihydropyrimidin-1(2H)-yl]phenoxy}phenoxy)acetate

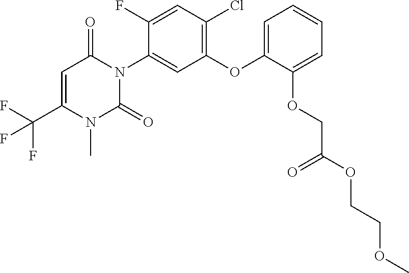 [SMILES: COCCOC(=O)COc1ccccc1Oc1cc(-n2c(=O)cc(C(F)(F)F)n(C)c2=O)c(F)cc1Cl]

Under a nitrogen atmosphere, indium(III) chloride (2.599 g, 11.749 mmol) was added to ethyl (2-{2-chloro-4-fluoro-5-[3-methyl-2,6-dioxo-4-(trifluoromethyl)-3,6-dihydropyrimidin-1(2H)-yl]phenoxy}phenoxy)acetate (5.520 g, 10.681 mmol) in 46.32 ml of 2-methoxyethanol, and the mixture was stirred at 115° C. for 3 h. After cooling to room temperature, the reaction mixture was poured onto water, ethyl acetate was added, the aqueous phase was extracted repeatedly with ethyl acetate, the combined organic phases were washed with saturated aqueous sodium chloride solution, the organic phase was dried over sodium sulfate, and the solvent was removed under reduced pressure. After purification by column chromatography on silica gel with an n-heptane/ethyl acetate gradient, 5.150 g (88% of theory) of a colorless solid was obtained. ¹H-NMR (CDCl₃ δ, ppm) 7.36 (d, 1H), 7.14-7.10 (m, 1H), 7.07-7.05 (m, 1H), 7.01-6.99 (m, 1H), 6.93-6.91 (m, 1H), 6.77 (d, 1H), 6.28 (s, 1H), 4.70 (s, 2H), 4.29-4.27 (m, 2H), 3.56-3.54 (m, 2H), 3.50 (s, 3H), 3.33 (s, 3H).

### Ex. I.7-94: 1-Methoxypropan-2-yl (2-{2-chloro-4-fluoro-5-[3-methyl-2,6-dioxo-4-(trifluoromethyl)-3,6-dihydropyrimidin-1(2H)-yl]phenoxy}phenoxy)acetate

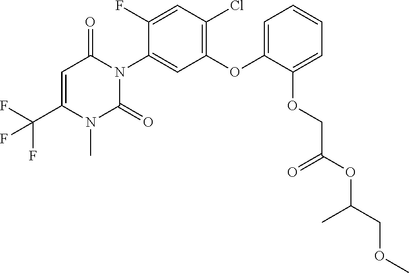 [SMILES: COCC(C)OC(=O)COc1ccccc1Oc1cc(-n2c(=O)cc(C(F)(F)F)n(C)c2=O)c(F)cc1Cl]

Under a nitrogen atmosphere, indium(III) chloride (0.116 g, 0.527 mmol) was added to 2-methoxyethyl (2-{2-chloro-4-fluoro-5-[3-methyl-2,6-dioxo-4-(trifluoromethyl)-3,6-dihydropyrimidin-1(2H)-yl]phenoxy}phenoxy)acetate (0.160 g, 0.293 mmol) in 4.0 ml of 1-methoxypropan-2-ol, and the reaction mixture was heated at 125° C. in a microwave reactor for 1.5 h. After cooling to room temperature, the reaction mixture was poured onto water, dichloromethane was added, the aqueous phase was extracted repeatedly with dichloromethane, the combined organic phases were dried over sodium sulfate, and the solvent was removed under reduced pressure. After purification by column chromatography with an n-heptane/ethyl acetate gradient, 0.110 g (66% of theory) of 1-methoxypropan-2-yl (2-{2-chloro-4-fluoro-5-[3-methyl-2,6-dioxo-4-(trifluoromethyl)-3,6-dihydropyrimidin-1(2H)-yl]phenoxy}phenoxy)acetate was obtained. ¹H-NMR (CDCl₃ δ, ppm) 7.36 (d, 1H), 7.14-7.10 (m, 1H), 7.07-7.04 (m, 1H), 7.01-6.96 (m, 1H), 6.93-6.90 (m, 1H), 6.79-6.76 (m, 1H), 6.28 (d, 1H), 5.16-5.12 (m, 1H), 4.65 (s, 2H), 3.50 (s, 3H), 3.44-3.36 (m, 2H), 3.33 (s, 3H), 1.23 (d, 3H).

### Ex. I.6-1: 2-Methoxyethyl (2-{4-fluoro-5-[3-methyl-2,6-dioxo-4-(trifluoromethyl)-3,6-dihydropyrimidin-1(2H)-yl]-2-nitrophenoxy}phenoxy)acetate

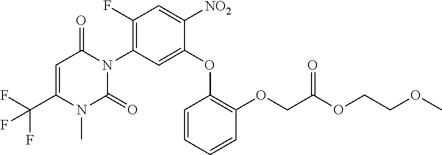 [SMILES: COCCOC(=O)COc1ccccc1Oc1cc(-n2c(=O)cc(C(F)(F)F)n(C)c2=O)c(F)cc1[N+](=O)[O-]]

To ethyl (2-{4-fluoro-5-[3-methyl-2,6-dioxo-4-(trifluoromethyl)-3,6-dihydropyrimidin-1(2H)-yl]-2-nitrophenoxy}phenoxy)acetate (100 mg, 0.19 mmol) in a microwave vessel under argon were added 2-methoxyethanol (505 mg, 6.63 mmol) and indium(III) chloride (51.2 mg, 0.23 mmol). The reaction mixture was stirred at a temperature of 115° C. under microwave conditions for 120 minutes. After cooling to room temperature, the reaction mixture was stirred with 50 ml of water, and 10 ml of dichloromethane was added. The organic phase was removed and concentrated. Column chromatography purification of the crude product obtained on silica gel with an n-heptane/ethyl acetate gradient afforded 2-methoxyethyl (2-{4-fluoro-5-[3-methyl-2,6-dioxo-4-(trifluoromethyl)-3,6-dihydropyrimidin-1(2H)-yl]-2-nitrophenoxy}phenoxy)acetate (78 mg, 73% of theory) in the form of a colorless oil.

¹H-NMR (CDCl₃ δ, ppm) 7.87 (d, 1H), 7.19 (m, 2H), 7.03 (t, 1H), 6.95 (d, 1H), 6.88 (d, 1H), 6.28 (s, 1H), 4.67 (s, 2H), 4.25 (t, 2H), 3.53 (t, 2H), 3.50 (s, 3H), 3.31 (s, 3H).

### Ex. I.6-176: Cyanomethyl (2-{4-fluoro-5-[3-methyl-2,6-dioxo-4-(trifluoromethyl)-3,6-dihydropyrimidin-1(2H)-yl]-2-nitrophenoxy}phenoxy)acetate

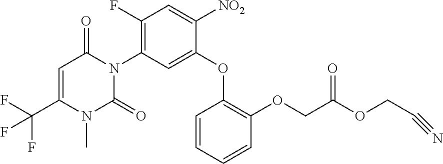 [SMILES: Cn1c(C(F)(F)F)cc(=O)n(-c2cc(Oc3ccccc3OCC(=O)OCC#N)c([N+](=O)[O-])cc2F)c1=O]

An initial charge of (2-{4-fluoro-5-[3-methyl-2,6-dioxo-4-(trifluoromethyl)-3,6-dihydropyrimidin-1(2H)-yl]-2-nitrophenoxy}phenoxy)acetic acid (100 mg, 0.20 mmol) together with bromoacetonitrile (38 mg, 0.30 mmol) and triethylamine (30 mg, 0.30 mmol) in 1 ml of acetone under argon was stirred at room temperature for 2.5 hours. After TLC monitoring, further bromoacetonitrile (13 mg, 0.10 mmol) was added and the mixture was stirred at room temperature for a further hour. The reaction mixture was concentrated, and 10 ml of water and 10 ml of dichloromethane were added. The organic phase was removed and concentrated. Column chromatography purification of the crude product obtained on silica gel with an n-heptane/ethyl acetate gradient afforded cyanomethyl (2-{4-fluoro-5-[3-methyl-2,6-dioxo-4-(trifluoromethyl)-3,6-dihydropyrimidin-1(2H)-yl]-2-nitrophenoxy}phenoxy)acetate (104 mg, 95% of theory) in the form of a colorless oil.

¹H-NMR (CDCl₃ δ, ppm) 7.89 (d, 1H), 7.24-7.20 (m, 2H), 7.09 (dt, 1H), 6.92 (dd, 1H), 6.82 (d, 1H), 6.30 (s, 1H), 4.75 (s, 2H), 4.71 (s, 2H), 3.51 (s, 3H).

### Ex. I.8-176: Cyanomethyl (2-{2-bromo-4-fluoro-5-[3-methyl-2,6-dioxo-4-(trifluoromethyl)-3,6-dihydropyrimidin-1(2H)-yl]phenoxy}phenoxy)acetate

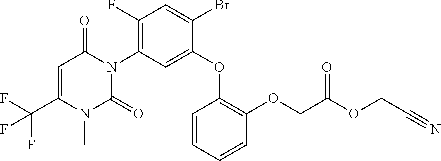 [SMILES: Cn1c(C(F)(F)F)cc(=O)n(-c2cc(Oc3ccccc3OCC(=O)OCC#N)c(Br)cc2F)c1=O]

An initial charge of (2-{2-bromo-4-fluoro-5-[3-methyl-2,6-dioxo-4-(trifluoromethyl)-3,6-dihydropyrimidin-1(2H)-yl]phenoxy}phenoxy)acetic acid (1.000 g, 1.86 mmol) together with bromoacetonitrile (337 mg, 2.81 mmol) and triethylamine (380 mg, 3.75 mmol) in 22 ml of dichloromethane under argon was stirred at room temperature for 5 hours. The mixture was left to stand overnight, 10 ml of water was added, and then the mixture was stirred. The organic phase was removed and concentrated. Column chromatography purification of the crude product obtained on silica gel with an n-heptane/ethyl acetate gradient afforded cyanomethyl (2-{2-bromo-4-fluoro-5-[3-methyl-2,6-dioxo-4-(trifluoromethyl)-3,6-dihydropyrimidin-1(2H)-yl]phenoxy}phenoxy)acetate (598 mg, 55% of theory) in the form of a colorless oil.

¹H-NMR (CDCl₃ δ, ppm) 7.55 (d, 1H), 7.18-7.03 (m, 3H), 6.95 (dd, 1H), 6.64 (d, 1H), 6.29 (s, 1H), 4.76 (s, 2H), 4.75 (s, 2H), 3.50 (s, 3H).

### Ex. I.9-176: Cyanomethyl (2-{2-cyano-4-fluoro-5-[3-methyl-2,6-dioxo-4-(trifluoromethyl)-3,6-dihydropyrimidin-1(2H)-yl]phenoxy}phenoxy)acetate

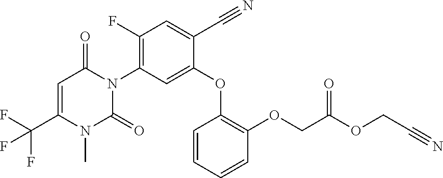 [SMILES: Cn1c(C(F)(F)F)cc(=O)n(-c2cc(Oc3ccccc3OCC(=O)OCC#N)c(C#N)cc2F)c1=O]

An initial charge of cyanomethyl (2-{2-bromo-4-fluoro-5-[3-methyl-2,6-dioxo-4-(trifluoromethyl)-3,6-dihydropyrimidin-1(2H)-yl]phenoxy}phenoxy)acetate (598 mg, 1.05 mmol) together with zinc cyanide (129 mg, 1.10 mmol) and tetrakis(triphenylphosphine)palladium (0) (121 mg, 0.10 mmol) in 15 ml of N,N-dimethylacetamide under argon was stirred at 180 degrees Celsius for 1 hour. After TLC monitoring, the reaction mixture was added to 10 ml of water and extracted with ethyl acetate. The combined organic phase was washed twice with saturated sodium chloride solution, then dried with sodium sulfate, filtered and then concentrated under reduced pressure. Column chromatography purification of the crude product obtained on silica gel with an n-heptane/ethyl acetate gradient afforded cyanomethyl (2-{2-cyano-4-fluoro-5-[3-methyl-2,6-dioxo-4-(trifluoromethyl)-3,6-dihydropyrimidin-1(2H)-yl]phenoxy}phenoxy)acetate (232 mg, 42% of theory) in the form of a colorless oil.

¹H-NMR (CDCl₃ δ, ppm) 7.53 (d, 1H), 7.23 (m, 2H), 7.09 (dt, 1H), 6.90 (d, 1H), 6.68 (d, 1H), 6.29 (s, 1H), 4.75 (s, 2H), 4.71 (s, 2H), 3.50 (s, 3H).

In analogy to the preparation examples cited above and recited at the appropriate point, and taking account of the general details relating to the preparation of substituted N-heterocyclyl- and N-heteroaryltetrahydropyrimidinones, the compounds cited below are obtained:

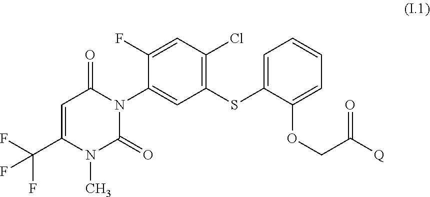

Table I.1: Preferred compounds of the formula (I.1) are the compounds I.1-1 to I.1-406 in which Q has the meanings from table 1 indicated in the respective row. Thus, the compounds I.1-1 to I.1-406 of table I.1 are defined by the meaning of the respective entry nos. 1 to 406 for Q from table 1.

TABLE 1

| No. | Q |
|---|---|
| 1 | Q-1 |
| 2 | Q-2 |
| 3 | Q-3 |
| 4 | Q-4 |
| 5 | Q-5 |
| 6 | Q-6 |
| 7 | Q-7 |
| 8 | Q-8 |
| 9 | Q-9 |
| 10 | Q-10 |
| 11 | Q-11 |
| 12 | Q-12 |
| 13 | Q-13 |
| 14 | Q-14 |
| 15 | Q-15 |
| 16 | Q-16 |
| 17 | Q-17 |
| 18 | Q-18 |
| 19 | Q-19 |
| 20 | Q-20 |
| 21 | Q-21 |
| 22 | Q-22 |
| 23 | Q-23 |
| 24 | Q-24 |
| 25 | Q-25 |
| 26 | Q-26 |
| 27 | Q-27 |
| 28 | Q-28 |
| 29 | Q-29 |
| 30 | Q-30 |
| 31 | Q-31 |
| 32 | Q-32 |
| 33 | Q-33 |
| 34 | Q-34 |
| 35 | Q-35 |
| 36 | Q-36 |
| 37 | Q-37 |
| 38 | Q-38 |
| 39 | Q-39 |
| 40 | Q-40 |
| 41 | Q-41 |
| 42 | Q-42 |
| 43 | Q-43 |
| 44 | Q-44 |
| 45 | Q-45 |
| 46 | Q-46 |
| 47 | Q-47 |
| 48 | Q-48 |
| 49 | Q-49 |
| 50 | Q-50 |
| 51 | Q-51 |
| 52 | Q-52 |
| 53 | Q-53 |
| 54 | Q-54 |
| 55 | Q-55 |
| 56 | Q-56 |
| 57 | Q-57 |
| 58 | Q-58 |
| 59 | Q-59 |
| 60 | Q-60 |
| 61 | Q-61 |
| 62 | Q-62 |
| 63 | Q-63 |
| 64 | Q-64 |
| 65 | Q-65 |
| 66 | Q-66 |
| 67 | Q-67 |
| 68 | Q-68 |
| 69 | Q-69 |
| 70 | Q-70 |
| 71 | Q-71 |
| 72 | Q-72 |
| 73 | Q-73 |
| 74 | Q-74 |
| 75 | Q-75 |
| 76 | Q-76 |
| 77 | Q-77 |
| 78 | Q-78 |
| 79 | Q-79 |
| 80 | Q-80 |
| 81 | Q-81 |
| 82 | Q-82 |
| 83 | Q-83 |
| 84 | Q-84 |
| 85 | Q-85 |
| 86 | Q-86 |
| 87 | Q-87 |
| 88 | Q-88 |
| 89 | Q-89 |
| 90 | Q-90 |
| 91 | Q-91 |
| 92 | Q-92 |
| 93 | Q-93 |
| 94 | Q-94 |
| 95 | Q-95 |
| 96 | Q-96 |
| 97 | Q-97 |
| 98 | Q-98 |
| 99 | Q-99 |
| 100 | Q-100 |
| 101 | Q-101 |
| 102 | Q-102 |
| 103 | Q-103 |
| 104 | Q-104 |
| 105 | Q-105 |
| 106 | Q-106 |
| 107 | Q-107 |
| 108 | Q-108 |
| 109 | Q-109 |
| 110 | Q-110 |
| 111 | Q-111 |
| 112 | Q-112 |
| 113 | Q-113 |
| 114 | Q-114 |
| 115 | Q-115 |
| 116 | Q-116 |
| 117 | Q-117 |
| 118 | Q-118 |
| 119 | Q-119 |
| 120 | Q-120 |
| 121 | Q-121 |
| 122 | Q-122 |
| 123 | Q-123 |
| 124 | Q-124 |
| 125 | Q-125 |
| 126 | Q-126 |
| 127 | Q-127 |
| 128 | Q-128 |
| 129 | Q-129 |
| 130 | Q-130 |
| 131 | Q-131 |
| 132 | Q-132 |
| 133 | Q-133 |
| 134 | Q-134 |
| 135 | Q-135 |
| 136 | Q-136 |
| 137 | Q-137 |
| 138 | Q-138 |
| 139 | Q-139 |
| 140 | Q-140 |
| 141 | Q-141 |
| 142 | Q-142 |
| 143 | Q-143 |
| 144 | Q-144 |
| 145 | Q-145 |
| 146 | Q-146 |
| 147 | Q-147 |
| 148 | Q-148 |
| 149 | Q-149 |
| 150 | Q-150 |
| 151 | Q-151 |
| 152 | Q-152 |
| 153 | Q-153 |
| 154 | Q-154 |
| 155 | Q-155 |
| 156 | Q-156 |
| 157 | Q-157 |
| 158 | Q-158 |
| 159 | Q-159 |
| 160 | Q-160 |
| 161 | Q-161 |
| 162 | Q-162 |
| 163 | Q-163 |
| 164 | Q-164 |
| 165 | Q-165 |
| 166 | Q-166 |
| 167 | Q-167 |
| 168 | Q-168 |
| 169 | Q-169 |
| 170 | Q-170 |
| 171 | Q-171 |
| 172 | Q-172 |
| 173 | Q-173 |
| 174 | Q-174 |
| 175 | Q-175 |
| 176 | Q-176 |
| 177 | Q-177 |
| 178 | Q-178 |
| 179 | Q-179 |
| 180 | Q-180 |
| 181 | Q-181 |
| 182 | Q-182 |
| 183 | Q-183 |
| 184 | Q-184 |
| 185 | Q-185 |
| 186 | Q-186 |
| 187 | Q-187 |
| 188 | Q-188 |
| 189 | Q-189 |
| 190 | Q-190 |
| 191 | Q-191 |
| 192 | Q-192 |
| 193 | Q-193 |
| 194 | Q-194 |
| 195 | Q-195 |
| 196 | Q-196 |
| 197 | Q-197 |
| 198 | Q-198 |
| 199 | Q-199 |
| 200 | Q-200 |
| 201 | Q-201 |
| 202 | Q-202 |
| 203 | Q-203 |
| 204 | Q-204 |
| 205 | Q-205 |
| 206 | Q-206 |
| 207 | Q-207 |
| 208 | Q-208 |
| 209 | Q-209 |
| 210 | Q-210 |
| 211 | Q-211 |
| 212 | Q-212 |
| 213 | Q-213 |
| 214 | Q-214 |
| 215 | Q-215 |
| 216 | Q-216 |
| 217 | Q-217 |
| 218 | Q-218 |
| 219 | Q-219 |
| 220 | Q-220 |
| 221 | Q-221 |
| 222 | Q-222 |
| 223 | Q-223 |
| 224 | Q-224 |
| 225 | Q-225 |
| 226 | Q-226 |
| 227 | Q-227 |
| 228 | Q-228 |
| 229 | Q-229 |
| 230 | Q-230 |
| 231 | Q-231 |
| 232 | Q-232 |
| 233 | Q-233 |
| 234 | Q-234 |
| 235 | Q-235 |
| 236 | Q-236 |
| 237 | Q-237 |
| 238 | Q-238 |
| 239 | Q-239 |
| 240 | Q-240 |
| 241 | Q-241 |
| 242 | Q-242 |
| 243 | Q-243 |
| 244 | Q-244 |
| 245 | Q-245 |
| 246 | Q-246 |
| 247 | Q-247 |
| 248 | Q-248 |
| 249 | Q-249 |
| 250 | Q-250 |
| 251 | Q-251 |
| 252 | Q-252 |
| 253 | Q-253 |
| 254 | Q-254 |
| 255 | Q-255 |
| 256 | Q-256 |
| 257 | Q-257 |
| 258 | Q-258 |
| 259 | Q-259 |
| 260 | Q-260 |
| 261 | Q-261 |
| 262 | Q-262 |
| 263 | Q-263 |
| 264 | Q-264 |
| 265 | Q-265 |
| 266 | Q-266 |
| 267 | Q-267 |
| 268 | Q-268 |
| 269 | Q-269 |
| 270 | Q-270 |
| 271 | Q-271 |
| 272 | Q-272 |
| 273 | Q-273 |
| 274 | Q-274 |
| 275 | Q-275 |
| 276 | Q-276 |
| 277 | Q-277 |
| 278 | Q-278 |
| 279 | Q-279 |
| 280 | Q-280 |
| 281 | Q-281 |
| 282 | Q-282 |
| 283 | Q-283 |
| 284 | Q-284 |
| 285 | Q-285 |
| 286 | Q-286 |
| 287 | Q-287 |
| 288 | Q-288 |
| 289 | Q-289 |
| 290 | Q-290 |
| 291 | Q-291 |
| 292 | Q-292 |
| 293 | Q-293 |
| 294 | Q-294 |
| 295 | Q-295 |
| 296 | Q-296 |
| 297 | Q-297 |
| 298 | Q-298 |
| 299 | Q-299 |
| 300 | Q-300 |
| 301 | Q-301 |
| 302 | Q-302 |
| 303 | Q-303 |
| 304 | Q-304 |
| 305 | Q-305 |
| 306 | Q-306 |
| 307 | Q-307 |
| 308 | Q-308 |
| 309 | Q-309 |
| 310 | Q-310 |
| 311 | Q-311 |
| 312 | Q-312 |
| 313 | Q-313 |
| 314 | Q-314 |
| 315 | Q-315 |
| 316 | Q-316 |
| 317 | Q-317 |
| 318 | Q-318 |
| 319 | Q-319 |
| 320 | Q-320 |
| 321 | Q-321 |
| 322 | Q-322 |
| 323 | Q-323 |
| 324 | Q-324 |
| 325 | Q-325 |
| 326 | Q-326 |
| 327 | Q-327 |
| 328 | Q-328 |
| 329 | Q-329 |
| 330 | Q-330 |
| 331 | Q-331 |
| 332 | Q-332 |
| 333 | Q-333 |
| 334 | Q-334 |
| 335 | Q-335 |
| 336 | Q-336 |
| 337 | Q-337 |
| 338 | Q-338 |
| 339 | Q-339 |
| 340 | Q-340 |
| 341 | Q-341 |
| 342 | Q-342 |
| 343 | Q-343 |
| 344 | Q-344 |
| 345 | Q-345 |
| 346 | Q-346 |
| 347 | Q-347 |
| 348 | Q-348 |
| 349 | Q-349 |
| 350 | Q-350 |
| 351 | Q-351 |
| 352 | Q-352 |
| 353 | Q-353 |
| 354 | Q-354 |
| 355 | Q-355 |
| 356 | Q-356 |
| 357 | Q-357 |
| 358 | Q-358 |
| 359 | Q-359 |
| 360 | Q-360 |
| 361 | Q-361 |
| 362 | Q-362 |
| 363 | Q-363 |
| 364 | Q-364 |
| 365 | Q-365 |
| 366 | Q-366 |
| 367 | Q-367 |
| 368 | Q-368 |
| 369 | Q-369 |
| 370 | Q-370 |
| 371 | Q-371 |
| 372 | Q-372 |
| 373 | Q-373 |
| 374 | Q-374 |
| 375 | Q-375 |
| 376 | Q-376 |
| 377 | Q-377 |
| 378 | Q-378 |
| 379 | Q-379 |
| 380 | Q-380 |
| 381 | Q-381 |
| 382 | Q-382 |
| 383 | Q-383 |
| 384 | Q-384 |
| 385 | Q-385 |
| 386 | Q-386 |
| 387 | Q-387 |
| 388 | Q-388 |
| 389 | Q-389 |
| 390 | Q-390 |
| 391 | Q-391 |
| 392 | Q-392 |
| 393 | Q-393 |
| 394 | Q-394 |
| 395 | Q-395 |
| 396 | Q-396 |
| 397 | Q-397 |
| 398 | Q-398 |
| 399 | Q-399 |
| 400 | Q-400 |
| 401 | Q-401 |
| 402 | Q-402 |
| 403 | Q-403 |
| 404 | Q-404 |
| 405 | Q-405 |
| 406 | Q-406 |

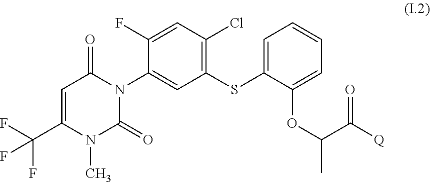

Table I.2: Preferred compounds of the formula (I.2) are the compounds I.2-1 to I.2-406 in which Q has the meanings from table 1 indicated in the respective row. Thus, the compounds I.2-1 to I.2-406 of table I.2 are defined by the meaning of the respective entry nos. 1 to 406 for Q from table 1.

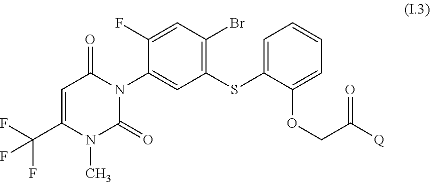

Table I.3: Preferred compounds of the formula (I.3) are the compounds I.3-1 to I.3-406 in which Q has the meanings from table 1 indicated in the respective row. Thus, the compounds I.3-1 to I.3-406 of table I.3 are defined by the meaning of the respective entry nos. 1 to 406 for Q from table 1.

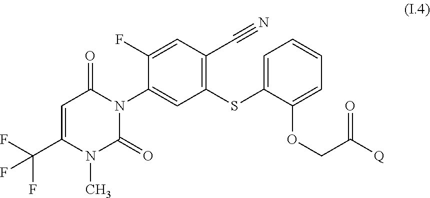

Table I.4: Preferred compounds of the formula (I.4) are the compounds I.4-1 to I.4-406 in which Q has the meanings from table 1 indicated in the respective row. Thus, the compounds I.4-1 to I.4-406 of table I.4 are defined by the meaning of the respective entry nos. 1 to 406 for Q from table 1.

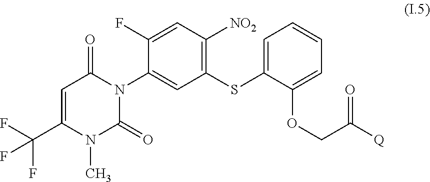

Table I.5: Preferred compounds of the formula (I.5) are the compounds I.5-1 to I.5-406 in which Q has the meanings from table 1 indicated in the respective row. Thus, the compounds I.5-1 to I.5-406 of table I.5 are defined by the meaning of the respective entry nos. 1 to 406 for Q from table 1.

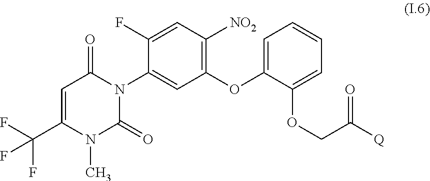

Table I.6: Preferred compounds of the formula (I.6) are the compounds I.6-1 to I.6-406 in which Q has the meanings from table 1 indicated in the respective row. Thus, the compounds I.6-1 to I.6-406 of table I.6 are defined by the meaning of the respective entry nos. 1 to 406 for Q from table 1.

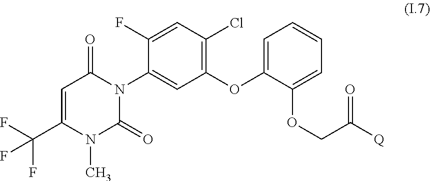

Table I.7: Preferred compounds of the formula (I.7) are the compounds I.7-1 to I.7-406 in which Q has the meanings from table 1 indicated in the respective row. Thus, the compounds I.7-1 to I.7-406 of table I.7 are defined by the meaning of the respective entry nos. 1 to 406 for Q from table 1.

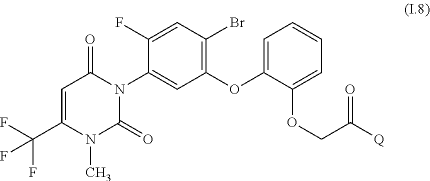

Table I.8: Preferred compounds of the formula (I.8) are the compounds I.8-1 to I.8-406 in which Q has the meanings from table 1 indicated in the respective row. Thus, the compounds I.8-1 to I.8-406 of table I.8 are defined by the meaning of the respective entry nos. 1 to 406 for Q from table 1.

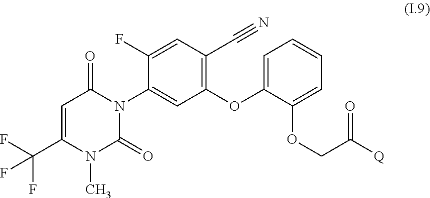

Table I.9: Preferred compounds of the formula (I.9) are the compounds I.9-1 to I.9-406 in which Q has the meanings from table 1 indicated in the respective row. Thus, the compounds I.9-1 to I.9-406 of table I.9 are defined by the meaning of the respective entry nos. 1 to 406 for Q from table 1.

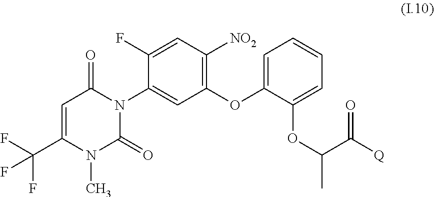

Table I.10: Preferred compounds of the formula (I.10) are the compounds I.10-1 to I.10-406 in which Q has the meanings from table 1 indicated in the respective row. Thus, the compounds I.10-1 to I.10-406 of table I.10 are defined by the meaning of the respective entry nos. 1 to 406 for Q from table 1.

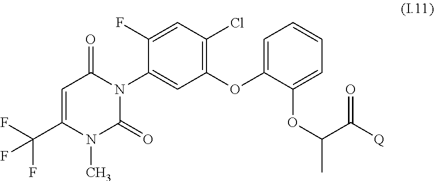

Table I.11: Preferred compounds of the formula (I.11) are the compounds I.11-1 to I.11-406 in which Q has the meanings from table 1 indicated in the respective row. Thus, the compounds I.11-1 to I.11-406 of table I.11 are defined by the meaning of the respective entry nos. 1 to 406 for Q from table 1.

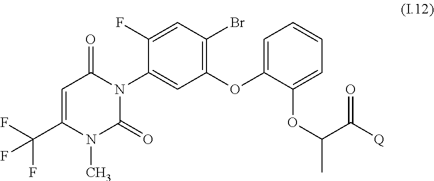

Table I.12: Preferred compounds of the formula (I.12) are the compounds I.12-1 to I.12-406 in which Q has the meanings from table 1 indicated in the respective row. Thus, the compounds I.12-1 to I.12-406 of table I.12 are defined by the meaning of the respective entry nos. 1 to 406 for Q from table 1.

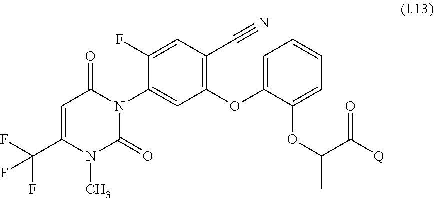

Table I.13: Preferred compounds of the formula (I.13) are the compounds I.13-1 to I.13-406 in which Q has the meanings from table 1 indicated in the respective row. Thus, the compounds I.13-1 to I.13-406 of table I.13 are defined by the meaning of the respective entry nos. 1 to 406 for Q from table 1.

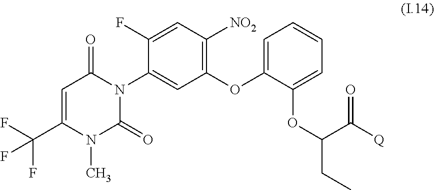

Table I.14: Preferred compounds of the formula (I.14) are the compounds I.14-1 to I.14-406 in which Q has the meanings from table 1 indicated in the respective row. Thus, the compounds I.14-1 to I.14-406 of table I.14 are defined by the meaning of the respective entry nos. 1 to 406 for Q from table 1.

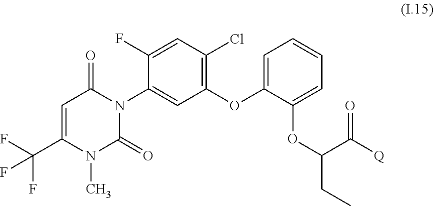

Table I.15: Preferred compounds of the formula (I.15) are the compounds I.15-1 to I.15-406 in which Q has the meanings from table 1 indicated in the respective row. Thus, the compounds I.15-1 to I.15-406 of table I.15 are defined by the meaning of the respective entry nos. 1 to 406 for Q from table 1.

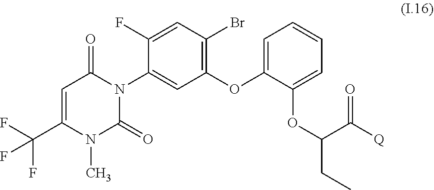

Table I.16: Preferred compounds of the formula (I.16) are the compounds I.16-1 to I.16-406 in which Q has the meanings from table 1 indicated in the respective row. Thus, the compounds I.16-1 to I.16-406 of table I.16 are defined by the meaning of the respective entry nos. 1 to 406 for Q from table 1.

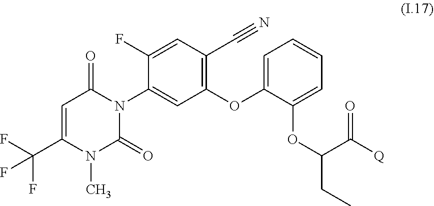

Table I.17: Preferred compounds of the formula (I.17) are the compounds I.17-1 to I.17-406 in which Q has the meanings from table 1 indicated in the respective row. Thus, the compounds I.17-1 to I.17-406 of table I.17 are defined by the meaning of the respective entry nos. 1 to 406 for Q from table 1.

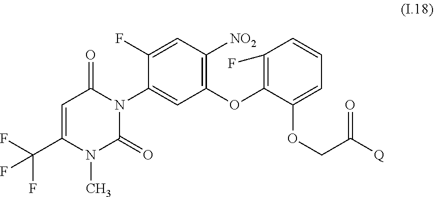

Table I.18: Preferred compounds of the formula (I.18) are the compounds I.18-1 to I.18-406 in which Q has the meanings from table 1 indicated in the respective row. Thus, the compounds I.18-1 to I.18-406 of table I.18 are defined by the meaning of the respective entry nos. 1 to 406 for Q from table 1.

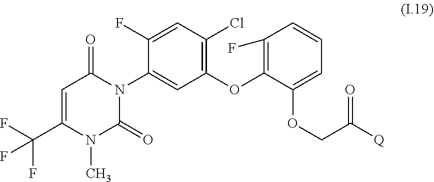

Table I.19: Preferred compounds of the formula (I.19) are the compounds I.19-1 to I.19-406 in which Q has the meanings from table 1 indicated in the respective row. Thus, the compounds I.19-1 to I.19-406 of table I.19 are defined by the meaning of the respective entry nos. 1 to 406 for Q from table 1.

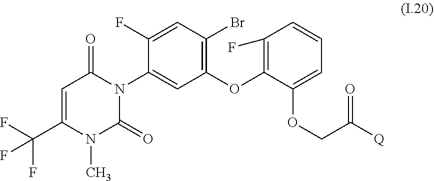

Table I.20: Preferred compounds of the formula (I.20) are the compounds I.20-1 to I.20-406 in which Q has the meanings from table 1 indicated in the respective row. Thus, the compounds I.20-1 to I.20-406 of table I.20 are defined by the meaning of the respective entry nos. 1 to 406 for Q from table 1.

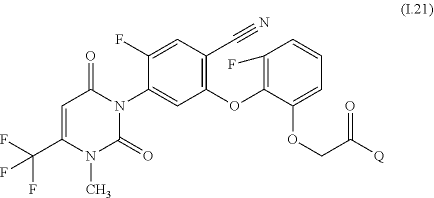

Table I.21: Preferred compounds of the formula (I.21) are the compounds I.21-1 to I.21-406 in which Q has the meanings from table 1 indicated in the respective row. Thus, the compounds I.21-1 to I.21-406 of table I.21 are defined by the meaning of the respective entry nos. 1 to 406 for Q from table 1.

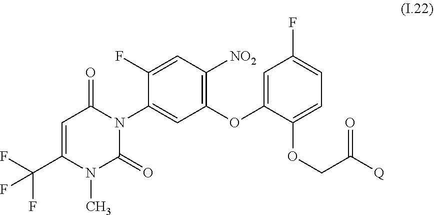

Table I.22: Preferred compounds of the formula (I.22) are the compounds I.22-1 to I.22-406 in which Q has the meanings from table 1 indicated in the respective row. Thus, the compounds I.22-1 to I.22-406 of table I.22 are defined by the meaning of the respective entry nos. 1 to 406 for Q from table 1.

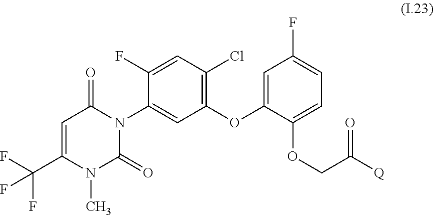

Table I.23: Preferred compounds of the formula (I.23) are the compounds I.23-1 to I.23-406 in which Q has the meanings from table 1 indicated in the respective row. Thus, the compounds I.23-1 to I.23-406 of table I.23 are defined by the meaning of the respective entry nos. 1 to 406 for Q from table 1.

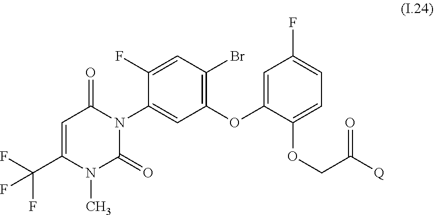

Table I.24: Preferred compounds of the formula (I.24) are the compounds I.24-1 to I.24-406 in which Q has the meanings from table 1 indicated in the respective row. Thus, the compounds I.24-1 to I.24-406 of table I.24 are defined by the meaning of the respective entry nos. 1 to 406 for Q from table 1.

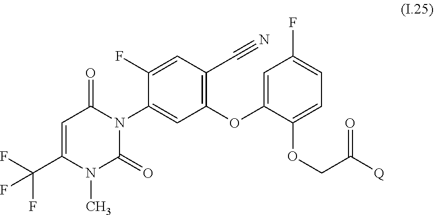

Table I.25: Preferred compounds of the formula (I.25) are the compounds I.25-1 to I.25-406 in which Q has the meanings from table 1 indicated in the respective row. Thus, the compounds I.25-1 to I.25-406 of table I.25 are defined by the meaning of the respective entry nos. 1 to 406 for Q from table 1.

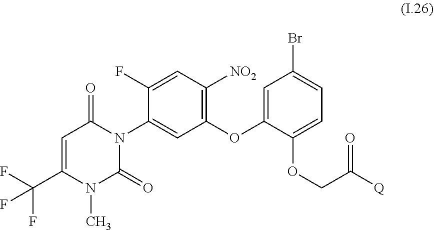

Table I.26: Preferred compounds of the formula (I.26) are the compounds I.26-1 to I.26-406 in which Q has the meanings from table 1 indicated in the respective row. Thus, the compounds I.26-1 to I.22-406 of table I.26 are defined by the meaning of the respective entry nos. 1 to 406 for Q from table 1.

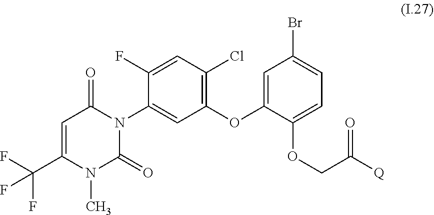

Table I.27: Preferred compounds of the formula (I.27) are the compounds I.27-1 to I.27-406 in which Q has the meanings from table 1 indicated in the respective row. Thus, the compounds I.27-1 to I.27-406 of table I.27 are defined by the meaning of the respective entry nos. 1 to 406 for Q from table 1.

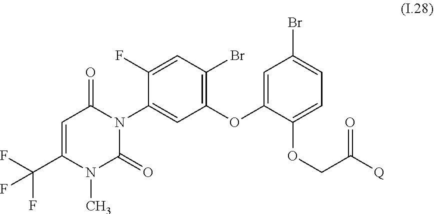

Table I.28: Preferred compounds of the formula (I.28) are the compounds I.28-1 to I.28-406 in which Q has the meanings from table 1 indicated in the respective row. Thus, the compounds I.28-1 to I.28-406 of table I.28 are defined by the meaning of the respective entry nos. 1 to 406 for Q from table 1.

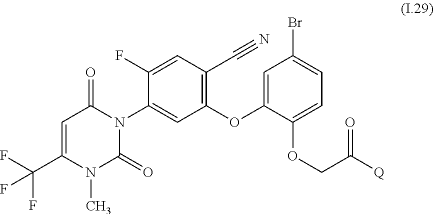

Table I.29: Preferred compounds of the formula (I.29) are the compounds I.29-1 to I.29-406 in which Q has the meanings from table 1 indicated in the respective row. Thus, the compounds I.29-1 to I.29-406 of table I.29 are defined by the meaning of the respective entry nos. 1 to 406 for Q from table 1.

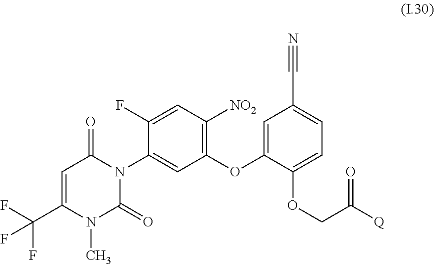

Table I.30: Preferred compounds of the formula (I.30) are the compounds I.30-1 to I.30-406 in which Q has the meanings from table 1 indicated in the respective row. Thus, the compounds I.30-1 to I.30-406 of table I.30 are defined by the meaning of the respective entry nos. 1 to 406 for Q from table 1.

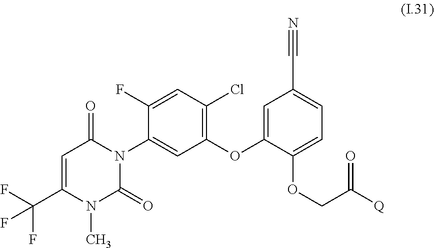

Table I.31: Preferred compounds of the formula (I.31) are the compounds I.31-1 to I.31-406 in which Q has the meanings from table 1 indicated in the respective row. Thus, the compounds I.31-1 to I.31-406 of table I.31 are defined by the meaning of the respective entry nos. 1 to 406 for Q from table 1.

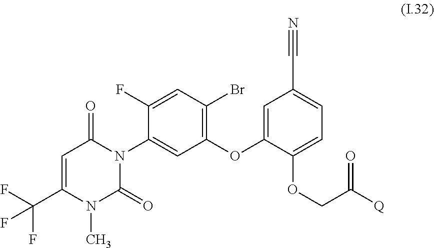

Table I.32: Preferred compounds of the formula (I.32) are the compounds I.32-1 to I.32-406 in which Q has the meanings from table 1 indicated in the respective row. Thus, the compounds I.32-1 to I.32-406 of table I.32 are defined by the meaning of the respective entry nos. 1 to 406 for Q from table 1.

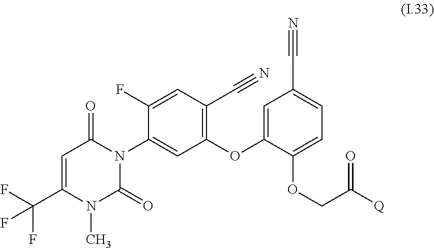

Table I.33: Preferred compounds of the formula (I.33) are the compounds I.33-1 to I.33-406 in which Q has the meanings from table 1 indicated in the respective row. Thus, the compounds I.33-1 to I.33-406 of table I.33 are defined by the meaning of the respective entry nos. 1 to 406 for Q from table 1.

NMR data of selected examples: The ¹H NMR data of selected examples of compounds of the general formula (I) are stated in two different ways, namely (a) conventional NMR interpretation or (b) in the form of 1H NMR peak lists according to the method described below.

a) Conventional NMR Interpretation

Ex. I.7-23: ¹H-NMR (CDCl₃ δ, ppm): 7.36 (d, 1H), 7.14-7.10 (m, 1H), 7.07-7.04 (m, 1H), 7.01-6.99 (m, 1H), 6.94-6.91 (m, 1H), 6.77 (d, 1H), 6.28 (s, 1H), 4.69 (s, 2H), 4.31-4.29 (m, 2H), 3.69-3.67 (m, 2H), 3.61-3.59 (m, 2H), 3.52-3.50 (m, 2H), 3.37 (s, 3H).

Ex. I.7-121: ¹H-NMR (CDCl₃ δ, ppm): 7.37 (d, 1H), 7.14-7.10 (m, 1H), 7.07-7.05 (m, 1H), 7.02-6.98 (m, 1H), 6.91 (d, 1H), 6.74-6.71 (m, 1H), 6.28 (s, 1H), 5.36-5.33 (m, 1H), 4.65 (s, 2H), 3.88-3.77 (m, 4H), 3.50 (s, 3H), 2.17-2.10 (m, 1H), 1.99-1.95 (m, 1H).

Ex. I.7-286: ¹H-NMR (CDCl₃ δ, ppm): 8.47 (d, 1H), 7.69 (d, 1H), 7.33 (d, 1H), 7.26-7.22 (m, 1H), 7.13-6.96 (m, 4H), 6.80 (d, 1H), 6.26 (s, 1H), 5.30 (s, 2H), 4.82 (s, 2H), 3.48 (s, 3H).

Ex. I.7-335: ¹H-NMR (CDCl₃ δ, ppm): 8.65 (s, 1H), 7.75 (d, 1H), 7.67 (d, 1H), 7.32 (d, 1H), 7.12 (t, 1H), 7.07 (d, 1H), 7.03 (t, 1H), 6.93 (d, 1H), 6.68 (d, 1H), 6.28 (s, 1H), 5.24 (s, 2H), 4.71 (s, 2H), 3.50 (s, 3H).

Ex. I.8-335: ¹H-NMR (CDCl₃ δ, ppm): 8.65 (s, 1H), 7.75 (d, 1H), 7.66 (d, 1H), 7.47 (d, 1H), 7.13 (t, 1H), 7.08 (d, 1H), 7.04 (t, 1H), 6.93 (d, 1H), 6.65 (d, 1H), 6.29 (s, 1H), 5.24 (s, 2H), 4.71 (s, 2H), 3.50 (s, 3H).

Ex. I.9-367: ¹H-NMR (CDCl₃ δ, ppm): 7.46 (d, 1H), 7.22 (d, 1H), 7.15 (t, 1H), 7.07 (s, 1H), 7.03 (t, 1H), 6.85 (d, 1H), 6.80 (d, 1H), 6.25 (s, 1H), 5.19 (s, 2H), 4.68 (s, 2H), 3.48 (s, 3H), 2.69 (s, 3H).

Ex. I.23-365: ¹H-NMR (CDCl₃ δ, ppm): 7.86 (s, 1H), 7.69 (s, 1H), 7.37 (d, 1H), 6.94-6.90 (m, 1H), 6.84 (d, 1H), 6.81-6.73 (m, 2H), 6.30 (s, 1H), 5.13 (s, 2H), 4.67 (s, 2H), 3.52 (s, 3H).

Ex. I.23-367: ¹H-NMR (CDCl₃ δ, ppm): 7.36 (d, 1H), 7.11 (s, 1H), 6.95-6.90 (m, 1H), 6.86 (d, 1H), 6.79-6.74 (m, 2H), 6.29 (s, 1H), 5.23 (s, 2H), 4.68 (s, 2H), 3.51 (s, 3H), 2.71 (s, 3H).

Ex. I.20-176: ¹H-NMR (CDCl₃ δ, ppm): 7.55 (d, 1H), 7.16-7.12 (m, 1H), 6.94-6.89 (m, 1H), 6.70-6.68 (m, 1H), 6.57 (d, 1H), 6.28 (s, 1H), 4.75 (s, 2H), 4.73 (s, 2H), 3.50 (s, 3H).

Ex. I.20-1: ¹H-NMR (CDCl₃ δ, ppm): 7.52 (d, 1H), 7.11-7.08 (m, 1H), 6.88-6.85 (m, 1H), 6.69 (d, 1H), 6.68-6.66 (m, 1H), 6.26 (s, 1H), 4.69 (s, 2H), 4-28-4.25 (m, 2H), 3.55-3.52 (m, 2H), 3.49 (s, 3H), 3.32 (s, 3H).

b) NMR Peak List Method

The 1H NMR data of selected examples are noted in the form of 1H NMR peak lists. For each signal peak, first the δ value in ppm and then the signal intensity in round brackets are listed. The δ value/signal intensity number pairs for different signal peaks are listed with separation from one another by semicolons.

The peak list for one example therefore has the form:

δ₁ (intensity₁); δ₂ (intensity₂); . . . ; δᵢ (intensityᵢ); . . . ; δₙ (intensityₙ)

The intensity of sharp signals correlates with the height of the signals in a printed example of an NMR spectrum in cm and shows the true ratios of the signal intensities. In the case of broad signals, several peaks or the middle of the signal and the relative intensity thereof may be shown in comparison to the most intense signal in the spectrum.

For calibration of the chemical shift of 1H NMR spectra we use tetramethylsilane and/or the chemical shift of the solvent, particularly in the case of spectra measured in DMSO. Therefore, the tetramethylsilane peak may but need not occur in NMR peak lists.

The lists of the 1H NMR peaks are similar to the conventional 1H NMR printouts and thus usually contain all peaks listed in a conventional NMR interpretation.

In addition, like conventional 1H NMR printouts, they may show solvent signals, signals of stereoisomers of the target compounds, which likewise form part of the subject matter of the invention, and/or peaks of impurities.

In the reporting of compound signals in the delta range of solvents and/or water, our lists of 1H NMR peaks show the usual solvent peaks, for example peaks of DMSO in DMSO-D₆ and the peak of water, which usually have a high intensity on average.

The peaks of stereoisomers of the target compounds and/or peaks of impurities usually have a lower intensity on average than the peaks of the target compounds (for example with a purity of >90%). Such stereoisomers and/or impurities may be typical of the particular preparation process. Their peaks can thus help in this case to identify reproduction of our preparation process with reference to “by-product fingerprints”.

An expert calculating the peaks of the target compounds by known methods (MestreC, ACD simulation, but also with empirically evaluated expected values) can, if required, isolate the peaks of the target compounds, optionally using additional intensity filters. This isolation would be similar to the relevant peak picking in conventional 1H NMR interpretation.

Further details of 1H NMR peak lists can be found in Research Disclosure Database Number 564025.

I.1-72: ¹H-NMR (400.0 MHz, CDCl3):

δ=7.5190 (1.5); 7.4207 (1.6); 7.4169 (2.3); 7.4142 (1.8); 7.4015 (1.8); 7.3983 (2.4); 7.3949 (1.8); 7.3405 (1.7); 7.3335 (5.3); 7.3198 (2.2); 7.3108 (5.3); 7.3013 (2.1); 7.2969 (1.8); 7.2601 (268.7); 7.0646 (2.8); 7.0579 (2.9); 7.0460 (2.9); 7.0392 (2.8); 7.0209 (1.8); 7.0181 (1.9); 7.0019 (3.1); 6.9992 (3.2); 6.9962 (1.7); 6.9830 (1.6); 6.9803 (1.5); 6.7737 (2.9); 6.7531 (2.7); 6.2969 (4.7); 6.2768 (4.6); 4.6694 (16.0); 4.1622 (1.0); 4.1457 (1.1); 4.1403 (1.0); 4.1353 (1.5); 4.1309 (1.8); 4.1239 (1.0); 4.1190 (1.5); 4.1132 (3.0); 4.0968 (1.6); 4.0500 (1.6); 4.0295 (3.0); 4.0231 (1.0); 4.0090 (1.6); 4.0027 (2.0); 3.9822 (1.1); 3.8550 (0.9); 3.8413 (1.0); 3.8340 (2.0); 3.8205 (2.1); 3.8137 (1.5); 3.7998 (1.4); 3.7678 (1.2); 3.7645 (1.2); 3.7457 (3.7); 3.7276 (3.6); 3.7082 (1.9); 3.6871 (1.1); 3.5296 (1.3); 3.5167 (1.5); 3.5016 (12.4); 3.4987 (12.9); 3.4875 (1.3); 3.4742 (1.1); 2.5594 (0.8); 2.5426 (1.0); 2.5246 (0.8); 2.0450 (7.2); 2.0196 (0.8); 2.0064 (1.0); 1.9876 (1.1); 1.9743 (0.8); 1.5985 (0.6); 1.5798 (1.0); 1.5668 (1.4); 1.5411 (10.0); 1.5160 (0.7); 1.3037 (0.8); 1.2773 (3.0); 1.2595 (6.0); 1.2416 (2.2); 0.8991 (1.9); 0.8821 (6.5); 0.8643 (2.4); 0.1459 (0.6); 0.0080 (4.7); −0.0002 (160.7); −0.0086 (4.6); −0.1496 (0.6)

I.2-72: ¹H-NMR (400.0 MHz, CDCl3):

δ=7.3961 (0.6); 7.3926 (0.5); 7.3849 (0.8); 7.3802 (0.9); 7.3765 (0.7); 7.3735 (0.6); 7.3658 (0.8); 7.3369 (1.3); 7.3311 (1.4); 7.3143 (1.3); 7.3085 (1.4); 7.2970 (0.5); 7.2930 (0.6); 7.2781 (1.0); 7.2738 (1.2); 7.2600 (35.9); 7.2549 (0.9); 7.1025 (0.8); 7.0913 (0.8); 7.0839 (0.7); 7.0727 (0.8); 7.0105 (0.7); 6.9969 (0.8); 6.9918 (0.8); 6.9881 (1.0); 6.9854 (1.0); 6.9785 (0.8); 6.9691 (1.6); 6.9664 (1.6); 6.9502 (0.8); 6.9475 (0.7); 6.7229 (0.7); 6.7091 (0.8); 6.7013 (0.7); 6.6886 (0.7); 6.2969 (1.3); 6.2825 (2.2); 6.2685 (1.2); 4.7637 (1.0); 4.7467 (1.1); 4.7323 (0.9); 4.1304 (0.7); 4.1126 (0.6); 4.0548 (0.6); 4.0495 (0.7); 4.0380 (0.6); 4.0298 (0.7); 4.0061 (0.8); 3.9791 (0.5); 3.8202 (0.7); 3.8072 (0.6); 3.7992 (0.7); 3.7287 (0.8); 3.7235 (1.1); 3.7114 (1.1); 3.7057 (1.1); 3.7009 (1.0); 3.6921 (0.9); 3.6827 (0.7); 3.4952 (5.7); 3.4829 (0.8); 3.4694 (0.7); 3.4601 (0.9); 3.4468 (0.9); 2.0449 (1.7); 1.5412 (16.0); 1.5152 (4.7); 1.5109 (4.3); 1.4981 (4.5); 1.4940 (4.1); 1.2771 (1.0); 1.2647 (1.9); 1.2594 (2.0); 1.2415 (0.6); 0.8990 (1.0); 0.8820 (3.6); 0.8643 (1.4); 0.0079 (1.4); −0.0002 (46.0); −0.0085 (1.3)

I.2-71: ¹H-NMR (400.0 MHz, CDCl3):

δ=7.4207 (0.5); 7.4095 (0.6); 7.4051 (0.7); 7.4014 (0.6); 7.3904 (0.6); 7.3234 (1.1); 7.3183 (1.1); 7.3008 (1.6); 7.2959 (1.2); 7.2813 (0.8); 7.2600 (37.4); 7.1072 (0.6); 7.0972 (0.6); 7.0885 (0.6); 7.0785 (0.6); 7.0224 (0.6); 7.0186 (0.6); 7.0037 (0.6); 6.9999 (0.6); 6.9792 (0.6); 6.9614 (1.1); 6.9423 (0.5); 6.7456 (0.8); 6.7254 (0.5); 6.2736 (1.7); 6.2666 (1.0); 6.2610 (1.0); 4.7857 (0.6); 4.7695 (0.6); 4.7570 (0.5); 4.1854 (0.5); 4.1308 (0.7); 4.1135 (0.6); 4.0803 (0.6); 4.0682 (0.8); 4.0633 (0.9); 4.0440 (0.9); 3.7892 (0.7); 3.7726 (0.8); 3.7601 (0.5); 3.7557 (0.5); 3.4894 (3.1); 3.4864 (3.4); 2.0452 (1.3); 1.8965 (0.6); 1.8910 (0.6); 1.8837 (0.6); 1.8774 (0.7); 1.8716 (0.8); 1.8605 (0.5); 1.8545 (0.5); 1.5387 (16.0); 1.5148 (3.3); 1.4977 (3.2); 1.2773 (0.8); 1.2644 (1.7); 1.2597 (1.8); 1.2417 (0.5); 0.8990 (0.9); 0.8820 (3.1); 0.8643 (1.2); 0.0080 (1.5); −0.0002 (47.9); −0.0085 (1.2)

I.2-23: ¹H-NMR (400.0 MHz, CDCl3):

δ=7.4172 (1.0); 7.4130 (1.1); 7.4071 (1.0); 7.4029 (1.1); 7.3980 (1.1); 7.3938 (1.1); 7.3879 (1.0); 7.3837 (1.1); 7.3265 (1.9); 7.3209 (1.9); 7.3038 (2.4); 7.2985 (2.4); 7.2845 (1.3); 7.2826 (1.4); 7.2806 (1.3); 7.2602 (32.9); 7.0934 (1.8); 7.0748 (1.8); 7.0059 (1.7); 6.9873 (1.8); 6.9828 (1.1); 6.9803 (1.1); 6.9639 (1.9); 6.9614 (1.9); 6.9451 (0.9); 6.9425 (0.9); 6.7504 (1.0); 6.7377 (1.0); 6.7323 (1.0); 6.7194 (0.9); 6.2753 (5.0); 4.7680 (1.2); 4.7547 (1.2); 4.7509 (1.2); 4.7377 (1.2); 4.2900 (0.7); 4.2784 (2.4); 4.2658 (2.8); 4.2563 (1.9); 3.6811 (2.9); 3.6691 (3.7); 3.6571 (2.6); 3.6049 (0.8); 3.6009 (1.0); 3.5991 (1.1); 3.5946 (2.3); 3.5887 (2.5); 3.5847 (1.4); 3.5812 (2.8); 3.5745 (2.1); 3.5202 (2.5); 3.5125 (3.5); 3.5071 (2.4); 3.4907 (7.0); 3.4887 (6.7); 3.3717 (16.0); 3.3690 (16.0); 1.5447 (13.3); 1.5076 (8.9); 1.4906 (8.8); 1.2648 (0.8); 0.8820 (1.4); 0.8642 (0.6); 0.0080 (1.2); −0.0002 (42.0); −0.0085 (1.2)

I.2-2: ¹H-NMR (400.0 MHz, CDCl3):

δ=7.4253 (0.8); 7.4210 (0.8); 7.4179 (0.8); 7.4135 (0.8); 7.4061 (0.8); 7.4020 (0.9); 7.3985 (0.8); 7.3945 (0.8); 7.3237 (1.5); 7.3191 (1.5); 7.3010 (1.9); 7.2966 (2.0); 7.2778 (1.2); 7.2600 (44.7); 7.0984 (1.4); 7.0798 (1.4); 7.0118 (1.4); 6.9932 (1.4); 6.9804 (0.9); 6.9638 (1.5); 6.9612 (1.5); 6.9425 (0.7); 6.7493 (0.8); 6.7401 (0.8); 6.7311 (0.7); 6.7192 (0.8); 6.2735 (2.3); 6.2657 (2.3); 4.7719 (1.0); 4.7579 (1.0); 4.7548 (1.0); 4.7410 (1.0); 4.2748 (0.6); 4.2635 (1.8); 4.2570 (1.2); 4.2512 (2.4); 4.2415 (1.2); 3.6034 (2.4); 3.5914 (2.8); 3.5793 (2.1); 3.5096 (0.8); 3.5045 (0.9); 3.4921 (5.8); 3.4870 (7.0); 3.4746 (2.6); 3.4695 (2.6); 3.4571 (0.8); 3.4519 (0.9); 1.5378 (16.0); 1.5110 (7.3); 1.4939 (7.1); 1.2647 (1.3); 1.1993 (2.6); 1.1936 (2.7); 1.1818 (5.4); 1.1761 (5.4); 1.1643 (2.6); 1.1586 (2.6); 0.8989 (0.7); 0.8820 (2.2); 0.8643 (0.9); 0.0080 (1.8); −0.0002 (58.2); −0.0085 (1.6)

I.2-58: ¹H-NMR (400.0 MHz, CDCl3):

δ=7.5189 (0.8); 7.4260 (1.6); 7.4218 (1.9); 7.4163 (1.6); 7.4122 (1.8); 7.4068 (2.0); 7.4027 (2.0); 7.3971 (1.8); 7.3930 (1.9); 7.3435 (3.2); 7.3365 (3.3); 7.3206 (4.0); 7.3141 (4.2); 7.2996 (2.4); 7.2975 (2.3); 7.2954 (2.2); 7.2792 (1.6); 7.2749 (1.6); 7.2601 (152.7); 7.0526 (3.0); 7.0340 (3.1); 7.0079 (1.3); 7.0050 (2.4); 7.0020 (1.3); 6.9937 (3.1); 6.9890 (2.2); 6.9861 (3.8); 6.9832 (2.1); 6.9751 (3.1); 6.9700 (1.1); 6.9672 (1.9); 6.9643 (1.0); 6.7996 (1.6); 6.7750 (2.2); 6.7532 (1.6); 6.2920 (5.2); 6.2876 (4.9); 4.8190 (0.6); 4.8021 (1.9); 4.7850 (2.0); 4.7797 (2.0); 4.7626 (2.0); 4.7456 (0.6); 4.2560 (0.6); 4.2360 (1.3); 4.2266 (2.2); 4.2224 (2.5); 4.2182 (2.0); 4.2133 (4.7); 4.2089 (1.9); 4.2049 (2.6); 4.2027 (2.1); 4.1994 (1.8); 4.1932 (1.5); 4.1729 (0.6); 4.1486 (0.6); 4.1308 (1.8); 4.1131 (1.8); 4.0950 (0.6); 3.7542 (1.7); 3.7448 (3.2); 3.7352 (2.5); 3.5010 (13.1); 3.4981 (13.4); 2.0449 (8.6); 1.5454 (16.0); 1.5420 (15.1); 1.5284 (11.0); 1.5250 (10.3); 1.2843 (1.4); 1.2772 (3.6); 1.2594 (7.5); 1.2415 (2.8); 0.8990 (2.1); 0.8820 (7.3); 0.8643 (2.8); 0.0080 (4.6); −0.0002 (153.4); −0.0085 (4.3); −0.1496 (0.5)

I.1-26: ¹H-NMR (400.0 MHz, CDCl3):

δ=7.4316 (1.0); 7.4275 (1.1); 7.4125 (1.1); 7.4083 (1.1); 7.3405 (0.5); 7.3362 (0.5); 7.3253 (2.0); 7.3218 (0.8); 7.3199 (0.8); 7.3175 (0.8); 7.3158 (0.7); 7.3025 (2.1); 7.2969 (0.6); 7.2600 (24.5); 7.0896 (1.8); 7.0710 (1.8); 7.0140 (0.6); 7.0113 (0.7); 6.9952 (1.2); 6.9923 (1.2); 6.9762 (0.5); 6.9734 (0.6); 6.7728 (1.0); 6.7703 (1.0); 6.7521 (0.9); 6.7496 (0.9); 6.2872 (3.0); 4.6610 (5.8); 4.2291 (0.7); 4.2235 (0.8); 4.2127 (1.6); 4.2073 (1.6); 4.1965 (0.8); 4.1911 (0.8); 3.5000 (4.1); 3.4971 (4.2); 3.3932 (1.7); 3.3779 (3.6); 3.3626 (1.8); 3.2982 (16.0); 2.0069 (0.6); 1.8938 (1.5); 1.8781 (2.2); 1.8624 (1.5); 1.5395 (9.6); 0.0080 (0.9); −0.0002 (32.4); −0.0085 (0.9)

I.1-23: ¹H-NMR (400.0 MHz, CDCl3):

δ=7.4227 (1.0); 7.4186 (1.1); 7.4036 (1.1); 7.3994 (1.1); 7.3379 (0.6); 7.3336 (0.6); 7.3253 (2.0); 7.3191 (0.8); 7.3173 (0.8); 7.3150 (0.8); 7.3131 (0.7); 7.3027 (2.0); 7.2986 (0.8); 7.2943 (0.6); 7.2606 (7.8); 7.0826 (1.8); 7.0639 (1.8); 7.0115 (0.7); 7.0086 (0.7); 6.9925 (1.2); 6.9897 (1.2); 6.9736 (0.6); 6.9708 (0.6); 6.7872 (1.0); 6.7847 (1.0); 6.7665 (1.0); 6.7640 (0.9); 6.2781 (3.0); 4.6947 (5.7); 4.3023 (1.5); 4.2935 (1.1); 4.2904 (1.6); 4.2867 (1.0); 4.2782 (1.6); 3.7020 (1.8); 3.6933 (1.1); 3.6899 (1.8); 3.6870 (1.1); 3.6780 (1.6); 3.6251 (1.2); 3.6172 (1.4); 3.6144 (1.6); 3.6093 (1.4); 3.6026 (2.4); 3.5366 (2.5); 3.5300 (1.4); 3.5244 (1.6); 3.5222 (1.5); 3.5135 (1.2); 3.4979 (4.2); 3.4951 (4.3); 3.3763 (16.0); 1.2844 (0.5); 1.2550 (1.5); −0.0002 (10.2)

I.1-71: ¹H-NMR (400.0 MHz, CDCl3):

δ=7.4740 (16.0); 7.4228 (0.8); 7.4186 (0.9); 7.4036 (0.9); 7.3995 (1.0); 7.3473 (0.7); 7.3453 (0.7); 7.3431 (0.7); 7.3412 (0.6); 7.3351 (1.7); 7.3267 (0.6); 7.3224 (0.6); 7.3126 (1.6); 7.1128 (0.9); 7.1103 (0.9); 7.0943 (0.9); 7.0916 (0.9); 7.0228 (0.6); 7.0200 (0.6); 7.0039 (1.0); 7.0011 (1.0); 6.9849 (0.5); 6.8135 (0.9); 6.7928 (0.8); 6.2879 (1.5); 6.2823 (1.4); 4.7135 (3.6); 4.1625 (0.6); 4.0841 (1.0); 4.0806 (0.7); 4.0775 (0.6); 4.0678 (0.9); 4.0534 (0.5); 3.8216 (0.6); 3.8165 (0.5); 3.7678 (0.6); 3.4989 (3.5); 2.7526 (7.9); 2.5968 (2.3); 2.5921 (4.7); 2.5874 (6.5); 2.5827 (4.5); 2.5780 (2.0); 1.9141 (0.7); 1.9107 (0.8); 1.8944 (0.9); 1.8778 (0.6); 0.0080 (0.8); −0.0002 (21.7); −0.0085 (0.6)

I.7-276: ¹H-NMR (400.6 MHz, CDCl3):

δ=8.5693 (1.3); 8.5653 (1.7); 8.5623 (1.3); 8.5577 (1.2); 8.5547 (1.6); 8.5505 (1.4); 7.6829 (1.2); 7.6785 (1.2); 7.6636 (2.1); 7.6592 (2.3); 7.6444 (1.4); 7.6400 (1.4); 7.3311 (4.6); 7.3089 (4.6); 7.2654 (0.9); 7.2611 (74.1); 7.2554 (0.9); 7.2396 (3.5); 7.2382 (3.7); 7.2262 (1.6); 7.2249 (1.5); 7.2195 (3.8); 7.2074 (1.3); 7.1368 (1.1); 7.1323 (1.3); 7.1183 (1.6); 7.1166 (1.7); 7.1139 (2.0); 7.1122 (2.0); 7.0983 (1.8); 7.0938 (2.3); 7.0860 (2.1); 7.0816 (2.1); 7.0659 (3.9); 7.0616 (2.8); 7.0194 (2.1); 7.0157 (2.6); 7.0010 (2.0); 6.9995 (1.7); 6.9975 (2.5); 6.9957 (1.9); 6.9810 (1.1); 6.9774 (1.1); 6.9599 (2.9); 6.9563 (2.5); 6.9396 (2.3); 6.9361 (2.0); 6.7627 (4.3); 6.7463 (4.5); 6.2439 (7.6); 5.2819 (12.6); 4.7754 (16.0); 4.7671 (0.8); 3.4786 (10.2); 3.4756 (10.6); 2.0451 (2.3); 1.5909 (1.2); 1.2772 (1.0); 1.2641 (1.1); 1.2594 (2.1); 1.2415 (0.8); 0.8988 (0.6); 0.8819 (2.2); 0.8643 (0.8); 0.0080 (1.3); 0.0041 (0.6); −0.0002 (46.0); −0.0058 (0.7); −0.0085 (1.3)

I.7-176: ¹H-NMR (400.6 MHz, CDCl3):

δ=7.3991 (3.0); 7.3770 (3.1); 7.2607 (35.1); 7.1781 (0.8); 7.1734 (0.9); 7.1599 (1.2); 7.1579 (1.2); 7.1553 (1.4); 7.1533 (1.2); 7.1398 (1.2); 7.1351 (1.5); 7.1160 (1.0); 7.1114 (1.3); 7.0960 (2.7); 7.0914 (2.0); 7.0672 (1.7); 7.0636 (1.7); 7.0490 (1.4); 7.0471 (1.2); 7.0455 (1.4); 7.0435 (1.0); 7.0290 (0.7); 7.0253 (0.6); 6.9524 (1.8); 6.9488 (1.7); 6.9321 (1.6); 6.9286 (1.4); 6.6854 (2.9); 6.6691 (2.9); 6.2899 (5.0); 4.7595 (16.0); 4.7450 (12.5); 4.1305 (0.6); 4.1127 (0.6); 3.5050 (7.2); 3.5020 (7.2); 2.0450 (3.0); 1.5507 (6.0); 1.2772 (1.1); 1.2594 (2.1); 1.2415 (0.9); 0.8820 (1.2); 0.0080 (0.6); −0.0002 (22.3); −0.0084 (0.7)

I.7-2: ¹H-NMR (400.2 MHz, d₆-DMSO):

δ=7.8200 (3.9); 7.7969 (3.9); 7.2054 (0.8); 7.2006 (0.9); 7.1878 (1.1); 7.1843 (1.7); 7.1801 (1.6); 7.1672 (1.6); 7.1625 (1.7); 7.1165 (2.5); 7.1133 (2.8); 7.0959 (1.7); 7.0924 (1.6); 7.0844 (1.3); 7.0797 (1.6); 7.0645 (3.7); 7.0598 (3.0); 7.0458 (2.1); 7.0424 (1.9); 7.0282 (1.8); 7.0249 (2.0); 7.0084 (0.8); 7.0047 (0.7); 6.9635 (3.7); 6.9468 (3.8); 6.5141 (7.5); 5.7551 (4.4); 4.7852 (11.1); 4.1967 (3.0); 4.1884 (2.7); 4.1850 (3.4); 4.1811 (2.6); 4.1731 (3.3); 3.5366 (3.6); 3.5283 (2.5); 3.5247 (3.6); 3.5130 (3.2); 3.4239 (2.3); 3.4064 (7.4); 3.3889 (7.6); 3.3714 (2.8); 3.3558 (10.5); 3.3270 (108.8); 2.6753 (0.5); 2.6707 (0.7); 2.6661 (0.5); 2.5242 (1.8); 2.5195 (2.8); 2.5108 (43.0); 2.5063 (87.8); 2.5018 (115.2); 2.4971 (81.0); 2.4926 (37.6); 2.3332 (0.5); 2.3286 (0.7); 2.3240 (0.5); 1.9796 (1.0); 1.2353 (0.4); 1.0801 (7.8); 1.0627 (16.0); 1.0451 (7.5); −0.0002 (2.7)

I.9-1: ¹H-NMR (400.6 MHz, CDCl3):

δ=7.5023 (1.5); 7.4812 (1.5); 7.2605 (20.7); 7.2282 (0.7); 7.2241 (1.0); 7.2152 (0.6); 7.2084 (0.8); 7.2046 (1.3); 7.1959 (1.0); 7.1908 (0.6); 7.1758 (0.8); 7.1717 (0.6); 7.0552 (0.7); 7.0516 (0.8); 7.0358 (1.1); 7.0322 (1.0); 7.0165 (0.5); 6.8821 (0.9); 6.8786 (0.9); 6.8616 (0.8); 6.8583 (0.8); 6.8085 (1.4); 6.7940 (1.5); 6.2670 (2.6); 4.6660 (4.2); 4.2621 (0.9); 4.2596 (0.9); 4.2527 (1.0); 4.2510 (1.0); 4.2476 (1.0); 4.2454 (1.0); 4.2388 (1.0); 4.2362 (0.9); 3.5452 (1.8); 3.5335 (1.8); 3.5218 (1.7); 3.4892 (3.7); 3.4862 (3.8); 3.3156 (16.0); 1.5412 (8.4); 0.0080 (0.9); −0.0002 (33.1); −0.0085 (0.9)

I.7-41: ¹H-NMR (400.6 MHz, CDCl3):

δ=7.3781 (4.2); 7.3559 (4.2); 7.2602 (38.3); 7.1490 (1.0); 7.1444 (1.1); 7.1306 (1.5); 7.1287 (1.5); 7.1261 (1.8); 7.1242 (1.6); 7.1105 (1.6); 7.1060 (1.9); 7.0834 (1.5); 7.0788 (1.8); 7.0633 (3.6); 7.0588 (2.8); 7.0303 (2.1); 7.0267 (2.3); 7.0120 (1.9); 7.0101 (1.6); 7.0085 (1.9); 7.0067 (1.4); 6.9921 (0.9); 6.9884 (0.9); 6.9363 (2.6); 6.9328 (2.4); 6.9160 (2.2); 6.9125 (1.9); 6.7255 (4.1); 6.7091 (4.1); 6.2769 (7.1); 4.7037 (16.0); 4.6945 (0.6); 4.3814 (2.2); 4.3701 (2.9); 4.3582 (2.7); 4.1411 (4.9); 4.1360 (1.2); 4.1318 (2.8); 4.1292 (4.7); 4.1231 (1.1); 4.1176 (4.0); 4.1128 (0.6); 3.4984 (9.9); 3.4955 (9.9); 2.0448 (1.9); 1.5500 (1.3); 1.2770 (0.9); 1.2728 (0.7); 1.2646 (1.2); 1.2593 (1.9); 1.2551 (0.8); 1.2414 (0.6); 0.8990 (0.6); 0.8820 (2.2); 0.8644 (0.8); 0.0079 (0.7); −0.0002 (24.0); −0.0085 (0.7)

I.7-278: ¹H-NMR (400.6 MHz, CDCl3):

δ=8.5830 (2.1); 8.5700 (2.1); 7.5193 (0.6); 7.3136 (4.2); 7.2916 (4.2); 7.2674 (0.7); 7.2666 (0.7); 7.2607 (106.8); 7.1909 (3.2); 7.1762 (3.1); 7.1503 (1.0); 7.1455 (1.2); 7.1322 (1.5); 7.1301 (1.5); 7.1275 (1.9); 7.1254 (1.7); 7.1121 (1.6); 7.1073 (2.1); 7.0927 (1.4); 7.0881 (1.7); 7.0726 (3.8); 7.0680 (2.8); 7.0469 (2.2); 7.0432 (2.6); 7.0288 (1.8); 7.0268 (1.5); 7.0253 (1.9); 7.0232 (1.5); 7.0088 (0.9); 7.0051 (0.9); 6.9972 (0.6); 6.9502 (2.5); 6.9466 (2.4); 6.9299 (2.1); 6.9264 (2.0); 6.7028 (3.9); 6.6864 (4.0); 6.2559 (7.0); 5.1861 (7.2); 4.7523 (16.0); 3.4885 (9.6); 3.4855 (10.2); 2.1716 (3.8); 2.0452 (1.3); 1.2773 (0.7); 1.2639 (1.2); 1.2595 (1.5); 0.8989 (0.6); 0.8820 (2.2); 0.8642 (0.8); 0.0080 (1.9); −0.0002 (68.0); −0.0085 (2.1)

I.7-277: ¹H-NMR (400.2 MHz, d6-DMSO):

δ=8.5668 (4.0); 8.5624 (4.0); 8.5424 (2.9); 8.5384 (3.0); 8.5305 (3.1); 8.5264 (3.0); 8.3150 (0.5); 7.7915 (6.0); 7.7684 (6.0); 7.7509 (1.6); 7.7457 (2.4); 7.7410 (1.5); 7.7312 (1.8); 7.7259 (2.7); 7.7215 (1.7); 7.4052 (2.4); 7.4035 (2.4); 7.3931 (2.4); 7.3914 (2.4); 7.3856 (2.2); 7.3839 (2.2); 7.3736 (2.1); 7.3719 (2.1); 7.1993 (0.9); 7.1946 (1.0); 7.1821 (1.1); 7.1784 (2.8); 7.1738 (2.4); 7.1617 (2.8); 7.1572 (3.2); 7.1466 (3.9); 7.1423 (4.6); 7.1258 (1.9); 7.1218 (1.3); 7.0877 (1.6); 7.0831 (2.3); 7.0676 (5.1); 7.0634 (4.0); 7.0497 (3.0); 7.0455 (2.8); 7.0326 (2.4); 7.0287 (2.8); 7.0254 (1.2); 7.0130 (1.1); 7.0085 (1.1); 6.9453 (5.7); 6.9286 (5.8); 6.5024 (11.5); 5.7552 (12.8); 5.1930 (16.0); 4.8511 (14.8); 3.3411 (16.8); 3.3293 (276.4); 3.1752 (0.4); 3.1620 (0.4); 2.6799 (0.3); 2.6754 (0.7); 2.6709 (1.1); 2.6663 (0.8); 2.6616 (0.3); 2.5244 (2.7); 2.5197 (4.2); 2.5110 (65.0); 2.5065 (134.8); 2.5019 (178.0); 2.4973 (125.5); 2.4927 (58.4); 2.3378 (0.4); 2.3333 (0.8); 2.3288 (1.0); 2.3241 (0.8); 2.3197 (0.4); −0.0002 (3.9)

I.7-291: ¹H-NMR (400.6 MHz, CDCl3):

δ=9.1487 (1.2); 9.1423 (1.2); 7.5195 (0.6); 7.4664 (1.8); 7.4547 (2.2); 7.4508 (2.7); 7.4458 (2.6); 7.4297 (0.7); 7.3276 (4.3); 7.3057 (4.3); 7.2882 (0.5); 7.2610 (105.3); 7.2529 (0.9); 7.2512 (0.8); 7.1562 (1.0); 7.1516 (1.2); 7.1379 (1.5); 7.1360 (1.6); 7.1333 (1.9); 7.1315 (1.8); 7.1178 (1.7); 7.1132 (2.1); 7.0885 (1.6); 7.0840 (1.9); 7.0685 (3.7); 7.0640 (2.9); 7.0362 (2.1); 7.0326 (2.5); 7.0180 (1.9); 7.0163 (1.5); 7.0144 (2.0); 7.0127 (1.6); 6.9977 (1.3); 6.9944 (1.0); 6.9717 (2.7); 6.9682 (2.4); 6.9515 (2.2); 6.9480 (2.0); 6.6995 (4.1); 6.6832 (4.1); 6.2643 (7.3); 5.5141 (7.7); 5.5114 (8.0); 4.7744 (16.0); 4.7157 (0.5); 3.4946 (10.0); 3.4917 (10.4); 2.1777 (1.1); 2.1717 (5.7); 2.0452 (0.8); 1.2643 (0.9); 1.2596 (1.1); 0.8819 (1.8); 0.8643 (0.7); 0.0079 (1.9); −0.0002 (64.1); −0.0085 (2.0)

I.7-115: ¹H-NMR (400.6 MHz, CDCl3):

δ=7.3890 (4.1); 7.3669 (4.1); 7.2606 (39.0); 7.1594 (0.9); 7.1548 (1.1); 7.1411 (1.5); 7.1392 (1.5); 7.1365 (1.8); 7.1347 (1.5); 7.1210 (1.4); 7.1164 (1.7); 7.0956 (1.4); 7.0911 (1.7); 7.0755 (3.5); 7.0710 (2.6); 7.0433 (1.9); 7.0396 (2.1); 7.0250 (1.8); 7.0230 (1.6); 7.0214 (1.8); 7.0198 (1.3); 7.0050 (0.8); 7.0014 (0.8); 6.9456 (2.4); 6.9420 (2.3); 6.9253 (2.1); 6.9218 (1.9); 6.7145 (3.9); 6.6981 (3.9); 6.2819 (6.9); 5.4984 (1.1); 5.4958 (1.1); 5.4826 (2.2); 5.4694 (1.2); 5.4668 (1.2); 5.4537 (0.5); 4.8761 (2.5); 4.8577 (4.0); 4.8550 (3.1); 4.8389 (2.7); 4.6917 (16.0); 4.6268 (1.6); 4.6230 (1.8); 4.6201 (1.4); 4.6138 (1.6); 4.6074 (2.6); 4.6026 (1.4); 4.6012 (1.4); 4.5945 (1.4); 4.5921 (1.5); 4.5881 (1.2); 3.5011 (9.7); 3.4984 (9.6); 2.1711 (1.4); 1.5530 (5.1); 1.2650 (1.0); 0.8990 (0.5); 0.8821 (1.7); 0.8644 (0.7); 0.0080 (0.7); −0.0002 (23.5); −0.0085 (0.6)

I.7-356: ¹H-NMR (400.2 MHz, d₆-DMSO):

δ=7.7947 (2.6); 7.7716 (2.6); 7.5769 (4.4); 7.1853 (0.6); 7.1798 (0.6); 7.1686 (0.7); 7.1637 (1.2); 7.1595 (1.0); 7.1477 (1.0); 7.1423 (1.1); 7.0728 (1.9); 7.0652 (0.6); 7.0598 (1.0); 7.0550 (1.3); 7.0519 (1.3); 7.0455 (2.6); 7.0398 (2.5); 7.0348 (1.6); 7.0207 (1.2); 7.0176 (1.4); 7.0009 (0.4); 6.9976 (0.4); 6.9548 (2.5); 6.9381 (2.6); 6.5087 (5.0); 5.7533 (7.5); 4.9652 (8.4); 4.7377 (7.3); 3.7087 (16.0); 3.3478 (328.3); 2.6766 (0.4); 2.6720 (0.6); 2.6673 (0.4); 2.5254 (1.4); 2.5204 (2.1); 2.5119 (34.8); 2.5075 (72.7); 2.5030 (96.8); 2.4985 (69.9); 2.4940 (33.8); 2.3343 (0.4); 2.3298 (0.6); 2.3254 (0.4); 2.0741 (15.8); −0.0002 (0.5)

I.7-394: ¹H-NMR (400.2 MHz, d₆-DMSO):

δ=8.3127 (0.3); 7.8198 (5.4); 7.7967 (5.5); 7.2189 (1.2); 7.2140 (1.4); 7.1975 (2.8); 7.1936 (2.4); 7.1806 (2.2); 7.1759 (2.4); 7.1150 (4.4); 7.0970 (3.0); 7.0942 (2.8); 7.0861 (10.8); 7.0814 (2.2); 7.0662 (5.2); 7.0616 (4.6); 7.0496 (3.2); 7.0465 (3.1); 7.0319 (2.9); 7.0291 (3.2); 7.0123 (1.2); 7.0089 (1.1); 6.9526 (5.4); 6.9359 (5.4); 6.5432 (0.3); 6.5118 (10.7); 5.7534 (7.2); 5.0458 (0.4); 5.0366 (3.3); 5.0277 (6.9); 5.0189 (3.3); 4.8538 (0.6); 4.8170 (16.0); 4.1382 (0.8); 4.1292 (0.8); 4.1237 (0.5); 4.1088 (5.7); 4.1006 (9.7); 4.0924 (5.5); 4.0718 (0.7); 4.0629 (0.7); 3.8509 (1.0); 3.8460 (1.5); 3.8409 (1.4); 3.8346 (2.3); 3.8292 (4.7); 3.8220 (4.8); 3.8160 (5.7); 3.8112 (3.1); 3.8062 (3.6); 3.7995 (2.8); 3.7912 (5.3); 3.7858 (5.3); 3.7813 (5.0); 3.7768 (4.0); 3.7730 (4.0); 3.7654 (1.2); 3.7568 (1.1); 3.3922 (1.5); 3.3436 (666.4); 3.1755 (0.5); 3.1624 (0.4); 2.6762 (0.8); 2.6718 (1.1); 2.6675 (0.8); 2.5112 (74.2); 2.5072 (141.9); 2.5028 (182.1); 2.4983 (134.0); 2.3339 (0.8); 2.3296 (1.1); 2.3251 (0.8); −0.0002 (1.5)

I.7-72: ¹H-NMR (400.2 MHz, d₆-DMSO):

δ=8.3126 (0.4); 7.8184 (6.1); 7.7953 (6.1); 7.2183 (1.2); 7.2138 (1.3); 7.1971 (2.9); 7.1934 (2.6); 7.1800 (2.5); 7.1755 (2.8); 7.1381 (4.0); 7.1347 (4.6); 7.1175 (2.6); 7.1140 (2.4); 7.0947 (2.0); 7.0903 (2.5); 7.0747 (5.0); 7.0704 (4.3); 7.0490 (3.0); 7.0456 (2.9); 7.0311 (2.8); 7.0281 (3.2); 7.0115 (1.4); 7.0079 (1.3); 6.9472 (6.0); 6.9305 (6.0); 6.5072 (9.2); 5.7535 (8.8); 4.8306 (0.6); 4.7867 (16.0); 4.0760 (1.1); 4.0661 (1.0); 4.0594 (1.2); 4.0493 (2.8); 4.0393 (2.1); 4.0326 (2.0); 4.0227 (2.0); 3.9972 (2.1); 3.9882 (2.0); 3.9773 (2.1); 3.9688 (2.4); 3.9615 (1.2); 3.9504 (1.1); 3.9415 (1.1); 3.7074 (1.2); 3.6933 (1.4); 3.6870 (3.0); 3.6729 (3.1); 3.6668 (2.1); 3.6528 (2.0); 3.6435 (2.5); 3.6222 (3.4); 3.6105 (2.1); 3.6040 (3.1); 3.5914 (3.4); 3.5735 (3.0); 3.5534 (1.3); 3.3870 (3.3); 3.3809 (4.0); 3.3730 (5.3); 3.3462 (870.4); 3.1756 (0.6); 3.1625 (0.5); 2.6809 (0.4); 2.6766 (0.9); 2.6720 (1.2); 2.6675 (0.9); 2.6631 (0.4); 2.5253 (3.5); 2.5204 (5.6); 2.5120 (75.1); 2.5075 (152.8); 2.5030 (200.4); 2.4984 (142.5); 2.4940 (67.6); 2.4741 (1.6); 2.4556 (1.7); 2.4385 (1.2); 2.4207 (0.6); 2.3389 (0.4); 2.3343 (0.9); 2.3298 (1.2); 2.3252 (0.8); 1.9371 (0.7); 1.9230 (0.8); 1.9168 (1.2); 1.9045 (1.4); 1.8965 (0.9); 1.8921 (1.0); 1.8854 (1.5); 1.8717 (1.3); 1.8656 (0.9); 1.8514 (0.7); 1.5429 (0.8); 1.5264 (1.3); 1.5113 (1.7); 1.4921 (1.7); 1.4773 (1.1); 1.4606 (0.7); −0.0002 (0.8)

I.7-179: ¹H-NMR (400.2 MHz, d₆-DMSO):

δ=8.3122 (0.4); 7.8187 (5.4); 7.7956 (5.5); 7.2124 (0.9); 7.2078 (1.0); 7.1915 (2.7); 7.1871 (2.3); 7.1748 (2.7); 7.1704 (3.0); 7.1598 (4.0); 7.1556 (4.6); 7.1391 (1.8); 7.1351 (1.4); 7.0978 (1.7); 7.0932 (2.2); 7.0777 (4.8); 7.0735 (4.0); 7.0585 (2.8); 7.0544 (2.6); 7.0414 (2.3); 7.0376 (2.7); 7.0219 (1.1); 7.0174 (1.1); 6.9510 (5.4); 6.9343 (5.4); 6.5443 (0.3); 6.5140 (10.5); 5.7532 (7.3); 4.8551 (0.6); 4.8103 (16.0); 4.2862 (5.1); 4.2712 (10.2); 4.2561 (5.2); 3.4082 (0.5); 3.3924 (1.4); 3.3467 (706.0); 3.1758 (0.8); 3.1627 (0.7); 2.8933 (5.6); 2.8783 (10.4); 2.8633 (5.3); 2.6811 (0.4); 2.6766 (0.7); 2.6720 (1.0); 2.6675 (0.7); 2.6631 (0.3); 2.5254 (2.7); 2.5206 (4.1); 2.5120 (63.6); 2.5076 (129.7); 2.5030 (169.6); 2.4985 (120.3); 2.4940 (56.8); 2.3388 (0.4); 2.3344 (0.7); 2.3298 (1.0); 2.3252 (0.8); 2.3207 (0.4); −0.0001 (1.0)

I.6-41: ¹H-NMR (400.6 MHz, CDCl3):

δ=7.8876 (1.8); 7.8661 (1.8); 7.2606 (86.1); 7.2177 (0.7); 7.2138 (1.6); 7.1952 (2.0); 7.1933 (2.5); 7.1886 (0.7); 7.1736 (1.0); 7.1695 (0.6); 7.0718 (0.9); 7.0682 (0.9); 7.0524 (1.1); 7.0488 (1.2); 7.0330 (0.6); 7.0295 (0.5); 6.9065 (1.1); 6.9033 (0.9); 6.8861 (1.1); 6.8810 (2.0); 6.8658 (1.8); 6.2766 (3.4); 4.6734 (6.6); 4.3621 (1.0); 4.3509 (1.4); 4.3390 (1.3); 4.1362 (2.3); 4.1310 (0.6); 4.1271 (1.3); 4.1242 (2.2); 4.1183 (0.5); 4.1128 (1.9); 3.5007 (4.8); 3.4978 (4.8); 1.5426 (16.0); 0.0080 (1.3); −0.0002 (53.5); −0.0085 (1.5)

I.6-276: ¹H-NMR (400.6 MHz, CDCl3):

δ=8.5648 (1.9); 8.5605 (2.5); 8.5505 (2.5); 8.5466 (1.8); 7.8353 (5.1); 7.8138 (5.0); 7.7077 (1.2); 7.7032 (1.2); 7.6884 (2.6); 7.6841 (2.3); 7.6692 (1.4); 7.6647 (1.3); 7.5196 (0.6); 7.3962 (0.6); 7.2611 (104.1); 7.2522 (2.4); 7.2413 (4.9); 7.2326 (1.9); 7.2220 (4.6); 7.2149 (3.0); 7.2109 (3.5); 7.1949 (4.2); 7.1911 (4.9); 7.1752 (3.2); 7.1710 (2.2); 7.1550 (2.6); 7.1508 (1.9); 7.0549 (2.4); 7.0513 (2.5); 7.0355 (3.4); 7.0320 (3.1); 7.0161 (1.6); 7.0126 (1.5); 6.9975 (0.6); 6.9453 (5.0); 6.9304 (8.2); 6.9104 (3.0); 6.9071 (2.5); 6.2323 (9.6); 5.6502 (1.6); 5.2532 (15.0); 4.7542 (16.0); 4.1308 (0.5); 4.1130 (0.6); 3.4747 (13.9); 3.4719 (13.3); 2.0452 (2.5); 1.6105 (0.5); 1.2773 (0.8); 1.2595 (1.7); 1.2416 (0.8); 0.0080 (1.8); 0.0063 (0.6); −0.0002 (63.7); −0.0085 (2.1)

I.6-23: ¹H-NMR (400.6 MHz, CDCl3):

δ=7.8800 (1.6); 7.8584 (1.6); 7.2609 (47.9); 7.2040 (1.6); 7.1850 (2.9); 7.1661 (0.9); 7.1620 (0.5); 7.0526 (0.7); 7.0491 (0.8); 7.0342 (0.7); 7.0297 (1.1); 6.9504 (1.6); 6.9354 (1.6); 6.9034 (1.0); 6.8832 (1.0); 6.8799 (0.6); 6.2812 (3.2); 4.6639 (5.0); 4.2800 (1.4); 4.2682 (1.5); 4.2644 (1.1); 4.2560 (1.6); 3.6798 (1.8); 3.6677 (1.8); 3.6558 (1.7); 3.6048 (1.1); 3.5959 (1.6); 3.5941 (1.6); 3.5881 (1.5); 3.5817 (2.6); 3.5217 (2.6); 3.5153 (1.5); 3.5091 (1.7); 3.5074 (1.6); 3.4994 (5.4); 3.3672 (16.0); 1.5495 (11.9); 0.0079 (0.8); −0.0002 (28.0); −0.0085 (0.8)

I.6-2: ¹H-NMR (400.6 MHz, CDCl3):

δ=7.8796 (0.8); 7.8580 (0.8); 7.2606 (87.6); 7.2052 (0.6); 7.1860 (0.7); 7.1822 (0.7); 7.0326 (0.6); 6.9970 (0.5); 6.9606 (0.8); 6.9455 (0.8); 6.2777 (1.5); 4.6715 (2.4); 4.2627 (0.7); 4.2511 (0.7); 4.2387 (0.8); 3.5981 (1.0); 3.5889 (0.6); 3.5861 (0.9); 3.5833 (0.5); 3.5741 (0.9); 3.5032 (1.3); 3.4994 (2.1); 3.4965 (2.1); 3.4859 (1.8); 3.4685 (1.8); 3.4510 (0.6); 1.5434 (16.0); 1.2195 (0.6); 1.1910 (1.9); 1.1735 (3.8); 1.1560 (1.8); 0.0079 (1.4); −0.0002 (53.4); −0.0085 (1.5)

I.7-339: ¹H-NMR (400.6 MHz, CDCl3):

δ=7.5189 (0.5); 7.3854 (4.2); 7.3633 (4.2); 7.2605 (93.0); 7.1661 (1.0); 7.1616 (1.2); 7.1477 (1.6); 7.1458 (1.6); 7.1432 (1.9); 7.1414 (1.6); 7.1275 (1.6); 7.1230 (1.9); 7.1003 (1.7); 7.0959 (1.8); 7.0803 (3.5); 7.0759 (2.7); 7.0411 (2.0); 7.0374 (2.2); 7.0227 (1.9); 7.0210 (1.6); 7.0191 (1.9); 7.0174 (1.4); 7.0027 (1.0); 6.9990 (1.0); 6.9969 (0.6); 6.9378 (2.5); 6.9342 (2.3); 6.9174 (2.2); 6.9139 (1.9); 6.7112 (4.0); 6.6949 (4.1); 6.2808 (7.0); 4.6550 (16.0); 4.3722 (4.3); 4.3553 (9.2); 4.3385 (4.5); 4.2666 (3.8); 4.2519 (6.3); 4.2369 (4.0); 3.4996 (9.9); 3.4967 (9.7); 2.3435 (1.0); 2.3270 (2.5); 2.3118 (3.3); 2.2968 (2.6); 2.2801 (0.9); 1.5432 (8.3); 0.0080 (1.6); −0.0002 (55.1); −0.0085 (1.4)

I.8-23: ¹H-NMR (400.6 MHz, CDCl3):

δ=7.5334 (1.8); 7.5119 (1.8); 7.2607 (38.9); 7.1243 (0.6); 7.1224 (0.6); 7.1198 (0.7); 7.1180 (0.7); 7.1041 (0.6); 7.0996 (0.8); 7.0716 (0.7); 7.0672 (0.8); 7.0516 (1.4); 7.0472 (1.1); 7.0128 (0.8); 7.0091 (0.9); 6.9944 (0.7); 6.9908 (0.8); 6.9410 (1.1); 6.9374 (1.0); 6.9206 (0.9); 6.9171 (0.8); 6.7414 (1.7); 6.7252 (1.7); 6.2774 (2.8); 4.6901 (5.7); 4.3106 (1.4); 4.3017 (1.0); 4.2987 (1.4); 4.2950 (1.0); 4.2865 (1.5); 3.6936 (1.7); 3.6846 (1.0); 3.6815 (1.7); 3.6788 (1.0); 3.6695 (1.6); 3.6100 (1.1); 3.6023 (1.4); 3.5995 (1.5); 3.5944 (1.5); 3.5878 (2.5); 3.5231 (2.4); 3.5165 (1.4); 3.5108 (1.5); 3.5086 (1.5); 3.4970 (4.0); 3.4941 (4.0); 3.3683 (16.0); 2.0454 (1.2); 1.5508 (14.1); 1.2774 (0.5); 1.2596 (1.1); 0.8820 (1.1); 0.0079 (0.8); −0.0002 (26.8); −0.0085 (0.8)

I.8-41: ¹H-NMR (400.6 MHz, CDCl3):

δ=7.5409 (2.4); 7.5192 (2.5); 7.2657 (0.6); 7.2600 (48.9); 7.2552 (0.7); 7.1519 (0.5); 7.1474 (0.6); 7.1336 (0.8); 7.1317 (0.8); 7.1291 (1.0); 7.1271 (0.9); 7.1134 (0.8); 7.1089 (1.0); 7.0889 (0.9); 7.0844 (1.0); 7.0688 (1.9); 7.0644 (1.4); 7.0326 (1.0); 7.0290 (1.2); 7.0143 (1.0); 7.0126 (0.8); 7.0108 (1.0); 7.0091 (0.8); 6.9944 (0.5); 6.9396 (1.4); 6.9360 (1.3); 6.9193 (1.2); 6.9158 (1.0); 6.6887 (2.3); 6.6726 (2.4); 6.2729 (3.8); 4.7046 (8.4); 4.3800 (1.2); 4.3687 (1.5); 4.3567 (1.4); 4.1413 (2.6); 4.1362 (0.7); 4.1321 (1.6); 4.1294 (2.4); 4.1233 (0.6); 4.1178 (2.1); 3.4960 (5.2); 3.4931 (5.2); 2.7789 (0.9); 2.5515 (2.0); 2.0452 (1.4); 1.5454 (16.0); 1.2597 (1.0); 0.0080 (0.9); −0.0002 (33.8); −0.0085 (1.0)

I.8-176: ¹H-NMR (400.6 MHz, CDCl3):

δ=7.2600 (61.4); 6.2864 (0.7); 4.7584 (2.5); 4.7467 (1.8); 3.5033 (1.0); 3.5003 (1.0); 1.5335 (16.0); 0.0080 (2.5); 0.0055 (0.5); −0.0002 (90.9); −0.0067 (0.7); −0.0085 (2.5)

I.9-23: ¹H-NMR (400.6 MHz, CDCl3):

δ=7.7003 (0.5); 7.6971 (0.7); 7.6798 (0.9); 7.6762 (0.8); 7.6703 (0.5); 7.6670 (0.7); 7.6498 (0.8); 7.6462 (0.8); 7.5367 (0.5); 7.5098 (1.5); 7.4891 (2.0); 7.4825 (0.7); 7.4742 (0.5); 7.4709 (0.9); 7.4670 (0.7); 7.4637 (0.9); 7.3525 (0.5); 7.3479 (0.9); 7.3428 (1.1); 7.3354 (1.5); 7.3311 (1.3); 7.3255 (1.1); 7.3072 (0.7); 7.3004 (0.6); 7.2626 (47.5); 7.2262 (0.7); 7.2224 (1.1); 7.2193 (0.7); 7.2062 (0.7); 7.2031 (1.3); 7.1996 (1.3); 7.1948 (0.6); 7.1798 (0.8); 7.1757 (0.5); 7.0565 (0.7); 7.0530 (0.7); 7.0372 (1.0); 7.0337 (1.0); 6.8886 (0.9); 6.8852 (0.8); 6.8680 (0.8); 6.8653 (0.6); 6.8010 (1.5); 6.7864 (1.5); 6.2750 (2.8); 4.6624 (3.4); 4.2847 (1.2); 4.2729 (1.2); 4.2691 (0.9); 4.2608 (1.3); 3.6858 (1.6); 3.6767 (1.0); 3.6738 (1.6); 3.6619 (1.4); 3.6097 (1.0); 3.6010 (1.4); 3.5989 (1.4); 3.5929 (1.4); 3.5866 (2.4); 3.5267 (2.4); 3.5204 (1.4); 3.5143 (1.4); 3.5124 (1.3); 3.5047 (1.1); 3.4917 (3.8); 3.4890 (3.8); 3.3712 (16.0); 3.3216 (0.5); 3.1281 (0.5); 3.0165 (15.4); 2.9442 (9.5); 2.0870 (9.8); 1.5729 (1.1); 1.2539 (0.7); 0.0080 (2.0); −0.0002 (70.5); −0.0085 (2.0)

I.9-41: ¹H-NMR (400.6 MHz, CDCl3):

δ=7.5186 (0.6); 7.5127 (5.1); 7.4917 (5.1); 7.2601 (86.1); 7.2321 (2.2); 7.2282 (3.7); 7.2245 (2.2); 7.2203 (1.3); 7.2122 (2.3); 7.2090 (4.4); 7.2049 (4.4); 7.2001 (2.2); 7.1852 (2.6); 7.1810 (1.8); 7.0736 (2.3); 7.0700 (2.5); 7.0542 (3.6); 7.0507 (3.3); 7.0349 (1.6); 7.0313 (1.5); 6.8939 (3.0); 6.8905 (2.5); 6.8735 (2.8); 6.8705 (2.2); 6.7282 (5.0); 6.7137 (5.1); 6.2665 (9.3); 4.6709 (16.0); 4.3676 (2.6); 4.3564 (3.5); 4.3443 (3.4); 4.1395 (6.5); 4.1347 (1.6); 4.1304 (3.6); 4.1276 (5.9); 4.1214 (1.5); 4.1162 (5.1); 3.4914 (12.8); 3.4885 (13.1); 3.1263 (1.1); 2.4064 (0.8); 2.0453 (0.6); 1.5376 (9.0); 1.2510 (0.9); 0.0079 (3.7); −0.0002 (129.1); −0.0085 (3.7)

I.6-183: ¹H-NMR (400.2 MHz, d6-DMSO):

δ=13.0005 (0.4); 8.3149 (0.6); 8.2785 (0.7); 8.2726 (5.6); 8.2627 (0.4); 8.2561 (0.8); 8.2502 (5.6); 7.5164 (2.0); 7.4980 (2.1); 7.4798 (2.2); 7.4613 (2.2); 7.2627 (1.3); 7.2587 (1.7); 7.2403 (3.1); 7.2383 (3.0); 7.2260 (4.5); 7.2222 (4.5); 7.2060 (4.8); 7.2022 (3.2); 7.1736 (5.3); 7.1584 (5.8); 7.1235 (0.7); 7.1171 (3.3); 7.1139 (3.8); 7.0963 (2.8); 7.0934 (2.8); 7.0706 (0.5); 7.0651 (2.4); 7.0617 (2.1); 7.0552 (0.4); 7.0453 (3.1); 7.0267 (1.6); 7.0232 (1.4); 6.6432 (2.5); 6.6370 (2.5); 6.6065 (2.3); 6.6003 (2.3); 6.5659 (0.3); 6.5441 (11.8); 6.1897 (1.2); 6.1849 (1.3); 6.1719 (1.3); 6.1670 (1.2); 5.7551 (13.5); 4.8430 (0.3); 4.8339 (0.4); 4.8188 (0.5); 4.7440 (1.0); 4.7300 (0.8); 4.7203 (0.5); 4.7179 (0.8); 4.7149 (0.5); 4.7056 (0.8); 4.6474 (0.8); 4.6342 (16.0); 4.5637 (0.6); 4.5519 (0.4); 4.5403 (0.4); 3.9594 (0.7); 3.9471 (0.6); 3.9350 (0.6); 3.6223 (0.7); 3.3584 (17.5); 3.3280 (105.6); 3.2698 (0.6); 3.1748 (0.4); 2.6798 (0.4); 2.6754 (1.0); 2.6708 (1.3); 2.6663 (1.0); 2.6616 (0.4); 2.5243 (3.6); 2.5195 (5.9); 2.5109 (87.6); 2.5065 (177.7); 2.5019 (231.9); 2.4973 (163.4); 2.4927 (76.2); 2.3380 (0.5); 2.3332 (1.0); 2.3287 (1.4); 2.3241 (1.0); 2.3197 (0.5); 1.2982 (0.4); 1.2588 (0.7); 1.2353 (1.1); −0.0002 (6.1)

I.8-2: ¹H-NMR (400.6 MHz, CDCl3):

δ=7.5323 (4.1); 7.5108 (4.1); 7.2605 (34.2); 7.1395 (0.9); 7.1351 (1.1); 7.1211 (1.4); 7.1192 (1.4); 7.1167 (1.6); 7.1149 (1.5); 7.1009 (1.4); 7.0965 (1.8); 7.0748 (1.6); 7.0704 (1.8); 7.0547 (3.3); 7.0504 (2.5); 7.0119 (1.8); 7.0082 (2.1); 6.9935 (1.6); 6.9919 (1.4); 6.9899 (1.7); 6.9883 (1.5); 6.9735 (0.8); 6.9699 (0.9); 6.9377 (2.4); 6.9341 (2.2); 6.9173 (2.0); 6.9138 (1.8); 6.7480 (3.8); 6.7319 (3.9); 6.2812 (0.6); 6.2749 (6.4); 4.6967 (13.1); 4.6903 (1.3); 4.6831 (0.6); 4.2926 (3.2); 4.2838 (2.1); 4.2807 (3.2); 4.2771 (2.1); 4.2687 (3.6); 3.6128 (4.2); 3.6057 (1.5); 3.6038 (2.2); 3.6007 (4.0); 3.5980 (2.3); 3.5888 (3.9); 3.5097 (2.5); 3.4963 (9.8); 3.4925 (14.4); 3.4747 (7.7); 3.4572 (2.5); 2.0302 (1.8); 1.5499 (9.7); 1.1949 (7.9); 1.1826 (1.0); 1.1774 (16.0); 1.1651 (0.5); 1.1599 (7.6); 0.0080 (0.9); −0.0002 (33.9); −0.0085 (1.0)

I.8-1: ¹H-NMR (400.6 MHz, CDCl3):

δ=7.5322 (2.4); 7.5107 (2.3); 7.2608 (10.9); 7.1432 (0.9); 7.1389 (0.9); 7.1205 (1.6); 7.1046 (1.3); 7.1003 (1.3); 7.0796 (1.4); 7.0754 (1.4); 7.0596 (2.3); 7.0555 (1.9); 7.0139 (1.5); 7.0104 (1.5); 6.9922 (1.6); 6.9755 (0.8); 6.9721 (0.8); 6.9330 (2.0); 6.9297 (1.8); 6.9127 (1.6); 6.9094 (1.4); 6.7448 (2.2); 6.7287 (2.2); 6.2738 (4.0); 4.6977 (8.4); 4.2895 (2.5); 4.2783 (3.0); 4.2663 (2.5); 3.5625 (3.2); 3.5508 (3.8); 3.5392 (3.1); 3.4949 (8.2); 3.3398 (1.8); 3.3269 (16.0); 1.5588 (2.4); −0.0002 (7.9)

I.8-276: ¹H-NMR (400.6 MHz, CDCl3):

δ=8.5684 (1.4); 8.5643 (1.9); 8.5611 (1.3); 8.5569 (1.2); 8.5535 (1.7); 8.5495 (1.5); 7.6827 (1.2); 7.6783 (1.2); 7.6634 (2.2); 7.6590 (2.2); 7.6442 (1.4); 7.6397 (1.4); 7.4937 (4.9); 7.4722 (4.9); 7.2613 (16.8); 7.2366 (4.3); 7.2248 (1.5); 7.2220 (1.3); 7.2177 (4.5); 7.2060 (1.2); 7.1384 (1.1); 7.1340 (1.4); 7.1198 (1.7); 7.1182 (1.7); 7.1154 (2.1); 7.1138 (2.2); 7.0998 (1.8); 7.0954 (2.5); 7.0906 (2.1); 7.0862 (2.1); 7.0705 (3.9); 7.0662 (2.8); 7.0201 (2.2); 7.0164 (2.6); 7.0016 (1.9); 7.0002 (1.7); 6.9980 (2.1); 6.9964 (1.9); 6.9817 (1.1); 6.9780 (1.1); 6.9622 (3.0); 6.9585 (2.6); 6.9419 (2.4); 6.9383 (2.1); 6.7293 (4.6); 6.7132 (4.6); 6.2384 (7.7); 5.2809 (12.8); 4.7759 (16.0); 4.7626 (0.6); 4.6490 (0.5); 3.4749 (10.4); 3.4720 (10.8); 2.1681 (0.5); 1.2539 (1.2); 0.0080 (0.6); −0.0002 (21.0); −0.0085 (0.6)

I.9-2: ¹H-NMR (400.2 MHz, d6-DMSO):

δ=8.1472 (4.0); 8.1245 (4.0); 8.1171 (0.4); 7.3039 (2.2); 7.3001 (2.7); 7.2843 (3.3); 7.2806 (3.7); 7.2662 (2.3); 7.2468 (2.0); 7.2429 (1.6); 7.1690 (2.7); 7.1659 (3.0); 7.1483 (2.0); 7.1453 (1.9); 7.1014 (1.7); 7.0981 (1.6); 7.0822 (2.7); 7.0792 (2.4); 7.0630 (1.3); 7.0597 (1.1); 6.9792 (3.9); 6.9645 (4.0); 6.5342 (8.2); 5.7554 (5.2); 4.8341 (0.4); 4.7614 (12.2); 4.1781 (3.5); 4.1666 (4.1); 4.1546 (3.7); 3.5236 (4.0); 3.5117 (4.3); 3.5000 (3.5); 3.4197 (2.5); 3.4022 (7.8); 3.3847 (7.9); 3.3672 (2.8); 3.3470 (13.3); 3.3270 (149.5); 2.6752 (0.6); 2.6708 (0.8); 2.6663 (0.6); 2.5240 (2.5); 2.5105 (56.5); 2.5063 (111.7); 2.5018 (143.9); 2.4973 (103.4); 2.4931 (50.1); 2.3332 (0.6); 2.3287 (0.8); 2.3243 (0.6); 1.9841 (1.6); 1.2983 (0.4); 1.2588 (0.7); 1.2352 (0.9); 1.0788 (7.9); 1.0613 (16.0); 1.0438 (7.6); −0.0002 (2.9)

I.9-276: ¹H-NMR (400.6 MHz, CDCl3):

δ=8.5790 (2.2); 8.5745 (2.5); 8.5655 (2.6); 7.7554 (0.9); 7.7357 (1.6); 7.7166 (1.0); 7.5261 (0.6); 7.5190 (0.9); 7.4480 (5.9); 7.4269 (5.9); 7.2805 (3.2); 7.2606 (137.6); 7.2429 (0.9); 7.2307 (3.4); 7.2266 (4.0); 7.2107 (4.5); 7.2069 (5.6); 7.1900 (3.5); 7.1851 (2.8); 7.1701 (2.9); 7.1659 (2.3); 7.0584 (2.7); 7.0550 (3.1); 7.0391 (3.9); 7.0356 (4.0); 7.0199 (1.9); 7.0163 (1.8); 6.9969 (0.8); 6.9319 (3.3); 6.9287 (3.4); 6.9116 (2.9); 6.9084 (2.8); 6.7843 (5.6); 6.7698 (5.6); 6.2943 (0.8); 6.2237 (10.8); 5.3194 (0.5); 5.2799 (11.2); 4.7564 (12.3); 4.7545 (12.5); 4.7128 (0.6); 4.6431 (1.6); 3.5271 (0.5); 3.4955 (1.2); 3.4675 (15.7); 3.4648 (16.0); 3.3193 (2.2); 2.9660 (0.8); 2.8924 (0.6); 2.0499 (2.0); 2.0453 (1.9); 1.5717 (0.9); 1.3330 (2.4); 1.2843 (3.3); 1.2773 (1.7); 1.2545 (4.6); 1.2416 (1.5); 1.1745 (1.1); 1.1590 (1.0); 0.8803 (1.3); 0.8627 (0.7); 0.1456 (0.6); 0.0079 (5.5); −0.0002 (181.1); −0.0085 (6.8); −0.1497 (0.6)

I.8-183: ¹H-NMR (400.6 MHz, CDCl3):

δ=7.5498 (4.0); 7.5283 (4.0); 7.2606 (33.7); 7.1661 (1.0); 7.1616 (1.2); 7.1478 (1.4); 7.1459 (1.4); 7.1433 (1.7); 7.1414 (1.5); 7.1276 (1.5); 7.1231 (1.8); 7.0989 (1.5); 7.0944 (1.6); 7.0789 (3.4); 7.0744 (2.6); 7.0444 (2.1); 7.0407 (2.3); 7.0261 (1.7); 7.0244 (1.4); 7.0225 (1.8); 7.0208 (1.4); 7.0061 (0.9); 7.0025 (0.9); 6.9177 (2.3); 6.9141 (2.2); 6.8973 (2.0); 6.8937 (1.8); 6.6412 (3.7); 6.6251 (3.8); 6.2790 (6.2); 4.6717 (16.0); 4.6646 (1.2); 4.6594 (3.8); 4.6534 (1.9); 4.6482 (2.5); 4.6456 (2.5); 4.6399 (1.8); 4.5932 (1.8); 4.5874 (3.1); 4.5848 (2.5); 4.5798 (2.3); 4.5758 (2.5); 4.5736 (3.6); 4.5688 (0.9); 4.5643 (1.9); 4.5624 (1.8); 3.4990 (8.6); 3.4960 (8.8); 3.3192 (0.8); 2.0497 (0.7); 2.0451 (1.9); 1.2771 (0.7); 1.2593 (1.4); 1.2414 (0.6); 0.0080 (1.0); −0.0002 (32.5); −0.0085 (0.9)

I.8-291: ¹H-NMR (400.6 MHz, CDCl3):

δ=9.1478 (2.0); 9.1430 (2.1); 9.1363 (2.0); 9.1314 (2.0); 7.5537 (1.2); 7.5325 (1.4); 7.5185 (0.6); 7.4864 (4.8); 7.4725 (1.2); 7.4647 (5.5); 7.4514 (4.3); 7.4449 (3.6); 7.4400 (4.2); 7.4236 (1.4); 7.4187 (1.4); 7.2600 (81.1); 7.1571 (1.0); 7.1527 (1.3); 7.1495 (0.7); 7.1387 (1.5); 7.1369 (1.6); 7.1343 (1.8); 7.1325 (1.7); 7.1287 (0.6); 7.1240 (0.6); 7.1187 (1.6); 7.1141 (1.9); 7.1086 (1.0); 7.1042 (0.8); 7.0925 (1.7); 7.0880 (1.9); 7.0724 (4.0); 7.0681 (3.3); 7.0488 (0.6); 7.0364 (2.0); 7.0328 (3.0); 7.0181 (1.8); 7.0130 (2.1); 6.9981 (1.0); 6.9946 (1.0); 6.9731 (2.8); 6.9695 (2.4); 6.9529 (2.3); 6.9493 (2.0); 6.6697 (1.2); 6.6624 (4.5); 6.6537 (1.2); 6.6463 (4.5); 6.3007 (1.8); 6.2590 (7.4); 5.5090 (7.7); 5.5062 (7.8); 5.4536 (1.8); 4.7710 (16.0); 4.6626 (4.0); 3.5068 (2.6); 3.5040 (2.8); 3.4905 (10.1); 3.4878 (10.2); 2.7757 (1.4); 2.6225 (0.5); 2.5515 (1.3); 2.1772 (4.5); 1.2556 (0.6); 0.0080 (3.3); −0.0002 (116.1); −0.0085 (3.5)

I.9-291: ¹H-NMR (400.6 MHz, CDCl3):

δ=9.1797 (2.2); 9.1751 (2.4); 9.1678 (2.4); 9.1632 (2.4); 7.5628 (1.6); 7.5508 (1.4); 7.5416 (4.1); 7.5296 (4.3); 7.5162 (3.7); 7.5115 (4.1); 7.4949 (1.5); 7.4903 (1.3); 7.4614 (5.1); 7.4404 (5.0); 7.2606 (83.8); 7.2264 (5.4); 7.2070 (8.1); 7.2029 (2.1); 7.1879 (2.6); 7.1837 (1.7); 7.0738 (2.1); 7.0702 (2.4); 7.0536 (2.2); 7.0508 (3.4); 7.0351 (1.6); 7.0315 (1.5); 6.9969 (0.6); 6.9408 (2.8); 6.9381 (2.1); 6.9203 (2.6); 6.9169 (1.7); 6.6968 (4.8); 6.6823 (4.8); 6.2522 (9.0); 5.5236 (1.2); 5.4892 (7.8); 5.4812 (7.8); 5.4472 (0.9); 4.7661 (0.6); 4.7475 (16.0); 3.4875 (11.8); 3.4846 (12.2); 0.0079 (3.6); −0.0002 (126.7); −0.0085 (3.7)

I.8-291: ¹H-NMR (400.6 MHz, CDCl3):

δ=9.1478 (2.0); 9.1429 (2.0); 9.1362 (2.0); 9.1313 (2.0); 7.4894 (4.5); 7.4749 (1.3); 7.4679 (4.6); 7.4634 (1.1); 7.4537 (3.8); 7.4421 (4.2); 7.4355 (3.4); 7.4306 (3.7); 7.4143 (1.1); 7.4093 (0.8); 7.2613 (35.0); 7.1581 (1.0); 7.1536 (1.2); 7.1397 (1.5); 7.1380 (1.6); 7.1353 (1.8); 7.1335 (1.7); 7.1197 (1.6); 7.1151 (2.0); 7.0944 (1.7); 7.0900 (1.9); 7.0744 (3.7); 7.0700 (2.8); 7.0378 (2.0); 7.0341 (2.4); 7.0194 (1.8); 7.0178 (1.5); 7.0158 (2.0); 7.0142 (1.6); 6.9995 (0.9); 6.9958 (1.0); 6.9742 (2.8); 6.9706 (2.4); 6.9539 (2.2); 6.9504 (2.0); 6.6634 (4.4); 6.6473 (4.4); 6.2602 (7.2); 5.5118 (7.6); 5.5089 (7.7); 4.7741 (16.0); 4.1308 (1.1); 4.1129 (1.0); 3.4917 (9.9); 3.4888 (10.1); 3.3194 (0.8); 2.0499 (0.8); 2.0453 (5.0); 1.5781 (9.0); 1.2774 (1.4); 1.2595 (3.1); 1.2417 (1.4); 0.0079 (1.5); −0.0002 (52.1); −0.0085 (1.4)

I.9-291: ¹H-NMR (400.6 MHz, CDCl3):

δ=9.1592 (2.3); 9.1545 (2.4); 9.1474 (2.4); 9.1426 (2.4); 7.7008 (2.9); 7.6976 (4.1); 7.6939 (1.7); 7.6855 (1.2); 7.6803 (5.5); 7.6767 (4.6); 7.6709 (2.9); 7.6676 (4.2); 7.6640 (1.8); 7.6556 (1.2); 7.6504 (5.4); 7.6468 (4.5); 7.5736 (0.6); 7.5700 (1.2); 7.5662 (1.3); 7.5627 (0.6); 7.5575 (0.7); 7.5518 (2.6); 7.5476 (2.6); 7.5422 (0.9); 7.5366 (1.4); 7.5329 (3.1); 7.5291 (3.0); 7.5254 (1.3); 7.5197 (0.6); 7.5169 (1.7); 7.5050 (1.4); 7.4957 (4.5); 7.4888 (2.7); 7.4869 (3.9); 7.4838 (6.9); 7.4817 (3.4); 7.4797 (4.0); 7.4763 (2.0); 7.4717 (6.6); 7.4674 (8.3); 7.4641 (4.6); 7.4606 (9.8); 7.4570 (1.9); 7.4542 (1.3); 7.4505 (3.8); 7.4465 (2.6); 7.4430 (2.7); 7.4393 (5.8); 7.2613 (69.0); 7.2285 (2.0); 7.2244 (5.3); 7.2199 (1.0); 7.2086 (2.2); 7.2061 (4.7); 7.2043 (7.7); 7.1998 (2.1); 7.1848 (2.7); 7.1807 (1.8); 7.0713 (2.3); 7.0677 (2.5); 7.0518 (2.9); 7.0483 (3.4); 7.0326 (1.6); 7.0291 (1.6); 6.9379 (2.9); 6.9348 (2.2); 6.9174 (2.6); 6.6980 (4.7); 6.6835 (4.8); 6.2524 (9.1); 5.5183 (1.1); 5.4844 (7.9); 5.4756 (8.0); 5.4576 (0.9); 5.4419 (1.0); 4.7656 (0.8); 4.7469 (16.0); 3.4875 (12.1); 3.4845 (12.7); 2.1800 (2.3); 2.0453 (1.6); 1.5901 (3.0); 1.3332 (0.6); 1.2844 (0.9); 1.2773 (0.6); 1.2594 (1.4); 1.2562 (1.6); 1.2416 (0.5); 0.0080 (3.1); −0.0002 (104.0); −0.0058 (1.4); −0.0085 (3.1)

I.19-276: ¹H-NMR (400.0 MHz, CDCl3):

δ=8.5628 (1.0); 8.5581 (1.0); 8.5494 (1.0); 8.5450 (0.8); 7.6964 (0.7); 7.6920 (0.7); 7.6771 (1.5); 7.6726 (1.3); 7.6578 (0.8); 7.6534 (0.8); 7.3225 (2.6); 7.3004 (2.6); 7.2613 (7.0); 7.2450 (1.2); 7.2406 (1.3); 7.2364 (1.7); 7.2260 (1.0); 7.2167 (1.7); 7.1197 (0.7); 7.1047 (0.7); 7.0983 (1.4); 7.0835 (1.3); 7.0771 (0.8); 7.0623 (0.7); 6.8814 (0.8); 6.8781 (0.9); 6.8575 (1.3); 6.8368 (0.7); 6.8335 (0.6); 6.7192 (1.9); 6.7124 (1.0); 6.7088 (1.5); 6.7037 (2.0); 6.6912 (0.8); 6.6875 (1.2); 6.2209 (4.3); 5.2986 (16.0); 5.2901 (0.7); 5.2642 (7.1); 4.7605 (8.7); 3.4659 (6.0); 3.4632 (6.0); 2.0445 (0.9); 1.2589 (0.8); −0.0002 (10.0)

I.19-176: ¹H-NMR (400.0 MHz, CDCl3):

δ=7.3919 (3.5); 7.3699 (3.4); 7.2606 (8.0); 7.1737 (0.8); 7.1589 (0.9); 7.1524 (1.8); 7.1377 (1.8); 7.1312 (1.1); 7.1165 (1.0); 6.9356 (1.1); 6.9325 (1.2); 6.9116 (1.8); 6.8910 (0.9); 6.8879 (0.9); 6.7009 (1.2); 6.6975 (2.0); 6.6797 (1.1); 6.6763 (1.7); 6.6076 (2.4); 6.5916 (2.3); 6.2789 (6.0); 5.2989 (14.6); 4.7544 (2.0); 4.7484 (16.0); 4.7319 (13.5); 4.7225 (1.2); 4.1299 (0.5); 4.1120 (0.5); 3.5337 (0.7); 3.4998 (8.6); 3.4972 (8.7); 2.0442 (2.4); 1.5500 (2.3); 1.2767 (0.7); 1.2589 (1.4); 1.2410 (0.6); −0.0002 (11.4)

I.23-176: ¹H-NMR (400.0 MHz, CDCl3):

δ=7.4170 (3.4); 7.3949 (3.3); 7.2606 (12.4); 6.9813 (1.4); 6.9685 (1.5); 6.9589 (2.0); 6.9460 (1.9); 6.8642 (1.2); 6.8568 (1.4); 6.8452 (1.3); 6.8418 (1.0); 6.8377 (1.5); 6.8345 (1.2); 6.8227 (0.8); 6.8152 (1.1); 6.8079 (2.2); 6.7985 (3.6); 6.7861 (2.4); 6.7821 (3.4); 6.7790 (1.6); 6.3117 (5.7); 5.2995 (11.5); 4.7712 (16.0); 4.7655 (1.3); 4.7221 (13.9); 4.6879 (1.0); 3.5431 (0.7); 3.5234 (8.2); 3.5206 (8.2); 2.0448 (1.8); 1.5462 (4.8); 1.2771 (0.6); 1.2592 (1.2); 0.0079 (0.7); −0.0002 (17.8); −0.0084 (0.6)

I.23-1: ¹H-NMR (400.0 MHz, CDCl3):

δ=7.3903 (2.1); 7.3681 (2.0); 7.2611 (5.9); 6.9572 (0.9); 6.9443 (0.9); 6.9347 (1.2); 6.9219 (1.1); 6.8684 (2.0); 6.8520 (2.0); 6.8288 (0.6); 6.8214 (0.8); 6.8095 (0.7); 6.8065 (0.5); 6.8020 (0.8); 6.7990 (0.7); 6.7795 (0.7); 6.7702 (1.2); 6.7628 (0.9); 6.7483 (1.3); 6.7409 (0.9); 6.3017 (3.5); 5.2992 (6.3); 4.6717 (7.8); 4.6510 (0.5); 4.3027 (2.0); 4.2942 (1.2); 4.2911 (1.9); 4.2873 (1.1); 4.2793 (2.0); 3.5791 (2.3); 3.5707 (1.2); 3.5673 (2.2); 3.5644 (1.2); 3.5557 (2.1); 3.5177 (4.9); 3.5150 (5.0); 3.3523 (1.5); 3.3463 (16.0); −0.0002 (8.3)

I.19-1: ¹H-NMR (400.0 MHz, CDCl3):

δ=7.3607 (2.2); 7.3386 (2.2); 7.2610 (4.8); 7.1336 (0.5); 7.1186 (0.6); 7.1124 (1.1); 7.0975 (1.1); 7.0911 (0.7); 7.0763 (0.6); 6.8806 (0.8); 6.8774 (0.8); 6.8566 (1.2); 6.8359 (0.6); 6.8329 (0.6); 6.7243 (1.7); 6.7081 (1.6); 6.6752 (1.2); 6.6540 (1.1); 6.2630 (3.9); 5.2988 (12.2); 4.6853 (8.1); 4.2734 (1.3); 4.2654 (1.7); 4.2602 (1.5); 4.2521 (1.3); 3.5476 (2.5); 3.5360 (2.9); 3.5293 (1.0); 3.5244 (2.3); 3.4899 (5.8); 3.4876 (5.8); 3.3370 (1.3); 3.3171 (16.0); 1.5622 (1.3); −0.0002 (6.8)

I.19-23: ¹H-NMR (400.6 MHz, CDCl3):

δ=7.3611 (1.6); 7.3390 (1.6); 7.2614 (5.8); 7.1139 (0.9); 7.0990 (0.9); 7.0927 (0.5); 6.8797 (0.5); 6.8764 (0.5); 6.8583 (0.5); 6.8562 (0.7); 6.8531 (0.6); 6.7148 (1.0); 6.6986 (1.0); 6.6833 (0.8); 6.6620 (0.8); 6.2666 (2.6); 5.2990 (8.8); 4.6768 (5.3); 4.2950 (1.2); 4.2864 (0.9); 4.2832 (1.2); 4.2794 (0.8); 4.2711 (1.3); 3.6802 (1.6); 3.6733 (0.6); 3.6711 (0.9); 3.6682 (1.6); 3.6656 (1.0); 3.6635 (0.6); 3.6563 (1.4); 3.6022 (0.9); 3.6009 (0.9); 3.5933 (1.3); 3.5912 (1.3); 3.5903 (1.3); 3.5853 (1.4); 3.5789 (2.3); 3.5182 (2.3); 3.5117 (1.4); 3.5068 (1.3); 3.5057 (1.3); 3.5038 (1.3); 3.4945 (2.2); 3.4915 (3.5); 3.4885 (3.5); 3.3683 (1.9); 3.3660 (16.0); 1.5679 (1.8); −0.0002 (8.7)

I.23-23: ¹H-NMR (400.6 MHz, CDCl3):

δ=7.3919 (1.8); 7.3698 (1.8); 7.2627 (3.6); 6.9669 (0.8); 6.9540 (0.8); 6.9444 (1.0); 6.9315 (1.0); 6.8704 (1.8); 6.8540 (1.8); 6.8294 (0.6); 6.8219 (0.7); 6.8100 (0.6); 6.8025 (0.7); 6.7994 (0.7); 6.7800 (0.6); 6.7630 (1.1); 6.7556 (0.8); 6.7411 (1.1); 6.7337 (0.8); 6.3040 (3.1); 5.2991 (12.5); 4.6645 (6.7); 4.3206 (1.6); 4.3119 (1.0); 4.3087 (1.7); 4.3049 (1.0); 4.2966 (1.7); 3.7012 (1.9); 3.6928 (1.1); 3.6892 (1.9); 3.6860 (1.1); 3.6773 (1.7); 3.6199 (1.3); 3.6114 (1.5); 3.6091 (1.8); 3.6036 (1.5); 3.5970 (2.6); 3.5297 (2.7); 3.5227 (2.6); 3.5182 (5.9); 3.5156 (5.7); 3.5072 (1.4); 3.3700 (16.0); 1.5867 (0.9); −0.0002 (5.1)

I.23-276: ¹H-NMR (400.6 MHz, CDCl3):

δ=8.6265 (1.6); 8.6142 (1.6); 7.7696 (1.1); 7.7652 (1.1); 7.7503 (2.2); 7.7459 (2.1); 7.7310 (1.2); 7.7266 (1.2); 7.3548 (4.4); 7.3326 (6.4); 7.3122 (2.3); 7.3064 (1.4); 7.2997 (1.2); 7.2872 (1.1); 7.2612 (18.1); 6.9784 (1.8); 6.9657 (1.8); 6.9561 (2.2); 6.9433 (2.2); 6.8537 (4.2); 6.8374 (4.2); 6.8231 (1.3); 6.8156 (1.6); 6.8038 (1.4); 6.8009 (1.0); 6.7963 (1.7); 6.7934 (1.6); 6.7814 (1.0); 6.7783 (2.9); 6.7740 (1.6); 6.7710 (1.8); 6.7564 (2.7); 6.7491 (1.6); 6.2783 (7.5); 5.3150 (12.4); 5.2991 (11.1); 4.7495 (16.0); 4.6352 (0.8); 3.5034 (10.0); 3.5006 (10.3); 0.0080 (0.8); −0.0002 (27.4); −0.0085 (0.7)

I.8-278: ¹H-NMR (400.6 MHz, CDCl3):

δ=8.5803 (3.6); 8.5766 (2.5); 8.5689 (2.6); 8.5653 (3.5); 7.4781 (4.6); 7.4565 (4.5); 7.2610 (24.0); 7.2138 (0.5); 7.1798 (0.5); 7.1653 (4.6); 7.1613 (2.6); 7.1540 (3.1); 7.1515 (4.5); 7.1503 (4.4); 7.1332 (1.7); 7.1312 (1.6); 7.1286 (1.9); 7.1266 (1.6); 7.1132 (1.7); 7.1085 (2.2); 7.0970 (1.6); 7.0924 (1.8); 7.0770 (3.8); 7.0724 (2.7); 7.0466 (2.3); 7.0428 (2.5); 7.0284 (1.9); 7.0264 (1.6); 7.0248 (1.9); 7.0228 (1.5); 7.0084 (0.9); 7.0047 (0.9); 6.9519 (2.6); 6.9482 (2.4); 6.9316 (2.2); 6.9280 (1.9); 6.6679 (4.4); 6.6518 (4.5); 6.2706 (0.6); 6.2497 (7.2); 5.2988 (7.6); 5.1755 (6.6); 4.7510 (16.0); 4.7439 (0.9); 4.7352 (0.6); 4.7212 (1.2); 3.4992 (1.0); 3.4839 (10.0); 3.4810 (10.0); 2.1154 (1.1); 2.0932 (0.6); 2.0447 (0.6); 1.4365 (0.6); 1.4224 (0.6); 1.2590 (0.8); 1.2547 (0.7); 0.0080 (0.9); 0.0040 (0.8); −0.0002 (34.3); −0.0085 (1.0)

I.8-356: ¹H-NMR (400.6 MHz, CDCl3):

δ=7.4988 (2.8); 7.4772 (2.8); 7.2928 (3.7); 7.2610 (14.5); 7.1134 (0.7); 7.1086 (0.7); 7.0953 (1.0); 7.0932 (1.0); 7.0906 (1.2); 7.0885 (1.0); 7.0752 (1.0); 7.0704 (1.3); 7.0532 (0.9); 7.0486 (1.1); 7.0332 (2.4); 7.0285 (1.8); 7.0076 (1.4); 7.0039 (1.6); 6.9896 (1.1); 6.9859 (1.2); 6.9840 (0.9); 6.9695 (0.6); 6.9658 (0.5); 6.8875 (1.6); 6.8839 (1.5); 6.8672 (1.4); 6.8637 (1.2); 6.6953 (2.7); 6.6792 (2.6); 6.2774 (4.6); 5.2988 (11.1); 5.0117 (5.6); 4.6215 (9.5); 4.5971 (0.7); 3.7941 (16.0); 3.5135 (0.6); 3.4996 (6.3); 3.4968 (6.4); 2.2141 (1.6); 2.2078 (15.0); 2.1979 (0.6); 2.1207 (0.9); 1.2590 (0.5); 0.0079 (0.7); −0.0002 (21.4); −0.0085 (0.6)

I.7-17: ¹H-NMR (400.6 MHz, CDCl3):

δ=7.3747 (4.3); 7.3526 (4.3); 7.2601 (11.6); 7.1456 (0.9); 7.1411 (1.1); 7.1253 (1.7); 7.1226 (2.0); 7.1071 (1.5); 7.1026 (1.7); 7.0758 (1.6); 7.0714 (1.8); 7.0558 (3.6); 7.0514 (2.8); 7.0209 (1.9); 7.0174 (2.1); 6.9990 (2.1); 6.9826 (0.8); 6.9791 (0.8); 6.9375 (2.7); 6.9342 (2.6); 6.9171 (2.3); 6.9139 (2.1); 6.7555 (4.1); 6.7390 (4.2); 6.2779 (7.7); 5.9527 (0.6); 5.9426 (1.2); 5.9326 (0.6); 5.8147 (1.2); 5.8046 (2.5); 5.7945 (1.2); 5.6766 (0.6); 5.6666 (1.2); 5.6565 (0.6); 5.2985 (14.4); 4.6913 (16.0); 4.3081 (2.9); 4.2975 (3.3); 4.2932 (2.8); 4.2849 (3.3); 3.7434 (3.9); 3.7315 (4.8); 3.7200 (3.6); 3.6784 (2.4); 3.6683 (2.4); 3.6436 (4.9); 3.6335 (4.8); 3.6087 (2.5); 3.5986 (2.4); 3.4976 (11.2); 3.4954 (11.7); 1.5496 (3.4); −0.0002 (15.3)

I.7-3: ¹H-NMR (400.6 MHz, CDCl3):

δ=7.3685 (3.7); 7.3464 (3.7); 7.2606 (9.4); 7.1337 (1.0); 7.1292 (1.0); 7.1153 (1.4); 7.1134 (1.4); 7.1108 (1.6); 7.1089 (1.4); 7.0951 (1.4); 7.0906 (1.7); 7.0665 (1.5); 7.0620 (1.6); 7.0464 (3.2); 7.0420 (2.4); 7.0087 (1.8); 7.0050 (2.0); 6.9904 (1.6); 6.9887 (1.3); 6.9868 (1.7); 6.9850 (1.3); 6.9704 (0.8); 6.9667 (0.8); 6.9339 (2.3); 6.9303 (2.0); 6.9135 (1.9); 6.9100 (1.6); 6.7822 (3.6); 6.7657 (3.8); 6.2780 (6.2); 5.2977 (16.0); 4.6911 (12.6); 4.2946 (3.2); 4.2857 (2.1); 4.2827 (3.2); 4.2790 (2.0); 4.2705 (3.4); 3.6140 (3.9); 3.6051 (2.1); 3.6019 (3.7); 3.5989 (2.1); 3.5899 (3.6); 3.4980 (8.3); 3.4950 (8.4); 3.3969 (4.4); 3.3800 (9.0); 3.3632 (4.5); 1.5926 (1.7); 1.5756 (3.7); 1.5571 (3.7); 1.5401 (1.9); 1.5219 (0.5); 0.9161 (6.9); 0.8976 (14.1); 0.8790 (6.2); −0.0002 (12.9)

I.7-18: ¹H-NMR (400.6 MHz, CDCl3):

δ=7.3744 (4.3); 7.3522 (4.3); 7.2600 (12.8); 7.1457 (1.0); 7.1412 (1.2); 7.1273 (1.6); 7.1254 (1.5); 7.1228 (1.8); 7.1210 (1.6); 7.1072 (1.6); 7.1026 (1.9); 7.0783 (1.7); 7.0739 (1.8); 7.0583 (3.6); 7.0539 (2.8); 7.0223 (2.1); 7.0187 (2.3); 7.0040 (1.8); 7.0023 (1.5); 7.0004 (1.9); 6.9988 (1.4); 6.9841 (0.9); 6.9804 (0.9); 6.9358 (2.6); 6.9322 (2.3); 6.9155 (2.2); 6.9119 (1.9); 6.7488 (4.2); 6.7324 (4.2); 6.2759 (7.1); 5.2981 (16.0); 4.6933 (15.7); 4.3195 (2.3); 4.3175 (2.3); 4.3106 (2.7); 4.3079 (2.7); 4.3061 (2.6); 4.3027 (2.5); 4.2960 (2.5); 4.2944 (2.4); 3.8290 (2.6); 3.8074 (10.3); 3.7970 (4.4); 3.7857 (10.6); 3.7640 (2.8); 3.4971 (9.7); 3.4942 (9.8); 1.5506 (2.4); −0.0002 (17.5)

I.7-8: ¹H-NMR (400.6 MHz, CDCl3):

δ=7.3708 (3.8); 7.3487 (3.9); 7.2605 (10.2); 7.1443 (0.9); 7.1399 (1.0); 7.1258 (1.4); 7.1240 (1.4); 7.1215 (1.6); 7.1197 (1.4); 7.1057 (1.4); 7.1013 (1.6); 7.0767 (1.6); 7.0723 (1.7); 7.0566 (3.2); 7.0523 (2.4); 7.0136 (1.7); 7.0099 (1.9); 6.9950 (1.6); 6.9936 (1.4); 6.9915 (1.7); 6.9752 (0.8); 6.9716 (0.8); 6.9317 (2.3); 6.9281 (2.1); 6.9114 (2.0); 6.9078 (1.7); 6.7793 (3.7); 6.7628 (3.8); 6.2812 (6.5); 5.2983 (16.0); 4.6971 (13.5); 4.3229 (1.6); 4.3139 (2.6); 4.3106 (2.1); 4.3043 (2.1); 4.3003 (2.9); 4.2915 (1.7); 4.1155 (5.9); 4.1137 (5.7); 4.1096 (5.9); 4.1077 (5.4); 3.7076 (2.2); 3.7042 (2.3); 3.6958 (3.6); 3.6928 (3.3); 3.6845 (1.9); 3.6803 (1.8); 3.4996 (9.1); 3.4968 (8.9); 2.4400 (2.6); 2.4341 (5.2); 2.4281 (2.4); 1.5566 (1.9); −0.0002 (13.0)

I.7-7: ¹H-NMR (400.6 MHz, CDCl3):

δ=7.3688 (4.6); 7.3467 (4.7); 7.2603 (11.8); 7.1364 (1.0); 7.1319 (1.2); 7.1179 (1.7); 7.1161 (1.6); 7.1135 (2.0); 7.1117 (1.7); 7.0978 (1.6); 7.0934 (1.9); 7.0713 (1.8); 7.0669 (2.0); 7.0512 (3.8); 7.0469 (2.9); 7.0113 (2.1); 7.0076 (2.3); 6.9928 (2.0); 6.9910 (1.7); 6.9892 (2.0); 6.9729 (0.9); 6.9693 (1.0); 6.9323 (2.8); 6.9288 (2.5); 6.9120 (2.4); 6.9085 (2.0); 6.7791 (4.5); 6.7626 (4.5); 6.2764 (7.8); 5.8987 (0.6); 5.8845 (1.3); 5.8727 (0.8); 5.8704 (0.7); 5.8586 (1.5); 5.8557 (0.8); 5.8443 (0.8); 5.8414 (1.5); 5.8297 (0.9); 5.8273 (0.8); 5.8155 (1.6); 5.8014 (0.8); 5.2980 (13.2); 5.2813 (0.9); 5.2773 (2.4); 5.2732 (2.4); 5.2692 (0.9); 5.2383 (0.8); 5.2343 (2.0); 5.2302 (2.1); 5.2262 (0.8); 5.2046 (1.0); 5.2015 (2.2); 5.1980 (1.8); 5.1943 (0.8); 5.1787 (0.9); 5.1756 (2.0); 5.1721 (1.7); 5.1683 (0.7); 4.6942 (16.0); 4.3068 (3.6); 4.2978 (2.7); 4.2951 (3.6); 4.2911 (2.5); 4.2829 (3.9); 3.9768 (3.3); 3.9735 (5.4); 3.9699 (3.2); 3.9627 (3.3); 3.9594 (5.2); 3.9557 (3.0); 3.6219 (4.8); 3.6154 (1.6); 3.6124 (2.9); 3.6100 (4.8); 3.6046 (1.4); 3.5981 (4.3); 3.4959 (10.8); 3.4931 (10.5); 1.5595 (1.7); 0.0079 (0.5); −0.0002 (15.6)

I.7-27: ¹H-NMR (400.6 MHz, CDCl3):

δ=7.3706 (4.6); 7.3484 (4.6); 7.2605 (12.1); 7.1398 (1.0); 7.1354 (10.2); 7.1194 (2.0); 7.1169 (2.3); 7.1012 (1.6); 7.0968 (1.8); 7.0698 (1.7); 7.0655 (2.0); 7.0498 (3.7); 7.0455 (3.0); 7.0108 (2.0); 7.0073 (2.2); 6.9889 (2.4); 6.9724 (0.9); 6.9690 (0.9); 6.9219 (2.9); 6.9188 (2.8); 6.9016 (2.4); 6.8985 (2.2); 6.7717 (4.4); 6.7553 (4.4); 6.2836 (8.2); 5.2984 (8.8); 4.6524 (15.8); 4.2493 (3.8); 4.2332 (8.0); 4.2171 (3.9); 3.4999 (12.6); 3.4980 (12.8); 3.4571 (2.5); 3.4396 (7.7); 3.4329 (4.4); 3.4221 (8.3); 3.4174 (9.1); 3.4021 (4.8); 1.9129 (1.1); 1.8972 (4.1); 1.8814 (6.0); 1.8656 (3.9); 1.8497 (1.0); 1.5598 (2.0); 1.1867 (8.0); 1.1692 (16.0); 1.1517 (7.6); 0.0079 (0.6); −0.0002 (15.0)

I.7-24: ¹H-NMR (400.6 MHz, CDCl3):

δ=7.3699 (4.4); 7.3478 (4.4); 7.2608 (12.9); 7.1389 (1.0); 7.1345 (1.1); 7.1162 (2.2); 7.1004 (1.6); 7.0960 (1.8); 7.0650 (1.8); 7.0607 (1.9); 7.0450 (3.8); 7.0408 (2.9); 7.0095 (2.2); 7.0061 (2.2); 6.9899 (2.3); 6.9880 (2.3); 6.9713 (1.0); 6.9678 (0.9); 6.9374 (3.2); 6.9171 (2.6); 6.7773 (4.4); 6.7609 (4.4); 6.2802 (8.3); 5.2987 (5.8); 4.6874 (15.9); 4.3094 (4.0); 4.2975 (4.6); 4.2853 (4.2); 3.7000 (4.5); 3.6878 (4.9); 3.6760 (4.1); 3.6185 (1.9); 3.6161 (1.9); 3.6080 (4.8); 3.6010 (3.4); 3.5944 (5.6); 3.5668 (5.7); 3.5591 (3.6); 3.5521 (4.6); 3.5417 (4.1); 3.5242 (7.8); 3.5066 (9.0); 3.4987 (13.8); 3.4894 (3.3); 1.5659 (2.8); 1.2231 (8.0); 1.2056 (16.0); 1.1881 (7.8); 0.0079 (0.6); −0.0002 (17.3); −0.0085 (0.6)

I.7-4: ¹H-NMR (400.6 MHz, CDCl3):

δ=7.3681 (2.0); 7.3459 (2.0); 7.2605 (5.9); 7.1296 (0.5); 7.1157 (0.7); 7.1138 (0.7); 7.1113 (0.8); 7.1094 (0.7); 7.0956 (0.7); 7.0911 (0.8); 7.0659 (0.8); 7.0614 (0.8); 7.0458 (1.7); 7.0414 (1.2); 7.0080 (0.9); 7.0043 (1.0); 6.9896 (0.9); 6.9879 (0.7); 6.9860 (0.9); 6.9845 (0.7); 6.9348 (1.2); 6.9312 (1.1); 6.9145 (1.0); 6.9109 (0.9); 6.7864 (1.9); 6.7699 (2.0); 6.2794 (3.3); 5.2983 (5.2); 4.6894 (6.6); 4.2766 (1.6); 4.2645 (1.7); 4.2613 (1.1); 4.2520 (1.8); 3.6077 (2.1); 3.5978 (1.4); 3.5953 (2.1); 3.5908 (0.7); 3.5834 (2.6); 3.5686 (1.4); 3.5533 (1.0); 3.4989 (4.6); 3.4960 (4.5); 1.5618 (0.8); 1.1430 (16.0); 1.1366 (0.5); 1.1277 (16.0); −0.0002 (7.9)

I.8-277: ¹H-NMR (400.6 MHz, CDCl3):

δ=8.7665 (1.9); 8.7508 (1.0); 8.1424 (1.0); 8.1226 (1.0); 7.7385 (0.8); 7.7246 (0.9); 7.7187 (0.8); 7.7048 (0.7); 7.4650 (3.0); 7.4436 (3.0); 7.2609 (17.2); 7.1381 (0.6); 7.1334 (0.7); 7.1199 (1.1); 7.1180 (1.0); 7.1153 (1.3); 7.1133 (1.0); 7.1011 (1.5); 7.0960 (2.0); 7.0813 (2.6); 7.0768 (1.5); 7.0502 (1.5); 7.0463 (1.6); 7.0320 (1.2); 7.0286 (1.1); 7.0261 (0.8); 7.0120 (0.6); 7.0083 (0.5); 6.9286 (1.7); 6.9249 (1.5); 6.9084 (1.4); 6.9050 (1.2); 6.6521 (2.9); 6.6361 (2.9); 6.2720 (4.8); 5.2994 (16.0); 5.2917 (6.8); 4.7088 (6.0); 4.7062 (5.6); 3.4912 (6.6); 3.4884 (6.4); 0.0080 (0.9); −0.0002 (24.1); −0.0086 (0.6)

I.9-278: ¹H-NMR (400.6 MHz, CDCl3):

δ=8.8366 (3.0); 8.8332 (1.6); 8.8233 (1.7); 8.8201 (3.0); 7.6770 (2.5); 7.6606 (2.4); 7.4601 (2.6); 7.4392 (2.6); 7.2610 (19.6); 7.2564 (0.7); 7.2520 (1.3); 7.2498 (1.7); 7.2455 (1.5); 7.2340 (1.6); 7.2296 (2.3); 7.2257 (1.8); 7.2163 (1.5); 7.2121 (0.6); 7.1255 (1.1); 7.1219 (1.2); 7.1073 (1.0); 7.1048 (1.0); 7.1026 (1.7); 7.0868 (0.7); 7.0832 (0.6); 6.9864 (1.6); 6.9833 (1.4); 6.9656 (1.4); 6.9615 (1.1); 6.6828 (2.5); 6.6684 (2.6); 6.2711 (4.7); 5.3773 (5.6); 5.2997 (16.0); 4.7614 (6.6); 3.4992 (6.3); 3.4966 (6.2); 0.0080 (0.8); −0.0002 (26.4); −0.0085 (0.8)

I.9-277: ¹H-NMR (400.6 MHz, CDCl3):

δ=8.5953 (1.9); 8.5917 (1.9); 8.5832 (2.0); 8.5796 (1.9); 8.5544 (2.6); 8.5498 (2.6); 7.6475 (1.2); 7.6431 (1.9); 7.6386 (1.1); 7.6280 (1.3); 7.6235 (2.1); 7.6190 (1.1); 7.4407 (4.9); 7.4197 (4.9); 7.3156 (1.8); 7.3032 (1.8); 7.2961 (1.6); 7.2840 (1.7); 7.2613 (18.2); 7.2226 (2.3); 7.2186 (2.8); 7.2029 (2.8); 7.1990 (4.6); 7.1800 (2.7); 7.1757 (2.0); 7.1602 (1.9); 7.1560 (1.5); 7.0674 (1.9); 7.0639 (2.0); 7.0481 (2.8); 7.0446 (2.7); 7.0287 (1.2); 7.0253 (1.2); 6.8720 (2.8); 6.8686 (2.7); 6.8516 (2.6); 6.8482 (2.4); 6.7039 (4.7); 6.6893 (4.7); 6.2618 (8.7); 5.2991 (9.6); 5.1686 (10.9); 4.6586 (16.0); 3.4825 (11.8); 3.4800 (11.9); 2.1182 (1.1); 2.0447 (1.1); 1.4367 (0.6); 1.4224 (0.6); 1.2592 (1.0); 0.0084 (0.8); −0.0002 (26.3); −0.0078 (0.7)

I.9-356: ¹H-NMR (400.6 MHz, CDCl3):

δ=7.4347 (2.6); 7.4135 (2.6); 7.3124 (3.7); 7.2616 (12.6); 7.2126 (1.3); 7.2085 (1.6); 7.1929 (1.6); 7.1890 (2.6); 7.1853 (0.8); 7.1703 (1.4); 7.1659 (1.0); 7.1501 (1.2); 7.1459 (0.9); 7.0529 (1.1); 7.0493 (1.2); 7.0336 (1.6); 7.0300 (1.5); 7.0143 (0.7); 7.0107 (0.7); 6.8429 (1.5); 6.8394 (1.5); 6.8224 (1.4); 6.8190 (1.3); 6.7170 (2.5); 6.7024 (2.6); 6.2694 (4.7); 5.2989 (12.6); 4.9953 (4.6); 4.9917 (4.6); 4.5763 (8.4); 3.8108 (16.0); 3.4931 (6.1); 3.4903 (6.2); 2.2034 (15.3); 2.1191 (1.0); 2.0444 (1.6); 1.4352 (0.5); 1.4211 (0.5); 1.2769 (0.6); 1.2591 (1.3); 1.2413 (0.5); 0.0079 (0.5); −0.0002 (18.1); −0.0085 (0.5)

I.7-43: ¹H-NMR (400.6 MHz, CDCl3):

δ=7.3750 (4.5); 7.3529 (4.5); 7.2600 (20.5); 7.1494 (1.1); 7.1449 (1.3); 7.1311 (1.6); 7.1292 (1.6); 7.1266 (1.9); 7.1247 (1.8); 7.1109 (1.8); 7.1064 (2.1); 7.0831 (1.8); 7.0786 (2.0); 7.0630 (3.9); 7.0586 (3.0); 7.0263 (2.2); 7.0226 (2.5); 7.0079 (1.9); 7.0062 (1.6); 7.0043 (2.0); 7.0026 (1.6); 6.9879 (1.0); 6.9843 (1.0); 6.9244 (2.7); 6.9208 (2.5); 6.9041 (2.3); 6.9005 (2.0); 6.7336 (4.3); 6.7172 (4.5); 6.2775 (7.2); 5.2988 (6.5); 4.6625 (16.0); 4.2628 (3.5); 4.2474 (7.5); 4.2319 (3.7); 3.9891 (4.2); 3.9737 (8.8); 3.9584 (4.4); 3.4989 (9.7); 3.4959 (10.1); 2.0383 (0.6); 2.0232 (2.4); 2.0079 (3.5); 1.9926 (2.3); 1.9772 (0.6); 1.5456 (1.8); 0.0080 (0.8); −0.0002 (30.6); −0.0084 (1.0)

I.7-9: ¹H-NMR (400.6 MHz, CDCl3):

δ=7.3814 (3.3); 7.3593 (3.4); 7.2604 (12.6); 7.1560 (0.8); 7.1514 (0.9); 7.1377 (1.4); 7.1357 (1.4); 7.1331 (1.5); 7.1312 (1.2); 7.1175 (1.2); 7.1129 (1.4); 7.0780 (1.2); 7.0736 (1.4); 7.0581 (2.9); 7.0536 (2.2); 7.0279 (1.7); 7.0243 (1.8); 7.0096 (1.6); 7.0077 (1.4); 7.0061 (1.6); 6.9897 (0.7); 6.9861 (0.7); 6.9467 (2.2); 6.9433 (1.9); 6.9264 (1.8); 6.9230 (1.5); 6.7492 (3.3); 6.7328 (3.4); 6.2844 (5.8); 5.2990 (10.3); 4.6995 (12.5); 4.3412 (2.0); 4.3389 (1.8); 4.3326 (2.3); 4.3298 (2.4); 4.3277 (2.2); 4.3244 (2.0); 4.3182 (2.2); 4.2275 (16.0); 3.7797 (3.2); 3.7685 (4.4); 3.7572 (2.7); 3.5032 (8.8); 3.5006 (8.2); 1.5497 (2.2); 0.0080 (0.6); −0.0002 (19.0); −0.0085 (0.6)

I.7-42: ¹H-NMR (400.6 MHz, CDCl3):

δ=7.3781 (4.5); 7.3559 (4.5); 7.2599 (18.4); 7.1409 (1.0); 7.1362 (1.1); 7.1226 (1.6); 7.1206 (1.5); 7.1180 (1.8); 7.1161 (1.5); 7.1025 (1.5); 7.0979 (1.8); 7.0779 (1.6); 7.0733 (1.8); 7.0579 (3.8); 7.0533 (2.8); 7.0278 (2.1); 7.0241 (2.2); 7.0095 (1.8); 7.0059 (1.8); 6.9896 (0.8); 6.9859 (0.8); 6.9292 (2.7); 6.9256 (2.4); 6.9089 (2.2); 6.9053 (2.0); 6.7264 (4.3); 6.7099 (4.3); 6.2764 (7.4); 5.2988 (4.6); 4.6998 (16.0); 4.3979 (3.3); 4.3865 (4.0); 4.3827 (2.3); 4.3743 (4.3); 4.2099 (3.9); 4.1977 (3.9); 4.1863 (2.9); 3.4983 (10.2); 3.4955 (10.1); 1.5426 (3.2); 0.0079 (0.8); −0.0002 (26.8); −0.0085 (0.8)

I.7-303: ¹H-NMR (400.6 MHz, CDCl3):

δ=8.4246 (1.5); 8.4193 (1.5); 7.7194 (1.6); 7.7142 (1.6); 7.3442 (1.3); 7.3221 (1.4); 7.2607 (1.4); 7.1166 (0.5); 7.1141 (0.5); 7.1005 (0.5); 7.0961 (0.6); 7.0700 (0.7); 7.0535 (0.8); 7.0495 (0.9); 7.0078 (1.9); 6.9881 (1.6); 6.9852 (0.6); 6.7857 (1.3); 6.7693 (1.3); 6.2609 (2.2); 5.3547 (4.6); 5.2951 (16.0); 4.7924 (4.1); 3.4840 (3.0); 3.4812 (3.0); 2.1676 (1.2); −0.0002 (2.1)

I.7-309: ¹H-NMR (400.6 MHz, CDCl3):

δ=8.0746 (1.0); 8.0728 (1.0); 8.0552 (1.2); 7.8496 (1.2); 7.8301 (2.3); 7.8106 (1.2); 7.4580 (1.4); 7.4382 (1.1); 7.4363 (1.1); 7.3314 (2.1); 7.3093 (2.1); 7.2622 (5.9); 7.1336 (0.5); 7.1195 (0.7); 7.1180 (0.7); 7.1150 (0.9); 7.0997 (0.7); 7.0952 (1.0); 7.0832 (0.8); 7.0788 (0.9); 7.0632 (1.7); 7.0588 (1.2); 7.0246 (0.9); 7.0209 (1.1); 7.0062 (0.8); 7.0026 (0.9); 6.9807 (1.4); 6.9770 (1.1); 6.9604 (1.0); 6.9569 (0.8); 6.7582 (2.0); 6.7417 (2.0); 6.2447 (3.5); 5.3906 (4.8); 5.2988 (16.0); 4.7815 (6.7); 3.9993 (16.0); 3.4804 (4.7); 3.4777 (4.7); 2.0447 (0.9); 1.2587 (0.8); −0.0002 (8.7)

I.7-301: ¹H-NMR (400.6 MHz, CDCl3):

δ=8.3203 (1.8); 8.3144 (1.8); 7.3445 (2.3); 7.3223 (2.3); 7.2606 (4.8); 7.2451 (0.6); 7.2392 (0.6); 7.2250 (0.7); 7.2223 (0.7); 7.2192 (0.7); 7.2164 (0.6); 7.2023 (0.6); 7.1963 (0.6); 7.1211 (0.5); 7.1165 (0.6); 7.1026 (0.8); 7.1009 (0.8); 7.0982 (1.0); 7.0965 (0.9); 7.0826 (0.8); 7.0781 (1.0); 7.0612 (0.9); 7.0568 (1.0); 7.0411 (1.9); 7.0367 (1.4); 7.0033 (1.0); 6.9996 (1.2); 6.9849 (0.9); 6.9834 (0.8); 6.9813 (1.0); 6.9797 (0.9); 6.9449 (1.4); 6.9413 (1.2); 6.9246 (1.1); 6.9210 (1.0); 6.7653 (2.2); 6.7489 (2.2); 6.2790 (3.8); 5.3230 (3.2); 5.3181 (3.1); 5.2977 (16.0); 4.7390 (7.7); 3.4966 (5.2); 3.4938 (5.3); −0.0002 (7.0)

I.7-302: ¹H-NMR (400.6 MHz, CDCl3):

δ=8.3761 (1.4); 8.3714 (1.4); 7.4775 (1.1); 7.4726 (1.0); 7.4552 (1.0); 7.4503 (1.0); 7.3427 (2.0); 7.3205 (2.0); 7.2613 (1.8); 7.1048 (0.7); 7.1003 (0.8); 7.0851 (0.6); 7.0806 (0.8); 7.0642 (0.8); 7.0597 (0.8); 7.0442 (1.6); 7.0397 (1.2); 7.0056 (0.8); 7.0020 (0.9); 6.9870 (0.8); 6.9833 (0.9); 6.9509 (1.2); 6.9473 (1.0); 6.9306 (1.0); 6.9271 (0.8); 6.7651 (1.9); 6.7486 (1.9); 6.2744 (3.4); 5.3193 (3.7); 5.3142 (3.6); 5.2959 (16.0); 4.7451 (6.6); 3.4927 (4.6); 3.4902 (4.6); −0.0002 (2.7)

I.7-308: ¹H-NMR (400.6 MHz, CDCl3):

δ=9.1442 (0.9); 9.1423 (0.9); 9.1389 (0.9); 9.1371 (0.8); 8.2643 (0.8); 8.2590 (0.8); 8.2440 (0.8); 8.2386 (0.8); 7.3285 (1.3); 7.3061 (1.7); 7.2837 (0.9); 7.2819 (0.8); 7.2640 (1.9); 7.1332 (0.5); 7.1315 (0.5); 7.1287 (0.6); 7.1271 (0.5); 7.1087 (0.6); 7.0956 (0.5); 7.0912 (0.6); 7.0756 (1.1); 7.0712 (0.8); 7.0350 (0.6); 7.0313 (0.7); 7.0166 (0.6); 7.0149 (0.5); 7.0129 (0.6); 6.9801 (0.9); 6.9766 (0.7); 6.9598 (0.7); 6.9563 (0.6); 6.7401 (1.3); 6.7238 (1.3); 6.2429 (2.3); 5.3351 (3.7); 5.2973 (16.0); 4.7933 (4.6); 3.9580 (10.0); 3.4795 (3.2); 3.4767 (3.1); −0.0002 (2.6)

I.7-327: ¹H-NMR (400.6 MHz, CDCl3):

δ=8.1776 (1.0); 8.1721 (1.0); 7.7344 (0.7); 7.7282 (0.6); 7.3379 (2.0); 7.3158 (2.0); 7.2625 (2.6); 7.1177 (0.5); 7.1047 (0.7); 7.1024 (0.7); 7.0998 (0.8); 7.0976 (0.7); 7.0846 (0.7); 7.0797 (0.9); 7.0636 (0.6); 7.0587 (0.8); 7.0435 (1.9); 7.0387 (1.3); 7.0239 (1.1); 7.0203 (1.1); 7.0061 (0.8); 7.0027 (0.8); 7.0005 (0.5); 6.9203 (0.9); 6.9098 (1.3); 6.9061 (1.2); 6.8993 (0.8); 6.8899 (1.3); 6.8859 (0.9); 6.6944 (1.9); 6.6780 (1.9); 6.2764 (3.4); 5.2975 (16.0); 5.1583 (4.7); 4.6825 (7.4); 3.4984 (4.8); 3.4956 (4.6); −0.0002 (3.8)

I.7-321: ¹H-NMR (400.6 MHz, CDCl3):

δ=9.1882 (2.7); 8.7020 (6.3); 7.3460 (2.2); 7.3239 (2.2); 7.2628 (5.7); 7.1322 (0.5); 7.1273 (0.6); 7.1142 (0.8); 7.1120 (0.7); 7.1094 (1.0); 7.1072 (0.8); 7.0942 (0.8); 7.0893 (1.1); 7.0802 (0.7); 7.0755 (0.8); 7.0601 (2.0); 7.0554 (1.3); 7.0360 (1.2); 7.0323 (1.3); 7.0180 (0.9); 7.0159 (0.7); 7.0145 (0.9); 7.0122 (0.6); 6.9193 (1.3); 6.9156 (1.2); 6.8990 (1.1); 6.8955 (1.0); 6.6873 (2.1); 6.6709 (2.1); 6.2822 (3.6); 5.2987 (16.0); 5.1788 (5.8); 4.6983 (8.2); 3.4974 (4.8); 3.4945 (4.8); −0.0002 (8.0)

I.7-275: ¹H-NMR (400.6 MHz, CDCl3):

δ=7.3510 (4.6); 7.3288 (4.7); 7.3222 (4.4); 7.3166 (4.3); 7.2606 (16.2); 7.1109 (1.0); 7.1064 (1.2); 7.0924 (1.6); 7.0907 (1.6); 7.0881 (1.9); 7.0863 (1.7); 7.0724 (1.5); 7.0679 (2.0); 7.0601 (1.9); 7.0557 (2.0); 7.0400 (3.8); 7.0357 (2.7); 6.9959 (2.0); 6.9922 (2.2); 6.9774 (1.9); 6.9758 (1.8); 6.9739 (2.0); 6.9575 (1.0); 6.9539 (0.9); 6.9002 (2.7); 6.8966 (2.5); 6.8799 (2.3); 6.8764 (2.1); 6.7910 (4.4); 6.7745 (4.5); 6.2809 (7.8); 6.2216 (4.8); 6.2160 (4.7); 5.2984 (4.0); 5.1674 (14.8); 4.6860 (14.6); 4.0606 (4.3); 4.0429 (6.1); 4.0249 (4.4); 3.4972 (10.6); 3.4944 (10.6); 1.9123 (0.5); 1.8938 (2.3); 1.8756 (4.2); 1.8577 (4.2); 1.8395 (2.5); 1.8213 (0.6); 1.6035 (0.6); 0.9285 (7.7); 0.9101 (16.0); 0.8915 (7.1); 0.0079 (0.7); −0.0002 (23.8); −0.0085 (0.7)

I.7-282: ¹H-NMR (400.6 MHz, CDCl3):

δ=8.5353 (1.6); 8.5216 (1.6); 8.5142 (1.5); 8.5006 (1.5); 7.3386 (4.5); 7.3164 (4.5); 7.2607 (18.9); 7.1491 (1.0); 7.1447 (1.2); 7.1307 (1.5); 7.1290 (1.6); 7.1262 (2.0); 7.1245 (1.9); 7.1106 (1.7); 7.1062 (2.2); 7.0959 (1.9); 7.0916 (2.0); 7.0758 (3.8); 7.0715 (2.8); 7.0333 (2.1); 7.0295 (2.4); 7.0149 (1.9); 7.0134 (1.6); 7.0113 (2.0); 7.0096 (1.8); 6.9949 (1.1); 6.9911 (2.0); 6.9849 (2.0); 6.9837 (2.0); 6.9761 (1.6); 6.9704 (3.5); 6.9669 (3.6); 6.9617 (3.1); 6.9553 (2.3); 6.9503 (2.7); 6.9469 (2.2); 6.9416 (1.4); 6.9354 (0.8); 6.7443 (4.4); 6.7280 (4.4); 6.2554 (7.5); 5.2987 (6.5); 5.2765 (12.6); 4.7837 (16.0); 3.4857 (10.1); 3.4828 (10.5); 2.0446 (0.9); 1.2591 (0.9); 0.8819 (0.5); 0.0080 (0.7); −0.0002 (28.3); −0.0085 (0.8)

I.7-283: ¹H-NMR (400.6 MHz, CDCl3):

δ=8.4132 (3.2); 8.4060 (3.1); 7.3993 (1.0); 7.3921 (1.0); 7.3783 (1.9); 7.3711 (1.9); 7.3576 (1.5); 7.3504 (1.4); 7.3347 (4.4); 7.3125 (4.6); 7.2753 (2.0); 7.2607 (15.1); 7.2538 (1.4); 7.2429 (1.3); 7.1340 (1.0); 7.1295 (1.2); 7.1157 (1.6); 7.1139 (1.6); 7.1112 (1.9); 7.1094 (1.6); 7.0957 (1.6); 7.0911 (2.0); 7.0802 (1.7); 7.0757 (1.8); 7.0601 (3.8); 7.0557 (2.7); 7.0234 (2.1); 7.0197 (2.3); 7.0051 (1.9); 7.0034 (1.6); 7.0016 (1.9); 6.9998 (1.5); 6.9852 (0.9); 6.9815 (0.9); 6.9488 (2.8); 6.9452 (2.4); 6.9286 (2.3); 6.9250 (1.9); 6.7450 (4.3); 6.7286 (4.4); 6.2580 (7.4); 5.2987 (6.0); 5.2560 (9.9); 4.7531 (16.0); 3.4885 (10.3); 3.4856 (10.2); 0.0079 (0.7); −0.0002 (21.9); −0.0085 (0.6)

I.7-272: ¹H-NMR (400.6 MHz, CDCl3):

δ=7.3497 (2.5); 7.3275 (2.5); 7.2987 (2.4); 7.2932 (2.3); 7.2606 (7.4); 7.2592 (6.0); 7.1135 (0.5); 7.1091 (0.6); 7.0936 (1.1); 7.0911 (1.1); 7.0893 (1.2); 7.0750 (0.8); 7.0706 (1.0); 7.0620 (1.0); 7.0577 (1.0); 7.0419 (2.0); 7.0376 (1.4); 6.9984 (1.0); 6.9949 (1.2); 6.9788 (1.2); 6.9769 (1.2); 6.9751 (1.2); 6.9010 (1.6); 6.8976 (1.4); 6.8808 (1.3); 6.8774 (1.1); 6.7889 (2.3); 6.7724 (2.3); 6.2783 (4.5); 6.2196 (2.6); 6.2141 (2.5); 5.2985 (1.9); 5.2971 (1.5); 5.1564 (8.6); 4.6843 (8.6); 3.8733 (16.0); 3.4954 (6.6); 3.4939 (6.6); 1.5823 (1.7); −0.0002 (10.3); −0.0017 (8.6)

I.7-281: ¹H-NMR (400.6 MHz, CDCl3):

δ=8.4081 (1.4); 8.4047 (2.6); 8.4013 (1.5); 8.3966 (1.5); 8.3931 (2.6); 8.3898 (1.4); 7.4353 (1.2); 7.4320 (1.2); 7.4142 (1.8); 7.4113 (2.6); 7.3908 (1.7); 7.3875 (1.7); 7.3390 (5.1); 7.3243 (1.6); 7.3168 (5.4); 7.3134 (2.9); 7.3027 (2.3); 7.2924 (1.8); 7.2814 (1.0); 7.2607 (10.8); 7.1198 (1.0); 7.1155 (1.2); 7.1004 (1.9); 7.0961 (2.7); 7.0812 (1.6); 7.0768 (2.1); 7.0642 (2.0); 7.0601 (2.2); 7.0445 (3.6); 7.0401 (2.9); 6.9937 (1.8); 6.9901 (2.5); 6.9749 (2.0); 6.9709 (3.1); 6.9655 (3.6); 6.9624 (2.2); 6.9553 (1.0); 6.9517 (1.3); 6.9455 (2.6); 6.9420 (1.9); 6.7865 (4.9); 6.7700 (4.9); 6.2739 (8.6); 5.3626 (8.4); 5.3576 (8.4); 5.2979 (8.6); 4.7639 (16.0); 3.4916 (11.6); 3.4890 (11.9); 1.5990 (0.5); 1.2583 (0.6); −0.0002 (15.5)

I.7-274: ¹H-NMR (400.6 MHz, CDCl3):

δ=7.3503 (4.5); 7.3409 (4.2); 7.3354 (4.2); 7.3281 (4.4); 7.2609 (10.3); 7.1130 (1.0); 7.1085 (1.1); 7.0943 (1.6); 7.0927 (1.6); 7.0900 (2.0); 7.0745 (1.4); 7.0700 (1.9); 7.0612 (1.8); 7.0568 (1.9); 7.0411 (3.6); 7.0368 (2.6); 6.9967 (1.9); 6.9931 (2.0); 6.9781 (1.9); 6.9764 (1.9); 6.9746 (2.0); 6.9583 (0.8); 6.9547 (0.9); 6.9027 (2.6); 6.8993 (2.5); 6.8824 (2.2); 6.8790 (2.0); 6.7927 (4.2); 6.7762 (4.2); 6.2806 (7.7); 6.2278 (4.5); 6.2221 (4.4); 5.2978 (5.0); 5.1687 (14.9); 4.6864 (14.5); 4.6540 (1.2); 4.1711 (2.2); 4.1528 (6.7); 4.1345 (6.8); 4.1162 (2.3); 3.4965 (10.8); 3.4943 (10.9); 1.4903 (7.8); 1.4720 (16.0); 1.4538 (7.6); 1.3176 (0.6); 1.2998 (1.1); 1.2819 (0.6); 1.2587 (0.5); 0.0079 (0.6); −0.0002 (14.7)

I.28-1: ¹H-NMR (400.6 MHz, CDCl3):

δ=7.5410 (1.9); 7.5196 (2.0); 7.2611 (5.0); 7.2457 (1.0); 7.2398 (1.3); 7.2241 (1.0); 7.2182 (1.6); 7.2019 (2.7); 7.1961 (1.8); 6.8176 (2.1); 6.7959 (1.9); 6.7884 (1.9); 6.7724 (1.9); 6.2910 (3.1); 5.2991 (7.9); 4.6734 (6.5); 4.2890 (1.5); 4.2807 (1.0); 4.2775 (1.4); 4.2737 (1.0); 4.2657 (1.7); 3.5648 (2.0); 3.5589 (0.6); 3.5561 (1.1); 3.5531 (1.9); 3.5504 (1.1); 3.5475 (0.6); 3.5415 (1.9); 3.5111 (4.3); 3.5083 (4.3); 3.3374 (16.0); 1.5621 (1.6); −0.0002 (7.3)

I.28-23: ¹H-NMR (400.6 MHz, CDCl3):

δ=7.5421 (1.8); 7.5206 (1.8); 7.2622 (3.9); 7.2457 (1.0); 7.2398 (1.2); 7.2240 (1.0); 7.2181 (1.5); 7.1937 (2.5); 7.1878 (1.8); 6.8275 (2.0); 6.8058 (1.8); 6.7877 (1.8); 6.7717 (1.8); 6.2939 (3.0); 5.2990 (9.0); 4.6669 (5.8); 4.3077 (1.4); 4.2990 (1.1); 4.2959 (1.5); 4.2920 (1.0); 4.2837 (1.5); 3.6901 (1.7); 3.6813 (1.1); 3.6781 (1.7); 3.6750 (1.0); 3.6662 (1.5); 3.6097 (1.2); 3.6008 (1.5); 3.5989 (1.5); 3.5929 (1.4); 3.5865 (2.4); 3.5234 (2.5); 3.5165 (2.3); 3.5118 (5.6); 3.5090 (5.2); 3.5013 (1.2); 3.3704 (16.0); 1.5840 (0.5); −0.0002 (5.8)

I.28-335: ¹H-NMR (400.6 MHz, CDCl3):

δ=8.6662 (1.8); 8.6628 (1.8); 7.7710 (0.9); 7.7657 (0.9); 7.7511 (1.4); 7.7457 (1.4); 7.6918 (2.1); 7.6900 (2.2); 7.6719 (1.3); 7.6700 (1.3); 7.5005 (2.3); 7.4792 (2.3); 7.2615 (7.8); 7.2472 (1.3); 7.2413 (1.6); 7.2257 (1.2); 7.2198 (2.0); 7.2030 (3.2); 7.1973 (2.3); 6.8286 (2.6); 6.8070 (2.3); 6.7010 (2.2); 6.6852 (2.2); 6.2907 (4.1); 5.2998 (16.0); 5.2346 (6.8); 4.6836 (8.4); 3.5174 (6.6); 3.5150 (6.8); 2.0447 (0.8); 1.5586 (3.6); 1.2593 (0.6); −0.0002 (11.4)

I.28-277: ¹H-NMR (400.6 MHz, CDCl3):

δ=8.5959 (0.9); 8.5922 (1.0); 8.5842 (2.0); 8.5804 (1.9); 7.6400 (0.6); 7.6357 (0.8); 7.6302 (0.5); 7.6204 (0.6); 7.6160 (0.9); 7.6106 (0.5); 7.5027 (2.3); 7.4813 (2.3); 7.3050 (0.8); 7.2930 (0.8); 7.2855 (0.7); 7.2734 (0.7); 7.2624 (5.4); 7.2241 (1.2); 7.2182 (1.7); 7.2028 (1.0); 7.1968 (2.4); 7.1900 (3.3); 7.1845 (1.7); 6.7955 (2.3); 6.7741 (2.1); 6.7217 (2.2); 6.7058 (2.2); 6.2901 (3.8); 5.2986 (16.0); 5.1798 (6.2); 4.6679 (8.1); 3.5074 (5.5); 3.5046 (5.3); 2.0442 (0.8); 1.2587 (0.5); −0.0002 (7.9)

I.28-329: ¹H-NMR (400.6 MHz, CDCl3):

δ=8.4757 (0.9); 8.4715 (0.9); 8.4635 (0.9); 8.4593 (0.9); 7.5548 (0.8); 7.5510 (0.8); 7.5358 (0.8); 7.5318 (0.8); 7.4923 (2.2); 7.4708 (2.3); 7.2617 (9.2); 7.2268 (1.3); 7.2209 (1.7); 7.2054 (1.1); 7.1995 (2.4); 7.1902 (3.3); 7.1845 (1.9); 7.1380 (0.7); 7.1257 (0.8); 7.1191 (0.7); 7.1065 (0.7); 6.7987 (2.3); 6.7772 (2.1); 6.7238 (2.2); 6.7078 (2.2); 6.2784 (3.6); 5.2989 (16.0); 5.1863 (5.5); 4.6794 (7.6); 3.5048 (4.7); 3.5019 (4.8); 2.5385 (0.6); 2.5314 (9.6); −0.0002 (13.7)

I.28-321: ¹H-NMR (400.6 MHz, CDCl3):

δ=9.2019 (5.7); 8.7214 (10.2); 7.5180 (4.4); 7.4966 (4.4); 7.2616 (18.5); 7.2408 (2.1); 7.2349 (3.0); 7.2195 (1.6); 7.2136 (4.2); 7.2073 (6.3); 7.2017 (3.3); 6.8113 (4.5); 6.7900 (4.0); 6.6988 (4.3); 6.6830 (4.3); 6.2974 (7.7); 5.2997 (13.6); 5.1755 (13.1); 4.6772 (0.8); 4.6664 (16.0); 3.5116 (10.7); 3.5092 (11.1); 2.1263 (1.0); 2.0448 (1.2); 1.5804 (2.0); 1.2592 (0.9); 0.0080 (0.9); −0.0002 (26.7); −0.0085 (0.8)

I.28-356: ¹H-NMR (400.6 MHz, CDCl3):

δ=7.5095 (2.6); 7.4880 (2.6); 7.2845 (3.5); 7.2620 (7.3); 7.2138 (1.3); 7.2079 (1.7); 7.1922 (1.3); 7.1863 (2.1); 7.1684 (3.5); 7.1626 (2.4); 6.7658 (2.7); 6.7441 (2.6); 6.7372 (2.4); 6.7212 (2.4); 6.2954 (4.3); 5.2987 (16.0); 5.0093 (6.2); 4.5969 (8.7); 3.8818 (0.6); 3.8037 (0.8); 3.7959 (14.4); 3.5158 (5.7); 3.5133 (6.1); 2.4816 (0.6); 2.2231 (0.6); 2.2132 (14.0); 2.0443 (0.6); 1.2590 (0.6); 0.8817 (0.5); −0.0002 (10.5)

I.28-176: ¹H-NMR (400.6 MHz, CDCl3):

δ=7.5695 (3.3); 7.5481 (3.3); 7.2844 (1.8); 7.2785 (2.4); 7.2604 (13.8); 7.2571 (3.3); 7.2440 (4.5); 7.2383 (2.9); 6.8418 (3.6); 6.8202 (3.3); 6.6984 (3.2); 6.6826 (3.3); 6.3033 (5.7); 5.2995 (9.4); 4.7599 (16.0); 4.7197 (12.8); 3.8618 (1.0); 3.5179 (8.3); 3.5153 (8.3); 2.0448 (0.7); 1.6761 (1.4); 1.5460 (1.7); 1.2592 (0.7); 0.0079 (0.5); −0.0002 (19.1); −0.0085 (0.5)

I.33-1: ¹H-NMR (400.6 MHz, CDCl3):

δ=7.5437 (1.4); 7.5373 (4.6); 7.5320 (0.6); 7.5164 (3.2); 7.5113 (1.0); 7.2616 (8.2); 6.9190 (1.6); 6.8982 (1.5); 6.8153 (1.7); 6.8011 (1.7); 6.2799 (3.3); 5.3000 (6.8); 4.7354 (5.7); 4.2711 (1.6); 4.2633 (1.0); 4.2596 (1.6); 4.2557 (1.0); 4.2480 (1.8); 3.5549 (1.9); 3.5469 (1.2); 3.5433 (2.0); 3.5402 (1.2); 3.5318 (1.9); 3.5053 (4.3); 3.5027 (4.6); 3.3558 (0.5); 3.3509 (0.9); 3.3327 (16.0); 3.3145 (0.6); 2.0447 (0.6); 1.5581 (2.1); −0.0002 (12.0)

I.7-367: ¹H-NMR (400.6 MHz, CDCl3):

δ=7.3490 (2.0); 7.3269 (2.0); 7.2604 (12.8); 7.1098 (0.5); 7.0959 (0.7); 7.0941 (0.7); 7.0915 (0.9); 7.0897 (0.8); 7.0759 (0.8); 7.0711 (1.4); 7.0679 (2.5); 7.0663 (3.9); 7.0496 (1.7); 7.0453 (1.1); 7.0068 (0.9); 7.0031 (1.0); 6.9885 (0.9); 6.9867 (0.8); 6.9850 (0.9); 6.9831 (0.7); 6.9106 (1.2); 6.9069 (1.1); 6.8902 (1.0); 6.8868 (0.9); 6.7768 (1.9); 6.7604 (2.0); 6.2667 (3.4); 5.2992 (0.6); 5.2201 (5.7); 4.7124 (6.9); 3.4909 (4.7); 3.4882 (4.6); 2.6900 (16.0); 1.5681 (0.6); 0.0079 (0.6); −0.0002 (18.1)

I.7-366: ¹H-NMR (400.6 MHz, CDCl3):

δ=8.7702 (4.4); 8.7652 (4.3); 7.3467 (4.4); 7.3246 (4.4); 7.2823 (3.7); 7.2805 (2.7); 7.2773 (3.6); 7.2606 (16.3); 7.1197 (0.9); 7.1152 (1.1); 7.1011 (1.7); 7.0967 (2.1); 7.0813 (1.5); 7.0768 (2.1); 7.0733 (1.9); 7.0688 (1.9); 7.0532 (3.7); 7.0489 (2.6); 7.0112 (2.0); 7.0075 (2.1); 6.9927 (2.0); 6.9892 (2.0); 6.9729 (0.9); 6.9692 (0.9); 6.9157 (2.7); 6.9122 (2.5); 6.8954 (2.3); 6.8920 (2.0); 6.7514 (4.2); 6.7349 (4.3); 6.2679 (7.7); 5.7139 (0.6); 5.3390 (13.4); 5.2989 (0.8); 4.7238 (16.0); 3.4906 (11.2); 3.4882 (11.0); 1.5757 (1.7); 0.0078 (0.8); −0.0002 (23.0); −0.0085 (0.7)

I.7-371: ¹H-NMR (400.6 MHz, CDCl3):

δ=7.8613 (5.2); 7.3599 (3.0); 7.3377 (3.0); 7.2612 (11.0); 7.1251 (4.0); 7.1099 (1.1); 7.1076 (1.0); 7.1050 (1.3); 7.1028 (1.1); 7.0897 (1.1); 7.0848 (1.4); 7.0655 (0.9); 7.0608 (1.2); 7.0455 (2.7); 7.0408 (1.9); 7.0240 (1.6); 7.0204 (1.8); 7.0061 (1.3); 7.0025 (1.3); 7.0003 (0.8); 6.9861 (0.6); 6.9824 (0.6); 6.9163 (1.8); 6.9128 (1.6); 6.8961 (1.5); 6.8925 (1.3); 6.7124 (2.9); 6.6960 (2.9); 6.2843 (4.9); 5.2988 (16.0); 5.1864 (8.4); 4.6786 (11.3); 3.5021 (6.7); 3.4992 (6.8); 2.0444 (1.4); 1.2589 (0.9); −0.0002 (15.3)

I.7-365: ¹H-NMR (400.6 MHz, CDCl3):

δ=7.8420 (2.0); 7.6585 (2.3); 7.6564 (2.1); 7.3567 (2.1); 7.3345 (2.1); 7.2623 (3.1); 7.1153 (0.5); 7.1016 (0.7); 7.0997 (0.7); 7.0970 (0.9); 7.0951 (0.7); 7.0815 (0.7); 7.0769 (0.9); 7.0637 (0.8); 7.0591 (0.8); 7.0436 (1.8); 7.0391 (1.3); 7.0104 (1.0); 7.0067 (1.0); 6.9921 (0.8); 6.9901 (0.8); 6.9885 (0.8); 6.9868 (0.6); 6.9106 (1.2); 6.9070 (1.1); 6.8903 (1.0); 6.8868 (0.9); 6.7496 (2.0); 6.7331 (2.0); 6.2801 (3.4); 5.2977 (16.0); 5.1215 (5.2); 4.6918 (7.4); 3.4973 (4.6); 3.4945 (4.6); 2.0437 (0.8); 1.2584 (0.5); −0.0002 (4.5)

I.7-328: ¹H-NMR (400.6 MHz, CDCl3):

δ=8.4421 (2.8); 8.4353 (2.8); 8.3820 (2.8); 7.3698 (0.9); 7.3638 (1.2); 7.3587 (0.9); 7.3477 (1.0); 7.3416 (1.3); 7.3342 (4.6); 7.3120 (4.4); 7.2610 (14.5); 7.1350 (0.9); 7.1303 (1.1); 7.1168 (1.4); 7.1149 (1.4); 7.1122 (1.7); 7.1103 (1.5); 7.0968 (1.4); 7.0921 (1.9); 7.0819 (1.4); 7.0772 (1.7); 7.0618 (3.8); 7.0572 (2.6); 7.0354 (2.1); 7.0317 (2.2); 7.0172 (1.7); 7.0140 (1.7); 6.9973 (0.8); 6.9936 (0.7); 6.9242 (2.6); 6.9206 (2.4); 6.9040 (2.2); 6.9004 (1.9); 6.6968 (4.2); 6.6804 (4.2); 6.2770 (7.3); 5.2990 (10.6); 5.1953 (11.7); 4.7058 (16.0); 3.4963 (9.9); 3.4936 (10.0); 0.0079 (0.7); −0.0002 (21.0); −0.0085 (0.6)

I.7-296: ¹H-NMR (400.6 MHz, CDCl3):

δ=8.4873 (1.6); 8.4775 (1.6); 7.5620 (1.4); 7.5445 (1.6); 7.3720 (0.7); 7.3498 (0.7); 7.3185 (4.0); 7.2963 (4.0); 7.2607 (13.9); 7.2563 (1.7); 7.2438 (1.6); 7.2368 (1.4); 7.2246 (1.4); 7.1189 (1.0); 7.1145 (1.1); 7.0996 (1.8); 7.0969 (1.5); 7.0936 (1.4); 7.0804 (1.4); 7.0759 (1.8); 7.0656 (0.5); 7.0577 (1.5); 7.0529 (1.7); 7.0366 (3.3); 7.0326 (2.5); 7.0072 (0.8); 6.9955 (1.3); 6.9919 (2.6); 6.9832 (2.9); 6.9797 (2.9); 6.9770 (1.8); 6.9740 (2.5); 6.9630 (1.9); 6.9591 (2.0); 6.9530 (0.5); 6.7818 (3.8); 6.7653 (3.8); 6.7415 (0.6); 6.7250 (0.6); 6.2709 (1.2); 6.2583 (6.8); 5.3176 (12.0); 5.2983 (14.0); 4.8921 (0.6); 4.7658 (9.3); 4.6549 (1.6); 3.4828 (10.4); 3.4800 (9.9); 2.4083 (1.0); 2.3277 (16.0); 0.0080 (0.7); −0.0002 (20.0); −0.0085 (0.6)

I.7-284: ¹H-NMR (400.6 MHz, CDCl3):

δ=8.3421 (2.8); 8.3360 (2.9); 7.5983 (1.9); 7.5922 (1.8); 7.5779 (2.2); 7.5716 (2.1); 7.3366 (4.3); 7.3143 (5.1); 7.3118 (4.0); 7.2912 (3.1); 7.2609 (16.2); 7.1270 (0.9); 7.1221 (1.1); 7.1091 (1.6); 7.1067 (1.5); 7.1042 (1.9); 7.1019 (1.5); 7.0891 (1.5); 7.0841 (2.0); 7.0696 (1.2); 7.0648 (1.6); 7.0496 (3.9); 7.0448 (2.7); 7.0292 (2.3); 7.0255 (2.4); 7.0113 (1.9); 7.0077 (1.8); 7.0055 (1.1); 6.9912 (0.8); 6.9876 (0.7); 6.9120 (2.6); 6.9086 (2.4); 6.8918 (2.2); 6.8884 (1.9); 6.6922 (4.2); 6.6758 (4.2); 6.2749 (7.3); 5.2989 (7.3); 5.1512 (12.0); 4.6840 (16.0); 3.9833 (0.5); 3.4973 (10.6); 3.4945 (10.1); 1.4107 (1.6); 1.2937 (0.6); 1.2555 (1.4); 0.0080 (0.6); −0.0002 (23.6); −0.0085 (0.7)

I.19-365: ¹H-NMR (400.6 MHz, CDCl3):

δ=7.8368 (4.6); 7.6722 (0.6); 7.6701 (0.6); 7.6644 (4.9); 7.6622 (5.2); 7.3495 (4.4); 7.3274 (4.4); 7.2619 (11.4); 7.1100 (1.0); 7.0952 (1.1); 7.0888 (2.3); 7.0740 (2.4); 7.0676 (1.4); 7.0527 (1.3); 6.8777 (1.3); 6.8744 (1.6); 6.8534 (2.2); 6.8512 (2.1); 6.8331 (1.2); 6.8298 (1.2); 6.6845 (2.9); 6.6684 (3.0); 6.6540 (1.3); 6.6503 (2.3); 6.6469 (1.4); 6.6328 (1.2); 6.6291 (2.1); 6.6258 (1.2); 6.3230 (0.6); 6.2666 (7.4); 5.2988 (16.0); 5.1279 (0.9); 5.1054 (11.5); 4.6768 (15.9); 3.5313 (0.8); 3.5285 (0.8); 3.4898 (10.0); 3.4870 (10.5); 2.0443 (1.6); 1.5868 (6.9); 1.2767 (0.5); 1.2589 (1.2); −0.0002 (9.9)

I.19-89: ¹H-NMR (400.6 MHz, CDCl3):

δ=7.3737 (4.6); 7.3516 (4.6); 7.2611 (15.7); 7.1447 (1.4); 7.1299 (1.2); 7.1235 (2.5); 7.1088 (2.4); 7.1023 (1.5); 7.0876 (1.3); 6.8975 (1.4); 6.8942 (1.4); 6.8739 (2.1); 6.8710 (1.8); 6.8529 (1.4); 6.8496 (1.2); 6.6832 (1.5); 6.6796 (2.4); 6.6762 (1.4); 6.6594 (4.2); 6.6551 (1.4); 6.6444 (2.8); 6.3291 (0.6); 6.2758 (7.6); 5.2990 (13.7); 4.7661 (3.8); 4.7500 (4.4); 4.7468 (4.5); 4.7306 (4.1); 4.6583 (16.0); 4.3980 (2.5); 4.3892 (2.7); 4.3830 (4.4); 4.3739 (4.3); 4.3677 (7.4); 4.3586 (2.5); 4.3510 (5.1); 3.5369 (0.8); 3.5341 (0.8); 3.4972 (10.5); 3.4943 (10.5); 3.2693 (0.9); 3.2666 (0.9); 3.2647 (0.8); 3.2497 (1.4); 3.2349 (0.8); 3.2329 (0.9); 3.2303 (0.7); 2.0445 (1.5); 1.5667 (5.5); 1.5633 (6.1); 1.2768 (0.5); 1.2590 (1.1); −0.0002 (16.0)

I.8-329: ¹H-NMR (400.6 MHz, CDCl3):

δ=8.4643 (0.9); 8.4601 (0.9); 8.4521 (0.9); 8.4478 (0.8); 7.5504 (0.8); 7.5465 (0.7); 7.5312 (0.8); 7.5274 (0.8); 7.4805 (2.2); 7.4590 (2.2); 7.2613 (8.2); 7.1240 (1.1); 7.1190 (0.7); 7.1129 (0.8); 7.1056 (1.4); 7.1038 (1.0); 7.1009 (1.0); 7.0988 (0.9); 7.0938 (0.7); 7.0856 (0.8); 7.0808 (1.1); 7.0730 (0.8); 7.0684 (0.9); 7.0529 (1.9); 7.0484 (1.3); 7.0248 (1.1); 7.0210 (1.2); 7.0067 (0.9); 7.0046 (0.8); 7.0031 (0.9); 7.0011 (0.7); 6.9146 (1.2); 6.9110 (1.2); 6.8944 (1.1); 6.8908 (0.9); 6.6774 (2.1); 6.6613 (2.2); 6.2586 (3.5); 5.2982 (16.0); 5.1874 (5.4); 4.7082 (7.5); 3.4869 (4.7); 3.4840 (4.8); 2.5169 (9.6); 2.0442 (0.6); 1.2588 (0.9); −0.0002 (11.5)

I.7-329: ¹H-NMR (400.6 MHz, CDCl3):

δ=8.4641 (0.8); 8.4599 (0.9); 8.4519 (0.9); 8.4477 (0.8); 7.5509 (0.8); 7.5469 (0.8); 7.5317 (0.8); 7.5277 (0.8); 7.3168 (2.2); 7.2946 (2.2); 7.2615 (6.5); 7.1234 (0.8); 7.1201 (0.7); 7.1151 (0.7); 7.1113 (0.8); 7.1022 (1.1); 7.1000 (0.8); 7.0972 (1.0); 7.0950 (1.0); 7.0819 (0.8); 7.0771 (1.0); 7.0674 (0.7); 7.0626 (0.9); 7.0473 (2.0); 7.0426 (1.3); 7.0224 (1.1); 7.0187 (1.2); 7.0044 (0.8); 7.0009 (0.9); 6.9988 (0.7); 6.9098 (1.3); 6.9061 (1.2); 6.8895 (1.1); 6.8860 (1.0); 6.7134 (2.1); 6.6971 (2.0); 6.2628 (3.6); 5.2980 (16.0); 5.1883 (5.5); 4.7070 (7.6); 3.4895 (4.8); 3.4867 (5.0); 2.5155 (9.7); 2.0440 (0.6); 1.2586 (0.8); −0.0002 (9.3)

I.8-321: ¹H-NMR (400.6 MHz, CDCl3):

δ=9.1886 (2.6); 8.7036 (5.9); 7.5061 (2.0); 7.4846 (2.0); 7.2617 (5.9); 7.1179 (0.6); 7.1161 (0.7); 7.1134 (0.8); 7.1116 (0.8); 7.0980 (0.6); 7.0933 (0.9); 7.0867 (0.7); 7.0820 (0.8); 7.0666 (1.7); 7.0621 (1.2); 7.0386 (0.9); 7.0350 (1.0); 7.0205 (0.8); 7.0171 (0.8); 6.9239 (1.2); 6.9203 (1.1); 6.9036 (1.0); 6.9002 (0.9); 6.6470 (2.0); 6.6310 (2.0); 6.2777 (3.4); 5.2989 (16.0); 5.1759 (5.8); 4.6975 (7.4); 3.4945 (4.7); 3.4919 (4.9); 1.2580 (0.8); −0.0002 (8.2)

I.8-367: ¹H-NMR (400.6 MHz, CDCl3):

δ=7.5108 (2.0); 7.4892 (2.0); 7.2614 (4.9); 7.1113 (0.5); 7.0974 (0.7); 7.0956 (0.7); 7.0930 (0.8); 7.0912 (0.7); 7.0774 (0.7); 7.0732 (1.6); 7.0693 (1.1); 7.0649 (3.2); 7.0536 (1.6); 7.0494 (1.0); 7.0074 (0.9); 7.0037 (1.0); 6.9890 (0.8); 6.9873 (0.7); 6.9856 (0.8); 6.9837 (0.6); 6.9135 (1.2); 6.9099 (1.0); 6.8932 (1.0); 6.8898 (0.8); 6.7433 (1.9); 6.7271 (1.9); 6.2620 (3.2); 5.2979 (16.0); 5.2182 (5.1); 4.7129 (6.5); 3.4875 (4.3); 3.4847 (4.4); 2.6988 (0.6); 2.6884 (15.2); 2.0440 (0.6); 1.2586 (0.5); −0.0002 (6.9)

I.19-367: ¹H-NMR (400.6 MHz, CDCl3):

δ=7.3414 (2.0); 7.3193 (1.9); 7.2612 (7.5); 7.0981 (0.5); 7.0832 (0.7); 7.0769 (1.2); 7.0706 (3.4); 7.0620 (1.1); 7.0557 (0.6); 7.0409 (0.6); 6.8707 (0.6); 6.8675 (0.7); 6.8467 (0.9); 6.8443 (0.8); 6.8262 (0.5); 6.8230 (0.5); 6.7339 (1.2); 6.7178 (1.2); 6.6532 (0.6); 6.6496 (1.1); 6.6461 (0.6); 6.6321 (0.6); 6.6284 (0.9); 6.2500 (3.3); 5.2987 (0.9); 5.2258 (0.5); 5.1993 (5.2); 4.6972 (6.8); 3.4813 (4.4); 3.4784 (4.4); 2.6971 (1.4); 2.6830 (16.0); 1.5873 (1.5); −0.0002 (7.3)

I.19-71: ¹H-NMR (400.6 MHz, CDCl3):

δ=7.3585 (3.6); 7.3364 (3.6); 7.2617 (8.4); 7.1322 (0.9); 7.1173 (1.0); 7.1110 (1.9); 7.0961 (2.0); 7.0897 (1.1); 7.0749 (1.1); 6.8779 (1.2); 6.8746 (1.2); 6.8564 (1.3); 6.8543 (1.7); 6.8513 (1.4); 6.8333 (1.0); 6.8300 (0.9); 6.7228 (2.1); 6.7067 (2.1); 6.6825 (1.0); 6.6792 (1.7); 6.6760 (0.9); 6.6613 (1.0); 6.6580 (1.6); 6.2654 (3.3); 6.2611 (3.1); 5.2984 (16.0); 4.6905 (10.2); 4.1880 (1.0); 4.1815 (0.8); 4.1689 (1.2); 4.1624 (1.4); 4.1543 (1.0); 4.1047 (0.9); 4.0893 (1.9); 4.0742 (1.7); 4.0685 (1.0); 4.0620 (1.0); 4.0574 (2.6); 4.0501 (0.8); 4.0412 (0.9); 3.8169 (1.0); 3.8124 (0.7); 3.8004 (1.1); 3.7959 (1.7); 3.7838 (0.6); 3.7794 (1.7); 3.7713 (0.6); 3.7631 (0.7); 3.7595 (0.6); 3.7544 (1.2); 3.7427 (1.1); 3.7379 (0.9); 3.7337 (0.6); 3.7262 (0.6); 3.7220 (0.5); 3.5321 (0.6); 3.4884 (7.9); 2.0441 (0.5); 1.9711 (0.6); 1.9540 (0.8); 1.9372 (0.5); 1.9016 (0.6); 1.8931 (0.6); 1.8855 (1.3); 1.8767 (1.3); 1.8665 (1.8); 1.8580 (1.6); 1.8497 (1.4); 1.8412 (1.2); 1.5827 (2.6); 1.5802 (2.4); 1.5542 (0.6); 1.5398 (0.5); 1.5362 (0.6); 1.5259 (0.5); 1.5196 (0.5); −0.0002 (8.4)

I.19-72: ¹H-NMR (400.6 MHz, CDCl3):

δ=7.3676 (4.6); 7.3455 (4.6); 7.2614 (13.6); 7.1443 (1.1); 7.1295 (1.1); 7.1231 (2.3); 7.1083 (2.2); 7.1019 (1.4); 7.0871 (1.2); 6.8928 (1.4); 6.8896 (1.5); 6.8690 (2.2); 6.8664 (1.9); 6.8483 (1.3); 6.8450 (1.2); 6.6725 (3.8); 6.6576 (2.5); 6.6553 (2.5); 6.6507 (2.0); 6.2834 (3.9); 6.2645 (3.9); 5.2988 (15.7); 4.6476 (16.0); 4.1663 (0.8); 4.1499 (0.9); 4.1396 (1.8); 4.1234 (1.9); 4.1130 (1.4); 4.0968 (1.3); 4.0482 (1.4); 4.0280 (1.5); 4.0227 (1.7); 4.0021 (1.7); 3.9961 (0.9); 3.9757 (0.9); 3.8465 (0.8); 3.8330 (0.9); 3.8256 (1.8); 3.8120 (1.8); 3.8052 (1.3); 3.7916 (1.2); 3.7564 (1.1); 3.7535 (1.0); 3.7384 (2.2); 3.7313 (1.4); 3.7173 (2.6); 3.7002 (1.6); 3.6800 (0.7); 3.5342 (0.7); 3.5314 (0.8); 3.5194 (1.1); 3.5063 (1.6); 3.4931 (10.5); 3.4906 (10.5); 3.4785 (1.1); 3.4654 (1.0); 2.5513 (0.7); 2.5343 (0.9); 2.5168 (0.7); 2.0443 (0.9); 2.0149 (0.6); 2.0020 (0.9); 1.9923 (0.6); 1.9830 (0.9); 1.9699 (0.7); 1.5696 (4.7); 1.5604 (1.2); 1.5423 (1.0); 1.5282 (0.7); 1.2589 (0.7); −0.0002 (13.9)

I.19-41: ¹H-NMR (400.6 MHz, CDCl3):

δ=7.3695 (4.2); 7.3474 (4.2); 7.2606 (7.4); 7.1430 (1.3); 7.1281 (1.2); 7.1217 (2.4); 7.1069 (2.3); 7.1005 (1.4); 7.0858 (1.2); 6.8983 (1.5); 6.8950 (1.4); 6.8748 (2.4); 6.8719 (1.7); 6.8537 (1.4); 6.8504 (1.2); 6.6826 (1.7); 6.6791 (2.4); 6.6756 (1.3); 6.6614 (1.7); 6.6579 (2.3); 6.6542 (1.4); 6.6485 (3.0); 6.6324 (2.9); 6.3194 (0.6); 6.2610 (7.2); 5.2979 (11.5); 4.6900 (16.0); 4.3688 (2.5); 4.3590 (3.4); 4.3457 (2.8); 4.1293 (5.0); 4.1246 (1.8); 4.1198 (4.0); 4.1174 (5.0); 4.1105 (1.7); 4.1060 (3.8); 3.5314 (1.0); 3.4909 (11.8); 3.4882 (10.6); 1.5608 (3.2); −0.0002 (7.3)

I.19-356: ¹H-NMR (400.6 MHz, CDCl3):

δ=7.3264 (2.7); 7.3036 (4.2); 7.3018 (4.0); 7.2621 (7.2); 7.1054 (0.6); 7.0905 (0.8); 7.0842 (1.4); 7.0694 (1.4); 7.0630 (0.9); 7.0482 (0.8); 6.8779 (0.8); 6.8746 (0.9); 6.8542 (1.3); 6.8515 (1.1); 6.8333 (0.7); 6.8300 (0.7); 6.6562 (1.7); 6.6400 (1.7); 6.6260 (0.9); 6.6224 (1.5); 6.6190 (0.9); 6.6048 (0.8); 6.6012 (1.4); 6.5978 (0.8); 6.2647 (4.6); 5.2982 (10.8); 5.0219 (0.6); 5.0032 (4.8); 4.9999 (4.7); 4.6046 (1.0); 4.5971 (9.5); 3.7989 (16.0); 3.4934 (6.2); 3.4907 (6.3); 2.2045 (15.4); 2.1957 (1.3); 2.0441 (1.6); 1.6165 (0.7); 1.2766 (0.5); 1.2588 (1.3); −0.0002 (7.9)

I.19-278: ¹H-NMR (400.6 MHz, CDCl3):

δ=8.5908 (3.1); 8.5758 (3.2); 7.3045 (3.6); 7.2823 (3.6); 7.2622 (10.0); 7.1647 (3.9); 7.1607 (2.4); 7.1537 (2.6); 7.1497 (3.6); 7.1421 (1.0); 7.1272 (1.0); 7.1208 (1.9); 7.1061 (2.0); 7.0996 (1.2); 7.0849 (1.1); 6.9141 (1.1); 6.9108 (1.2); 6.8906 (1.7); 6.8877 (1.4); 6.8695 (1.0); 6.8662 (0.9); 6.6965 (1.2); 6.6930 (2.0); 6.6895 (1.1); 6.6754 (1.2); 6.6718 (1.8); 6.6684 (1.0); 6.6335 (2.3); 6.6175 (2.3); 6.2289 (6.2); 5.2983 (16.0); 5.1763 (0.8); 5.1640 (5.8); 4.7277 (13.4); 3.5208 (0.6); 3.5180 (0.6); 3.4734 (8.4); 3.4705 (8.5); 2.0440 (1.5); 1.2765 (0.5); 1.2586 (1.5); 1.2408 (0.5); −0.0002 (10.2)

I.19-321: ¹H-NMR (400.6 MHz, CDCl3):

δ=9.1915 (6.2); 8.6951 (14.7); 7.3396 (4.0); 7.3176 (4.1); 7.2621 (11.5); 7.1265 (0.9); 7.1194 (0.5); 7.1116 (1.0); 7.1052 (2.2); 7.0905 (2.2); 7.0843 (1.6); 7.0693 (1.2); 6.9045 (1.9); 6.8815 (2.9); 6.8600 (1.5); 6.6615 (2.8); 6.6403 (2.6); 6.5965 (3.3); 6.5805 (3.4); 6.3217 (0.6); 6.2683 (7.9); 5.2993 (11.2); 5.1770 (1.2); 5.1659 (14.4); 4.6747 (16.0); 3.5259 (1.2); 3.4877 (15.0); 2.1254 (0.8); 2.0443 (1.1); 1.5879 (8.5); 1.2767 (0.5); 1.2562 (1.8); 1.2412 (0.6); −0.0002 (12.3); −0.0021 (7.5); −0.0081 (0.8)

I.19-277: ¹H-NMR (400.6 MHz, CDCl3):

δ=8.5914 (1.6); 8.5878 (1.7); 8.5794 (1.7); 8.5757 (1.7); 8.5610 (2.2); 8.5562 (2.2); 7.6358 (1.0); 7.6303 (1.6); 7.6259 (1.1); 7.6162 (1.2); 7.6108 (1.8); 7.6063 (1.2); 7.3208 (4.4); 7.2988 (5.1); 7.2901 (1.6); 7.2826 (1.5); 7.2705 (1.3); 7.2688 (1.3); 7.2617 (14.3); 7.1162 (1.1); 7.1014 (1.1); 7.0950 (2.4); 7.0802 (2.4); 7.0738 (1.5); 7.0590 (1.3); 6.8933 (1.3); 6.8900 (1.4); 6.8692 (2.0); 6.8668 (1.8); 6.8487 (1.3); 6.8454 (1.2); 6.6563 (1.4); 6.6526 (2.4); 6.6492 (1.3); 6.6316 (4.9); 6.6157 (2.7); 6.3175 (0.6); 6.2567 (7.4); 5.2985 (15.0); 5.1873 (1.0); 5.1718 (10.9); 4.6738 (16.0); 3.5267 (0.8); 3.5238 (0.9); 3.4834 (9.8); 3.4805 (10.2); 2.0441 (1.3); 1.6578 (0.8); 1.2588 (1.3); −0.0002 (14.2)

I.28-276: ¹H-NMR (400.6 MHz, CDCl3):

δ=8.5994 (1.0); 8.5950 (1.3); 8.5905 (0.6); 8.5852 (1.3); 8.5811 (0.9); 7.7361 (0.8); 7.7316 (0.8); 7.7168 (1.6); 7.7124 (1.3); 7.6975 (0.9); 7.6931 (0.9); 7.5037 (3.1); 7.4822 (3.1); 7.2866 (1.1); 7.2765 (2.6); 7.2673 (1.2); 7.2605 (19.9); 7.2571 (2.8); 7.2314 (1.5); 7.2255 (2.4); 7.2072 (3.6); 7.2053 (8.6); 7.2016 (1.7); 6.8495 (0.6); 6.8456 (2.1); 6.8434 (1.6); 6.8255 (1.5); 6.8233 (1.8); 6.7760 (3.0); 6.7600 (3.0); 6.2617 (5.0); 5.2992 (16.0); 5.2877 (8.1); 4.7507 (10.0); 3.4939 (6.7); 3.4910 (6.6); 0.0079 (0.8); −0.0002 (26.9); −0.0085 (0.7)

I.28-331: ¹H-NMR (400.6 MHz, CDCl3):

δ=8.3728 (0.9); 8.3680 (0.9); 8.3608 (0.9); 8.3560 (0.8); 7.6663 (0.9); 7.6615 (0.9); 7.6474 (1.0); 7.6426 (0.9); 7.5070 (2.1); 7.4856 (2.1); 7.2607 (10.8); 7.2489 (1.3); 7.2432 (1.8); 7.2384 (1.1); 7.2271 (1.6); 7.2216 (1.9); 7.2110 (3.0); 7.2053 (1.7); 6.8330 (2.2); 6.8114 (2.0); 6.7375 (2.0); 6.7216 (2.0); 6.2751 (3.6); 5.2994 (16.0); 5.2623 (5.5); 4.7243 (7.1); 3.5053 (5.0); 3.5027 (5.1); 0.0079 (0.6); −0.0002 (15.5)

I.28-278: ¹H-NMR (400.6 MHz, CDCl3):

δ=8.6615 (1.8); 7.4808 (4.5); 7.4594 (4.5); 7.3411 (3.2); 7.3284 (3.0); 7.2607 (34.2); 7.2442 (2.0); 7.2383 (3.7); 7.2257 (6.1); 7.2200 (3.9); 6.8507 (4.9); 6.8292 (4.4); 6.7067 (4.3); 6.6908 (4.4); 6.2779 (8.1); 5.2996 (14.3); 5.2322 (11.2); 4.7403 (0.6); 4.7290 (16.0); 3.5080 (12.2); 1.2548 (0.9); 0.0079 (1.3); −0.0002 (47.4); −0.0084 (1.7)

I.33-176: ¹H-NMR (400.6 MHz, CDCl3):

δ=7.5787 (0.8); 7.5739 (3.4); 7.5673 (2.6); 7.5625 (2.7); 7.5562 (2.0); 7.5534 (2.2); 7.2610 (10.4); 6.9575 (1.8); 6.9350 (1.7); 6.7064 (1.8); 6.6923 (1.8); 6.3030 (3.4); 5.3000 (16.0); 4.8011 (0.5); 4.7816 (6.8); 4.7641 (9.1); 3.5165 (4.7); 3.5139 (4.9); 0.0080 (0.5); −0.0002 (15.0)

I.33-276: ¹H-NMR (400.6 MHz, CDCl3):

δ=8.5751 (1.7); 8.5713 (2.2); 8.5685 (1.7); 8.5633 (1.7); 8.5593 (2.1); 8.5567 (1.7); 7.7441 (1.4); 7.7397 (1.4); 7.7249 (3.1); 7.7205 (3.0); 7.7056 (1.9); 7.7012 (1.8); 7.5346 (5.7); 7.5296 (7.4); 7.5229 (0.8); 7.5199 (0.6); 7.5063 (3.8); 7.5012 (2.7); 7.4899 (5.3); 7.4849 (4.0); 7.4798 (3.3); 7.4691 (5.1); 7.2901 (2.0); 7.2777 (4.2); 7.2753 (4.3); 7.2616 (26.1); 6.9698 (5.2); 6.9484 (4.9); 6.8246 (4.9); 6.8103 (5.0); 6.7850 (0.5); 6.2396 (9.4); 6.2222 (0.9); 5.3041 (0.6); 5.2480 (11.8); 4.8297 (0.7); 4.8111 (15.6); 4.7169 (1.0); 3.5329 (0.5); 3.4815 (12.4); 3.4789 (12.8); 2.0068 (16.0); 0.0080 (1.0); −0.0002 (36.2); −0.0085 (1.1)

I.23-89: ¹H-NMR (400.6 MHz, CDCl3):

δ=7.4027 (3.1); 7.3807 (3.1); 7.2613 (12.8); 6.9584 (1.2); 6.9455 (1.4); 6.9359 (1.8); 6.9231 (1.8); 6.8450 (3.0); 6.8346 (1.2); 6.8286 (3.4); 6.8154 (1.2); 6.8122 (0.8); 6.8079 (1.4); 6.8048 (1.1); 6.7930 (0.8); 6.7855 (1.0); 6.7682 (1.8); 6.7609 (1.4); 6.7463 (1.9); 6.7390 (1.4); 6.3106 (5.2); 5.2995 (16.0); 4.7838 (3.5); 4.7679 (4.0); 4.7642 (3.9); 4.7483 (3.8); 4.6525 (11.8); 4.6313 (0.7); 4.4265 (1.7); 4.4211 (1.6); 4.4112 (2.9); 4.4059 (2.7); 4.3957 (1.8); 4.3892 (5.8); 4.3722 (5.4); 3.5230 (7.0); 3.5201 (7.0); 3.2919 (0.6); 3.2891 (0.6); 3.2872 (0.6); 3.2723 (1.0); 3.2575 (0.6); 3.2555 (0.6); 3.2528 (0.5); 1.5639 (2.5); 1.2592 (0.5); 0.0079 (0.5); −0.0002 (19.5); −0.0085 (0.5)

I.23-356: ¹H-NMR (400.6 MHz, CDCl3):

δ=7.3589 (2.6); 7.3368 (2.6); 7.3039 (3.5); 7.2620 (9.5); 6.9067 (1.0); 6.8939 (1.1); 6.8843 (1.5); 6.8714 (1.5); 6.8260 (2.5); 6.8096 (2.6); 6.7994 (0.9); 6.7920 (1.1); 6.7802 (0.9); 6.7770 (0.6); 6.7727 (1.1); 6.7697 (1.0); 6.7578 (0.6); 6.7503 (0.8); 6.7424 (1.5); 6.7351 (1.1); 6.7205 (1.6); 6.7131 (1.1); 6.3052 (4.3); 5.2990 (16.0); 5.0302 (0.7); 5.0223 (7.7); 4.5958 (9.9); 4.5769 (0.7); 3.8026 (0.5); 3.7952 (15.3); 3.5214 (5.8); 3.5185 (5.9); 2.2193 (14.8); 2.2150 (1.4); −0.0002 (14.0)

I.8-331: ¹H-NMR (400.6 MHz, CDCl3):

δ=8.3728 (0.6); 7.6768 (1.2); 7.6721 (1.1); 7.6578 (1.3); 7.6557 (1.0); 7.6531 (1.2); 7.4953 (3.2); 7.4738 (3.2); 7.2611 (13.8); 7.2525 (1.0); 7.2455 (0.9); 7.2335 (0.8); 7.1500 (0.7); 7.1454 (0.8); 7.1316 (1.1); 7.1297 (1.1); 7.1271 (1.4); 7.1253 (1.1); 7.1116 (1.1); 7.1071 (1.4); 7.0969 (1.2); 7.0924 (1.3); 7.0768 (2.6); 7.0723 (1.9); 7.0420 (1.4); 7.0383 (1.6); 7.0237 (1.3); 7.0217 (1.2); 7.0201 (1.3); 7.0183 (1.0); 7.0037 (0.6); 7.0000 (0.6); 6.9528 (1.8); 6.9492 (1.7); 6.9326 (1.5); 6.9290 (1.3); 6.6888 (3.1); 6.6727 (3.1); 6.2592 (5.2); 5.2993 (16.0); 5.2691 (7.9); 4.7544 (10.8); 3.4888 (7.2); 3.4860 (7.0); 0.0078 (0.6); −0.0002 (20.9); −0.0021 (1.1); −0.0029 (0.7); −0.0085 (0.6)

I.23-41: ¹H-NMR (400.6 MHz, CDCl3):

δ=7.3988 (4.1); 7.3767 (4.0); 7.2606 (12.5); 6.9624 (1.6); 6.9496 (1.7); 6.9399 (2.3); 6.9271 (2.3); 6.8355 (1.4); 6.8297 (4.2); 6.8163 (1.8); 6.8134 (4.8); 6.8089 (1.9); 6.8058 (1.5); 6.7939 (1.0); 6.7864 (1.3); 6.7780 (2.4); 6.7707 (1.7); 6.7561 (2.4); 6.7488 (1.7); 6.3014 (6.6); 5.2989 (13.5); 4.6813 (16.0); 4.6568 (1.0); 4.3906 (2.1); 4.3794 (2.8); 4.3677 (2.6); 4.1536 (4.8); 4.1483 (1.6); 4.1453 (2.2); 4.1418 (4.2); 4.1393 (2.4); 4.1362 (1.4); 4.1304 (4.0); 3.5423 (0.6); 3.5393 (0.6); 3.5184 (9.0); 3.5154 (9.2); 1.5519 (2.6); 0.0079 (0.5); −0.0002 (18.7); −0.0027 (0.8); −0.0085 (0.5)

I.23-71: ¹H-NMR (400.6 MHz, CDCl3):

δ=7.3889 (3.1); 7.3668 (3.2); 7.2618 (10.7); 6.9580 (1.2); 6.9451 (1.2); 6.9354 (1.7); 6.9226 (1.6); 6.8708 (2.3); 6.8544 (2.3); 6.8273 (1.1); 6.8198 (1.3); 6.8079 (1.2); 6.8048 (0.9); 6.8004 (1.3); 6.7973 (1.2); 6.7855 (0.7); 6.7780 (1.1); 6.7648 (1.9); 6.7574 (1.4); 6.7428 (1.9); 6.7354 (1.4); 6.3028 (3.1); 6.3008 (3.1); 5.2994 (16.0); 4.6765 (11.1); 4.6550 (0.6); 4.2193 (0.9); 4.2172 (1.0); 4.2002 (1.2); 4.1981 (1.2); 4.1836 (0.6); 4.1024 (0.7); 4.0981 (1.5); 4.0962 (1.8); 4.0907 (1.2); 4.0860 (1.7); 4.0841 (1.4); 4.0782 (1.0); 4.0716 (1.9); 3.8467 (0.6); 3.8425 (0.7); 3.8405 (0.7); 3.8364 (0.6); 3.8257 (1.1); 3.8195 (1.1); 3.8095 (0.6); 3.8032 (0.5); 3.7743 (0.9); 3.7692 (1.1); 3.7576 (0.6); 3.7525 (0.9); 3.7484 (0.5); 3.5175 (6.6); 3.5152 (6.8); 1.9663 (0.6); 1.9183 (0.6); 1.9145 (0.6); 1.9019 (1.4); 1.8982 (1.6); 1.8821 (1.8); 1.8789 (1.5); 1.8655 (1.1); 1.8620 (1.1); 1.5824 (0.6); 1.5731 (1.1); 1.5620 (0.6); 1.5510 (0.6); −0.0002 (15.1)

I.7-288: ¹H-NMR (400.6 MHz, CDCl3):

δ=8.5218 (0.7); 7.6636 (1.4); 7.6577 (1.4); 7.6428 (1.6); 7.6368 (1.5); 7.3329 (3.7); 7.3108 (3.7); 7.2608 (18.0); 7.2138 (0.8); 7.1931 (0.7); 7.1407 (0.8); 7.1361 (1.0); 7.1224 (1.3); 7.1204 (1.2); 7.1178 (1.5); 7.1159 (1.4); 7.1023 (1.4); 7.0977 (1.8); 7.0864 (1.4); 7.0820 (1.6); 7.0664 (3.2); 7.0619 (2.3); 7.0301 (1.8); 7.0264 (2.0); 7.0118 (1.5); 7.0100 (1.2); 7.0082 (1.6); 7.0063 (1.2); 6.9918 (0.8); 6.9881 (0.8); 6.9536 (2.1); 6.9500 (1.9); 6.9334 (1.8); 6.9298 (1.5); 6.7376 (3.5); 6.7212 (3.6); 6.2551 (5.9); 5.2992 (16.0); 5.2524 (7.7); 4.7596 (12.0); 3.4865 (7.8); 3.4836 (8.0); 0.0080 (0.8); −0.0002 (27.0); −0.0085 (0.7)

I.23-278: ¹H-NMR (400.6 MHz, CDCl3):

δ=8.5963 (4.5); 8.5923 (2.8); 8.5852 (2.8); 8.5812 (4.7); 7.3460 (3.7); 7.3239 (3.7); 7.2620 (12.4); 7.1945 (4.1); 7.1905 (2.5); 7.1834 (2.6); 7.1794 (4.1); 6.9662 (1.5); 6.9533 (1.6); 6.9438 (2.0); 6.9309 (2.1); 6.8334 (1.1); 6.8259 (1.5); 6.8142 (1.4); 6.8106 (4.2); 6.8068 (1.9); 6.8037 (1.7); 6.7942 (3.8); 6.7843 (1.4); 6.7815 (2.3); 6.7742 (1.6); 6.7596 (2.3); 6.7523 (1.6); 6.2867 (6.3); 5.2989 (16.0); 5.1832 (9.3); 4.7238 (14.3); 4.7002 (1.0); 3.5368 (0.6); 3.5340 (0.6); 3.5108 (8.5); 3.5079 (8.9); 2.0445 (0.8); 1.2589 (1.5); 1.2569 (1.3); −0.0002 (17.4); −0.0085 (0.6)

I.23-277: ¹H-NMR (400.6 MHz, CDCl3):

δ=8.5926 (3.1); 8.5887 (2.9); 8.5809 (1.7); 8.5767 (1.5); 7.6570 (0.8); 7.6525 (1.1); 7.6515 (1.1); 7.6470 (0.8); 7.6374 (0.9); 7.6330 (1.2); 7.6318 (1.3); 7.6274 (0.9); 7.3553 (3.2); 7.3331 (3.3); 7.3039 (1.1); 7.3020 (1.1); 7.2918 (1.1); 7.2898 (1.2); 7.2844 (1.1); 7.2823 (1.1); 7.2722 (1.0); 7.2702 (1.1); 7.2619 (13.4); 6.9332 (1.3); 6.9204 (1.4); 6.9109 (1.8); 6.8980 (1.9); 6.8142 (3.2); 6.8089 (1.3); 6.8014 (1.7); 6.7979 (3.4); 6.7897 (1.2); 6.7866 (0.8); 6.7823 (1.8); 6.7791 (1.3); 6.7673 (0.7); 6.7597 (3.0); 6.7522 (1.3); 6.7375 (2.0); 6.7303 (1.3); 6.3005 (5.5); 5.2990 (16.0); 5.1926 (8.8); 4.6698 (13.2); 4.6475 (0.9); 3.5357 (0.5); 3.5143 (7.4); 3.5113 (7.6); 2.0444 (0.5); 1.2589 (0.9); 1.2566 (0.8); 0.0079 (0.5); −0.0002 (19.1); −0.0085 (0.6)

I.7-331: ¹H-NMR (400.6 MHz, CDCl3):

δ=7.6719 (1.0); 7.6530 (1.1); 7.3331 (2.6); 7.3110 (2.6); 7.2612 (10.3); 7.1461 (0.6); 7.1414 (0.7); 7.1278 (0.9); 7.1259 (0.9); 7.1232 (1.1); 7.1213 (1.0); 7.1078 (0.9); 7.1031 (1.2); 7.0900 (1.0); 7.0855 (1.1); 7.0700 (2.3); 7.0655 (1.7); 7.0387 (1.2); 7.0351 (1.4); 7.0205 (1.0); 7.0186 (0.9); 7.0170 (1.1); 7.0151 (0.8); 7.0005 (0.5); 6.9970 (0.5); 6.9485 (1.4); 6.9449 (1.4); 6.9283 (1.2); 6.9247 (1.1); 6.7264 (2.5); 6.7100 (2.6); 6.2623 (4.3); 5.2991 (16.0); 5.2717 (5.1); 4.7541 (9.0); 3.4911 (5.8); 3.4883 (6.0); −0.0002 (14.4)

I.23-72: ¹H-NMR (400.6 MHz, CDCl3):

δ=7.3975 (3.6); 7.3755 (3.6); 7.2615 (13.1); 6.9469 (1.0); 6.9348 (1.0); 6.9245 (1.5); 6.9117 (1.5); 6.8496 (3.4); 6.8333 (3.6); 6.8281 (1.6); 6.8164 (1.4); 6.8132 (1.0); 6.8089 (1.6); 6.8057 (1.3); 6.7940 (0.9); 6.7865 (1.1); 6.7722 (1.4); 6.7711 (10.3); 6.7638 (1.0); 6.7504 (1.4); 6.7491 (10.4); 6.7430 (10.0); 6.7419 (1.0); 6.3151 (3.1); 6.3040 (3.1); 5.2994 (16.0); 4.6400 (13.7); 4.6172 (0.9); 4.1871 (0.6); 4.1714 (0.9); 4.1602 (1.0); 4.1565 (0.7); 4.1448 (1.4); 4.1296 (1.0); 4.0670 (1.1); 4.0546 (1.0); 4.0469 (1.2); 4.0401 (0.7); 4.0345 (1.1); 4.0277 (0.7); 4.0199 (0.7); 4.0075 (0.7); 3.8603 (0.7); 3.8467 (0.7); 3.8393 (1.4); 3.8256 (1.4); 3.8190 (1.1); 3.8053 (1.0); 3.7900 (0.9); 3.7687 (1.3); 3.7514 (1.9); 3.7332 (1.6); 3.7149 (1.3); 3.6938 (0.8); 3.5446 (1.4); 3.5324 (1.4); 3.5205 (9.2); 3.5176 (8.6); 3.4980 (0.8); 2.5736 (0.5); 2.5565 (0.7); 2.5392 (0.5); 2.0447 (0.6); 2.0311 (0.5); 2.0187 (0.7); 1.9990 (0.7); 1.9861 (0.6); 1.6001 (0.6); 1.5864 (0.9); 1.5668 (2.4); 1.5540 (0.6); 1.2591 (0.7); 0.0080 (0.5); −0.0002 (19.0)

I.23-321: ¹H-NMR (400.6 MHz, CDCl3):

δ=9.1982 (3.5); 8.7287 (8.0); 7.3716 (2.9); 7.3495 (2.9); 7.2627 (9.8); 6.9473 (1.1); 6.9345 (1.2); 6.9250 (1.6); 6.9121 (1.6); 6.8231 (0.9); 6.8156 (1.1); 6.8039 (1.0); 6.7973 (3.5); 6.7934 (1.2); 6.7813 (3.4); 6.7741 (2.6); 6.7668 (1.2); 6.7523 (1.7); 6.7449 (1.1); 6.3069 (4.8); 5.2998 (16.0); 5.2972 (0.5); 5.1888 (8.2); 4.6717 (11.2); 4.6483 (0.8); 3.5181 (6.4); 3.5153 (6.6); 1.2568 (0.8); −0.0002 (14.5); −0.0029 (0.6)

The present invention further provides for the use of one or more inventive compounds of the general formula (I) and/or salts thereof, as defined above, preferably in one of the embodiments identified as preferred or particularly preferred, in particular one or more compounds of the formulae (I.1) to (I.33) and/or salts thereof, in each case as defined above, as herbicide and/or plant growth regulator, preferably in crops of useful plants and/or ornamentals.

The present invention further provides a method of controlling harmful plants and/or for regulating the growth of plants, characterized in that an effective amount
of one or more inventive compounds of the general formula (I) and/or salts thereof, as defined above, preferably in one of the embodiments identified as preferred or particularly preferred, in particular one or more compounds of the formulae (I.1) to (I.33) and/or salts thereof, in each case as defined above, or
of a composition of the invention, as defined below,

is applied to the (harmful) plants, seeds of (harmful) plants, the soil in which or on which the (harmful) plants grow or the area under cultivation.

The present invention also provides a method for controlling unwanted plants, preferably in crops of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.33) and/or salts thereof, in each case as defined above, or
of a composition of the invention, as defined below,

is applied to unwanted plants (for example harmful plants such as mono- or dicotyledonous weeds or unwanted crop plants), the seed of the unwanted plants (i.e. plant seeds, for example grains, seeds or vegetative propagation organs such as tubers or shoot parts with buds), the soil in which or on which the unwanted plants grow (for example the soil of crop-growing land or non-crop-growing land) or the area under cultivation (i.e. the area on which the unwanted plants will grow).

The present invention also further provides methods for controlling for regulating the growth of plants, preferably of useful plants, characterized in that an effective amount
of one or more compounds of the general formula (I) and/or salts thereof, as defined above, preferably in one of the embodiments identified as preferred or particularly preferred, in particular one or more compounds of the formulae (I.1) to (I.33) and/or salts thereof, in each case as defined above, or
of a composition of the invention, as defined below,

is applied to the plant, the seed of the plant (i.e. plant seed, for example grains, seeds or vegetative propagation organs such as tubers or shoot parts with buds), the soil in which or on which the plants grow (for example the soil of crop land or non-crop land) or the area under cultivation (i.e. the area on which the plants will grow).

In this context, the inventive compounds of the general formula (I) or the compositions of the invention can be deployed, for example, by pre-sowing (if appropriate also by incorporation into the soil), pre-emergence and/or post-emergence methods. Specific examples of some representatives of the monocotyledonous and dicotyledonous weed flora which can be controlled by the compounds according to the invention are as follows, though there is no intention to restrict the enumeration to particular species.

In a method of the invention for controlling harmful plants or for regulating the growth of plants, preference is given to using one or more compounds of the general formula (I) and/or salts thereof for control of harmful plants or for regulation of growth in crops of useful plants or ornamental plants, where the useful plants or ornamental plants in a preferred configuration are transgenic plants.

The inventive compounds of the general formula (I) and/or salts thereof are suitable for controlling the following genera of monocotyledonous and dicotyledonous harmful plants:

Monocotyledonous harmful plants of the genera: Aegilops, Agropyron, Agrostis, Alopecurus, Apera, Avena, Brachiaria, Bromus, Cenchrus, Commelina, Cynodon, Cyperus, Dactyloctenium, Digitaria, Echinochloa, Eleocharis, Eleusine, Eragrostis, Eriochloa, Festuca, Fimbristylis, Heteranthera, Imperata, Ischaemum, Leptochloa, Lolium, Monochoria, Panicum, Paspalum, Phalaris, Phleum, Poa, Rottboellia, Sagittaria, Scirpus, Setaria, Sorghum.

Dicotyledonous harmful plants of the genera: Abutilon, Amaranthus, Ambrosia, Anoda, Anthemis, Aphanes, Artemisia, Atriplex, Bellis, Bidens, Capsella, Carduus, Cassia, Centaurea, Chenopodium, Cirsium, Convolvulus, Datura, Desmodium, Emex, Erysimum, Euphorbia, Galeopsis, Galinsoga, Galium, Hibiscus, Ipomoea, Kochia, Lamium, Lepidium, Lindernia, Matricaria, Mentha, Mercurialis, Mullugo, Myosotis, Papaver, Pharbitis, Plantago, Polygonum, Portulaca, Ranunculus, Raphanus, Rorippa, Rotala, Rumex, Salsola, Senecio, Sesbania, Sida, Sinapis, Solanum, Sonchus, Sphenoclea, Stellaria, Taraxacum, Thlaspi, Trifolium, Urtica, Veronica, Viola, Xanthium.

When the inventive compounds of the general formula (I) are applied to the soil surface before germination of the harmful plants (weed grasses and/or broad-leaved weeds) (pre-emergence method), either the seedlings of the weed grasses or broad-leaved weeds are prevented completely from emerging or they grow until they have reached the cotyledon stage, but then stop growing and eventually, after three to four weeks have elapsed, die completely.

When the inventive compounds of the general formula (I) are applied post-emergence to the green parts of the plants, growth stops after the treatment, and the harmful plants remain at the growth stage at the time of application, or they die completely after a certain time, so that in this manner competition by the weeds, which is harmful to the crop plants, is eliminated very early and in a sustained manner.

Although the inventive compounds of the general formula (I) display outstanding herbicidal activity against monocotyledonous and dicotyledonous weeds, crop plants of economically important crops, for example dicotyledonous crops of the genera Arachis, Beta, Brassica, Cucumis, Cucurbita, Helianthus, Daucus, Glycine, Gossypium, Ipomoea, Lactuca, Linum, Lycopersicon, Miscanthus, Nicotiana, Phaseolus, Pisum, Solanum, Vicia, or monocotyledonous crops of the genera Allium, Ananas, Asparagus, Avena, Hordeum, Oryza, Panicum, Saccharum, Secale, Sorghum, Triticale, Triticum, Zea, are damaged only to an insignificant extent, or not at all, depending on the structure of the respective compound according to the invention and its application rate. For these reasons, the present compounds are very suitable for selective control of unwanted plant growth in plant crops such as agriculturally useful plants or ornamental plants.

In addition, the inventive compounds of the general formula (I) (depending on their particular structure and the application rate deployed) have outstanding growth-regulating properties in crop plants. They intervene in the plants' own metabolism with regulatory effect, and can thus be used for the controlled influencing of plant constituents and to facilitate harvesting, for example by triggering desiccation and stunted growth. Furthermore, they are also suitable for the general control and inhibition of unwanted vegetative growth without killing the plants in the process. Inhibition of vegetative growth plays a major role for many mono- and dicotyledonous crops since, for example, this can reduce or completely prevent lodging.

By virtue of their herbicidal and plant growth regulatory properties, the inventive compounds of the general formula (I) can also be used to control harmful plants in crops of genetically modified plants or plants modified by conventional mutagenesis. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material.

It is preferred with a view to transgenic crops to use the inventive compounds of the general formula (I) and/or salts thereof in economically important transgenic crops of useful plants and ornamentals, for example of cereals such as wheat, barley, rye, oats, millet, rice and corn or else crops of sugar beet, cotton, soybean, oilseed rape, potato, tomato, peas and other vegetables.

It is preferable to employ the inventive compounds of the general formula (I) also as herbicides in crops of useful plants which are resistant, or have been made resistant by recombinant means, to the phytotoxic effects of the herbicides.

By virtue of their herbicidal and plant growth regulatory properties, the inventive compounds of the general formula (I) can also be used to control harmful plants in crops of genetically modified plants which are known or are yet to be developed. In general, the transgenic plants are characterized by particular advantageous properties, for example by resistances to certain pesticides, in particular certain herbicides, resistances to plant diseases or pathogens of plant diseases, such as certain insects or microorganisms such as fungi, bacteria or viruses. Other specific characteristics relate, for example, to the harvested material with regard to quantity, quality, storability, composition and specific constituents. For instance, there are known transgenic plants with an elevated starch content or altered starch quality, or those with a different fatty acid composition in the harvested material. Further special properties may be tolerance or resistance to abiotic stressors, for example heat, cold, drought, salinity and ultraviolet radiation.

Preference is given to the use of the inventive compounds of the general formula (I) or salts thereof in economically important transgenic crops of useful plants and ornamentals, for example of cereals such as wheat, barley, rye, oats, triticale, millet, rice, cassava and corn, or else crops of sugar beet, cotton, soybean, oilseed rape, potatoes, tomatoes, peas and other vegetables.

It is preferable to employ the compounds of the general formula (I) as herbicides in crops of useful plants which are resistant, or have been made resistant by recombinant means, to the phytotoxic effects of the herbicides.

Conventional ways of producing novel plants which have modified properties in comparison to existing plants consist, for example, in traditional cultivation methods and the generation of mutants. Alternatively, novel plants with altered properties can be generated with the aid of recombinant methods.

A large number of molecular-biological techniques by means of which novel transgenic plants with modified properties can be generated are known to the person skilled in the art. For such genetic manipulations, nucleic acid molecules which allow mutagenesis or sequence alteration by recombination of DNA sequences can be introduced into plasmids. With the aid of standard methods, it is possible, for example, to undertake base exchanges, remove part sequences or add natural or synthetic sequences. To connect the DNA fragments to each other, adapters or linkers may be added to the fragments.

For example, the generation of plant cells with a reduced activity of a gene product can be achieved by expressing at least one corresponding antisense RNA, a sense RNA for achieving a cosuppression effect, or by expressing at least one suitably constructed ribozyme which specifically cleaves transcripts of the abovementioned gene product.

To this end, it is firstly possible to use DNA molecules which encompass the entire coding sequence of a gene product inclusive of any flanking sequences which may be present, and also DNA molecules which only encompass portions of the coding sequence, in which case it is necessary for these portions to be long enough to have an antisense effect in the cells. It is also possible to use DNA sequences which have a high degree of homology to the coding sequences of a gene product, but are not completely identical to them.

When expressing nucleic acid molecules in plants, the protein synthesized may be localized in any desired compartment of the plant cell. However, to achieve localization in a particular compartment, it is possible, for example, to join the coding region to DNA sequences which ensure localization in a particular compartment. Sequences of this kind are known to the person skilled in the art (see, for example, Braun et al., EMBO J. 11 (1992), 3219-3227). The nucleic acid molecules can also be expressed in the organelles of the plant cells.

The transgenic plant cells can be regenerated by known techniques to give rise to entire plants. In principle, the transgenic plants may be plants of any desired plant species, i.e. not only monocotyledonous but also dicotyledonous plants.

Obtainable in this way are transgenic plants having properties altered by overexpression, suppression or inhibition of homologous (=natural) genes or gene sequences or expression of heterologous (=foreign) genes or gene sequences.

It is preferable to employ the inventive compounds of the general formula (I) in transgenic crops which are resistant to growth regulators such as, for example, dicamba, or to herbicides which inhibit essential plant enzymes, for example acetolactate synthases (ALS), EPSP synthases, glutamine synthases (GS) or hydroxyphenylpyruvate dioxygenases (HPPD), or to herbicides from the group of the sulfonylureas, glyphosates, glufosinates or benzoylisoxazoles and analogous active ingredients.

When the inventive compounds of the general formula (I) are employed in transgenic crops, not only do the effects toward harmful plants observed in other crops occur, but frequently also effects which are specific to application in the particular transgenic crop, for example an altered or specifically widened spectrum of weeds which can be controlled, altered application rates which can be used for the application, preferably good combinability with the herbicides to which the transgenic crop is resistant, and influencing of growth and yield of the transgenic crop plants.

The invention therefore also relates to the use of the inventive compounds of the general formula (I) and/or salts thereof as herbicides for controlling harmful plants in crops of useful plants or ornamentals, optionally in transgenic crop plants.

Preference is given to the use in cereals, here preferably corn, wheat, barley, rye, oats, millet or rice, by the pre- or post-emergence method.

Preference is also given to the use in soybeans by the pre- or post-emergence method.

The use according to the invention for the control of harmful plants or for growth regulation of plants also includes the case in which a compound of the general formula (I) or its salt is not formed from a precursor substance (“prodrug”) until after application on the plant, in the plant or in the soil.

The invention also provides for the use of one or more compounds of the general formula (I) or salts thereof or of a composition of the invention (as defined below) (in a method) for controlling harmful plants or for regulating the growth of plants which comprises applying an effective amount of one or more compounds of the general formula (I) or salts thereof onto the plants (harmful plants, if appropriate together with the useful plants), plant seeds, the soil in which or on which the plants grow or the area under cultivation.

The invention also provides a herbicidal and/or plant growth-regulating composition, characterized in that the composition comprises
(a) one or more compounds of the general formula (I) and/or salts thereof, as defined above, preferably in one of the embodiments identified as preferred or particularly preferred, in particular one or more compounds of the formulae (I.1) to (I.33) and/or salts thereof, in each case as defined above,
and
(b) one or more further substances selected from groups (i) and/or (ii):
   (i) one or more further agrochemically active substances, preferably selected from the group consisting of insecticides, acaricides, nematicides, further herbicides (i.e. those not conforming to the general formula (I) defined above), fungicides, safeners, fertilizers and/or further growth regulators,
   (ii) one or more formulation auxiliaries customary in crop protection.

The further agrochemically active substances of component (i) of a composition of the invention are preferably selected from the group of substances mentioned in “The Pesticide Manual”, 16th edition, The British Crop Protection Council and the Royal Soc. of Chemistry, 2012.

A herbicidal or plant growth-regulating composition of the invention comprises preferably one, two, three or more formulation auxiliaries (ii) customary in crop protection selected from the group consisting of surfactants, emulsifiers, dispersants, film formers, thickeners, inorganic salts, dusting agents, carriers that are solid at 25° C. and 1013 mbar, preferably adsorptive granulated inert materials, wetting agents, antioxidants, stabilizers, buffer substances, antifoam agents, water, organic solvents, preferably organic solvents miscible with water in any ratio at 25° C. and 1013 mbar.

The inventive compounds of the general formula (I) can be used in the form of wettable powders, emulsifiable concentrates, sprayable solutions, dusting products or granules in the customary formulations. The invention therefore also provides herbicidal and plant growth-regulating compositions which comprise compounds of the general formula (I) and/or salts thereof.

The inventive compounds of the general formula (I) and/or salts thereof can be formulated in various ways according to which biological and/or physicochemical parameters are specified. Possible formulations include, for example: wettable powders (WP), water-soluble powders (SP), water-soluble concentrates, emulsifiable concentrates (EC), emulsions (EW), such as oil-in-water and water-in-oil emulsions, sprayable solutions, suspension concentrates (SC), dispersions based on oil or water, oil-miscible solutions, capsule suspensions (CS), dusting products (DP), dressings, granules for scattering and soil application, granules (GR) in the form of microgranules, spray granules, absorption and adsorption granules, water-dispersible granules (WG), water-soluble granules (SG), ULV formulations, microcapsules and waxes.

These individual formulation types and the formulation auxiliaries, such as inert materials, surfactants, solvents and further additives, are known to the person skilled in the art and are described, for example, in: Watkins, “Handbook of Insecticide Dust Diluents and Carriers”, 2nd ed., Darland Books, Caldwell N.J., H.v. Olphen, “Introduction to Clay Colloid Chemistry”, 2nd ed., J. Wiley & Sons, N.Y., C. Marsden, “Solvents Guide”, 2nd ed., Interscience, N.Y. 1963, McCutcheon's “Detergents and Emulsifiers Annual”, MC Publ. Corp., Ridgewood N.J., Sisley and Wood, “Encyclopedia of Surface Active Agents”, Chem. Publ. Co. Inc., N.Y. 1964, Schönfeldt, “Grenzflächenaktive Äthylenoxidaddukte” [Interface-active Ethylene Oxide Adducts], Wiss. Verlagsgesellschaft, Stuttgart 1976, Winnacker-Küchler, “Chemische Technologie”, Volume 7, C. Hanser Verlag Munich, 4th ed. 1986.

Wettable powders are preparations which can be dispersed uniformly in water and, in addition to the active ingredient, apart from a diluent or inert substance, also comprise surfactants of the ionic and/or nonionic type (wetting agents, dispersants), for example polyoxyethylated alkylphenols, polyoxyethylated fatty alcohols, polyoxyethylated fatty amines, fatty alcohol polyglycol ether sulfates, alkanesulfonates, alkylbenzenesulfonates, sodium lignosulfonate, sodium 2,2′-dinaphthylmethane-6,6′-disulfonate, sodium dibutylnaphthalenesulfonate or else sodium oleoylmethyltaurate. To produce the wettable powders, the active herbicidal ingredients are finely ground, for example in customary apparatuses such as hammer mills, blower mills and air-jet mills, and simultaneously or subsequently mixed with the formulation auxiliaries.

Emulsifiable concentrates are produced by dissolving the active ingredient in an organic solvent, for example butanol, cyclohexanone, dimethylformamide, xylene, or else relatively high-boiling aromatics or hydrocarbons or mixtures of the organic solvents, with addition of one or more ionic and/or nonionic surfactants (emulsifiers). Examples of emulsifiers which may be used are: calcium alkylarylsulfonate salts, for example calcium dodecylbenzenesulfonate, or nonionic emulsifiers such as fatty acid polyglycol esters, alkylaryl polyglycol ethers, fatty alcohol polyglycol ethers, propylene oxide-ethylene oxide condensation products, alkyl polyethers, sorbitan esters, for example sorbitan fatty acid esters, or polyoxyethylene sorbitan esters, for example polyoxyethylene sorbitan fatty acid esters.

Dusting products are obtained by grinding the active ingredient with finely distributed solids, for example talc, natural clays, such as kaolin, bentonite and pyrophyllite, or diatomaceous earth.

Suspension concentrates may be water- or oil-based. They can be produced, for example, by wet grinding by means of standard commercial bead mills and optionally the addition of surfactants, as have already been listed e.g. above for the other types of formulation.

Emulsions, e.g. oil-in-water emulsions (EW), can be prepared, for example, by means of stirrers, colloid mills and/or static mixers using aqueous organic solvents and optionally surfactants, as have already been listed e.g. above for the other formulation types.

Granules can be produced either by spraying the active ingredient onto granular inert material capable of adsorption or by applying active ingredient concentrates to the surface of carrier substances, such as sand, kaolinites or granular inert material, by means of adhesives, for example polyvinyl alcohol, sodium polyacrylate or else mineral oils. Suitable active ingredients can also be granulated in the manner customary for the production of fertilizer granules—if desired as a mixture with fertilizers.

Water-dispersible granules are produced generally by the customary processes such as spray-drying, fluidized-bed granulation, pan granulation, mixing with high-speed mixers and extrusion without solid inert material.

For the production of pan granules, fluidized bed granules, extruder granules and spray granules, see, for example, processes in “Spray-Drying Handbook” 3rd ed. 1979, G. Goodwin Ltd., London; J. E. Browning, “Agglomeration”, Chemical and Engineering 1967, pages 147 ff.; “Perry's Chemical Engineer's Handbook”, 5th Ed., McGraw-Hill, New York 1973, pp. 8-57.

For further details regarding the formulation of crop protection compositions, see, for example, G. C. Klingman, “Weed Control as a Science”, John Wiley and Sons, Inc., New York, 1961, pages 81-96 and J. D. Freyer, S. A. Evans, “Weed Control Handbook”, 5th Ed., Blackwell Scientific Publications, Oxford, 1968, pages 101-103.

The agrochemical preparations, preferably herbicidal or plant growth-regulating compositions, of the present invention preferably comprise a total amount of 0.1 to 99% by weight, preferably 0.5 to 95% by weight, more preferably 1 to 90% by weight, especially preferably 2 to 80% by weight, of active ingredients of the general formula (I) and salts thereof.

In wettable powders, the active ingredient concentration is, for example, about 10% to 90% by weight, the remainder to 100% by weight consisting of customary formulation constituents. In emulsifiable concentrates, the active ingredient concentration may be about 1% to 90% and preferably 5% to 80% by weight. Formulations in the form of dusts comprise 1% to 30% by weight of active ingredient, preferably usually 5% to 20% by weight of active ingredient; sprayable solutions contain about 0.05% to 80% by weight, preferably 2% to 50% by weight of active ingredient. In the case of water-dispersible granules, the active ingredient content depends partly on whether the active ingredient is in liquid or solid form and on which granulation auxiliaries, fillers, and so forth are used. In the water-dispersible granules, the content of active ingredient is, for example, between 1% and 95% by weight, preferably between 10% and 80% by weight.

In addition, the active ingredient formulations mentioned optionally comprise the respective customary stickers, wetters, dispersants, emulsifiers, penetrants, preservatives, antifreeze agents and solvents, fillers, carriers and dyes, defoamers, evaporation inhibitors and agents which influence the pH and the viscosity. Examples of formulation auxiliaries are described, inter alia, in “Chemistry and Technology of Agrochemical Formulations”, ed. D. A. Knowles, Kluwer Academic Publishers (1998).

The inventive compounds of the general formula (I) or salts thereof can be used as such or in the form of their preparations (formulations) in a combination with other pesticidally active substances, for example insecticides, acaricides, nematicides, herbicides, fungicides, safeners, fertilizers and/or growth regulators, for example in the form of a finished formulation or of a tankmix. The combination formulations can be produced on the basis of the abovementioned formulations, taking account of the physical properties and stabilities of the active ingredients to be combined.

Combination partners usable for the inventive compounds of the general formula (I) in mixed formulations or in a tankmix are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II, protoporphyrinogen oxidase, as described, for example, in Weed Research 26 (1986) 441-445 or “The Pesticide Manual”, 16th edition, The British Crop Protection Council and the Royal Soc. of Chemistry, 2012, and the literature cited therein.

Of particular interest is the selective control of harmful plants in crops of useful plants and ornamentals. Although the inventive compounds of the general formula (I) have already demonstrated very good to adequate selectivity in a large number of crops, in principle, in some crops and in particular also in the case of mixtures with other, less selective herbicides, phytotoxicities on the crop plants may occur. In this connection, combinations of inventive compounds of the general formula (I) that are of particular interest are those comprising the compounds (I) or combinations thereof with other herbicides or pesticides and safeners. The safeners, which are used in an antidotically effective amount, reduce the phytotoxic side effects of the herbicides/pesticides employed, for example in economically important crops, such as cereals (wheat, barley, rye, corn, rice, millet), sugarbeet, sugarcane, oilseed rape, cotton and soybeans, preferably cereals.

The weight ratios of herbicide (mixture) to safener depend generally on the herbicide application rate and the efficacy of the safener in question and may vary within wide limits, for example in the range from 200:1 to 1:200, preferably 100:1 to 1:100, in particular 20:1 to 1:20. Analogously to the compounds of the general formula (I) or mixtures thereof, the safeners can be formulated with further herbicides/pesticides and be provided and employed as a finished formulation or tank mix with the herbicides.

For application, the herbicide formulations or herbicide-safener formulations in the commercial form are diluted if appropriate in a customary manner, for example with water in the case of wettable powders, emulsifiable concentrates, dispersions and water-dispersible granules. Preparations in dust form, granules for soil application or granules for scattering and sprayable solutions are not normally diluted further with other inert substances prior to application.

The application rate of the inventive compounds of the general formula (I) and/or salts thereof is affected to a certain extent by external conditions such as temperature, humidity, etc. The application rate may vary within wide limits. For application as a herbicide for controlling harmful plants, the total amount of inventive compounds of the general formula (I) and salts thereof is preferably in the range from 0.001 to 10.0 kg/ha, with preference in the range from 0.005 to 5 kg/ha, more preferably in the range from 0.01 to 1.5 kg/ha, particularly preferably in the range from 0.05 to 1 kg/ha. This applies both to pre-emergence and to post-emergence application.

When the inventive compounds of the general formula (I) and/or salts thereof are used as plant growth regulator, for example as culm stabilizer for crop plants like those mentioned above, preferably cereal plants, such as wheat, barley, rye, triticale, millet, rice or corn, the total application rate is preferably in the range of from 0.001 to 2 kg/ha, preferably in the range of from 0.005 to 1 kg/ha, in particular in the range of from 10 to 500 g/ha, very particularly preferably in the range from 20 to 250 g/ha. This applies both to pre-emergence and to post-emergence application.

The application as culm stabilizer may take place at various stages of the growth of the plants. Preferred is, for example, the application after the tillering phase, at the beginning of the longitudinal growth.

As an alternative, application as plant growth regulator is also possible by treating the seed, which includes various techniques for dressing and coating seed. The application rate depends on the particular techniques and can be determined in preliminary tests.

Combination partners usable for the inventive compounds of the general formula (I) in compositions of the invention (e.g. mixed formulations or in a tankmix) are, for example, known active ingredients based on inhibition of, for example, acetolactate synthase, acetyl-CoA carboxylase, cellulose synthase, enolpyruvylshikimate-3-phosphate synthase, glutamine synthetase, p-hydroxyphenylpyruvate dioxygenase, phytoene desaturase, photosystem I, photosystem II or protoporphyrinogen oxidase, as described, for example, from Weed Research 26 (1986) 441-445 or “The Pesticide Manual”, 16th edition, The British Crop Protection Council and the Royal Soc. of Chemistry, 2012, and literature cited therein. Known herbicides or plant growth regulators which can be combined with the compounds of the invention are, for example, the following, where said active ingredients are referred to either by their “common name” in accordance with the International Organization for Standardization (ISO) or by the chemical name or by the code number. They always encompass all the use forms, for example acids, salts, esters and also all isomeric forms such as stereoisomers and optical isomers, even if they are not mentioned explicitly.

Examples of such herbicidal mixing partners are:
acetochlor, acifluorfen, acifluorfen-sodium, aclonifen, alachlor, allidochlor, alloxydim, alloxydim-sodium, ametryn, amicarbazone, amidochlor, amidosulfuron, 4-amino-3-chloro-6-(4-chloro-2-fluoro-3-methylphenyl)-5-fluoropyridine-2-carboxylic acid, aminocyclopyrachlor, aminocyclopyrachlor-potassium, aminocyclopyrachlor-methyl, aminopyralid, amitrole, ammoniumsulfamate, anilofos, asulam, atrazine, azafenidin, azimsulfuron, beflubutamid, benazolin, benazolin-ethyl, benfluralin, benfuresate, bensulfuron, bensulfuron-methyl, bensulide, bentazone, benzobicyclon, benzofenap, bicyclopyron, bifenox, bilanafos, bilanafos-sodium, bispyribac, bispyribac-sodium, bromacil, bromobutide, bromofenoxim, bromoxynil, bromoxynil-butyrate, -potassium, -heptanoate and -octanoate, busoxinone, butachlor, butafenacil, butamifos, butenachlor, butralin, butroxydim, butylate, cafenstrole, carbetamide, carfentrazone, carfentrazone-ethyl, chloramben, chlorbromuron, chlorfenac, chlorfenac-sodium, chlorfenprop, chlorflurenol, chlorflurenol-methyl, chloridazon, chlorimuron, chlorimuron-ethyl, chlorophthalim, chlorotoluron, chlorthal-dimethyl, chlorsulfuron, cinidon, cinidon-ethyl, cinmethylin, cinosulfuron, clacyfos, clethodim, clodinafop, clodinafop-propargyl, clomazone, clomeprop, clopyralid, cloransulam, cloransulam-methyl, cumyluron, cyanamide, cyanazine, cycloate, cyclopyrimorate, cyclosulfamuron, cycloxydim, cyhalofop, cyhalofop-butyl, cyprazine, 2,4-D, 2,4-D-butotyl, -butyl, -dimethylammonium, -diolamine, -ethyl, 2-ethylhexyl, -isobutyl, -isooctyl, -isopropylammonium, -potassium, -triisopropanolammonium and -trolamine, 2,4-DB, 2,4-DB-butyl, -dimethylammonium, isooctyl, -potassium and -sodium, daimuron (dymron), dalapon, dazomet, n-decanol, desmedipham, detosyl-pyrazolate (DTP), dicamba, dichlobenil, 2-(2,4-dichlorobenzyl)-4,4-dimethyl-1,2-oxazolidin-3-one, 2-(2,5-dichlorobenzyl)-4,4-dimethyl-1,2-oxazolidin-3-one, dichlorprop, dichlorprop-P, diclofop, diclofop-methyl, diclofop-P-methyl, diclosulam, difenzoquat, diflufenican, diflufenzopyr, diflufenzopyr-sodium, dimefuron, dimepiperate, dimethachlor, dimethametryn, dimethenamid, dimethenamid-P, dimetrasulfuron, dinitramine, dinoterb, diphenamid, diquat, diquat-dibromid, dithiopyr, diuron, DNOC, endothal, EPTC, esprocarb, ethalfluralin, ethametsulfuron, ethametsulfuron-methyl, ethiozin, ethofumesate, ethoxyfen, ethoxyfen-ethyl, ethoxysulfuron, etobenzanid, F-9600, F-5231, i.e. N-[2-chloro-4-fluoro-5-[4-(3-fluoropropyl)-4,5-dihydro-5-oxo-1H-tetrazol-1-yl]-phenyl]ethanesulfonamide, F-7967, i.e. 3-[7-chloro-5-fluoro-2-(trifluoromethyl)-1H-benzimidazol-4-yl]-1-methyl-6-(trifluoromethyl)pyrimidine-2,4(1H,3H)-dione, fenoxaprop, fenoxaprop-P, fenoxaprop-ethyl, fenoxaprop-P-ethyl, fenoxasulfone, fenquinotrione, fentrazamide, flamprop, flamprop-M-isopropyl, flamprop-M-methyl, flazasulfuron, florasulam, fluazifop, fluazifop-P, fluazifop-butyl, fluazifop-P-butyl, flucarbazone, flucarbazone-sodium, flucetosulfuron, fluchloralin, flufenacet, flufenpyr, flufenpyr-ethyl, flumetsulam, flumiclorac, flumiclorac-pentyl, flumioxazin, fluometuron, flurenol, flurenol-butyl, -dimethylammonium and -methyl, fluoroglycofen, fluoroglycofen-ethyl, flupropanate, flupyrsulfuron, flupyrsulfuron-methyl-sodium, fluridone, flurochloridone, fluroxypyr, fluroxypyr-meptyl, flurtamone, fluthiacet, fluthiacet-methyl, fomesafen, fomesafen-sodium, foramsulfuron, fosamine, glufosinate, glufosinate-ammonium, glufosinate-P-sodium, glufosinate-P-ammonium, glufosinate-P-sodium, glyphosate, glyphosate-ammonium, -isopropylammonium, -diammonium, -dimethylammonium, -potassium, -sodium and -trimesium, H-9201, i.e. O-(2,4-dimethyl-6-nitrophenyl)O-ethyl isopropylphosphoramidothioate, halauxifen, halauxifen-methyl, halosafen, halosulfuron, halosulfuron-methyl, haloxyfop, haloxyfop-P, haloxyfop-ethoxyethyl, haloxyfop-P-ethoxyethyl, haloxyfop-methyl, haloxyfop-P-methyl, hexazinone, HW-02, i.e. 1-(dimethoxyphosphoryl)ethyl (2,4-dichlorophenoxy)acetate, imazamethabenz, Imazamethabenz-methyl, imazamox, imazamox-ammonium, imazapic, imazapic-ammonium, imazapyr, imazapyr-isopropylammonium, imazaquin, imazaquin-ammonium, imazethapyr, imazethapyr-immonium, imazosulfuron, indanofan, indaziflam, iodosulfuron, iodosulfuron-methyl-sodium, ioxynil, ioxynil-octanoate, -potassium and sodium, ipfencarbazone, isoproturon, isouron, isoxaben, isoxaflutole, karbutilate, KUH-043, i.e. 3-({[5-(difluoromethyl)-1-methyl-3-(trifluoromethyl)-1H-pyrazol-4-yl]methyl}sulfonyl)-5,5-dimethyl-4,5-dihydro-1,2-oxazole, ketospiradox, lactofen, lenacil, linuron, MCPA, MCPA-butotyl, -dimethylammonium, -2-ethylhexyl, -isopropylammonium, -potassium and -sodium, MCPB, MCPB-methyl, -ethyl and -sodium, mecoprop, mecoprop-sodium, and -butotyl, mecoprop-P, mecoprop-P-butotyl, -dimethylammonium, -2-ethylhexyl and -potassium, mefenacet, mefluidide, mesosulfuron, mesosulfuron-methyl, mesotrione, methabenzthiazuron, metam, metamifop, metamitron, metazachlor, metazosulfuron, methabenzthiazuron, methiopyrsulfuron, methiozolin, methyl isothiocyanate, metobromuron, metolachlor, S-metolachlor, metosulam, metoxuron, metribuzin, metsulfuron, metsulfuron-methyl, molinat, monolinuron, monosulfuron, monosulfuron-ester, MT-5950, i.e. N-[3-chloro-4-(1-methylethyl)phenyl]-2-methylpentanamide, NGGC-011, napropamide, NC-310, i.e. 4-(2,4-dichlorobenzoyl)-1-methyl-5-benzyloxypyrazole, neburon, nicosulfuron, nonanoic acid (pelargonic acid), norflurazon, oleic acid (fatty acids), orbencarb, orthosulfamuron, oryzalin, oxadiargyl, oxadiazon, oxasulfuron, oxaziclomefon, oxyfluorfen, paraquat, paraquat dichloride, pebulate, pendimethalin, penoxsulam, pentachlorphenol, pentoxazone, pethoxamid, petroleum oils, phenmedipham, picloram, picolinafen, pinoxaden, piperophos, pretilachlor, primisulfuron, primisulfuron-methyl, prodiamine, profoxydim, prometon, prometryn, propachlor, propanil, propaquizafop, propazine, propham, propisochlor, propoxycarbazone, propoxycarbazone-sodium, propyrisulfuron, propyzamide, prosulfocarb, prosulfuron, pyraclonil, pyraflufen, pyraflufen-ethyl, pyrasulfotole, pyrazolynate (pyrazolate), pyrazosulfuron, pyrazosulfuron-ethyl, pyrazoxyfen, pyribambenz, pyribambenz-isopropyl, pyribambenz-propyl, pyribenzoxim, pyributicarb, pyridafol, pyridate, pyriftalid, pyriminobac, pyriminobac-methyl, pyrimisulfan, pyrithiobac, pyrithiobac-sodium, pyroxasulfone, pyroxsulam, quinclorac, quinmerac, quinoclamine, quizalofop, quizalofop-ethyl, quizalofop-P, quizalofop-P-ethyl, quizalofop-P-tefuryl, rimsulfuron, saflufenacil, sethoxydim, siduron, simazine, simetryn, SL-261, sulcotrion, sulfentrazone, sulfometuron, sulfometuron-methyl, sulfosulfuron, SYN-523, SYP-249, i.e. 1-ethoxy-3-methyl-1-oxobut-3-en-2-yl 5-[2-chloro-4-(trifluoromethyl)phenoxy]-2-nitrobenzoate, SYP-300, i.e. 1-[7-fluoro-3-oxo-4-(prop-2-yn-1-yl)-3,4-dihydro-2H-1,4-benzoxazin-6-yl]-3-propyl-2-thioxoimidazolidine-4,5-dione, 2,3,6-TBA, TCA (trifluoroacetic acid), TCA-sodium, tebuthiuron, tefuryltrione, tembotrione, tepraloxydim, terbacil, terbucarb, terbumeton, terbuthylazin, terbutryn, thenylchlor, thiazopyr, thiencarbazone, thiencarbazone-methyl, thifensulfuron, thifensulfuron-methyl, thiobencarb, tiafenacil, tolpyralate, topramezone, tralkoxydim, triafamone, tri-allate, triasulfuron, triaziflam, tribenuron, tribenuron-methyl, triclopyr, trietazine, trifloxysulfuron, trifloxysulfuron-sodium, trifludimoxazin, trifluralin, triflusulfuron, triflusulfuron-methyl, tritosulfuron, urea sulfate, vernolate, XDE-848, ZJ-0862, i.e. 3,4-dichloro-N-{2-[(4,6-dimethoxypyrimidin-2-yl)oxy]benzyl}aniline, and the following compounds:

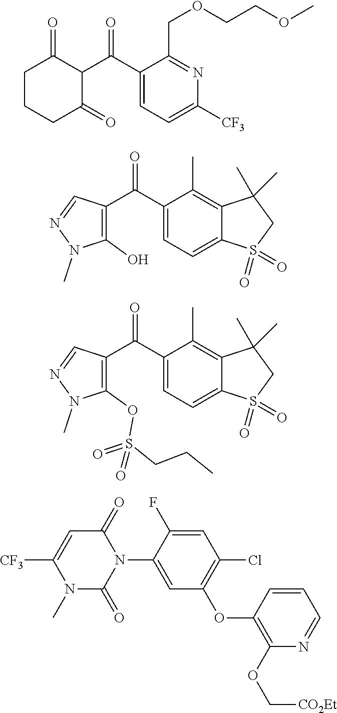

Examples of plant growth regulators as possible mixing partners are:
acibenzolar, acibenzolar-S-methyl, 5-aminolevulinic acid, ancymidol, 6-benzylaminopurine, brassinolide, catechol, chlormequat chloride, cloprop, cyclanilide, 3-(cycloprop-1-enyl)propionic acid, daminozide, dazomet, n-decanol, dikegulac, dikegulac-sodium, endothal, endothal-dipotassium, -disodium, and mono(N,N-dimethylalkylammonium), ethephon, flumetralin, flurenol, flurenol-butyl, flurprimidol, forchlorfenuron, gibberellic acid, inabenfide, indole-3-acetic acid (IAA), 4-indol-3-ylbutyric acid, isoprothiolane, probenazole, jasmonic acid, jasmonic acid methyl ester, maleic hydrazide, mepiquat chloride, 1-methylcyclopropene, 2-(1-naphthyl)acetamide, 1-naphthylacetic acid, 2-naphthyloxyacetic acid, nitrophenolate mixture, 4-oxo-4[(2-phenylethyl)amino]butyric acid, paclobutrazole, N-phenylphthalamic acid, prohexadione, prohexadione-calcium, prohydrojasmone, salicylic acid, strigolactone, tecnazene, thidiazuron, triacontanol, trinexapac, trinexapac-ethyl, tsitodef, uniconazole, uniconazole-P.

Useful combination partners for the inventive compounds of the general formula (I) also include, for example, the following safeners:
S1) Compounds from the group of heterocyclic carboxylic acid derivatives:
S1^{a}) Compounds of the dichlorophenylpyrazoline-3-carboxylic acid type (S1^{a}), preferably compounds such as 1-(2,4-dichlorophenyl)-5-(ethoxycarbonyl)-5-methyl-2-pyrazoline-3-carboxylic acid, ethyl 1-(2,4-dichlorophenyl)-5-(ethoxycarbonyl)-5-methyl-2-pyrazoline-3-carboxylate (S1-1) (“mefenpyr-diethyl”), and related compounds as described in WO-A-91/07874;
S1^{b}) Derivatives of dichlorophenylpyrazolecarboxylic acid (S1^{b}), preferably compounds such as ethyl 1-(2,4-dichlorophenyl)-5-methylpyrazole-3-carboxylate (S1-2), ethyl 1-(2,4-dichlorophenyl)-5-isopropylpyrazole-3-carboxylate (S1-3), ethyl 1-(2,4-dichlorophenyl)-5-(1,1-dimethylethyl)pyrazole-3-carboxylate (S1-4) and related compounds as described in EP-A-333131 and EP-A-269806;
S1^{c}) Derivatives of 1,5-diphenylpyrazole-3-carboxylic acid (S1^{c}), preferably compounds such as ethyl 1-(2,4-dichlorophenyl)-5-phenylpyrazole-3-carboxylate (S1-5), methyl 1-(2-chlorophenyl)-5-phenylpyrazole-3-carboxylate (S1-6) and related compounds as described, for example, in EP-A-268554;
S1^{d}) Compounds of the triazolecarboxylic acid type (S1^{d}), preferably compounds such as fenchlorazole(-ethyl ester), i.e. ethyl 1-(2,4-dichlorophenyl)-5-trichloromethyl-1H-1,2,4-triazole-3-carboxylate (S1-7), and related compounds, as described in EP-A-174562 and EP-A-346620;
S1^{e}) Compounds of the 5-benzyl- or 5-phenyl-2-isoxazoline-3-carboxylic acid or of the 5,5-diphenyl-2-isoxazoline-3-carboxylic acid type (S1^{e}), preferably compounds such as ethyl 5-(2,4-dichlorobenzyl)-2-isoxazoline-3-carboxylate (S1-8) or ethyl 5-phenyl-2-isoxazoline-3-carboxylate (S1-9) and related compounds as described in WO-A-91/08202, or 5,5-diphenyl-2-isoxazolinecarboxylic acid (S1-10) or ethyl 5,5-diphenyl-2-isoxazoline-3-carboxylate (S1-11) (“isoxadifen-ethyl”) or n-propyl 5,5-diphenyl-2-isoxazoline-3-carboxylate (S1-12) or ethyl 5-(4-fluorophenyl)-5-phenyl-2-isoxazoline-3-carboxylate (S1-13) as described in patent application WO-A-95/07897.
S2) Compounds from the group of the 8-quinolinoxy derivatives (S2):
S2^{a}) Compounds of the 8-quinolinoxyacetic acid type (S2^{a}), preferably 1-methylhexyl (5-chloro-8-quinolinoxy)acetate (“cloquintocet-mexyl”) (S2-1), 1,3-dimethylbut-1-yl (5-chloro-8-quinolinoxy)acetate (S2-2), 4-allyloxybutyl (5-chloro-8-quinolinoxy)acetate (S2-3), 1-allyloxyprop-2-yl (5-chloro-8-quinolinoxy)acetate (S2-4), ethyl (5-chloro-8-quinolinoxy)acetate (S2-5),
methyl (5-chloro-8-quinolinoxy)acetate (S2-6),
allyl (5-chloro-8-quinolinoxy)acetate (S2-7), 2-(2-propylideneiminoxy)-1-ethyl (5-chloro-8-quinolinoxy)acetate (S2-8), 2-oxoprop-1-yl (5-chloro-8-quinolinoxy)acetate (S2-9) and related compounds, as described in EP-A-86750, EP-A-94349 and EP-A-191736 or EP-A-0 492 366, and also (5-chloro-8-quinolinoxy)acetic acid (S2-10), hydrates and salts thereof, for example the lithium, sodium, potassium, calcium, magnesium, aluminum, iron, ammonium, quaternary ammonium, sulfonium or phosphonium salts thereof, as described in WO-A-2002/34048;
S2^{b}) Compounds of the (5-chloro-8-quinolinoxy)malonic acid type (S2^{b}), preferably compounds such as diethyl (5-chloro-8-quinolinoxy)malonate, diallyl (5-chloro-8-quinolinoxy)malonate, methyl ethyl (5-chloro-8-quinolinoxy)malonate and related compounds, as described in EP-A-0 582 198.
S3) Active ingredients of the dichloroacetamide type (S3), which are frequently used as pre-emergence safeners (soil-acting safeners), for example
“dichlormid” (N,N-diallyl-2,2-dichloroacetamide) (S3-1),
“R-29148” (3-dichloroacetyl-2,2,5-trimethyl-1,3-oxazolidine) from Stauffer (S3-2),
“R-28725” (3-dichloroacetyl-2,2-dimethyl-1,3-oxazolidine) from Stauffer (S3-3),
“benoxacor” (4-dichloroacetyl-3,4-dihydro-3-methyl-2H-1,4-benzoxazine) (S3-4),
“PPG-1292” (N-allyl-N-[(1,3-dioxolan-2-yl)methyl]dichloroacetamide) from PPG Industries (S3-5),
“DKA-24” (N-allyl-N-[(allylaminocarbonyl)methyl]dichloroacetamide) from Sagro-Chem (S3-6),
“AD-67” or “MON 4660” (3-dichloroacetyl-1-oxa-3-azaspiro[4.5]decane) from Nitrokemia or Monsanto (S3-7),
“TI-35” (1-dichloroacetylazepane) from TRI-Chemical RT (S3-8),
“diclonon” (dicyclonon) or “BAS145138” or “LAB145138” (S3-9)
((RS)-1-dichloroacetyl-3,3,8a-trimethylperhydropyrrolo[1,2-a]pyrimidin-6-one) from BASF,
“furilazole” or “MON 13900” ((RS)-3-dichloroacetyl-5-(2-furyl)-2,2-dimethyloxazolidine) (S3-10), and the (R) isomer thereof (S3-11).
S4) Compounds from the class of the acylsulfonamides (S4):
S4^{a}) N-Acylsulfonamides of the formula (S4^{a}) and salts thereof, as described in WO-A-97/45016,

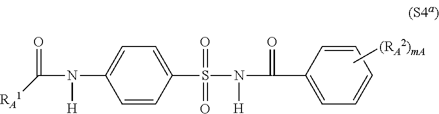

in which
R_{A}¹ is (C₁-C₆)-alkyl, (C₃-C₆)-cycloalkyl, where the 2 latter radicals are substituted by v_{A} substituents from the group of halogen, (C₁-C₄)-alkoxy, (C₁-C₆)-haloalkoxy and (C₁-C₄)-alkylthio and, in the case of cyclic radicals, also by (C₁-C₄)-alkyl and (C₁-C₄)-haloalkyl;
R_{A}² is halogen, (C₁-C₄)-alkyl, (C₁-C₄)-alkoxy, CF₃;
m_{A} is 1 or 2;
v_{A} is 0, 1, 2 or 3;
S4^{b}) Compounds of the 4-(benzoylsulfamoyl)benzamide type of the formula (S4^{b}) and salts thereof, as described in WO-A-99/16744,

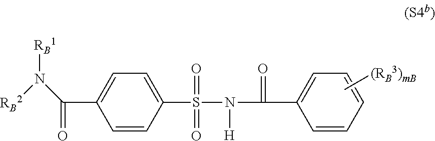

in which
R_{B}¹, R_{B}² are independently hydrogen, (C₁-C₆)-alkyl, (C₃-C₆)-cycloalkyl, (C₃-C₆)-alkenyl, (C₃-C₆)-alkynyl,
R_{B}³ is halogen, (C₁-C₄)-alkyl, (C₁-C₄)-haloalkyl or (C₁-C₄)-alkoxy and
m_{B} is 1 or 2,
e.g. those in which
R_{B}¹=cyclopropyl, R_{B}²=hydrogen and (R_{B}³)=2-OMe (“cyprosulfamide”, S4-1),
R_{B}¹=cyclopropyl, R_{B}²=hydrogen and (R_{B}³)=5-Cl-2-OMe (S4-2),
R_{B}¹=ethyl, R_{B}²=hydrogen and (R_{B}³)=2-OMe (S4-3),
R_{B}¹=isopropyl, R_{B}²=hydrogen and (R_{B}³)=5-Cl-2-OMe (S4-4) and
R_{B}¹=isopropyl, R_{B}²=hydrogen and (R_{B}³)=2-OMe (S4-5);
S4^{c}) Compounds from the class of the benzoylsulfamoylphenylureas of the formula (S4^{c}), as described in EP-A-365484,

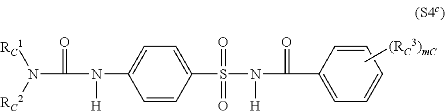

in which
R_{C}¹, R_{C}² are independently hydrogen, (C₁-C₈)-alkyl, (C₃-C₈)-cycloalkyl, (C₃-C₆)-alkenyl, (C₃-C₆)-alkynyl,
R_{C}³ is halogen, (C₁-C₄)-alkyl, (C₁-C₄)-alkoxy, CF₃ and
m_{C} is 1 or 2;
for example
1-[4-(N-2-methoxybenzoylsulfamoyl)phenyl]-3-methylurea,
1-[4-(N-2-methoxybenzoylsulfamoyl)phenyl]-3,3-dimethylurea,
1-[4-(N-4,5-dimethylbenzoylsulfamoyl)phenyl]-3-methylurea;
S4^{d}) Compounds of the N-phenylsulfonylterephthalamide type of the formula (S4^{d}) and salts thereof, which are known, for example, from CN 101838227,

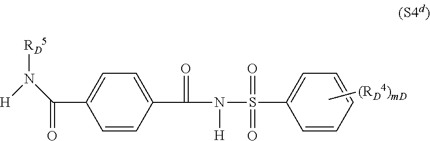

in which
R_{D}⁴ is halogen, (C₁-C₄)-alkyl, (C₁-C₄)-alkoxy, CF₃;
m_{D} is 1 or 2;
R_{D}⁵ is hydrogen, (C₁-C₆)-alkyl, (C₃-C₆)-cycloalkyl, (C₂-C₆)-alkenyl, (C₂-C₆)-alkynyl, (C₅-C₆)-cycloalkenyl.
S5) Active ingredients from the class of the hydroxyaromatics and the aromatic-aliphatic carboxylic acid derivatives (S5), for example
ethyl 3,4,5-triacetoxybenzoate, 3,5-dimethoxy-4-hydroxybenzoic acid, 3,5-dihydroxybenzoic acid, 4-hydroxysalicylic acid, 4-fluorosalicylic acid, 2-hydroxycinnamic acid, 2,4-dichlorocinnamic acid, as described in WO-A-2004/084631, WO-A-2005/015994, WO-A-2005/016001.
S6) Active ingredients from the class of the 1,2-dihydroquinoxalin-2-ones (S6), for example
1-methyl-3-(2-thienyl)-1,2-dihydroquinoxalin-2-one, 1-methyl-3-(2-thienyl)-1,2-dihydroquinoxaline-2-thione, 1-(2-aminoethyl)-3-(2-thienyl)-1,2-dihydroquinoxalin-2-one hydrochloride, 1-(2-methylsulfonylaminoethyl)-3-(2-thienyl)-1,2-dihydroquinoxalin-2-one, as described in WO-A-2005/112630.
S7) Compounds from the class of the diphenylmethoxyacetic acid derivatives (S7), e.g. methyl diphenylmethoxyacetate (CAS Reg. No. 41858-19-9) (S7-1), ethyl diphenylmethoxyacetate or diphenylmethoxyacetic acid, as described in WO-A-98/38856.
S8) Compounds of the formula (S8), as described in WO-A-98/27049,

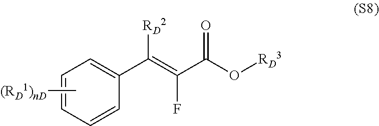

in which the symbols and indices are defined as follows:
R_{D}¹ is halogen, (C₁-C₄)-alkyl, (C₁-C₄)-haloalkyl, (C₁-C₄)-alkoxy, (C₁-C₄)-haloalkoxy,
R_{D}² is hydrogen or (C₁-C₄)-alkyl,
R_{D}³ is hydrogen, (C₁-C₈)-alkyl, (C₂-C₄)-alkenyl, (C₂-C₄)-alkynyl or aryl, where each of the aforementioned carbon-containing radicals is unsubstituted or substituted by one or more, preferably up to three, identical or different radicals from the group consisting of halogen and alkoxy; or salts thereof,
n_{D} is an integer from 0 to 2.
S9) Active ingredients from the class of the 3-(5-tetrazolylcarbonyl)-2-quinolones (S9), for example
1,2-dihydro-4-hydroxy-1-ethyl-3-(5-tetrazolylcarbonyl)-2-quinolone (CAS Reg. No.: 219479-18-2), 1,2-dihydro-4-hydroxy-1-methyl-3-(5-tetrazolylcarbonyl)-2-quinolone (CAS Reg. No. 95855-00-8), as described in WO-A-1999/000020.
S10) Compounds of the formulae (S10^{a}) or (S10^{b})
   as described in WO-A-2007/023719 and WO-A-2007/023764,
in which

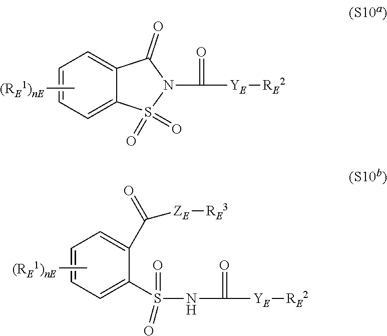

R_{E}¹ is halogen, (C₁-C₄)-alkyl, methoxy, nitro, cyano, CF₃, OCF₃,
Y_{E}, Z_{E} are independently O or S,
n_{E} is an integer from 0 to 4,
R_{E}² is (C₁-C₁₆)-alkyl, (C₂-C₆)-alkenyl, (C₃-C₆)-cycloalkyl, aryl; benzyl, halobenzyl,
R_{E}³ is hydrogen or (C₁-C₆)-alkyl.
S11) Active ingredients of the oxyimino compounds type (S11), which are known as seed-dressing agents, for example
“oxabetrinil” ((Z)-1,3-dioxolan-2-ylmethoxyimino(phenyl)acetonitrile) (S11-1), which is known as a seed-dressing safener for millet/sorghum against metolachlor damage,
   “fluxofenim” (1-(4-chlorophenyl)-2,2,2-trifluoro-1-ethanone O-(1,3-dioxolan-2-ylmethyl)oxime) (S11-2), which is known as a seed-dressing safener for millet/sorghum against metolachlor damage, and
   “cyometrinil” or “CGA-43089” ((Z)-cyanomethoxyimino(phenyl)acetonitrile) (S11-3), which is known as a seed-dressing safener for millet/sorghum against metolachlor damage.
S12) Active ingredients from the class of the isothiochromanones (S12), for example methyl [(3-oxo-1H-2-benzothiopyran-4(3H)-ylidene)methoxy]acetate (CAS Reg. No. 205121-04-6) (S12-1) and related compounds from WO-A-1998/13361.
S13) One or more compounds from group (S13):
   “naphthalic anhydride” (1,8-naphthalenedicarboxylic anhydride) (S13-1), which is known as a seed-dressing safener for corn against thiocarbamate herbicide damage,
   “fenclorim” (4,6-dichloro-2-phenylpyrimidine) (S13-2), which is known as a safener for pretilachlor in sown rice,
   “flurazole” (benzyl 2-chloro-4-trifluoromethyl-1,3-thiazole-5-carboxylate) (S13-3), which is known as a seed-dressing safener for millet/sorghum against alachlor and metolachlor damage, “CL 304415” (CAS Reg. No. 31541-57-8)
   (4-carboxy-3,4-dihydro-2H-1-benzopyran-4-acetic acid) (S13-4) from American Cyanamid, which is known as a safener for corn against damage by imidazolinones,
   “MG 191” (CAS Reg. No. 96420-72-3) (2-dichloromethyl-2-methyl-1,3-dioxolane) (S13-5) from Nitrokemia, which is known as a safener for corn,
   “MG 838” (CAS Reg. No. 133993-74-5)
   (2-propenyl 1-oxa-4-azaspiro[4.5]decane-4-carbodithioate) (S13-6) from Nitrokemia
   “disulfoton” (O,O-diethyl S-2-ethylthioethyl phosphorodithioate) (S13-7),
   “dietholate” (O,O-diethyl O-phenyl phosphorothioate) (S13-8),
   “mephenate” (4-chlorophenyl methylcarbamate) (S13-9).
S14) Active ingredients which, in addition to herbicidal action against harmful plants, also have safener action on crop plants such as rice, for example
“dimepiperate” or “MY-93” (S-1-methyl 1-phenylethylpiperidine-1-carbothioate), which is known as a safener for rice against damage by the herbicide molinate,
   “daimuron” or “SK 23” (1-(1-methyl-1-phenylethyl)-3-p-tolylurea), which is known as a safener for rice against damage by the herbicide imazosulfuron,
   “cumyluron”=“JC-940” (3-(2-chlorophenylmethyl)-1-(1-methyl-1-phenylethyl)urea, see JP-A-60087254), which is known as a safener for rice against damage by some herbicides,
   “methoxyphenone” or “NK 049” (3,3′-dimethyl-4-methoxybenzophenone), which is known as a safener for rice against damage by some herbicides,
   “CSB” (1-bromo-4-(chloromethylsulfonyl)benzene) from Kumiai, (CAS Reg. No. 54091-06-4), which is known as a safener against damage by some herbicides in rice.
S15) Compounds of the formula (S15) or tautomers thereof

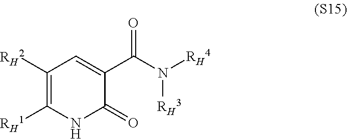

as described in WO-A-2008/131861 and WO-A-2008/131860
in which
R_{H}¹ is a (C₁-C₆)-haloalkyl radical and
R_{H}² is hydrogen or halogen and
R_{H}³, R_{H}⁴ are independently hydrogen, (C₁-C₁₆)-alkyl, (C₂-C₁₆)-alkenyl or (C₂-C₁₆)-alkynyl, where each of the 3 latter radicals is unsubstituted or substituted by one or more radicals from the group of halogen, hydroxyl, cyano, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
   or (C₃-C₆)-cycloalkyl, (C₄-C₆)-cycloalkenyl, (C₃-C₆)-cycloalkyl fused on one side of the ring to a 4 to 6-membered saturated or unsaturated carbocyclic ring, or (C₄-C₆)-cycloalkenyl fused on one side of the ring to a 4 to 6-membered saturated or unsaturated carbocyclic ring,
      where each of the 4 latter radicals is unsubstituted or substituted by one or more radicals from the group of halogen, hydroxyl, cyano, (C₁-C₄)-alkyl, (C₁-C₄)-haloalkyl, (C₁-C₄)-alkoxy, (C₁-C₄)-haloalkoxy, (C₁-C₄)-alkylthio, (C₁-C₄)-alkylamino, di[(C₁-C₄)-alkyl]amino, [(C₁-C₄)-alkoxy]carbonyl, [(C₁-C₄)-haloalkoxy]carbonyl, (C₃-C₆)-cycloalkyl which is unsubstituted or substituted, phenyl which is unsubstituted or substituted, and heterocyclyl which is unsubstituted or substituted,
      or
R_{H}³ is (C₁-C₄)-alkoxy, (C₂-C₄)-alkenyloxy, (C₂-C₆)-alkynyloxy or (C₂-C₄)-haloalkoxy and
R_{H}⁴ is hydrogen or (C₁-C₄)-alkyl or
R_{H}³ and R_{H}⁴ together with the directly bonded nitrogen atom are a four- to eight-membered heterocyclic ring which, as well as the nitrogen atom, may also contain further ring heteroatoms, preferably up to two further ring heteroatoms from the group of N, O and S, and which is unsubstituted or substituted by one or more radicals from the group of halogen, cyano, nitro, (C₁-C₄)-alkyl, (C₁-C₄)-haloalkyl, (C₁-C₄)-alkoxy, (C₁-C₄)-haloalkoxy and (C₁-C₄)-alkylthio.
S16) Active ingredients which are used primarily as herbicides but also have safener action on crop plants, for example
   (2,4-dichlorophenoxy)acetic acid (2,4-D),
   (4-chlorophenoxy)acetic acid,
   (R,S)-2-(4-chloro-o-tolyloxy)propionic acid (mecoprop),
   4-(2,4-dichlorophenoxy)butyric acid (2,4-DB),
   (4-chloro-o-tolyloxy)acetic acid (MCPA),
   4-(4-chloro-o-tolyloxy)butyric acid,
   4-(4-chlorophenoxy)butyric acid,
   3,6-dichloro-2-methoxybenzoic acid (dicamba),
   1-(ethoxycarbonyl)ethyl 3,6-dichloro-2-methoxybenzoate (lactidichloro-ethyl).

Preferred safeners in combination with the inventive compounds of the general formula (I) and/or salts thereof, especially with the compounds of the formulae (I.1) to (I.34) and/or salts thereof, are: cloquintocet-mexyl, cyprosulfamide, fenchlorazole ethyl ester, isoxadifen-ethyl, mefenpyr-diethyl, fenclorim, cumyluron, S4-1 and S4-5, and particularly preferred safeners are: cloquintocet-mexyl, cyprosulfamide, isoxadifen-ethyl and mefenpyr-diethyl.

### BIOLOGICAL EXAMPLES

### A. Post-Emergence Herbicidal Action and Crop Plant Compatibility

Seeds of monocotyledonous and dicotyledonous weeds and crop plants were placed in sandy loam in plastic or wood-fiber pots, covered with soil and cultivated in a greenhouse under controlled growth conditions. 2 to 3 weeks after sowing, the test plants were treated at the one-leaf stage. The compounds of the invention, formulated in the form of wettable powders (WP) or as emulsion concentrates (EC), were then sprayed onto the green parts of the plants as aqueous suspension or emulsion with addition of 0.5% additive at a water application rate of 600 l/ha (converted). After the test plants had been kept in the greenhouse under optimum growth conditions for about 3 weeks, the activity of the preparations was rated visually in comparison to untreated controls. For example, 100% activity=the plants have died, 0% activity=like control plants.

Tables A1 to A14 below show the effects of selected compounds of the general formula (I) according to Tables I.1 to I.33 on various harmful plants and at an application rate corresponding to 20 g/ha and less, which were obtained by the experimental method specified above. The appendices “a”, “b” and “c” give differentiation by different dosage with otherwise the same harmful plants tested.

TABLE A1a
Post-emergence action at 1.25 g/ha against ABUTH in %

| Example | Dosage | |
|---|---|---|
| number | [g/ha] | ABUTH |
| I.1-72 | 1.25 | 100 |
| I.1-1 | 1.25 | 100 |
| I.2-23 | 1.25 | 100 |
| I.2-2 | 1.25 | 80 |
| I.2-58 | 1.25 | 100 |
| I.1-26 | 1.25 | 100 |
| I.1-23 | 1.25 | 100 |
| I.1-71 | 1.25 | 100 |
| I.7-23 | 1.25 | 100 |
| I.7-276 | 1.25 | 100 |
| I.7-2 | 1.25 | 100 |
| I.9-1 | 1.25 | 100 |
| I.7-41 | 1.25 | 100 |
| I.7-278 | 1.25 | 100 |
| I.7-277 | 1.25 | 90 |
| I.7-291 | 1.25 | 100 |
| I.7-71 | 1.25 | 100 |
| I.7-115 | 1.25 | 80 |
| I.7-356 | 1.25 | 90 |
| I.7-394 | 1.25 | 100 |
| I.7-72 | 1.25 | 100 |
| I.7-179 | 1.25 | 100 |
| I.7-177 | 1.25 | 100 |
| I.7-94 | 1.25 | 100 |
| I.6-1 | 1.25 | 100 |
| I.6-176 | 1.25 | 100 |
| I.6-41 | 1.25 | 100 |
| I.6-276 | 1.25 | 100 |
| I.6-23 | 1.25 | 100 |
| I.6-2 | 1.25 | 100 |
| I.7-339 | 1.25 | 90 |
| I.8-23 | 1.25 | 100 |
| I.8-41 | 1.25 | 80 |
| I.8-176 | 1.25 | 100 |
| I.9-23 | 1.25 | 80 |
| I.9-41 | 1.25 | 100 |
| I.8-2 | 1.25 | 100 |
| I.8-1 | 1.25 | 100 |
| I.8-276 | 1.25 | 100 |
| I.7-183 | 1.25 | 100 |
| I.9-2 | 1.25 | 100 |
| I.9-176 | 1.25 | 100 |
| I.9-276 | 1.25 | 100 |
| I.8-291 | 1.25 | 100 |
| I.9-291 | 1.25 | 100 |
| I.19-276 | 1.25 | 100 |
| I.19-176 | 1.25 | 100 |
| I.23-176 | 1.25 | 80 |
| I.23-1 | 1.25 | 100 |
| I.19-1 | 1.25 | 100 |
| I.23-276 | 1.25 | 90 |

TABLE A1b
Post-emergence action at 5 g/ha against ABUTH in %

| Example | Dosage | |
|---|---|---|
| number | [g/ha] | ABUTH |
| I.1-72 | 5 | 100 |
| I.1-1 | 5 | 100 |
| I.2-72 | 5 | 100 |
| I.2-71 | 5 | 100 |
| I.2-1 | 5 | 100 |
| I.2-23 | 5 | 100 |
| I.2-2 | 5 | 100 |
| I.2-58 | 5 | 100 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 100 |
| I.6-41 | 5 | 100 |
| I.6-276 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 100 |
| I.8-176 | 5 | 100 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A1c
Post-emergence action at 20 g/ha against ABUTH in %

| Example | Dosage | |
|---|---|---|
| number | [g/ha] | ABUTH |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A2a
Post-emergence action at 1.25 g/ha against ALOMY in %

| Example | Dosage | |
|---|---|---|
| number | [g/ha] | ALOMY |
| I.7-23 | 1.25 | 80 |
| I.7-276 | 1.25 | 90 |
| I.7-41 | 1.25 | 100 |
| I.8-2 | 1.25 | 80 |
| I.8-291 | 1.25 | 80 |
| I.9-291 | 1.25 | 80 |

TABLE A2b
Post-emergence action at 5 g/ha against ALOMY in %

| Example | Dosage | |
|---|---|---|
| number | [g/ha] | ALOMY |
| I.1-26 | 5 | 80 |
| I.1-23 | 5 | 90 |
| I.1-71 | 5 | 80 |
| I.7-23 | 5 | 100 |
| I.7-276 | 5 | 100 |
| I.7-176 | 5 | 100 |
| I.7-1 | 5 | 80 |
| I.7-2 | 5 | 80 |
| I.7-41 | 5 | 100 |
| I.7-278 | 5 | 90 |
| I.7-291 | 5 | 80 |
| I.7-71 | 5 | 80 |
| I.7-115 | 5 | 100 |
| I.7-356 | 5 | 100 |
| I.7-394 | 5 | 80 |
| I.7-177 | 5 | 90 |
| I.7-94 | 5 | 80 |
| I.6-1 | 5 | 80 |
| I.6-176 | 5 | 80 |
| I.6-41 | 5 | 80 |
| I.6-276 | 5 | 80 |
| I.6-23 | 5 | 80 |
| I.7-339 | 5 | 100 |
| I.8-23 | 5 | 80 |
| I.8-41 | 5 | 80 |
| I.8-176 | 5 | 80 |
| 1.8-2 | 5 | 100 |
| I.8-1 | 5 | 100 |
| I.8-276 | 5 | 80 |
| I.7-183 | 5 | 80 |
| I.9-2 | 5 | 80 |
| I.9-176 | 5 | 100 |
| I.9-276 | 5 | 100 |
| I.8-183 | 5 | 80 |
| I.8-291 | 5 | 90 |
| I.9-291 | 5 | 90 |
| I.19-23 | 5 | 80 |

TABLE A2c
Post-emergence action at 20 g/ha against ALOMY in %

| Example | Dosage | |
|---|---|---|
| number | [g/ha] | ALOMY |
| I.1-72 | 20 | 80 |
| I.1-1 | 20 | 80 |
| I.2-72 | 20 | 90 |
| I.2-71 | 20 | 80 |
| I.2-1 | 20 | 80 |
| I.2-23 | 20 | 80 |
| I.2-2 | 20 | 80 |
| I.2-58 | 20 | 80 |
| I.1-26 | 20 | 90 |
| I.1-23 | 20 | 90 |
| I.1-71 | 20 | 90 |
| I.7-1 | 20 | 100 |
| I.7-276 | 20 | 100 |
| I.7-176 | 20 | 100 |
| I.7-2 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 80 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 80 |
| I.6-1 | 20 | 100 |
| I.6-176 | 20 | 90 |
| I.6-41 | 20 | 100 |
| I.6-276 | 20 | 100 |
| I.6-23 | 20 | 90 |
| I.6-2 | 20 | 90 |
| I.7-339 | 20 | 100 |
| I.8-23 | 20 | 100 |
| I.8-41 | 20 | 100 |
| I.8-176 | 20 | 100 |
| I.9-23 | 20 | 80 |
| I.9-41 | 20 | 80 |
| I.6-183 | 20 | 90 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 90 |
| I.19-176 | 20 | 80 |
| I.23-176 | 20 | 100 |
| I.23-1 | 20 | 90 |
| I.19-1 | 20 | 100 |
| I.19-23 | 20 | 100 |
| I.23-23 | 20 | 100 |
| I.23-276 | 20 | 100 |

TABLE A3a
Post-emergence action at 1.25 g/ha against AMARE in %

| Example | Dosage | |
|---|---|---|
| number | [g/ha] | AMARE |
| I.1-72 | 1.25 | 80 |
| I.1-1 | 1.25 | 90 |
| I.2-72 | 1.25 | 90 |
| I.2-71 | 1.25 | 100 |
| I.2-1 | 1.25 | 100 |
| I.2-23 | 1.25 | 80 |
| I.2-2 | 1.25 | 100 |
| I.2-58 | 1.25 | 90 |
| I.1-26 | 1.25 | 100 |
| I.1-23 | 1.25 | 100 |
| I.1-71 | 1.25 | 100 |
| I.7-1 | 1.25 | 100 |
| I.7-23 | 1.25 | 100 |
| I.7-276 | 1.25 | 100 |
| I.7-2 | 1.25 | 100 |
| I.9-1 | 1.25 | 100 |
| I.7-41 | 1.25 | 100 |
| I.7-278 | 1.25 | 100 |
| I.7-277 | 1.25 | 100 |
| I.7-291 | 1.25 | 100 |
| I.7-71 | 1.25 | 100 |
| I.7-115 | 1.25 | 100 |
| I.7-356 | 1.25 | 100 |
| I.7-394 | 1.25 | 100 |
| I.7-72 | 1.25 | 100 |
| I.7-179 | 1.25 | 100 |
| I.7-177 | 1.25 | 100 |
| I.7-94 | 1.25 | 100 |
| I.6-1 | 1.25 | 100 |
| I.6-176 | 1.25 | 100 |
| I.6-41 | 1.25 | 100 |
| I.6-276 | 1.25 | 100 |
| I.6-23 | 1.25 | 100 |
| I.6-2 | 1.25 | 100 |
| I.7-339 | 1.25 | 100 |
| I.8-23 | 1.25 | 100 |
| I.8-41 | 1.25 | 100 |
| I.8-176 | 1.25 | 100 |
| I.9-23 | 1.25 | 100 |
| I.9-41 | 1.25 | 100 |
| I.6-183 | 1.25 | 100 |
| I.8-2 | 1.25 | 100 |
| I.8-1 | 1.25 | 100 |
| I.8-276 | 1.25 | 100 |
| I.7-183 | 1.25 | 100 |
| I.9-2 | 1.25 | 100 |
| I.9-176 | 1.25 | 100 |
| I.9-276 | 1.25 | 100 |
| I.8-183 | 1.25 | 100 |
| I.8-291 | 1.25 | 100 |
| I.9-291 | 1.25 | 100 |
| I.19-276 | 1.25 | 100 |
| I.19-176 | 1.25 | 100 |
| I.23-176 | 1.25 | 100 |
| I.19-1 | 1.25 | 100 |
| I.19-23 | 1.25 | 100 |
| I.23-23 | 1.25 | 100 |
| I.23-276 | 1.25 | 90 |

TABLE A3b
Post-emergence action at 5 g/ha against AMARE in %

| Example | Dosage | |
|---|---|---|
| number | [g/ha] | AMARE |
| I.1-72 | 5 | 100 |
| I.1-1 | 5 | 100 |
| I.2-72 | 5 | 100 |
| I.2-71 | 5 | 100 |
| I.2-23 | 5 | 100 |
| I.2-2 | 5 | 100 |
| I.2-58 | 5 | 100 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 100 |
| I.6-41 | 5 | 100 |
| I.6-276 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 100 |
| I.8-176 | 5 | 100 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A3c
Post-emergence action at 20 g/ha against AMARE in %

| Example | Dosage | |
|---|---|---|
| number | [g/ha] | AMARE |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A4a
Post-emergence action at 5 g/ha against AVEFA in %

| Example | Dosage | |
|---|---|---|
| number | [g/ha] | AVEFA |
| I.7-176 | 5 | 80 |
| I.7-41 | 5 | 80 |
| I.9-176 | 5 | 80 |
| I.9-276 | 5 | 80 |
| I.9-291 | 5 | 80 |

TABLE A4b
Post-emergence action at 20 g/ha against AVEFA in %

| Example | Dosage | |
|---|---|---|
| number | [g/ha] | AVEFA |
| I.1-1 | 20 | 80 |
| I.1-26 | 20 | 80 |
| I.1-23 | 20 | 80 |
| I.1-71 | 20 | 80 |
| I.7-1 | 20 | 80 |
| I.7-276 | 20 | 90 |
| I.7-176 | 20 | 100 |
| I.7-2 | 20 | 90 |
| I.7-41 | 20 | 80 |
| I.7-278 | 20 | 80 |
| I.7-291 | 20 | 100 |
| I.7-71 | 20 | 90 |
| I.7-115 | 20 | 100 |
| I.7-356 | 20 | 80 |
| I.7-72 | 20 | 100 |
| I.7-179 | 20 | 80 |
| I.7-177 | 20 | 80 |
| I.7-94 | 20 | 80 |
| I.6-1 | 20 | 100 |
| I.6-176 | 20 | 80 |
| I.6-41 | 20 | 80 |
| I.6-276 | 20 | 80 |
| I.6-23 | 20 | 90 |
| I.8-23 | 20 | 80 |
| I.8-41 | 20 | 80 |
| I.8-176 | 20 | 80 |
| I.9-41 | 20 | 80 |
| I.8-2 | 20 | 100 |
| I.8-1 | 20 | 80 |
| I.8-276 | 20 | 80 |
| I.7-183 | 20 | 90 |
| I.9-2 | 20 | 90 |
| I.9-176 | 20 | 80 |
| I.9-276 | 20 | 90 |
| I.8-183 | 20 | 100 |
| I.8-291 | 20 | 90 |
| I.9-291 | 20 | 80 |

TABLE A5a
Post-emergence action at 1.25 g/ha against ECHCG in %

| Example | Dosage | |
|---|---|---|
| number | [g/ha] | ECHCG |
| I.1-26 | 1.25 | 90 |
| I.1-23 | 1.25 | 90 |
| I.1-71 | 1.25 | 100 |
| I.7-1 | 1.25 | 90 |
| I.7-276 | 1.25 | 90 |
| I.7-41 | 1.25 | 90 |
| I.7-278 | 1.25 | 80 |
| I.7-71 | 1.25 | 100 |
| I.7-115 | 1.25 | 100 |
| I.7-356 | 1.25 | 90 |
| I.7-394 | 1.25 | 100 |
| I.7-72 | 1.25 | 80 |
| I.6-1 | 1.25 | 90 |
| I.6-176 | 1.25 | 100 |
| I.6-23 | 1.25 | 80 |
| I.8-1 | 1.25 | 80 |
| I.9-2 | 1.25 | 80 |
| I.9-176 | 1.25 | 100 |
| I.8-291 | 1.25 | 100 |
| I.19-1 | 1.25 | 100 |
| I.23-276 | 1.25 | 100 |

TABLE A5b
Post-emergence action at 5 g/ha against ECHCG in %

| Example | Dosage | |
|---|---|---|
| number | [g/ha] | ECHCG |
| I.2-72 | 5 | 80 |
| I.2-23 | 5 | 100 |
| I.1-26 | 5 | 90 |
| I.1-23 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 90 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 90 |
| I.6-176 | 5 | 100 |
| I.6-41 | 5 | 100 |
| I.6-276 | 5 | 90 |
| I.6-23 | 5 | 90 |
| I.6-2 | 5 | 90 |
| I.7-339 | 5 | 100 |
| I.8-23 | 5 | 100 |
| I.8-41 | 5 | 90 |
| I.8-176 | 5 | 100 |
| I.9-41 | 5 | 80 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 90 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A5c
Post-emergence action at 20 g/ha against ECHCG in %

| Example | Dosage | |
|---|---|---|
| number | [g/ha] | ECHCG |
| I.1-72 | 20 | 100 |
| I.1-1 | 20 | 80 |
| I.2-72 | 20 | 80 |
| I.2-71 | 20 | 80 |
| I.2-1 | 20 | 100 |
| I.2-23 | 20 | 100 |
| I.2-2 | 20 | 90 |
| I.1-26 | 20 | 100 |
| I.1-23 | 20 | 100 |
| I.1-71 | 20 | 100 |
| I.7-1 | 20 | 90 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 80 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A6a
Post-emergence action at 5 g/ha against LOLRI in %

| Example | Dosage | |
|---|---|---|
| number | [g/ha] | LOLRI |
| I.1-26 | 5 | 80 |
| I.7-276 | 5 | 80 |

TABLE A6b
Post-emergence action at 20 g/ha against LOLRI in %

| Example | Dosage | |
|---|---|---|
| number | [g/ha] | LOLRI |
| I.1-23 | 20 | 80 |
| I.1-26 | 20 | 90 |
| I.6-1 | 20 | 80 |
| I.6-176 | 20 | 90 |
| I.6-2 | 20 | 90 |
| I.6-23 | 20 | 90 |
| I.6-276 | 20 | 90 |
| I.7-1 | 20 | 80 |
| I.7-176 | 20 | 80 |
| I.7-177 | 20 | 80 |
| I.7-179 | 20 | 100 |
| I.7-183 | 20 | 80 |
| I.7-2 | 20 | 80 |
| I.7-276 | 20 | 100 |
| I.7-278 | 20 | 80 |
| I.7-41 | 20 | 80 |
| I.7-71 | 20 | 80 |
| I.7-72 | 20 | 80 |
| I.8-1 | 20 | 80 |
| I.8-2 | 20 | 90 |
| I.8-276 | 20 | 100 |
| I.8-291 | 20 | 80 |
| I.9-291 | 20 | 90 |

TABLE A7a
Post-emergence action at 1.25 g/ha against MATIN in %

| Example | Dosage | |
|---|---|---|
| number | [g/ha] | MATIN |
| I.1-26 | 1.25 | 80 |
| I.1-23 | 1.25 | 80 |
| I.1-71 | 1.25 | 80 |
| I.7-1 | 1.25 | 100 |
| I.7-23 | 1.25 | 90 |
| I.7-276 | 1.25 | 100 |
| I.7-2 | 1.25 | 80 |
| I.7-41 | 1.25 | 90 |
| I.7-71 | 1.25 | 80 |
| I.7-115 | 1.25 | 100 |
| I.7-356 | 1.25 | 80 |
| I.7-179 | 1.25 | 80 |
| I.7-177 | 1.25 | 80 |
| I.7-94 | 1.25 | 80 |
| I.6-23 | 1.25 | 80 |
| I.8-41 | 1.25 | 90 |
| I.9-41 | 1.25 | 100 |
| I.8-2 | 1.25 | 100 |
| I.8-1 | 1.25 | 100 |
| I.8-276 | 1.25 | 80 |
| I.7-183 | 1.25 | 80 |

TABLE A7b
Post-emergence action at 5 g/ha against MATIN in %

| Example | Dosage | |
|---|---|---|
| number | [g/ha] | MATIN |
| I.1-72 | 5 | 90 |
| I.1-1 | 5 | 100 |
| I.2-71 | 5 | 80 |
| I.2-1 | 5 | 80 |
| I.2-23 | 5 | 80 |
| I.2-2 | 5 | 80 |
| I.2-58 | 5 | 80 |
| I.1-26 | 5 | 80 |
| I.1-23 | 5 | 100 |
| I.1-71 | 5 | 90 |
| I.7-1 | 5 | 100 |
| I.7-23 | 5 | 100 |
| I.7-276 | 5 | 100 |
| I.7-176 | 5 | 90 |
| I.7-2 | 5 | 80 |
| I.9-1 | 5 | 80 |
| I.7-41 | 5 | 90 |
| I.7-278 | 5 | 80 |
| I.7-277 | 5 | 80 |
| I.7-291 | 5 | 100 |
| 1.7-71 | 5 | 100 |
| I.7-115 | 5 | 100 |
| I.7-356 | 5 | 100 |
| I.7-394 | 5 | 80 |
| I.7-72 | 5 | 100 |
| I.7-179 | 5 | 100 |
| I.7-177 | 5 | 80 |
| I.7-94 | 5 | 80 |
| I.6-1 | 5 | 100 |
| I.6-176 | 5 | 80 |
| I.6-41 | 5 | 80 |
| I.6-276 | 5 | 100 |
| I.6-23 | 5 | 100 |
| I.6-2 | 5 | 90 |
| I.7-339 | 5 | 100 |
| I.8-23 | 5 | 100 |
| I.8-41 | 5 | 90 |
| I.8-176 | 5 | 100 |
| I.9-23 | 5 | 80 |
| I.9-41 | 5 | 100 |
| I.6-183 | 5 | 80 |
| I.8-2 | 5 | 100 |
| I.8-1 | 5 | 100 |
| I.8-276 | 5 | 80 |
| I.7-183 | 5 | 100 |
| I.9-2 | 5 | 100 |
| I.9-176 | 5 | 100 |
| I.9-276 | 5 | 100 |
| I.8-183 | 5 | 80 |
| I.9-291 | 5 | 80 |
| I.19-276 | 5 | 100 |
| I.19-176 | 5 | 100 |
| I.23-1 | 5 | 80 |
| I.19-1 | 5 | 90 |
| I.19-23 | 5 | 100 |

TABLE A7c
Post-emergence action at 20 g/ha against MATIN in %

| Example | Dosage | |
|---|---|---|
| number | [g/ha] | MATIN |
| I.1-72 | 20 | 100 |
| I.1-1 | 20 | 100 |
| I.2-72 | 20 | 100 |
| I.2-71 | 20 | 90 |
| I.2-1 | 20 | 100 |
| I.2-23 | 20 | 100 |
| I.2-2 | 20 | 100 |
| I.2-58 | 20 | 90 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 90 |
| I.7-72 | 20 | 100 |
| I.7-179 | 20 | 100 |
| I.7-177 | 20 | 80 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 80 |
| I.23-1 | 20 | 100 |
| I.19-1 | 20 | 100 |
| I.19-23 | 20 | 100 |
| I.23-23 | 20 | 100 |
| I.23-276 | 20 | 90 |

TABLE A8a
Post-emergence action at 1.25 g/ha against PHBPU in %

| Example | Dosage | |
|---|---|---|
| number | [g/ha] | PHBPU |
| I.1-72 | 1.25 | 80 |
| I.1-1 | 1.25 | 100 |
| I.2-72 | 1.25 | 100 |
| I.2-71 | 1.25 | 80 |
| I.2-1 | 1.25 | 100 |
| I.2-23 | 1.25 | 100 |
| I.2-2 | 1.25 | 100 |
| I.1-26 | 1.25 | 100 |
| I.1-23 | 1.25 | 100 |
| I.1-71 | 1.25 | 100 |
| I.7-1 | 1.25 | 100 |
| I.7-276 | 1.25 | 100 |
| I.7-2 | 1.25 | 100 |
| I.7-41 | 1.25 | 100 |
| I.7-278 | 1.25 | 100 |
| I.7-291 | 1.25 | 100 |
| I.7-71 | 1.25 | 100 |
| I.7-115 | 1.25 | 100 |
| I.7-394 | 1.25 | 100 |
| I.7-72 | 1.25 | 100 |
| I.7-179 | 1.25 | 100 |
| I.7-177 | 1.25 | 90 |
| I.7-94 | 1.25 | 100 |
| I.6-176 | 1.25 | 80 |
| I.6-41 | 1.25 | 100 |
| I.6-276 | 1.25 | 100 |
| I.6-23 | 1.25 | 80 |
| I.6-2 | 1.25 | 100 |
| I.7-339 | 1.25 | 100 |
| I.8-23 | 1.25 | 100 |
| I.8-41 | 1.25 | 100 |
| I.8-176 | 1.25 | 80 |
| I.9-23 | 1.25 | 80 |
| I.9-41 | 1.25 | 100 |
| I.6-183 | 1.25 | 100 |
| I.8-2 | 1.25 | 100 |
| I.8-1 | 1.25 | 100 |
| I.8-276 | 1.25 | 100 |
| I.7-183 | 1.25 | 80 |
| I.9-2 | 1.25 | 100 |
| I.9-276 | 1.25 | 100 |
| I.8-291 | 1.25 | 100 |
| I.9-291 | 1.25 | 100 |
| I.19-176 | 1.25 | 100 |
| I.23-176 | 1.25 | 90 |
| I.19-1 | 1.25 | 100 |
| I.19-23 | 1.25 | 100 |
| I.23-23 | 1.25 | 100 |
| I.23-276 | 1.25 | 90 |

TABLE A8b
Post-emergence action at 5 g/ha against PHBPU in %

| Example | Dosage | |
|---|---|---|
| number | [g/ha] | PHBPU |
| I.1-72 | 5 | 100 |
| I.1-1 | 5 | 100 |
| I.2-72 | 5 | 100 |
| I.2-71 | 5 | 100 |
| I.2-1 | 5 | 100 |
| I.2-23 | 5 | 100 |
| I.2-2 | 5 | 100 |
| I.2-58 | 5 | 80 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 100 |
| I.6-41 | 5 | 100 |
| I.6-276 | 5 | 100 |
| I.6-23 | 5 | 90 |
| I.6-2 | 5 | 100 |
| I.7-339 | 5 | 100 |
| I.8-23 | 5 | 100 |
| I.8-41 | 5 | 100 |
| I.8-176 | 5 | 80 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 90 |
| I.23-1 | 5 | 80 |
| I.19-1 | 5 | 100 |
| I.19-23 | 5 | 100 |
| I.23-23 | 5 | 100 |
| I.23-276 | 5 | 100 |

TABLE A8c
Post-emergence action at 20 g/ha against PHBPU in %

| Example | Dosage | |
|---|---|---|
| number | [g/ha] | PHBPU |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 80 |
| I.19-1 | 20 | 100 |
| I.19-23 | 20 | 100 |
| I.23-23 | 20 | 100 |
| I.23-276 | 20 | 100 |

TABLE 9a
Post-emergence action at 1.25 g/ha against POLCO in %

| Example | Dosage | |
|---|---|---|
| number | [g/ha] | POLCO |
| I.1-72 | 1.25 | 80 |
| I.2-58 | 1.25 | 100 |
| I.1-26 | 1.25 | 100 |
| I.1-23 | 1.25 | 100 |
| I.1-71 | 1.25 | 100 |
| I.7-72 | 1.25 | 80 |
| I.6-23 | 1.25 | 90 |
| I.8-41 | 1.25 | 80 |
| I.8-1 | 1.25 | 90 |
| I.9-291 | 1.25 | 80 |
| I.19-176 | 1.25 | 100 |
| I.23-1 | 1.25 | 90 |
| I.19-23 | 1.25 | 100 |
| I.23-23 | 1.25 | 100 |

TABLE A9b
Post-emergence action at 5 g/ha against POLCO in %

| Example | Dosage | |
|---|---|---|
| number | [g/ha] | POLCO |
| I.1-72 | 5 | 100 |
| I.1-1 | 5 | 100 |
| I.2-72 | 5 | 80 |
| I.2-71 | 5 | 100 |
| I.2-1 | 5 | 80 |
| I.2-23 | 5 | 100 |
| I.2-2 | 5 | 100 |
| I.2-58 | 5 | 100 |
| I.1-26 | 5 | 100 |
| I.1-23 | 5 | 100 |
| I.1-71 | 5 | 100 |
| I.7-1 | 5 | 80 |
| I.7-23 | 5 | 100 |
| I.7-176 | 5 | 100 |
| I.7-41 | 5 | 100 |
| I.7-278 | 5 | 80 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 80 |
| I.6-1 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 90 |
| I.8-176 | 5 | 80 |
| I.9-23 | 5 | 100 |
| I.9-41 | 5 | 90 |
| I.8-2 | 5 | 100 |
| I.8-1 | 5 | 100 |
| I.8-276 | 5 | 100 |
| I.9-2 | 5 | 100 |
| I.8-183 | 5 | 100 |
| I.8-291 | 5 | 100 |
| I.9-291 | 5 | 90 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |

TABLE A9c
Post-emergence action at 20 g/ha against POLCO in %

| Example | Dosage | |
|---|---|---|
| number | [g/ha] | POLCO |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A10a
Post-emergence action at 1.25 g/ha against SETVI in %

| Example | Dosage | |
|---|---|---|
| number | [g/ha] | SETVI |
| I.1-72 | 1.25 | 100 |
| I.1-1 | 1.25 | 100 |
| I.2-72 | 1.25 | 90 |
| I.2-71 | 1.25 | 90 |
| I.2-1 | 1.25 | 100 |
| I.2-2 | 1.25 | 100 |
| I.2-58 | 1.25 | 100 |
| I.1-26 | 1.25 | 100 |
| I.1-23 | 1.25 | 100 |
| I.1-71 | 1.25 | 100 |
| I.7-1 | 1.25 | 100 |
| I.7-23 | 1.25 | 100 |
| I.7-276 | 1.25 | 100 |
| I.7-2 | 1.25 | 100 |
| I.9-1 | 1.25 | 80 |
| I.7-41 | 1.25 | 100 |
| I.7-278 | 1.25 | 100 |
| I.7-277 | 1.25 | 100 |
| I.7-291 | 1.25 | 100 |
| I.7-71 | 1.25 | 100 |
| I.7-115 | 1.25 | 100 |
| I.7-356 | 1.25 | 100 |
| I.7-394 | 1.25 | 100 |
| I.7-72 | 1.25 | 100 |
| I.7-179 | 1.25 | 100 |
| I.7-177 | 1.25 | 100 |
| I.7-94 | 1.25 | 100 |
| I.6-1 | 1.25 | 100 |
| I.6-176 | 1.25 | 100 |
| I.6-41 | 1.25 | 100 |
| I.6-276 | 1.25 | 100 |
| I.6-23 | 1.25 | 100 |
| I.6-2 | 1.25 | 100 |
| I.7-339 | 1.25 | 100 |
| I.8-23 | 1.25 | 100 |
| I.8-41 | 1.25 | 100 |
| I.8-176 | 1.25 | 100 |
| I.9-23 | 1.25 | 100 |
| I.9-41 | 1.25 | 100 |
| I.8-2 | 1.25 | 100 |
| I.8-1 | 1.25 | 100 |
| I.8-276 | 1.25 | 100 |
| I.7-183 | 1.25 | 100 |
| I.9-2 | 1.25 | 100 |
| I.9-176 | 1.25 | 100 |
| I.9-276 | 1.25 | 100 |
| I.8-183 | 1.25 | 100 |
| I.8-291 | 1.25 | 100 |
| I.9-291 | 1.25 | 100 |
| I.19-276 | 1.25 | 100 |
| I.19-176 | 1.25 | 90 |
| I.23-176 | 1.25 | 100 |
| I.23-1 | 1.25 | 100 |
| I.19-1 | 1.25 | 100 |
| I.19-23 | 1.25 | 100 |
| I.23-23 | 1.25 | 100 |
| I.23-276 | 1.25 | 100 |

TABLE A10b
Post-emergence action at 5 g/ha against SETVI in %

| Example | Dosage | |
|---|---|---|
| number | [g/ha] | SETVI |
| I.1-72 | 5 | 100 |
| I.1-1 | 5 | 100 |
| I.2-72 | 5 | 100 |
| I.2-71 | 5 | 100 |
| I.2-1 | 5 | 100 |
| I.2-23 | 5 | 100 |
| I.2-2 | 5 | 100 |
| I.2-58 | 5 | 100 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 100 |
| I.6-41 | 5 | 100 |
| I.6-276 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 100 |
| I.8-176 | 5 | 100 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A10c
Post-emergence action at 20 g/ha against SETVI in %

| Example | Dosage | |
|---|---|---|
| number | [g/ha] | SETVI |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A11a
Post-emergence action at 1.25 g/ha against VERPE in %

| Example | Dosage | |
|---|---|---|
| number | [g/ha] | VERPE |
| I.1-72 | 1.25 | 100 |
| I.1-1 | 1.25 | 80 |
| I.2-72 | 1.25 | 100 |
| I.2-71 | 1.25 | 100 |
| I.2-1 | 1.25 | 90 |
| I.2-23 | 1.25 | 80 |
| I.2-2 | 1.25 | 100 |
| I.1-26 | 1.25 | 100 |
| I.1-23 | 1.25 | 100 |
| I.1-71 | 1.25 | 100 |
| I.7-1 | 1.25 | 100 |
| I.7-23 | 1.25 | 90 |
| I.7-276 | 1.25 | 100 |
| I.7-2 | 1.25 | 80 |
| I.9-1 | 1.25 | 100 |
| I.7-41 | 1.25 | 80 |
| I.7-278 | 1.25 | 100 |
| I.7-277 | 1.25 | 100 |
| I.7-71 | 1.25 | 100 |
| I.7-115 | 1.25 | 100 |
| I.7-356 | 1.25 | 100 |
| I.7-394 | 1.25 | 100 |
| I.7-72 | 1.25 | 90 |
| I.7-179 | 1.25 | 100 |
| I.7-177 | 1.25 | 90 |
| I.7-94 | 1.25 | 100 |
| I.6-176 | 1.25 | 80 |
| I.6-23 | 1.25 | 100 |
| I.6-2 | 1.25 | 100 |
| I.7-339 | 1.25 | 80 |
| I.8-23 | 1.25 | 80 |
| I.8-176 | 1.25 | 100 |
| I.9-23 | 1.25 | 80 |
| I.8-2 | 1.25 | 100 |
| I.8-1 | 1.25 | 100 |
| I.8-276 | 1.25 | 100 |
| I.9-2 | 1.25 | 100 |
| I.9-176 | 1.25 | 100 |
| I.9-276 | 1.25 | 80 |
| I.8-291 | 1.25 | 100 |
| I.9-291 | 1.25 | 100 |
| I.19-276 | 1.25 | 100 |
| I.19-176 | 1.25 | 100 |
| I.23-176 | 1.25 | 100 |
| I.23-1 | 1.25 | 80 |
| I.19-1 | 1.25 | 100 |
| I.19-23 | 1.25 | 100 |
| I.23-276 | 1.25 | 80 |

TABLE A11b
Post-emergence action at 5 g/ha against VERPE in %

| Example | Dosage | |
|---|---|---|
| number | [g/ha] | VERPE |
| I.1-72 | 5 | 100 |
| I.1-1 | 5 | 100 |
| I.2-72 | 5 | 100 |
| I.2-71 | 5 | 100 |
| I.2-1 | 5 | 100 |
| I.2-23 | 5 | 100 |
| I.2-2 | 5 | 100 |
| I.2-58 | 5 | 80 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 90 |
| I.6-41 | 5 | 100 |
| I.6-276 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 100 |
| I.8-176 | 5 | 100 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A11c
Post-emergence action at 20 g/ha against VERPE in %

| Example | Dosage | |
|---|---|---|
| number | [g/ha] | VERPE |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A12a
Post-emergence action at 1.25 g/ha against VIOTR in %

| Example | Dosage | |
|---|---|---|
| number | [g/ha] | VIOTR |
| I.1-72 | 1.25 | 100 |
| I.1-1 | 1.25 | 100 |
| I.2-72 | 1.25 | 100 |
| I.2-71 | 1.25 | 100 |
| I.2-1 | 1.25 | 100 |
| I.2-2 | 1.25 | 100 |
| I.2-58 | 1.25 | 100 |
| I.1-26 | 1.25 | 100 |
| I.1-23 | 1.25 | 100 |
| I.1-71 | 1.25 | 100 |
| I.7-1 | 1.25 | 100 |
| I.7-23 | 1.25 | 100 |
| I.7-276 | 1.25 | 100 |
| I.7-2 | 1.25 | 100 |
| I.9-1 | 1.25 | 100 |
| I.7-41 | 1.25 | 100 |
| I.7-278 | 1.25 | 90 |
| I.7-277 | 1.25 | 80 |
| I.7-291 | 1.25 | 100 |
| I.7-71 | 1.25 | 100 |
| I.7-115 | 1.25 | 100 |
| I.7-356 | 1.25 | 100 |
| I.7-394 | 1.25 | 100 |
| I.7-72 | 1.25 | 100 |
| I.7-179 | 1.25 | 100 |
| I.7-177 | 1.25 | 100 |
| I.7-94 | 1.25 | 80 |
| I.6-1 | 1.25 | 100 |
| I.6-176 | 1.25 | 100 |
| I.6-41 | 1.25 | 100 |
| I.6-276 | 1.25 | 100 |
| I.6-23 | 1.25 | 100 |
| I.6-2 | 1.25 | 100 |
| I.7-339 | 1.25 | 100 |
| I.8-23 | 1.25 | 100 |
| I.8-41 | 1.25 | 100 |
| I.8-176 | 1.25 | 90 |
| I.9-23 | 1.25 | 100 |
| I.9-41 | 1.25 | 90 |
| I.6-183 | 1.25 | 90 |
| I.8-2 | 1.25 | 100 |
| I.8-1 | 1.25 | 100 |
| I.8-276 | 1.25 | 100 |
| I.7-183 | 1.25 | 100 |
| I.9-2 | 1.25 | 100 |
| I.9-176 | 1.25 | 100 |
| I.9-276 | 1.25 | 100 |
| I.8-183 | 1.25 | 100 |
| I.8-291 | 1.25 | 100 |
| I.9-291 | 1.25 | 100 |
| I.19-276 | 1.25 | 100 |
| I.19-176 | 1.25 | 80 |
| I.23-176 | 1.25 | 90 |
| I.23-1 | 1.25 | 80 |
| I.19-1 | 1.25 | 100 |
| I.23-23 | 1.25 | 100 |
| I.23-276 | 1.25 | 80 |

TABLE A12b
Post-emergence action at 5 g/ha against VIOTR in %

| Example | Dosage | |
|---|---|---|
| number | [g/ha] | VIOTR |
| I.1-72 | 5 | 100 |
| I.1-1 | 5 | 100 |
| I.2-72 | 5 | 100 |
| I.2-71 | 5 | 100 |
| I.2-1 | 5 | 100 |
| I.2-23 | 5 | 100 |
| I.2-2 | 5 | 100 |
| I.2-58 | 5 | 100 |
| I.1-26 | 5 | 100 |
| I.1-23 | 5 | 100 |
| I.1-71 | 5 | 100 |
| I.7-1 | 5 | 100 |
| I.7-23 | 5 | 100 |
| I.7-276 | 5 | 100 |
| I.7-176 | 5 | 100 |
| I.7-2 | 5 | 100 |
| I.9-1 | 5 | 100 |
| I.7-41 | 5 | 100 |
| I.7-278 | 5 | 100 |
| I.7-277 | 5 | 100 |
| I.7-291 | 5 | 100 |
| I.7-71 | 5 | 100 |
| I.7-115 | 5 | 100 |
| I.7-356 | 5 | 100 |
| I.7-394 | 5 | 100 |
| I.7-72 | 5 | 100 |
| I.7-179 | 5 | 100 |
| I.7-177 | 5 | 100 |
| I.7-94 | 5 | 100 |
| I.6-1 | 5 | 100 |
| I.6-176 | 5 | 100 |
| I.6-41 | 5 | 100 |
| I.6-276 | 5 | 100 |
| I.6-23 | 5 | 100 |
| I.6-2 | 5 | 100 |
| I.7-339 | 5 | 100 |
| I.8-23 | 5 | 100 |
| I.8-41 | 5 | 100 |
| I.8-176 | 5 | 100 |
| I.9-23 | 5 | 100 |
| I.9-41 | 5 | 100 |
| I.6-183 | 5 | 100 |
| I.8-2 | 5 | 100 |
| I.8-1 | 5 | 100 |
| I.8-276 | 5 | 100 |
| I.7-183 | 5 | 100 |
| I.9-2 | 5 | 100 |
| I.9-176 | 5 | 100 |
| I.9-276 | 5 | 100 |
| I.8-183 | 5 | 100 |
| I.8-291 | 5 | 100 |
| I.9-291 | 5 | 100 |
| I.19-276 | 5 | 100 |
| I.19-176 | 5 | 100 |
| I.23-176 | 5 | 100 |
| I.23-1 | 5 | 100 |
| I.19-1 | 5 | 100 |
| I.19-23 | 5 | 100 |
| I.23-23 | 5 | 100 |
| I.23-276 | 5 | 100 |

TABLE A12c
Post-emergence action at 20 g/ha against VIOTR in %

| Example | Dosage | |
|---|---|---|
| number | [g/ha] | VIOTR |
| I.1-72 | 20 | 100 |
| I.1-1 | 20 | 100 |
| I.2-72 | 20 | 100 |
| I.2-71 | 20 | 100 |
| I.2-1 | 20 | 100 |
| I.2-23 | 20 | 100 |
| I.2-2 | 20 | 100 |
| I.2-58 | 20 | 100 |
| I.1-26 | 20 | 100 |
| I.1-23 | 20 | 100 |
| I.1-71 | 20 | 100 |
| I.7-1 | 20 | 100 |
| I.7-276 | 20 | 100 |
| I.7-176 | 20 | 100 |
| I.7-2 | 20 | 100 |
| I.9-1 | 20 | 100 |
| I.7-41 | 20 | 100 |
| I.7-278 | 20 | 100 |
| I.7-277 | 20 | 100 |
| I.7-291 | 20 | 100 |
| I.7-71 | 20 | 100 |
| I.7-115 | 20 | 100 |
| I.7-356 | 20 | 100 |
| I.7-394 | 20 | 100 |
| I.7-72 | 20 | 100 |
| I.7-179 | 20 | 100 |
| I.7-177 | 20 | 100 |
| I.7-94 | 20 | 100 |
| I.6-1 | 20 | 100 |
| I.6-176 | 20 | 100 |
| I.6-41 | 20 | 100 |
| I.6-276 | 20 | 100 |
| I.6-23 | 20 | 100 |
| I.6-2 | 20 | 100 |
| I.7-339 | 20 | 100 |
| I.8-23 | 20 | 100 |
| I.8-41 | 20 | 100 |
| I.8-176 | 20 | 100 |
| I.9-23 | 20 | 100 |
| I.9-41 | 20 | 100 |
| I.6-183 | 20 | 100 |
| I.8-2 | 20 | 100 |
| I.8-1 | 20 | 100 |
| I.8-276 | 20 | 100 |
| I.7-183 | 20 | 100 |
| I.9-2 | 20 | 100 |
| I.9-176 | 20 | 100 |
| I.9-276 | 20 | 100 |
| I.8-183 | 20 | 100 |
| I.8-291 | 20 | 100 |
| I.9-291 | 20 | 100 |
| I.19-276 | 20 | 100 |
| I.19-176 | 20 | 100 |
| I.23-176 | 20 | 100 |
| I.23-1 | 20 | 100 |
| I.19-1 | 20 | 100 |
| I.19-23 | 20 | 100 |
| I.23-23 | 20 | 100 |
| I.23-276 | 20 | 100 |

TABLE A13a
Post-emergence action at 5 g/ha against HORMU in %

| Example | Dosage | |
|---|---|---|
| number | [g/ha] | HORMU |
| I.1-23 | 5 | 80 |
| I.1-26 | 5 | 80 |
| I.1-71 | 5 | 80 |

TABLE A13b
Post-emergence action at 20 g/ha against HORMU in %

| Example | Dosage | |
|---|---|---|
| number | [g/ha] | HORMU |
| I.1-1 | 20 | 100 |
| I.1-23 | 20 | 100 |
| I.1-26 | 20 | 90 |
| I.1-71 | 20 | 90 |

TABLE A14a
Post-emergence action at 1.25 g/ha against STEME in %

| Example | Dosage | |
|---|---|---|
| number | [g/ha] | STEME |
| I.1-1 | 1.25 | 80 |
| I.1-23 | 1.25 | 80 |
| I.1-26 | 1.25 | 100 |
| I.1-71 | 1.25 | 90 |
| I.2-1 | 1.25 | 80 |

TABLE A14b
Post-emergence action at 5 g/ha against STEME in %

| Example | Dosage | |
|---|---|---|
| number | [g/ha] | STEME |
| I.1-1 | 5 | 100 |
| I.1-23 | 5 | 100 |
| I.1-26 | 5 | 100 |
| I.1-71 | 5 | 100 |
| I.1-72 | 5 | 100 |
| I.2-1 | 5 | 100 |
| I.2-2 | 5 | 100 |
| I.2-23 | 5 | 100 |
| I.2-58 | 5 | 100 |
| I.2-71 | 5 | 100 |
| I.2-72 | 5 | 100 |

TABLE A14c
Post-emergence action at 20 g/ha against STEME in %

| Example | Dosage | |
|---|---|---|
| number | [g/ha] | STEME |
| I.1-1 | 20 | 100 |
| I.1-23 | 20 | 100 |
| I.1-26 | 20 | 100 |
| I.1-71 | 20 | 100 |
| I.1-72 | 20 | 100 |
| I.2-1 | 20 | 100 |
| I.2-2 | 20 | 100 |
| I.2-23 | 20 | 100 |
| I.2-58 | 20 | 100 |
| I.2-71 | 20 | 100 |
| I.2-72 | 20 | 100 |

Tables A15 to A19 below show the crop plant compatibilities of selected compounds of the general formula (I) according to tables I.1 to I.33 at an application rate corresponding to 5 g/ha or less, which were observed in trials by the experimental procedure mentioned above. The observed effects on selected crop plants are reported here in comparison to the untreated controls (values in %). The appendices “a”, “b” and “c” give differentiation by different dosage with otherwise the same crop plants tested.

TABLE A15a
Post-emergence action at 1.25 g/ha against BRSNW in %

| Example | Dosage | |
|---|---|---|
| number | [g/ha] | BRSNW |
| I.1-23 | 1.25 | 20 |
| I.1-26 | 1.25 | 20 |
| I.1-71 | 1.25 | 20 |
| I.2-2 | 1.25 | 10 |
| I.2-23 | 1.25 | 20 |
| I.2-58 | 1.25 | 20 |
| I.2-71 | 1.25 | 10 |
| I.2-72 | 1.25 | 10 |
| I.7-1 | 1.25 | 20 |
| I.7-23 | 1.25 | 20 |
| I.7-115 | 1.25 | 20 |
| I.7-276 | 1.25 | 20 |

TABLE A15b
Post-emergence action at 5 g/ha against BRSNW in %

| Example | Dosage | |
|---|---|---|
| number | [g/ha] | BRSNW |
| I.2-23 | 5 | 20 |
| I.7-176 | 5 | 20 |
| I.7-276 | 5 | 20 |

TABLE A15c
Post-emergence action at 20 g/ha against BRSNW in %

| Example | Dosage | |
|---|---|---|
| number | [g/ha] | BRSNW |
| I.7-176 | 20 | 20 |
| I.7-276 | 20 | 20 |

TABLE A16a
Post-emergence action at 1.25 g/ha against ZEAMX in %

| Example | Dosage | |
|---|---|---|
| number | [g/ha] | ZEAMX |
| I.1-1 | 1.25 | 20 |
| I.1-23 | 1.25 | 10 |
| I.1-26 | 1.25 | 10 |
| I.1-71 | 1.25 | 20 |
| I.1-72 | 1.25 | 10 |
| I.19-276 | 1.25 | 20 |
| I.2-1 | 1.25 | 10 |
| I.23-1 | 1.25 | 20 |
| I.23-176 | 1.25 | 20 |
| I.23-23 | 1.25 | 20 |
| I.2-58 | 1.25 | 10 |
| I.6-1 | 1.25 | 20 |
| I.6-176 | 1.25 | 10 |
| I.6-183 | 1.25 | 20 |
| I.6-2 | 1.25 | 20 |
| I.6-23 | 1.25 | 10 |
| I.6-276 | 1.25 | 10 |
| I.6-41 | 1.25 | 20 |
| I.7-115 | 1.25 | 20 |
| I.7-179 | 1.25 | 0 |
| I.7-1 | 1.25 | 20 |
| I.7-2 | 1.25 | 0 |
| I.7-277 | 1.25 | 0 |
| I.7-278 | 1.25 | 0 |
| I.7-291 | 1.25 | 10 |
| I.7-339 | 1.25 | 20 |
| I.7-356 | 1.25 | 20 |
| I.7-394 | 1.25 | 10 |
| I.7-72 | 1.25 | 20 |
| I.7-94 | 1.25 | 10 |
| I.8-1 | 1.25 | 20 |
| I.8-2 | 1.25 | 20 |
| I.8-276 | 1.25 | 20 |
| I.8-41 | 1.25 | 20 |
| I.9-1 | 1.25 | 10 |
| I.9-176 | 1.25 | 0 |
| I.9-276 | 1.25 | 0 |
| I.9-41 | 1.25 | 10 |

TABLE A16b
Post-emergence action at 5 g/ha against ZEAMX in %

| Example | Dosage | |
|---|---|---|
| number | [g/ha] | ZEAMX |
| I.1-72 | 5 | 20 |
| I.2-1 | 5 | 10 |
| I.1-26 | 5 | 20 |
| I.6-23 | 5 | 20 |
| I.6-183 | 5 | 20 |
| I.9-276 | 5 | 0 |

TABLE A16c
Post-emergence action at 20 g/ha against ZEAMX in %

| Example | Dosage | |
|---|---|---|
| number | [g/ha] | ZEAMX |
| I.2-1 | 20 | 20 |
| I.9-276 | 20 | 20 |

TABLE A17a
Post-emergence action at 1.25 g/ha against TRZAS in %

| Example | Dosage | |
|---|---|---|
| number | [g/ha] | TRZAS |
| I.1-1 | 1.25 | 20 |
| I.1-23 | 1.25 | 20 |
| I.1-71 | 1.25 | 10 |
| I.1-72 | 1.25 | 20 |
| I.19-1 | 1.25 | 20 |
| I.19-176 | 1.25 | 20 |
| I.19-23 | 1.25 | 10 |
| I.19-276 | 1.25 | 20 |
| I.2-1 | 1.25 | 20 |
| I.2-2 | 1.25 | 20 |
| I.2-23 | 1.25 | 20 |
| I.23-1 | 1.25 | 20 |
| I.23-176 | 1.25 | 10 |
| I.23-23 | 1.25 | 20 |
| I.23-276 | 1.25 | 20 |
| I.2-71 | 1.25 | 20 |
| I.2-72 | 1.25 | 20 |
| I.6-1 | 1.25 | 10 |
| I.6-176 | 1.25 | 20 |
| I.6-183 | 1.25 | 0 |
| I.6-2 | 1.25 | 20 |
| I.6-23 | 1.25 | 10 |
| I.6-276 | 1.25 | 10 |
| I.6-41 | 1.25 | 10 |
| I.7-1 | 1.25 | 20 |
| I.7-115 | 1.25 | 0 |
| I.7-177 | 1.25 | 0 |
| I.7-179 | 1.25 | 0 |
| I.7-183 | 1.25 | 20 |
| I.7-2 | 1.25 | 20 |
| I.7-23 | 1.25 | 10 |
| I.7-276 | 1.25 | 20 |
| I.7-277 | 1.25 | 0 |
| I.7-291 | 1.25 | 0 |
| I.7-339 | 1.25 | 0 |
| I.7-356 | 1.25 | 10 |
| I.7-394 | 1.25 | 0 |
| I.7-41 | 1.25 | 0 |
| I.7-71 | 1.25 | 0 |
| I.7-72 | 1.25 | 10 |
| I.7-94 | 1.25 | 20 |
| I.8-1 | 1.25 | 20 |
| I.8-176 | 1.25 | 0 |
| I.8-183 | 1.25 | 0 |
| I.8-23 | 1.25 | 10 |
| I.8-276 | 1.25 | 20 |
| I.8-291 | 1.25 | 10 |
| I.8-41 | 1.25 | 0 |
| I.9-1 | 1.25 | 0 |
| I.9-176 | 1.25 | 0 |
| I.9-2 | 1.25 | 20 |
| I.9-23 | 1.25 | 0 |
| I.9-276 | 1.25 | 0 |
| I.9-291 | 1.25 | 20 |
| I.9-41 | 1.25 | 0 |

TABLE A17b
Post-emergence action at 5 g/ha against TRZAS in %

| Example | Dosage | |
|---|---|---|
| number | [g/ha] | TRZAS |
| I.2-72 | 5 | 20 |
| I.2-71 | 5 | 20 |
| I.2-1 | 5 | 20 |
| I.2-2 | 5 | 20 |
| I.1-71 | 5 | 20 |
| I.7-276 | 5 | 20 |
| I.7-2 | 5 | 20 |
| I.9-1 | 5 | 10 |
| I.7-41 | 5 | 20 |
| I.7-277 | 5 | 20 |
| I.7-291 | 5 | 0 |
| I.7-179 | 5 | 0 |
| I.6-1 | 5 | 20 |
| I.6-176 | 5 | 20 |
| I.6-41 | 5 | 20 |
| I.6-276 | 5 | 10 |
| I.6-23 | 5 | 20 |
| I.6-2 | 5 | 20 |
| I.7-339 | 5 | 20 |
| I.8-23 | 5 | 20 |
| I.8-41 | 5 | 20 |
| I.8-176 | 5 | 20 |
| I.9-23 | 5 | 20 |
| I.9-41 | 5 | 10 |
| I.6-183 | 5 | 20 |
| I.8-276 | 5 | 20 |
| I.9-176 | 5 | 20 |
| I.8-183 | 5 | 20 |
| I.8-291 | 5 | 20 |
| I.9-291 | 5 | 20 |
| I.23-276 | 5 | 20 |
| I.2-72 | 5 | 20 |
| I.2-71 | 5 | 20 |
| I.2-1 | 5 | 20 |
| I.2-2 | 5 | 20 |
| I.1-71 | 5 | 20 |
| I.7-276 | 5 | 20 |
| I.7-2 | 5 | 20 |
| I.9-1 | 5 | 10 |
| I.7-41 | 5 | 20 |
| I.7-277 | 5 | 20 |
| I.7-291 | 5 | 0 |
| I.7-179 | 5 | 0 |
| I.6-1 | 5 | 20 |
| I.6-176 | 5 | 20 |
| I.6-41 | 5 | 20 |
| I.6-276 | 5 | 10 |
| I.6-23 | 5 | 20 |
| I.6-2 | 5 | 20 |
| I.7-339 | 5 | 20 |
| I.8-23 | 5 | 20 |
| I.8-41 | 5 | 20 |
| I.8-176 | 5 | 20 |
| I.9-23 | 5 | 20 |
| I.9-41 | 5 | 10 |
| I.6-183 | 5 | 20 |
| I.8-276 | 5 | 20 |
| I.9-176 | 5 | 20 |
| I.8-183 | 5 | 20 |
| I.8-291 | 5 | 20 |
| I.9-291 | 5 | 20 |
| I.23-276 | 5 | 20 |

TABLE A17c
Post-emergence action at 20 g/ha against TRZAS in %

| Example | Dosage | |
|---|---|---|
| number | [g/ha] | TRZAS |
| I.9-1 | 20 | 10 |
| I.6-176 | 20 | 20 |
| I.6-41 | 20 | 20 |
| I.6-23 | 20 | 20 |
| I.9-23 | 20 | 20 |
| I.9-41 | 20 | 20 |

TABLE A18a
Post-emergence action at 1.25 g/ha against ORYSA in %

| Example | Dosage | |
|---|---|---|
| number | [g/ha] | ORYSA |
| I.1-72 | 1.25 | 10 |
| I.1-1 | 1.25 | 10 |
| I.2-72 | 1.25 | 0 |
| I.2-71 | 1.25 | 20 |
| I.2-1 | 1.25 | 10 |
| I.2-23 | 1.25 | 10 |
| I.2-2 | 1.25 | 10 |
| I.2-58 | 1.25 | 10 |
| I.1-26 | 1.25 | 10 |
| I.1-23 | 1.25 | 20 |
| I.7-23 | 1.25 | 10 |
| I.7-276 | 1.25 | 0 |
| I.7-2 | 1.25 | 0 |
| I.9-1 | 1.25 | 0 |
| I.7-41 | 1.25 | 0 |
| I.7-278 | 1.25 | 0 |
| I.7-277 | 1.25 | 0 |
| I.7-291 | 1.25 | 20 |
| I.7-71 | 1.25 | 20 |
| I.7-115 | 1.25 | 0 |
| I.7-356 | 1.25 | 0 |
| I.7-394 | 1.25 | 0 |
| I.7-72 | 1.25 | 0 |
| I.7-179 | 1.25 | 0 |
| I.7-177 | 1.25 | 0 |
| I.7-94 | 1.25 | 0 |
| I.6-1 | 1.25 | 0 |
| I.6-176 | 1.25 | 0 |
| I.6-41 | 1.25 | 10 |
| I.6-276 | 1.25 | 0 |
| I.6-23 | 1.25 | 0 |
| I.6-2 | 1.25 | 0 |
| I.7-339 | 1.25 | 0 |
| I.8-23 | 1.25 | 0 |
| I.8-41 | 1.25 | 0 |
| I.8-176 | 1.25 | 0 |
| I.9-23 | 1.25 | 0 |
| I.9-41 | 1.25 | 0 |
| I.6-183 | 1.25 | 0 |
| I.8-2 | 1.25 | 0 |
| I.8-1 | 1.25 | 0 |
| I.8-276 | 1.25 | 20 |
| I.7-183 | 1.25 | 0 |
| I.9-2 | 1.25 | 0 |
| I.9-176 | 1.25 | 0 |
| I.9-276 | 1.25 | 0 |
| I.8-183 | 1.25 | 0 |
| I.8-291 | 1.25 | 0 |
| I.9-291 | 1.25 | 0 |
| I.19-276 | 1.25 | 0 |
| I.19-176 | 1.25 | 0 |
| I.23-176 | 1.25 | 0 |
| I.23-1 | 1.25 | 0 |
| I.19-23 | 1.25 | 10 |
| I.23-276 | 1.25 | 0 |

TABLE A18b
Post-emergence action at 5 g/ha against ORYSA in %

| Example | Dosage | |
|---|---|---|
| number | [g/ha] | ORYSA |
| I.1-72 | 5 | 20 |
| I.1-1 | 5 | 10 |
| I.2-72 | 5 | 20 |
| I.2-71 | 5 | 20 |
| I.2-1 | 5 | 10 |
| I.2-23 | 5 | 10 |
| I.2-2 | 5 | 10 |
| I.2-58 | 5 | 10 |
| I.7-23 | 5 | 20 |
| I.9-1 | 5 | 20 |
| I.7-41 | 5 | 20 |
| I.7-277 | 5 | 0 |
| I.7-356 | 5 | 20 |
| I.7-394 | 5 | 20 |
| I.7-94 | 5 | 20 |
| I.6-1 | 5 | 10 |
| I.6-176 | 5 | 20 |
| I.6-41 | 5 | 10 |
| I.6-276 | 5 | 10 |
| I.6-23 | 5 | 10 |
| I.6-2 | 5 | 0 |
| I.7-339 | 5 | 10 |
| I.8-23 | 5 | 0 |
| I.8-41 | 5 | 20 |
| I.8-176 | 5 | 20 |
| I.9-23 | 5 | 0 |
| I.9-41 | 5 | 0 |
| I.6-183 | 5 | 0 |
| I.8-2 | 5 | 0 |
| I.8-1 | 5 | 20 |
| I.9-176 | 5 | 20 |
| I.9-276 | 5 | 0 |
| I.8-183 | 5 | 0 |
| I.8-291 | 5 | 20 |
| I.9-291 | 5 | 0 |
| I.19-276 | 5 | 0 |
| I.23-176 | 5 | 20 |
| I.23-276 | 5 | 10 |

TABLE A18c
Post-emergence action at 20 g/ha against ORYSA in %

| Example | Dosage | |
|---|---|---|
| number | [g/ha] | ORYSA |
| I.2-2 | 20 | 20 |
| I.2-58 | 20 | 20 |
| I.7-277 | 20 | 20 |
| I.6-1 | 20 | 20 |
| I.6-41 | 20 | 20 |
| I.6-276 | 20 | 20 |
| I.6-23 | 20 | 20 |
| I.6-2 | 20 | 20 |
| I.8-23 | 20 | 20 |
| I.9-23 | 20 | 20 |
| I.6-183 | 20 | 0 |
| I.9-276 | 20 | 0 |
| I.8-183 | 20 | 0 |
| I.9-291 | 20 | 20 |
| I.23-276 | 20 | 10 |

TABLE A19a
Post-emergence action at 1.25 g/ha against GLXMA in %

| Example | Dosage | |
|---|---|---|
| number | [g/ha] | GLXMA |
| I.1-72 | 1.25 | 10 |
| I.1-1 | 1.25 | 0 |
| I.2-72 | 1.25 | 0 |
| I.2-71 | 1.25 | 10 |
| I.2-1 | 1.25 | 0 |
| I.2-23 | 1.25 | 0 |
| I.2-2 | 1.25 | 0 |
| I.2-58 | 1.25 | 10 |
| I.7-1 | 1.25 | 10 |
| I.7-356 | 1.25 | 0 |
| I.7-394 | 1.25 | 20 |
| I.7-177 | 1.25 | 0 |
| I.6-1 | 1.25 | 20 |
| I.6-176 | 1.25 | 20 |
| I.6-276 | 1.25 | 20 |
| I.6-23 | 1.25 | 20 |
| I.6-2 | 1.25 | 20 |
| I.9-276 | 1.25 | 0 |

TABLE A19b
Post-emergence action at 5 g/ha against GLXMA in %

| Example | Dosage | |
|---|---|---|
| number | [g/ha] | GLXMA |
| I.1-72 | 5 | 20 |
| I.1-1 | 5 | 20 |
| I.2-72 | 5 | 10 |
| I.2-1 | 5 | 10 |
| I.2-23 | 5 | 10 |
| I.2-2 | 5 | 0 |
| I.2-58 | 5 | 10 |
| I.7-1 | 5 | 10 |
| I.7-356 | 5 | 0 |
| I.6-176 | 5 | 20 |

TABLE A19c
Post-emergence action at 20 g/ha against GLXMA in %

| Example | Dosage | |
|---|---|---|
| number | [g/ha] | GLXMA |
| I.2-1 | 20 | 20 |
| I.2-23 | 20 | 20 |
| I.2-2 | 20 | 10 |
| I.7-1 | 20 | 10 |

As the results show, inventive compounds of the general formula (I) in the case of post-emergence treatment have good herbicidal efficacy against harmful plants, for example Abutilon theophrasti (ABUTH), Alopecurus myosuroides (ALOMY), Amaranthus retroflexus (AMARE), Avena fatua (AVEFA), Echinochloa crus-galli (ECHCG), Hordeum murinum (HORMU), Lolium rigidum (LOLRI), Matricaria inodora (MATIN), Pharbitis purpurea (PHBPU), Polygonum convolvulus (POLCO), Setaria viridis (SETVI), Stellaria media (STEME), Veronica persica (VERPE) and Viola tricolor (VIOTR) at an application rate of 0.02 kg of active substance or less per hectare, and good crop plant compatibility in the case of organisms such as Oryza sativa (ORYSA), Zea mays (ZEAMX), Brassica napus (BRSNW), Glycine max (GLXMA) and Triticum aestivum (TRZAS) at an application rate of 0.02 kg or less per hectare.

### B. Pre-Emergence Herbicidal Action and Crop Plant Compatibility

Seeds of monocotyledonous and dicotyledonous weed plants and crop plants were placed in plastic or organic planting pots and covered with soil. The compounds of the invention, formulated in the form of wettable powders (WP) or as emulsion concentrates (EC), were then applied to the surface of the covering soil as aqueous suspension or emulsion with addition of 0.5% additive at a water application rate equivalent to 600 l/ha (converted). After the treatment, the pots were placed in a greenhouse and kept under good growth conditions for the test plants. After about 3 weeks, the effect of the preparations was scored visually in comparison with untreated controls as percentages. For example, 100% activity=the plants have died, 0% activity=like control plants.

Tables B1 to B10 below show the effects of selected compounds of the general formula (I) according to Tables I.1 to I.33 on various harmful plants and at an application rate corresponding to 80 g/ha and less, which were obtained by the experimental method specified above. The appendices “a”, “b” and “c” give differentiation by different dosage with otherwise the same harmful plants tested.

TABLE B1a
Pre-emergence action at 20 g/ha against ABUTH in %

| Example | Dosage | |
|---|---|---|
| number | [g/ha] | ABUTH |
| I.7-1 | 20 | 100 |
| I.7-23 | 20 | 100 |

TABLE B1b
Pre-emergence action at 80 g/ha against ABUTH in %

| Example | Dosage | |
|---|---|---|
| number | [g/ha] | ABUTH |
| I.7-1 | 80 | 100 |
| I.7-23 | 80 | 100 |

TABLE B2a
Pre-emergence action at 20 g/ha against ALOMY in %

| Example | Dosage | |
|---|---|---|
| number | [g/ha] | ALOMY |
| I.7-1 | 20 | 80 |
| I.7-23 | 20 | 90 |

TABLE B2b
Pre-emergence action at 80 g/ha against ALOMY in %

| Example | Dosage | |
|---|---|---|
| number | [g/ha] | ALOMY |
| I.7-1 | 80 | 100 |
| I.7-23 | 80 | 100 |

TABLE B3a
Pre-emergence action at 20 g/ha against AMARE in %

| Example | Dosage | |
|---|---|---|
| number | [g/ha] | AMARE |
| I.7-1 | 20 | 100 |
| I.7-23 | 20 | 100 |

TABLE B3b
Pre-emergence action at 80 g/ha against AMARE in %

| Example | Dosage | |
|---|---|---|
| number | [g/ha] | AMARE |
| I.7-1 | 80 | 100 |
| I.7-23 | 80 | 100 |

TABLE B4
Pre-emergence action at 80 g/ha against AVEFA in %

| Example | Dosage | |
|---|---|---|
| number | [g/ha] | AVEFA |
| I.7-1 | 80 | 100 |
| I.7-23 | 80 | 90 |

TABLE B5a
Pre-emergence action at 20 g/ha against DIGSA in %

| Example | Dosage | |
|---|---|---|
| number | [g/ha] | DIGSA |
| I.7-1 | 20 | 100 |
| I.7-23 | 20 | 100 |

TABLE B5b
Pre-emergence action at 80 g/ha against DIGSA in %

| Example | Dosage | |
|---|---|---|
| number | [g/ha] | DIGSA |
| I.7-1 | 80 | 100 |
| I.7-23 | 80 | 100 |

TABLE B6a
Pre-emergence action at 20 g/ha against ECHCG in %

| Example | Dosage | |
|---|---|---|
| number | [g/ha] | ECHCG |
| I.7-23 | 20 | 80 |

TABLE B6b
Pre-emergence action at 80 g/ha against ECHCG in %

| Example | Dosage | |
|---|---|---|
| number | [g/ha] | ECHCG |
| I.7-1 | 80 | 90 |
| I.7-23 | 80 | 90 |

TABLE B7a
Pre-emergence action at 20 g/ha against LOLRI in %

| Example | Dosage | |
|---|---|---|
| number | [g/ha] | LOLRI |
| I.7-23 | 20 | 90 |

TABLE B7b
Pre-emergence action at 80 g/ha against LOLRI in %

| Example | Dosage | |
|---|---|---|
| number | [g/ha] | LOLRI |
| I.7-1 | 80 | 100 |
| I.7-23 | 80 | 100 |

TABLE B8a
Pre-emergence action at 20 g/ha against MATIN in %

| Example | Dosage | |
|---|---|---|
| number | [g/ha] | MATIN |
| I.7-1 | 20 | 100 |
| I.7-23 | 20 | 100 |

TABLE B8b
Pre-emergence action at 80 g/ha against MATIN in %

| Example | Dosage | |
|---|---|---|
| number | [g/ha] | MATIN |
| I.7-1 | 80 | 100 |
| I.7-23 | 80 | 100 |

TABLE B9a
Pre-emergence action at 20 g/ha against PHBPU in %

| Example | Dosage | |
|---|---|---|
| number | [g/ha] | PHBPU |
| I.7-1 | 20 | 100 |
| I.7-23 | 20 | 100 |

TABLE B9b
Pre-emergence action at 80 g/ha against PHBPU in %

| Example | Dosage | |
|---|---|---|
| number | [g/ha] | PHBPU |
| I.7-1 | 80 | 100 |
| I.7-23 | 80 | 100 |

TABLE B10a
Pre-emergence action at 20 g/ha against VERPE in %

| Example | Dosage | |
|---|---|---|
| number | [g/ha] | VERPE |
| I.7-1 | 20 | 100 |
| I.7-23 | 20 | 100 |

TABLE B10b
Pre-emergence action at 80 g/ha against VERPE in %

| Example | Dosage | |
|---|---|---|
| number | [g/ha] | VERPE |
| I.7-1 | 80 | 100 |
| I.7-23 | 80 | 100 |

Tables B11 and B12 below show the crop plant compatibilities of selected compounds of the general formula (I) according to tables I.1 to I.33 at an application rate corresponding to 80 g/ha or less, which were observed in trials by the experimental procedure mentioned above. The observed effects on selected crop plants are reported here in comparison to the untreated controls (values in %).

TABLE B11
Pre-emergence action at 20 g/ha against ZEAMX in %

| Example | Dosage | |
|---|---|---|
| number | [g/ha] | ZEAMX |
| I.7-1 | 20 | 0 |
| I.7-23 | 20 | 20 |

TABLE B12
Pre-emergence action at 20 g/ha against GLXMA in %

| Example | Dosage | |
|---|---|---|
| number | [g/ha] | GLXMA |
| I.7-23 | 20 | 10 |

As the results show, inventive compounds of the general formula (I), in pre-emergence treatment, have good herbicidal efficacy against harmful plants, for example against harmful plants such as Abutilon theophrasti (ABUTH), Alopecurus myosuroides (ALOMY), Amaranthus retroflexus (AMARE), Avena fatua (AVEFA), Digitaria sanguinalis (DIGSA), Echinochloa crus-galli (ECHCG), Lolium rigidum (LOLRI), Matricaria inodora (MATIN), Pharbitis purpurea (PHBPU) and Veronica persica (VERPE) at an application rate of 0.08 kg of active substance or less per hectare, and good crop plant compatibility in the case of organisms such as Zea mays (ZEAMX) Glycine max (GLXMA) at an application rate of 0.02 kg per hectare.

C. Comparative Herbicidal Action and Crop Plant Compatibility of Two Inventive Compounds (I.7-1 and I.7-115) with a Compound of Similar Structure Known from the Literature (WO2002/098227, a-17 and EP1106607, 3-14) in Post-Emergence Treatment.

Tables C1-C4 below show the effects of two inventive compounds (I.7-1 and I.7-115) together with a compound of similar structure known from the literature (a-17 in WO2002/098227, and 3-14 in EP1106607) in different crop plants at an application rate corresponding to 5 g/ha or less, which have been obtained by the aforementioned experimental procedure.

The two inventive compounds (I.7-1 and I.7-115) are distinguished here by variance in a significant structural feature from the compounds known from the literature with regard to the ester unit, with the same “chain length” of the ester functionality, via the incorporation of a heteroatom or via the incorporation of a heteroatom with simultaneous ring formation.

TABLE C1

| | | Application |
|---|---|---|
| | ALOMY | rate |
| Compound | (efficacy in %) | [g/ha] |
| 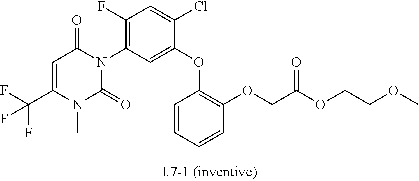 [SMILES: COCCOC(=O)COc1ccccc1Oc1cc(-n2c(=O)cc(C(F)(F)F)n(C)c2=O)c(F)cc1Cl] | 80 | 5 |
| 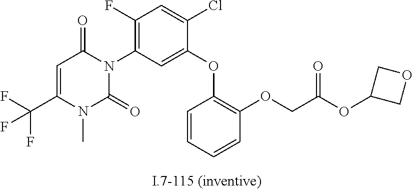 [SMILES: Cn1c(C(F)(F)F)cc(=O)n(-c2cc(Oc3ccccc3OCC(=O)OC3COC3)c(Cl)cc2F)c1=O] | 100 | 5 |
| 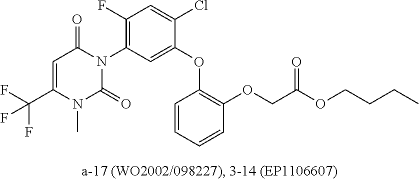 [SMILES: CCCCOC(=O)COc1ccccc1Oc1cc(-n2c(=O)cc(C(F)(F)F)n(C)c2=O)c(F)cc1Cl] | 30 | 5 |

TABLE C2

| | ECHCG | Application rate |
|---|---|---|
| Compound | (efficacy in %) | [g/ha] |
| I.7-1 (inventive) | 90 | 1.25 |
| I.7-115 (inventive) | 100 | 1.25 |
| a-17 (WO2002/098227), 3-14 | 60 | 1.25 |
| (EP1106607) | | |

TABLE C3

| | MATIN | Application rate |
|---|---|---|
| Compound | (efficacy in %) | [g/ha] |
| I.7-1 (inventive) | 100 | 1.25 |
| I.7-115 (inventive) | 100 | 1.25 |
| a-17 (WO2002/098227), 3-14 | 30 | 1.25 |
| (EP1106607) | | |

TABLE C4

| | SETVI | Application rate |
|---|---|---|
| Compound | (efficacy in %) | [g/ha] |
| I.7-1 (inventive) | 100 | 1.25 |
| I.7-115 (inventive) | 100 | 1.25 |
| a-17 (WO2002/098227), 3-14 | 80 | 1.25 |
| (EP1106607) | | |

As the results shown in tables C1 to C4 show, inventive compounds I.7-1 and I.7-115, by comparison with literature compound a-17 (WO2002/098227) or 3-14 (EP1106607) show a distinct improvement in herbicidal efficacy against harmful plants, such as Alopecurus myosuroides (ALOMY), Echinochloa crus-galli (ECHCG), Matricaria inodora (MATIN) and Setaria viridis (SETVI) at an application rate of 5 g or less per hectare.

Table C5 below shows the effects of two inventive compounds (I.7-1 and I.7-115) together with a compound of similar structure known from the literature (a-17 in WO2002/098227 and 3-14 in EP1106607) in the crop plant Oryza sativa (ORYSA) at an application rate corresponding to 1.25 g/ha, which have been obtained by the aforementioned experimental procedure.

TABLE C5

| | ORYSA | Application rate |
|---|---|---|
| Compound | (efficacy in %) | [g/ha] |
| I.7-1 (inventive) | 0 | 1.25 |
| I.7-115 (inventive) | 0 | 1.25 |
| a-17 (WO2002/098227), 3-14 | 20 | 1.25 |
| (EP1106607) | | |

As the results shown in table C5 show, the inventive compounds I.7-1 and I.7-115, by comparison with the literature compound a-17 (WO2002/098227) or 3-14 (EP1106607), show a distinct improvement in compatibility with respect to the crop plant Oryza sativa (ORYSA) at an application rate of 1.25 g per hectare.

1. A substituted N-phenyluracil of formula (I) and/or salt[[s thereof 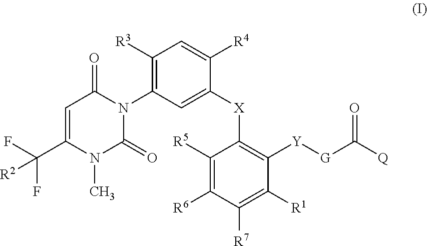
in which
R¹ is hydrogen, halogen, cyano, (C₁-C₈)-alkyl, (C₁-C₈)-haloalkyl, (C₁-C₈)-alkoxy, (C₁-C₈)-haloalkoxy
R² is hydrogen, fluorine, chlorine, bromine, trifluoromethyl, (C₁-C₈)-alkoxy,
R³ is hydrogen, halogen, (C₁-C₈)-alkoxy,
R⁴ is halogen, cyano, NO₂, C(O)NH₂, C(S)NH₂, (C₁-C₈)-haloalkyl, (C₂-C₈)-alkynyl,
R⁵, R⁶ and R⁷ are independently hydrogen, halogen, cyano, (C₁-C₈)-alkyl, (C₁-C₈)-haloalkyl, (C₁-C₈)-alkoxy, (C₁-C₈)-haloalkoxy,
G is unbranched or branched (C₁-C₈)-alkylene,
Q is a radical of formula 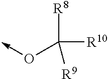
R⁸ is hydrogen, (C₁-C₈)-alkyl, (C₁-C₈)-haloalkyl, aryl, aryl-(C₁-C₈)-alkyl, heteroaryl, (C₂-C₈)-alkynyl, (C₂-C₈)-alkenyl, C(O)R¹³, C(O)OR¹³, (C₁-C₈)-alkoxy-(C₁-C₈)-alkyl,
R⁹ is hydrogen or (C₁-C₈)-alkyl,
R¹⁰ is cyano, NO₂, heteroaryl, heteroaryl-(C₁-C₈)-alkyl, heterocyclyl, heterocyclyl-(C₁-C₈)-alkyl, R¹¹R¹²N—(C₁-C₈)-alkyl, R¹³O—(C₁-C₈)-alkyl, cyano-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, OR¹³, NR¹¹R¹², SR¹⁴, S(O)R¹⁴, SO₂R¹⁴, R¹⁴S—(C₁-C₈)-alkyl, R¹⁴(O)S—(C₁-C₈)-alkyl, R¹⁴O₂S—(C₁-C₈)-alkyl, tris[(C₁-C₈)-alkyl]silyl-(C₁-C₈)-alkyl, bis[(C₁-C₈)-alkyl](aryl)silyl(C₁-C₈)-alkyl, [(C₁-C₈)-alkyl]bis(aryl)silyl-(C₁-C₈)-alkyl, tris[(C₁-C₈)-alkyl]silyl, bishydroxyboryl-(C₁-C₈)-alkyl, bis[(C₁-C₈)-alkoxy]boryl-(C₁-C₈)-alkyl, tetramethyl-1,3,2-dioxaborolan-2-yl, tetramethyl-1,3,2-dioxaborolan-2-yl-(C₁-C₈)-alkyl, nitro-(C₁-C₈)-alkyl, C(O)R¹⁴, bis(C₁-C₈)-alkoxymethyl, bis(C₁-C₈)-alkoxymethyl-(C₁-C₈)-alkyl, or
R⁸ and R¹⁰ together with the carbon atom to which they are bonded form a fully saturated or partly saturated 3- to 10-membered monocyclic or bicyclic heterocyclyl optionally having further substitution,
R¹¹ and R¹² are the same or different and are independently hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkylthio-(C₁-C₈)-alkyl, (C₁-C₈)-haloalkylthio-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, COR¹³, SO₂R¹⁴, heterocyclyl, (C₁-C₈)-alkoxycarbonyl, bis[(C₁-C₈)-alkyl]aminocarbonyl-(C₁-C₈)-alkyl, (C₁-C₈)-alkylaminocarbonyl-(C₁-C₈)-alkyl, aryl-(C₁-C₈)-alkylaminocarbonyl-(C₁-C₈)-alkyl, aryl-(C₁-C₈)-alkoxycarbonyl, heteroaryl-(C₁-C₈)-alkoxycarbonyl, (C₂-C₈)-alkenyloxycarbonyl, (C₂-C₈)-alkynyloxycarbonyl, heterocyclyl-(C₁-C₈)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-haloalkoxy-(C₁-C₈)-alkyl, (C₁-C₈)-alkoxy-(C₁-C₈)-haloalkyl, (C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkyl, (C₁-C₈)-alkoxy-(C₁-C₈)-alkoxy-(C₁-C₈)-alkoxy-(C₁-C₈)-alkoxy-(C₁-C₈)-alkyl, aryl, aryl-(C₁-C₈)-alkyl, aryl-(C₁-C₈)-alkoxy-(C₁-C₈)-alkyl, heteroaryl, heteroaryl-(C₁-C₈)-alkyl, (C₃-C₈)-cycloalkyl-(C₁-C₈)-alkyl, (C₄-C₁₀)-cycloalkenyl-(C₁-C₈)-alkyl, bis[(C₁-C₈)-alkyl]aminocarbonyl-(C₁-C₈)-alkyl, (C₁-C₈)-alkylaminocarbonyl-(C₁-C₈)-alkyl, aryl-(C₁-C₈)-alkylaminocarbonyl-(C₁-C₈)-alkyl, bis[(C₁-C₈)-alkyl]amino-(C₂-C₆)-alkyl, (C₁-C₈)-alkylamino-(C₂-C₆)-alkyl, aryl-(C₁-C₈)-alkylamino-(C₂-C₆)-alkyl, R¹⁴S—(C₁-C₈)-alkyl, R¹⁴(O)S—(C₁-C₈)-alkyl, R¹⁴O₂S—(C₁-C₈)-alkyl, hydroxycarbonyl-(C₁-C₈)-alkyl, heterocyclyl, heterocyclyl-(C₁-C₈)-alkyl, tris[(C₁-C₈)-alkyl]silyl-(C₁-C₈)-alkyl, bis[(C₁-C₈)-alkyl](aryl)silyl(C₁-C₈)-alkyl, [(C₁-C₈)-alkyl]bis(aryl)silyl-(C₁-C₈)-alkyl, (C₁-C₈)-alkylcarbonyloxy-(C₁-C₈)-alkyl, (C₃-C₈)-cycloalkylcarbonyloxy-(C₁-C₈)-alkyl, arylcarbonyloxy-(C₁-C₈)-alkyl, heteroarylcarbonyloxy-(C₁-C₈)-alkyl, heterocyclylcarbonyloxy-(C₁-C₈)-alkyl, aryloxy-(C₁-C₈)-alkyl, heteroaryloxy-(C₁-C₈)-alkyl, (C₁-C₈)-alkoxycarbonyl,
R¹⁴ is hydrogen, (C₁-C₈)-alkyl, (C₂-C₈)-alkenyl, (C₂-C₈)-alkynyl, (C₁-C₈)-cyanoalkyl, (C₁-C₁₀)-haloalkyl, (C₂-C₈)-haloalkenyl, (C₃-C₈)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₈)-alkoxy-(C₁-C₈)-alkyl, (C₁-C₈)-alkoxy-(C₁-C₈)-haloalkyl, aryl, aryl-(C₁-C₈)-alkyl, heteroaryl, heteroaryl-(C₁-C₈)-alkyl, heterocyclyl-(C₁-C₈)-alkyl, (C₃-C₈)-cycloalkyl-(C₁-C₈)-alkyl, (C₄-C₁₀)-cycloalkenyl-(C₁-C₈)-alkyl, bis[(C₁-C₈)-alkyl]amino, (C₁-C₈)-alkylamino, aryl-(C₁-C₈)-amino, aryl-(C₁-C₆)-alkylamino, aryl-[(C₁-C₈)-alkyl]amino; (C₃-C₈)-cycloalkylamino, (C₃-C₈)-cycloalkyl-[(C₁-C₈)-alkyl]amino; N-azetidinyl, N-pyrrolidinyl, N-piperidinyl, N-morpholinyl,
and
X and Y are independently O (oxygen) or S (sulfur).

2. The compound of formula (I) as claimed in claim 1 and/or salt thereof, wherein
R¹ is hydrogen, halogen, cyano, (C₁-C₆)-alkyl, (C₁-C₆)-haloalkyl, (C₁-C₆)-alkoxy, (C₁-C₆)-haloalkoxy,
R² is hydrogen, fluorine, chlorine, bromine, trifluoromethyl, (C₁-C₆)-alkoxy,
R³ is hydrogen, halogen, (C₁-C₆)-alkoxy,
R⁴ is halogen, cyano, NO₂, C(O)NH₂, C(S)NH₂, (C₁-C₆)-haloalkyl, (C₂-C₆)-alkynyl,
R⁵, R⁶ and R⁷ are independently hydrogen, halogen, cyano, (C₁-C₆)-alkyl, (C₁-C₆)-haloalkyl, (C₁-C₆)-alkoxy, (C₁-C₆)-haloalkoxy,
G is unbranched or branched (C₁-C₆)-alkylene,
Q is a radical of formula 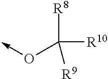
R⁸ is hydrogen, (C₁-C₆)-alkyl, (C₁-C₆)-haloalkyl, aryl, aryl-(C₁-C₆)-alkyl, heteroaryl, (C₂-C₆)-alkynyl, (C₂-C₆)-alkenyl, C(O)R¹³, C(O)OR¹³, (C₁-C₆)-alkoxy-(C₁-C₆)-alkyl,
R⁹ is hydrogen or (C₁-C₄)-alkyl,
R¹⁰ is cyano, NO₂, heteroaryl, heteroaryl-(C₁-C₆)-alkyl, heterocyclyl, heterocyclyl-(C₁-C₆)-alkyl, R¹¹R¹²N—(C₁-C₆)-alkyl, R¹³O—(C₁-C₆)-alkyl, cyano-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, OR¹³, NR¹¹R¹², SR¹⁴, S(O)R¹⁴, SO₂R¹⁴, R¹⁴S—(C₁-C₆)-alkyl, R¹⁴(O)S—(C₁-C₆)-alkyl, R¹⁴O₂S—(C₁-C₆)-alkyl, tris[(C₁-C₆)-alkyl]silyl-(C₁-C₆)-alkyl, bis[(C₁-C₆)-alkyl](aryl)silyl(C₁-C₆)-alkyl, [(C₁-C₆)-alkyl]bis(aryl)silyl-(C₁-C₆)-alkyl, tris[(C₁-C₆)-alkyl]silyl, bishydroxyboryl-(C₁-C₆)-alkyl, bis[(C₁-C₆)-alkoxy]boryl-(C₁-C₆)-alkyl, tetramethyl-1,3,2-dioxaborolan-2-yl, tetramethyl-1,3,2-dioxaborolan-2-yl-(C₁-C₆)-alkyl, nitro-(C₁-C₆)-alkyl, C(O)R¹³, bis(C₁-C₆)-alkoxymethyl, bis(C₁-C₆)-alkoxymethyl-(C₁-C₆)-alkyl,
R⁸ and R¹⁰ together with the carbon atom to which they are bonded form a fully saturated or partly saturated 3- to 10-membered monocyclic or bicyclic heterocyclyl optionally having further substitution,
R¹¹ and R¹² are the same or different and are independently hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkylthio-(C₁-C₆)-alkyl, (C₁-C₆)-haloalkylthio-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, COR¹³, SO₂R¹⁴, heterocyclyl, (C₁-C₆)-alkoxycarbonyl, bis[(C₁-C₆)-alkyl]aminocarbonyl-(C₁-C₆)-alkyl, (C₁-C₆)-alkylaminocarbonyl-(C₁-C₆)-alkyl, aryl-(C₁-C₆)-alkylaminocarbonyl-(C₁-C₆)-alkyl, aryl-(C₁-C₆)-alkoxycarbonyl, heteroaryl-(C₁-C₆)-alkoxycarbonyl, (C₂-C₆)-alkenyloxycarbonyl, (C₂-C₆)-alkynyloxycarbonyl, heterocyclyl-(C₁-C₆)-alkyl, or
R¹¹ and R¹² together with the nitrogen atom to which they are bonded form a fully saturated or partly saturated 3- to 10-membered monocyclic or bicyclic ring optionally interrupted by heteroatoms and optionally having further substitution,
R¹³ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁₀)-halocycloalkenyl, (C₁-C₆)-alkoxy-(C₁-C₆)-alkyl, (C₁-C₆)-haloalkoxy-(C₁-C₆)-alkyl, (C₁-C₆)-alkoxy-(C₁-C₆)-haloalkyl, (C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkyl, (C₁-C₆)-alkoxy-(C₁-C₆)-alkoxy-(C₁-C₆)-alkoxy-(C₁-C₆)-alkoxy-(C₁-C₆)-alkyl, aryl, aryl-(C₁-C₆)-alkyl, aryl-(C₁-C₆)-alkoxy-(C₁-C₆)-alkyl, heteroaryl, heteroaryl-(C₁-C₆)-alkyl, (C₃-C₆)-cycloalkyl-(C₁-C₆)-alkyl, (C₄-C₁₀)-cycloalkenyl-(C₁-C₆)-alkyl, bis[(C₁-C₆)-alkyl]aminocarbonyl-(C₁-C₆)-alkyl, (C₁-C₆)-alkylaminocarbonyl-(C₁-C₆)-alkyl, aryl-(C₁-C₆)-alkylaminocarbonyl-(C₁-C₆)-alkyl, bis[(C₁-C₆)-alkyl]amino-(C₂-C₄)-alkyl, (C₁-C₆)-alkylamino-(C₂-C₄)-alkyl, aryl-(C₁-C₆)-alkylamino-(C₂-C₄)-alkyl, R¹⁴S—(C₁-C₆)-alkyl, R¹⁴(O)S—(C₁-C₆)-alkyl, R¹⁴O₂S—(C₁-C₆)-alkyl, hydroxycarbonyl-(C₁-C₆)-alkyl, heterocyclyl, heterocyclyl-(C₁-C₆)-alkyl, tris[(C₁-C₆)-alkyl]silyl-(C₁-C₆)-alkyl, bis[(C₁-C₆)-alkyl](aryl)silyl(C₁-C₆)-alkyl, [(C₁-C₆)-alkyl]bis(aryl)silyl-(C₁-C₆)-alkyl, (C₁-C₆)-alkylcarbonyloxy-(C₁-C₆)-alkyl, (C₃-C₆)-cycloalkylcarbonyloxy-(C₁-C₆)-alkyl, arylcarbonyloxy-(C₁-C₆)-alkyl, heteroarylcarbonyloxy-(C₁-C₆)-alkyl, heterocyclylcarbonyloxy-(C₁-C₆)-alkyl, aryloxy-(C₁-C₆)-alkyl, heteroaryloxy-(C₁-C₆)-alkyl, (C₁-C₆)-alkoxycarbonyl,
R¹⁴ is hydrogen, (C₁-C₆)-alkyl, (C₂-C₆)-alkenyl, (C₂-C₆)-alkynyl, (C₁-C₆)-cyanoalkyl, (C₁-C₁₀)-haloalkyl, (C₂-C₆)-haloalkenyl, (C₃-C₆)-haloalkynyl, (C₃-C₁₀)-cycloalkyl, (C₃-C₁₀)-halocycloalkyl, (C₄-C₁₀)-cycloalkenyl, (C₄-C₁)-halocycloalkenyl, (C₁-C₆)-alkoxy-(C₁-C₆)-alkyl, (C₁-C₆)-alkoxy-(C₁-C₆)-haloalkyl, aryl, aryl-(C₁-C₆)-alkyl, heteroaryl, heteroaryl-(C₁-C₆)-alkyl, heterocyclyl-(C₁-C₆)-alkyl, (C₃-C₆)-cycloalkyl-(C₁-C₆)-alkyl, (C₄-C₁)-cycloalkenyl-(C₁-C₆)-alkyl, bis[(C₁-C₆)-alkyl]amino, (C₁-C₆)-alkylamino, aryl-(C₁-C₆)-amino, aryl-(C₁-C₂)-alkylamino, aryl-[(C₁-C₆)-alkyl]amino; (C₃-C₆)-cycloalkylamino, (C₃-C₆)-cycloalkyl-[(C₁-C₆)-alkyl]amino; N-azetidinyl, N-pyrrolidinyl, N-piperidinyl, N-morpholinyl,
and
X and Y are independently O (oxygen) or S (sulfur).

3. The compound of formula (I) as claimed in claim 1 and/or salt thereof, wherein
R¹ is hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
R² is hydrogen, fluorine, chlorine, bromine, trifluoromethyl, methoxy, ethoxy, prop-1-yloxy, but-1-yloxy,
R³ is hydrogen, fluorine, chlorine, bromine, methoxy, ethoxy, prop-1-yloxy, prop-2-yloxy, but-1-yloxy, but-2-yloxy, 2-methylprop-1-yloxy, 1,1-dimethyleth-1-yloxy,
R⁴ is fluorine, chlorine, bromine, cyano, NO₂, C(O)NH₂, C(S)NH₂, trifluoromethyl, difluoromethyl, pentafluoroethyl, ethynyl, propyn-1-yl, 1-butyn-1-yl, pentyn-1-yl, hexyn-1-yl,
R⁵, R⁶ and R⁷ are independently hydrogen, fluorine, chlorine, bromine, iodine, cyano, methyl, ethyl, prop-1-yl, 1-methylethyl, but-1-yl, 1-methylpropyl, 2-methylpropyl, 1,1-dimethylethyl, n-pentyl, 1-methylbutyl, 2-methylbutyl, 3-methylbutyl, 1,1-dimethylpropyl, 1,2-dimethylpropyl, 2,2-dimethylpropyl, 1-ethylpropyl, n-hexyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl, 1-ethyl-2-methylpropyl, trifluoromethyl, difluoromethyl, pentafluoroethyl, 2,2-difluoroethyl, 2,2,2-trifluoroethyl, methoxy, ethoxy, prop-1-yloxy, prop-2-yloxy, but-1-yloxy, but-2-yloxy, 2-methylprop-1-yloxy, 1,1-dimethyleth-1-yloxy, difluoromethoxy, trifluoromethoxy, pentafluoroethoxy, 2,2-difluoroethoxy, 2,2,2-trifluoroethoxy,
G is methylene, (methyl)methylene, (ethyl)methylene, (prop-1-yl)methylene, (prop-2-yl)methylene, (but-1-yl)methylene, (but-2-yl)methylene, (pent-1-yl)methylene, (pent-2-yl)methylene, (pent-3-yl)methylene, (dimethyl)methylene, (diethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, 1,1,2-trimethylethyl-1-ene, 1,2,2-trimethylethyl-1-ene, 1,1,2,2-tetramethylethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene, 1-methylpentyl-1-ene, 2-methylpentyl-1-ene, 3-methylpentyl-1-ene, 4-methylpentyl-1-ene, 1,1-dimethylbutyl-1-ene, 1,2-dimethylbutyl-1-ene, 1,3-dimethylbutyl-1-ene, 2,2-dimethylbutyl-1-ene, 2,3-dimethylbutyl-1-ene, 3,3-dimethylbutyl-1-ene, 1-ethylbutyl-1-ene, 2-ethylbutyl-1-ene, 1,1,2-trimethylpropyl-1-ene, 1,2,2-trimethylpropyl-1-ene, 1-ethyl-1-methylpropyl-1-ene, 1-ethyl-2-methylpropyl-1-ene,
X and Y are independently O (oxygen) or S (sulfur)
and
Q is one of the moieties Q-1 to Q-406 specified below, where the arrow in the structural formulae in the table below represents a bond of the respective Q group to a carbonyl group in formula (I):

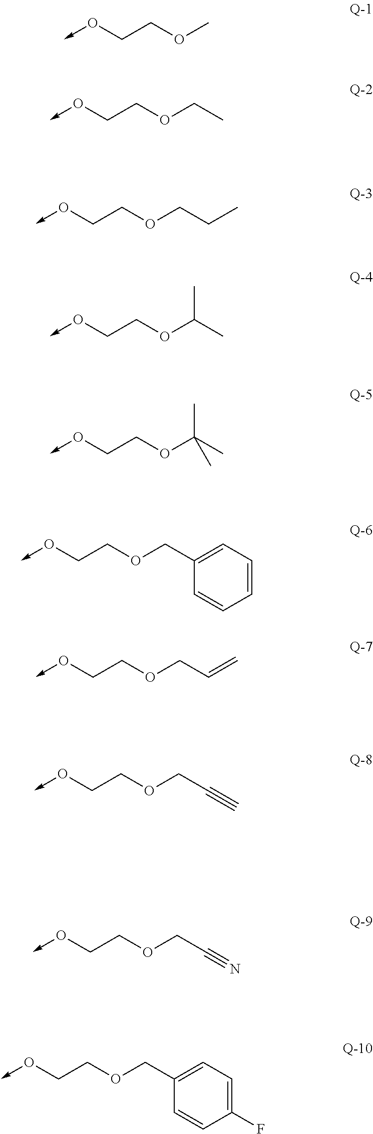

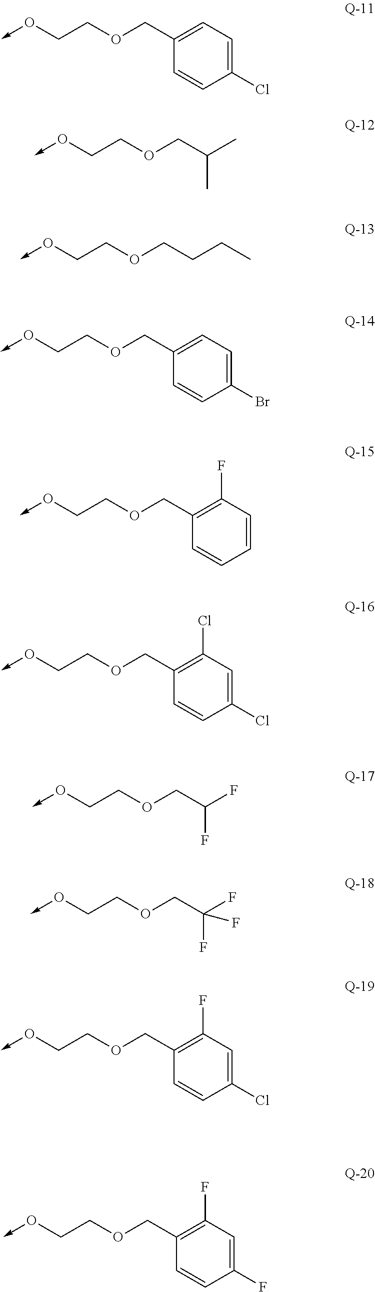

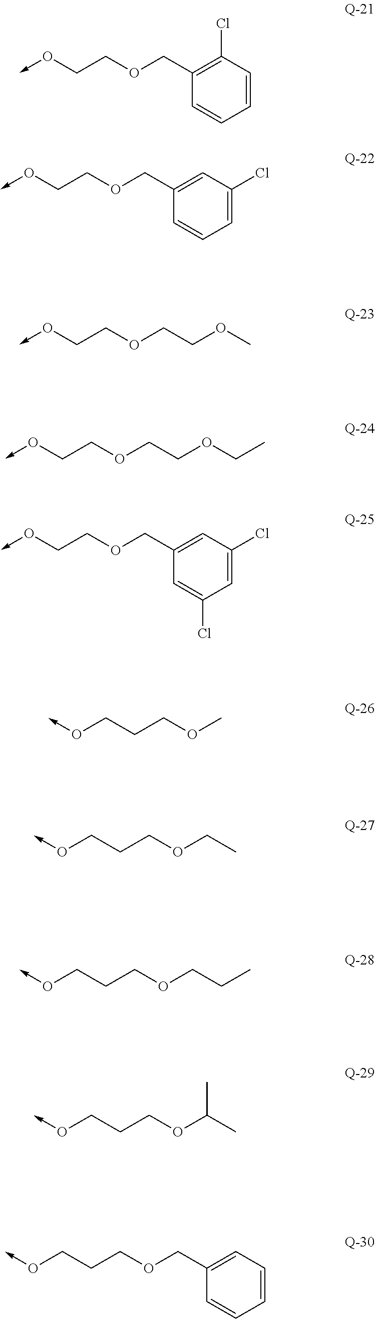

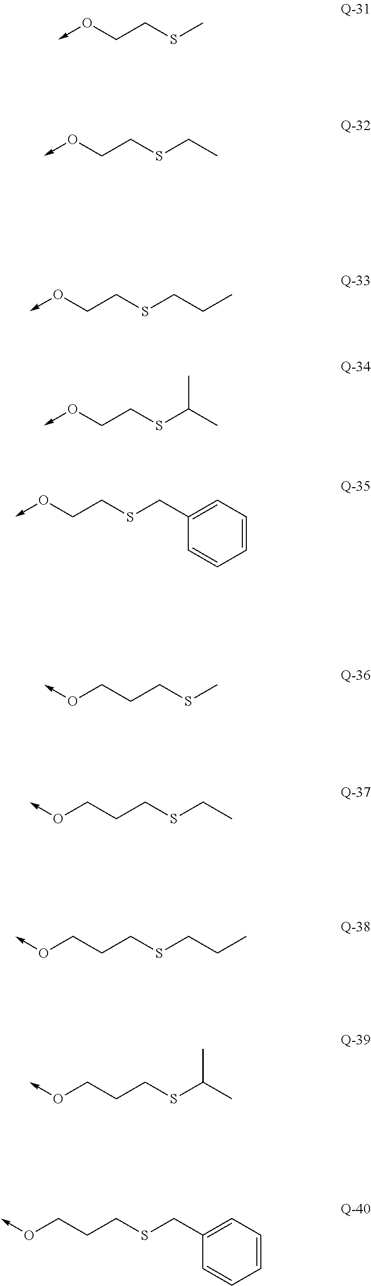

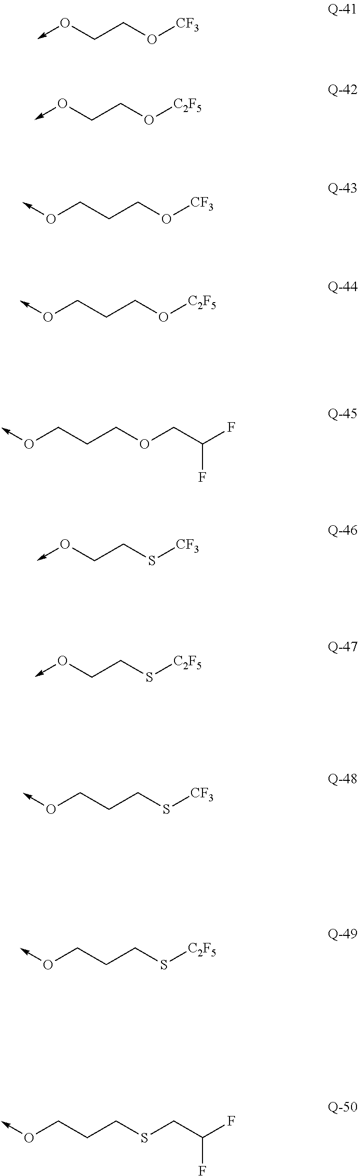

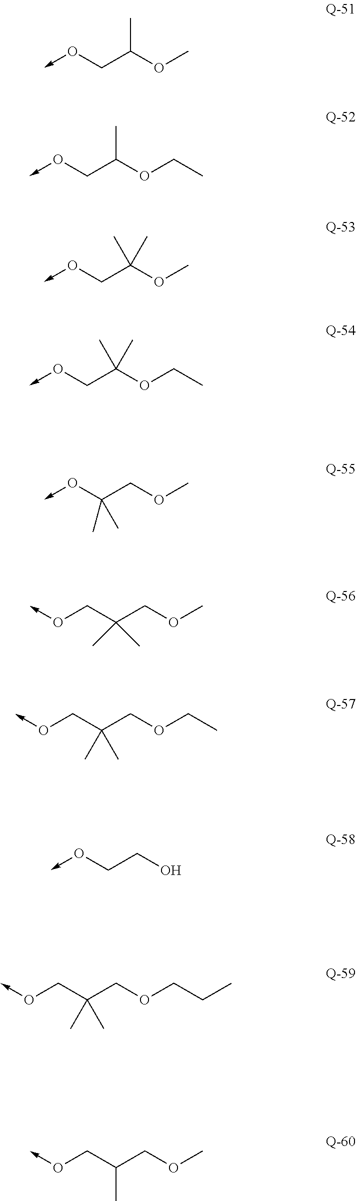

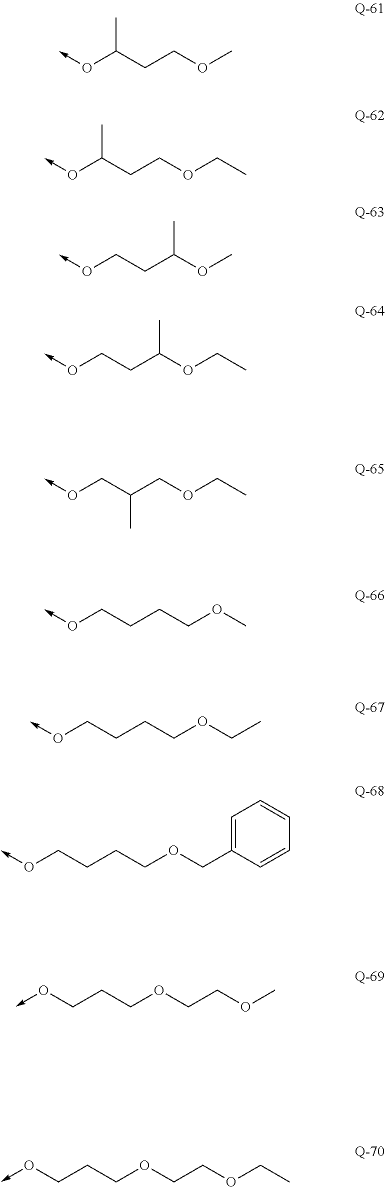

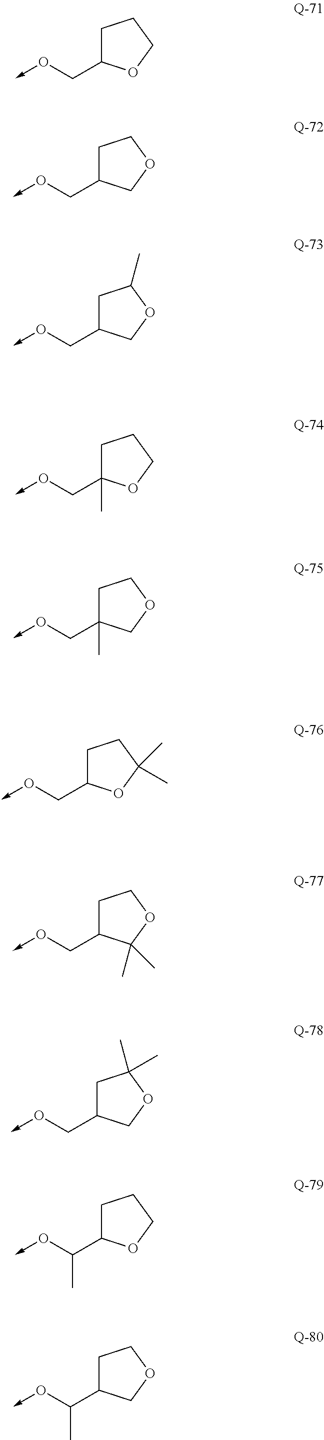

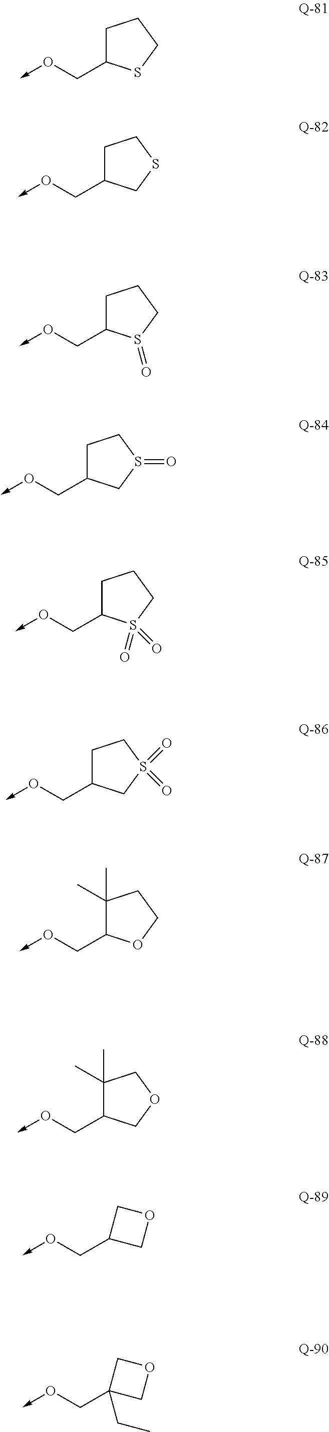

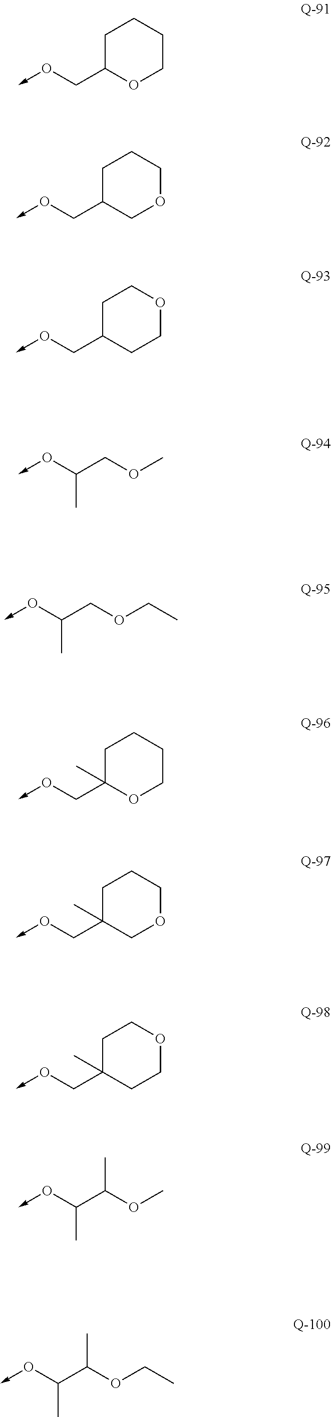

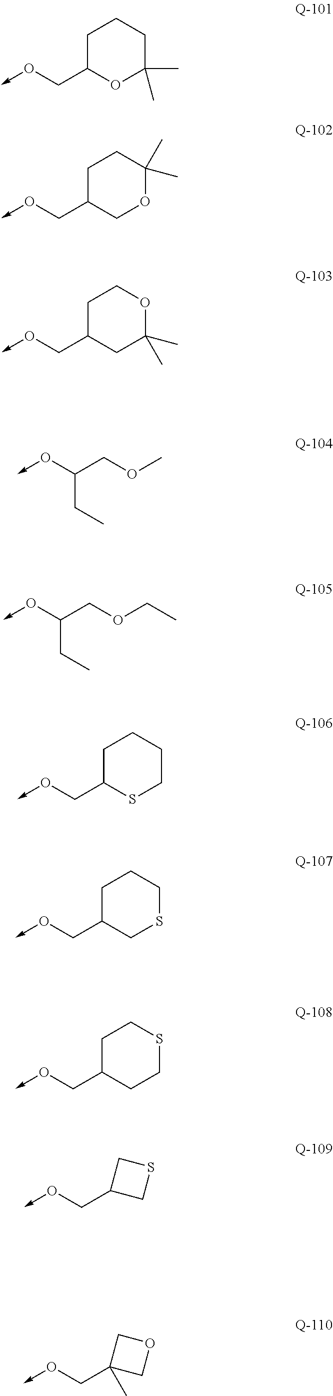

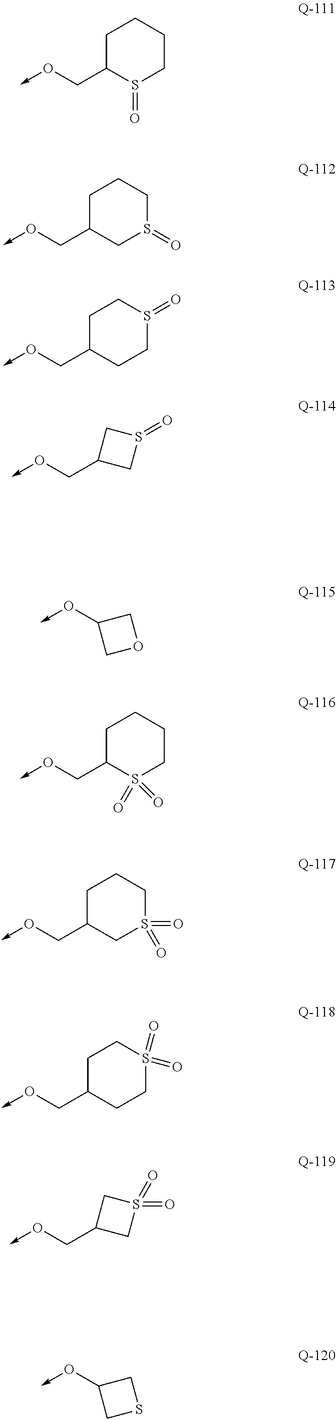

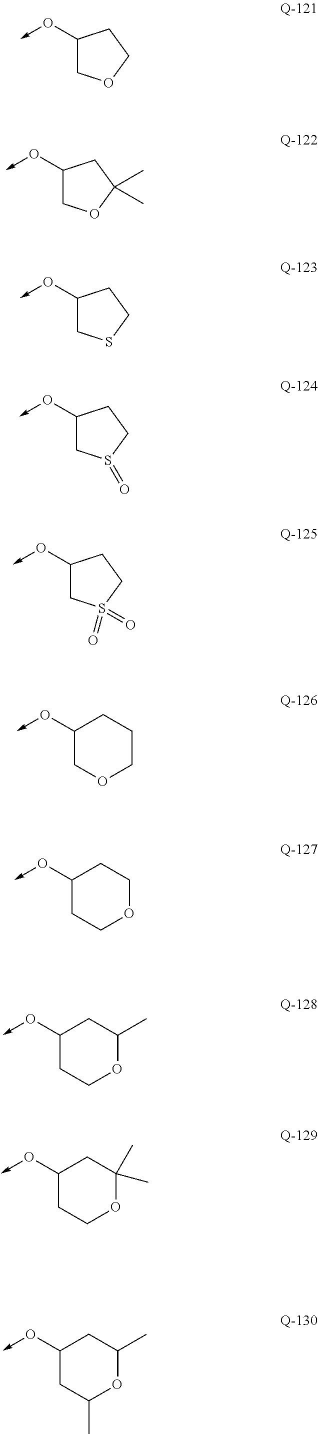

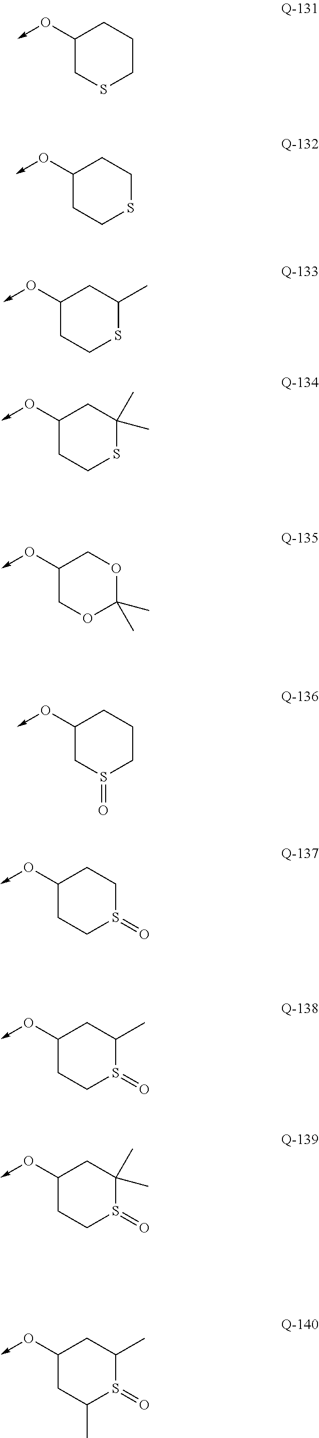

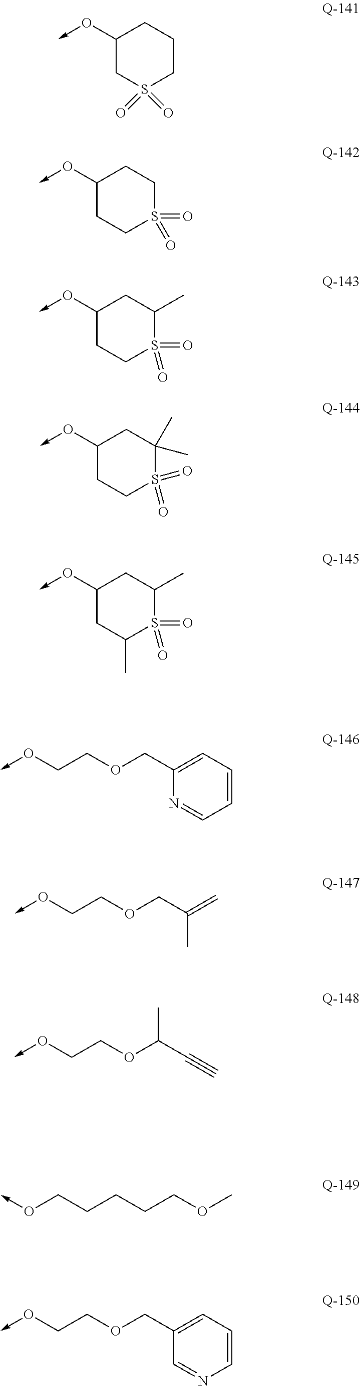

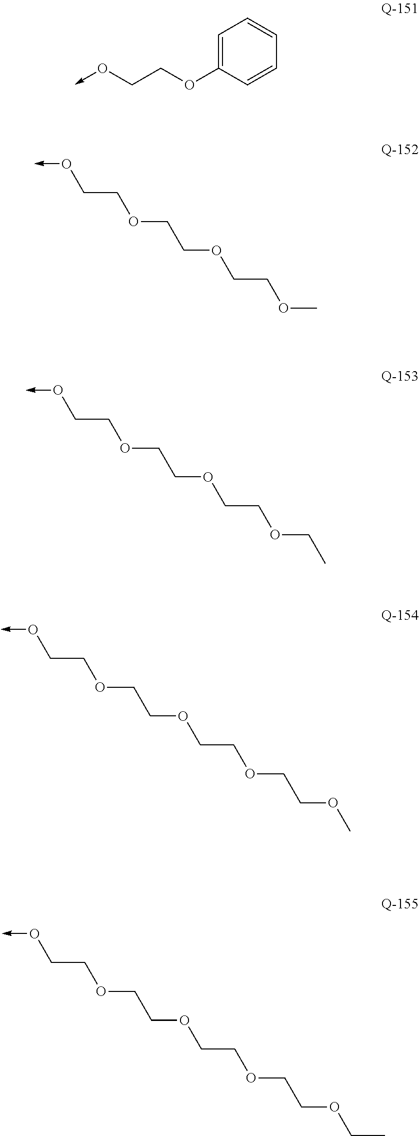

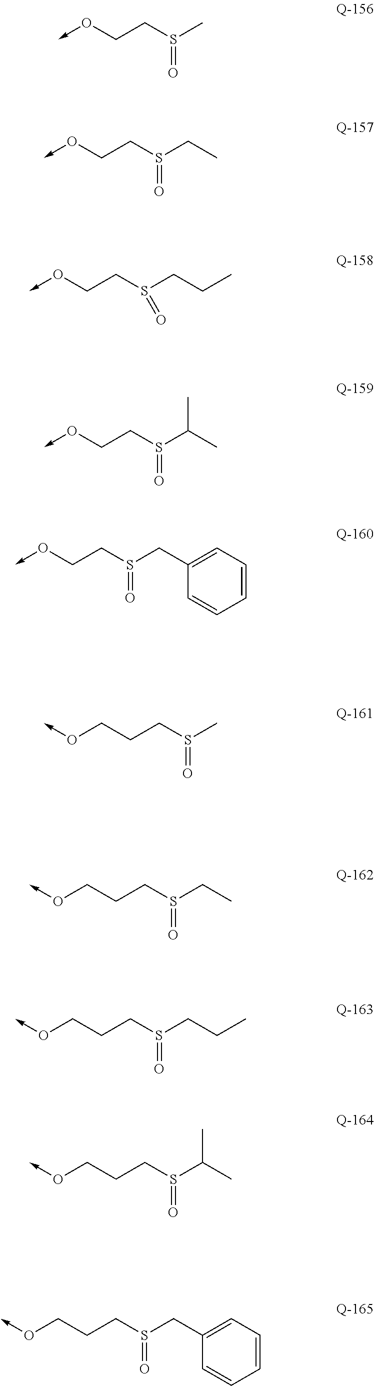

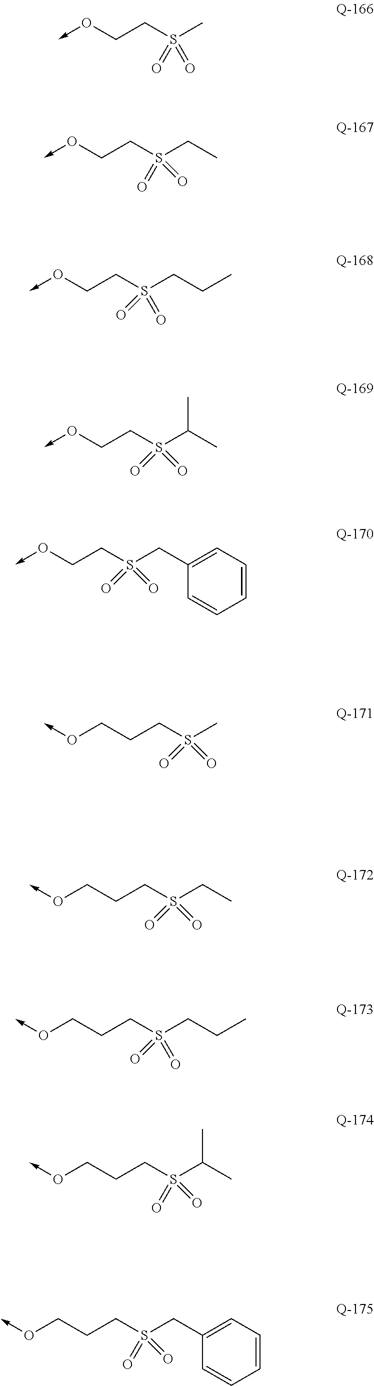

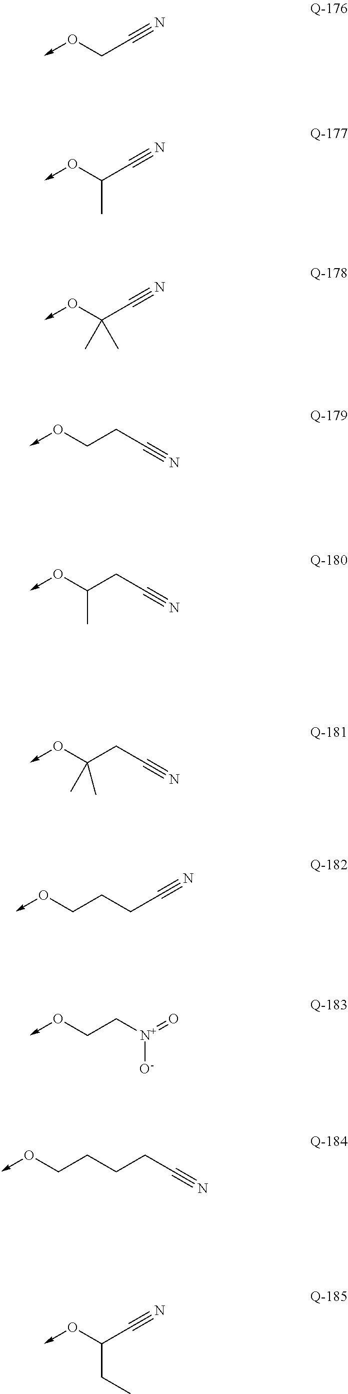

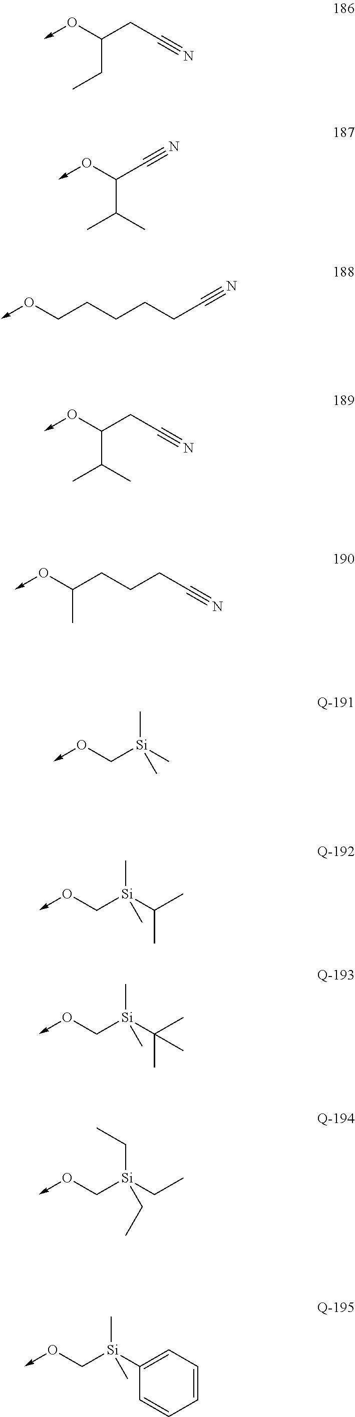

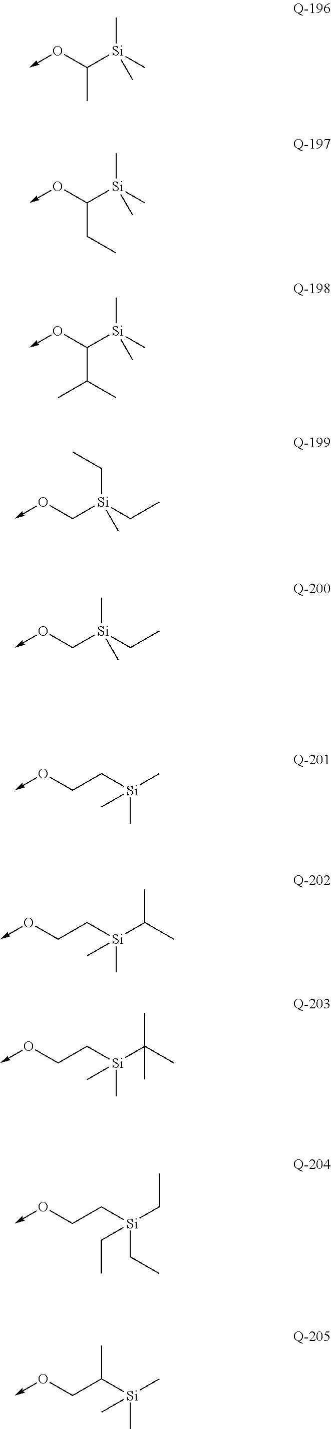

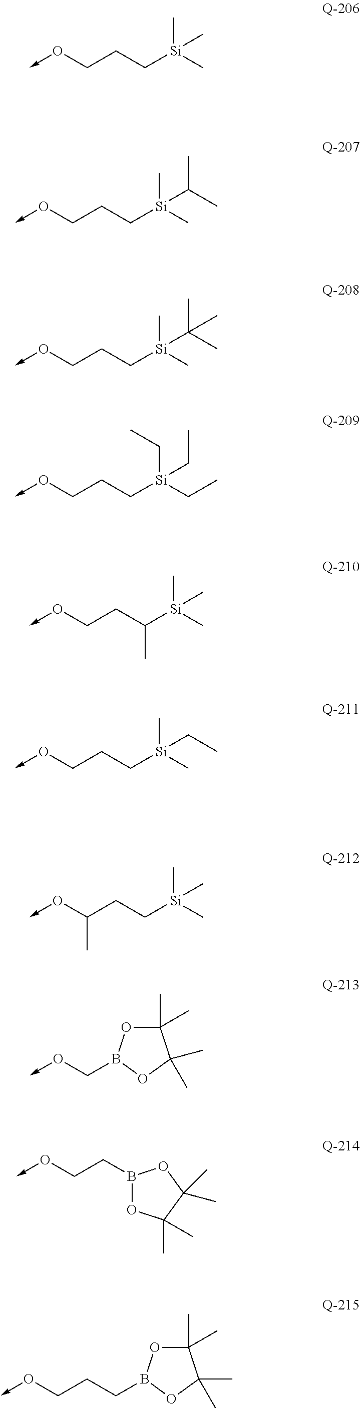

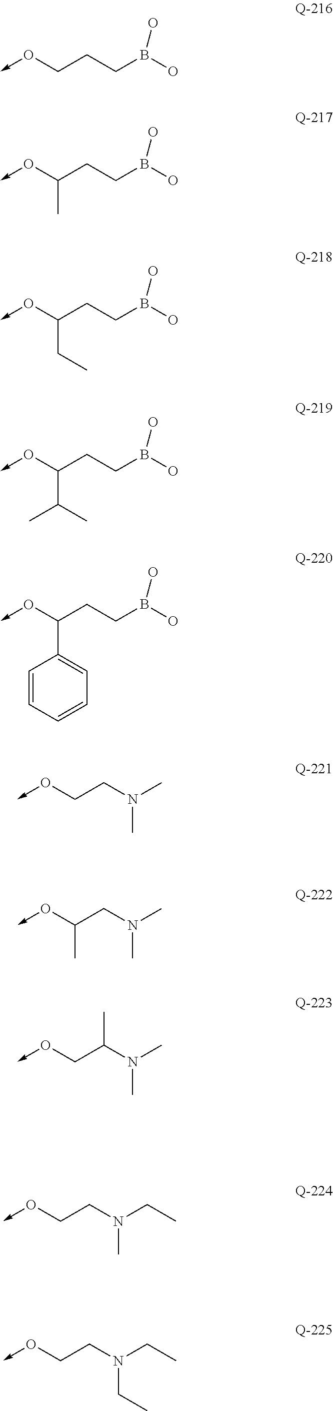

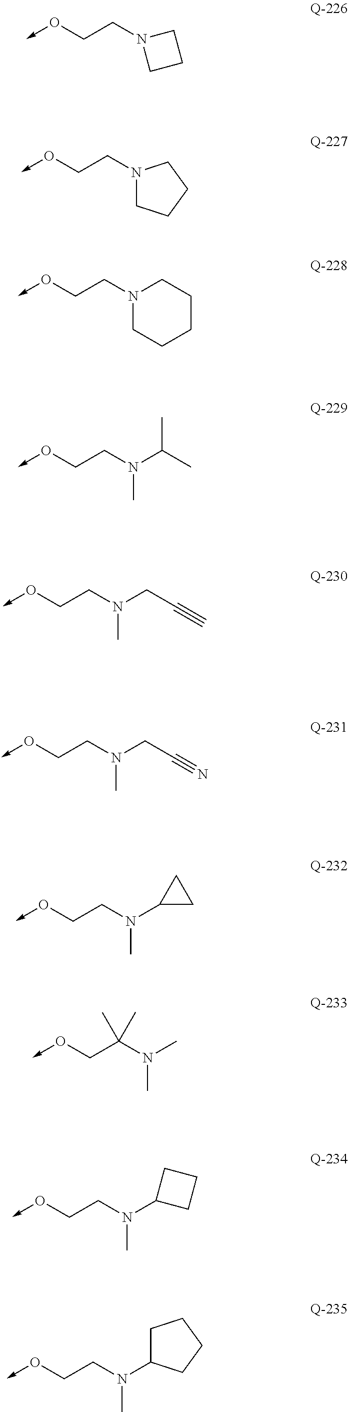

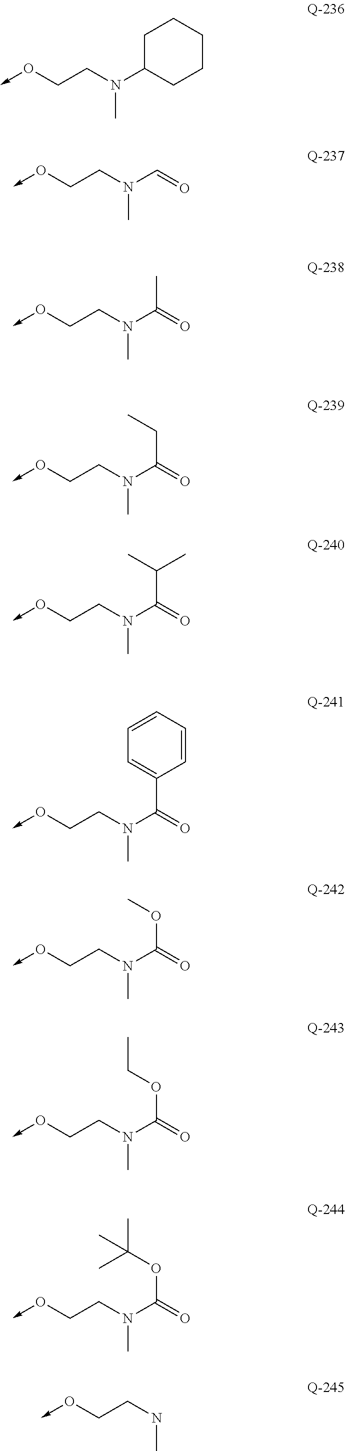

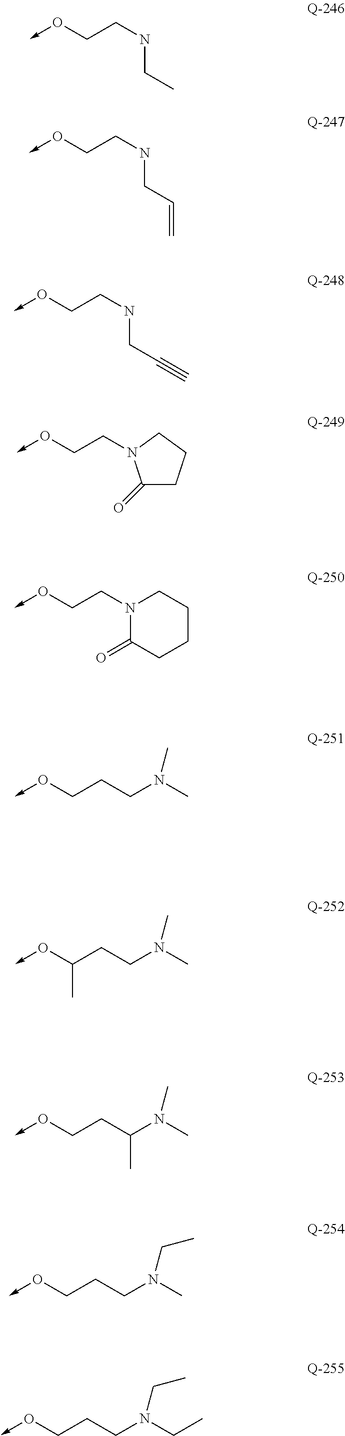

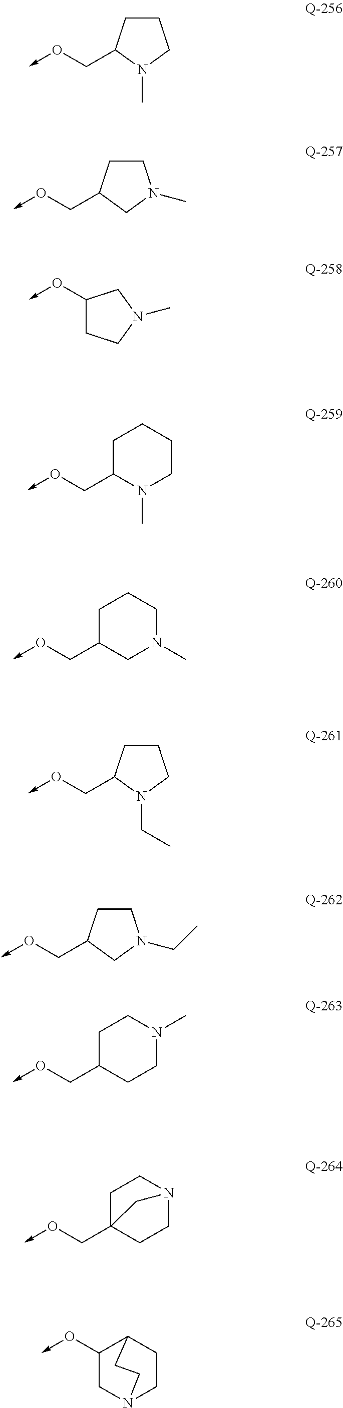

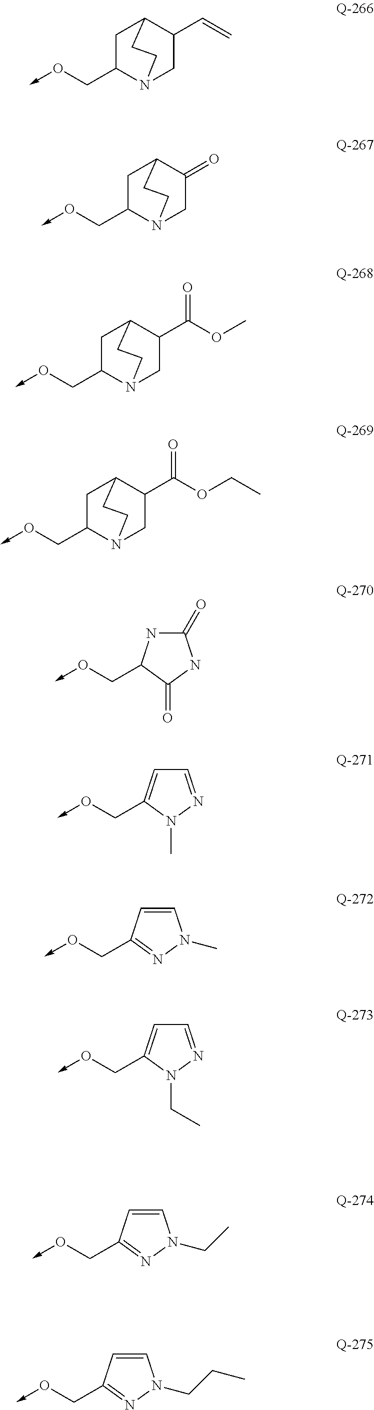

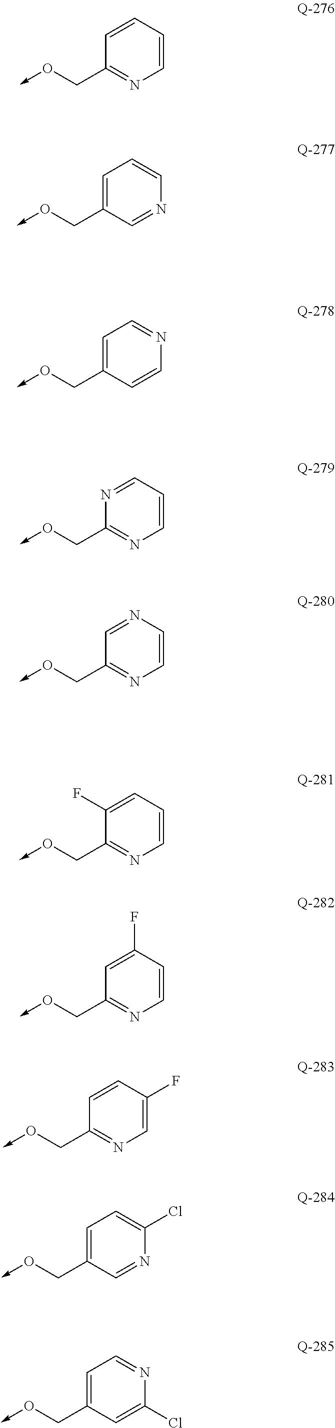

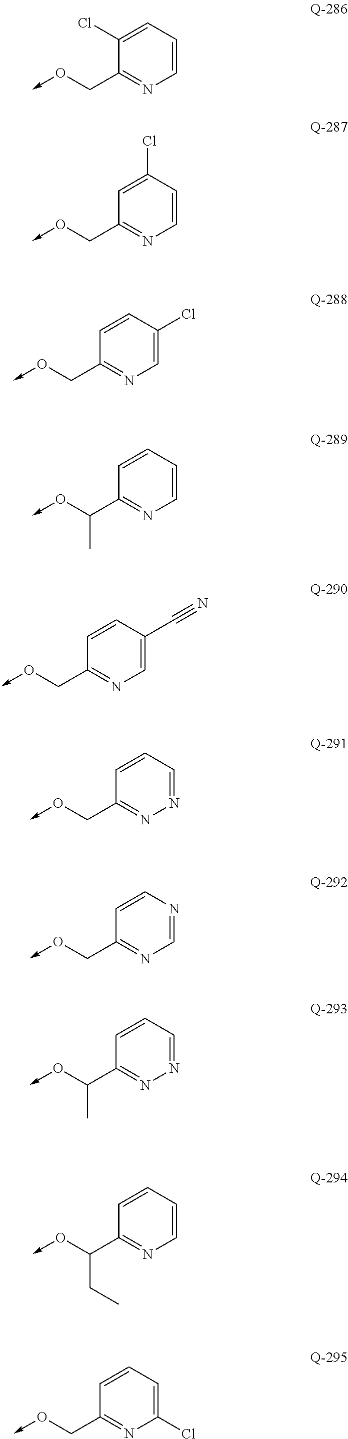

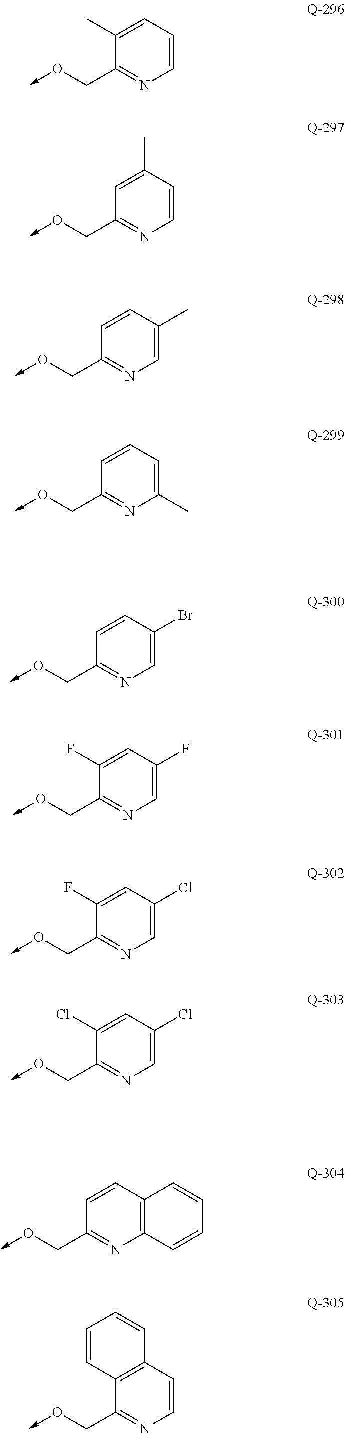

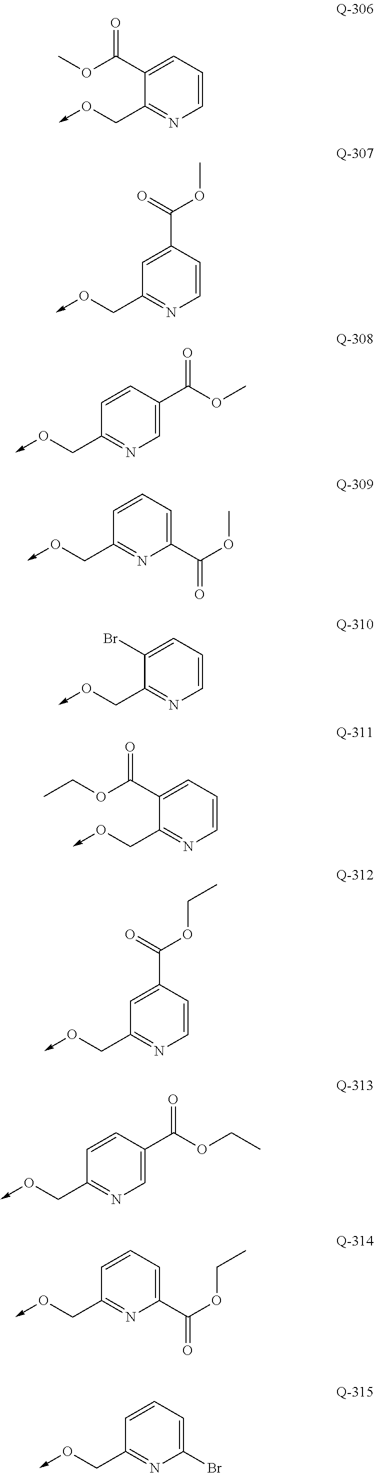

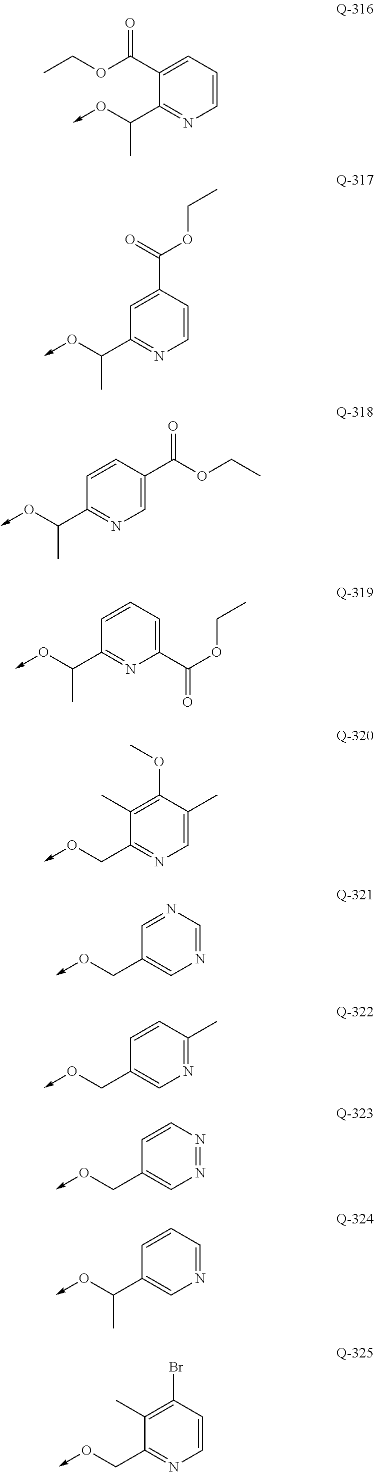

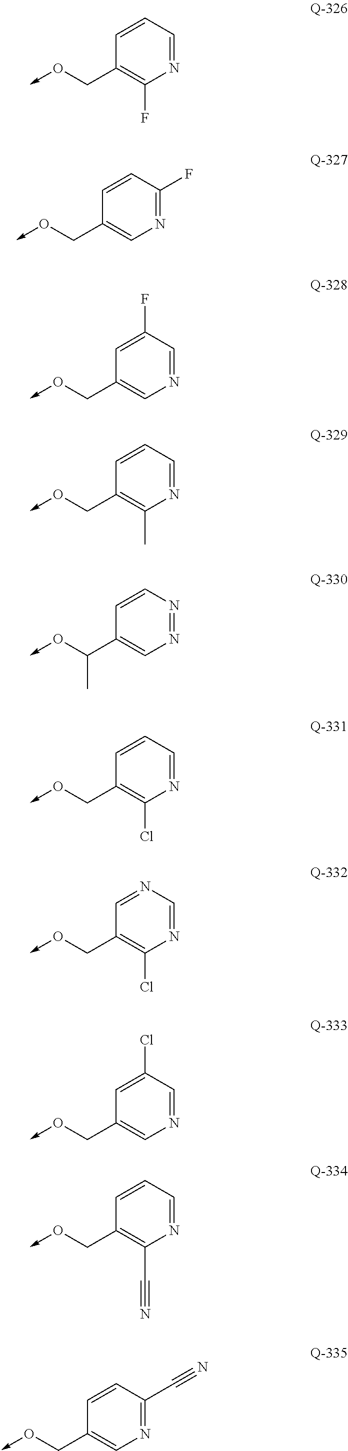

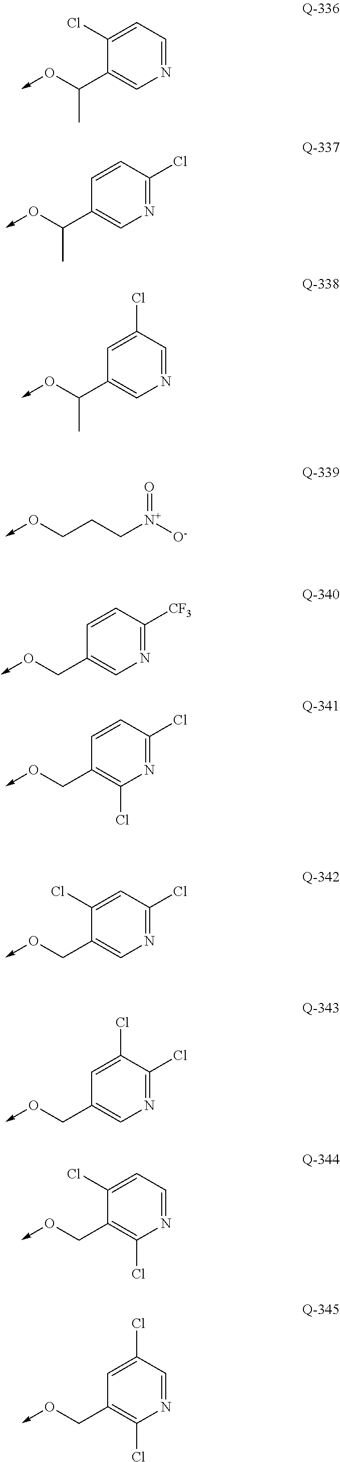

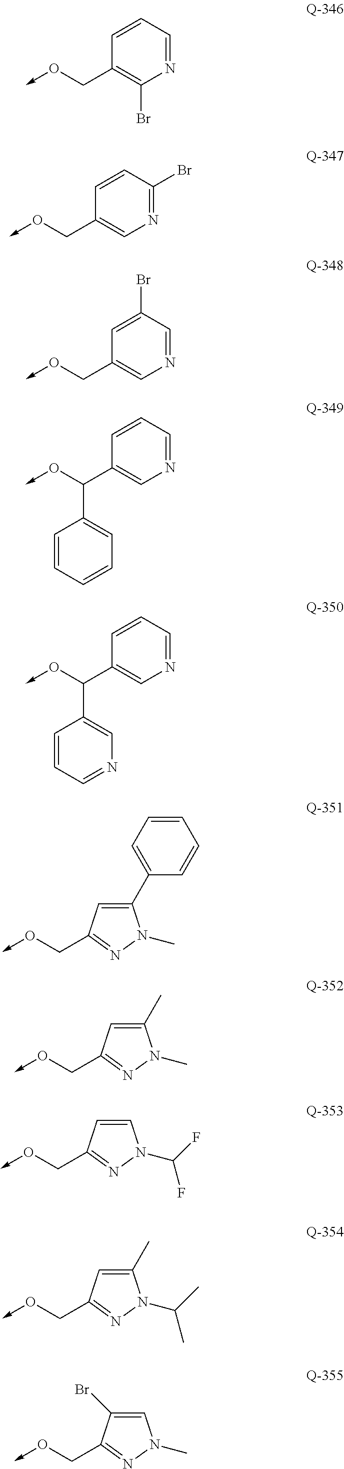

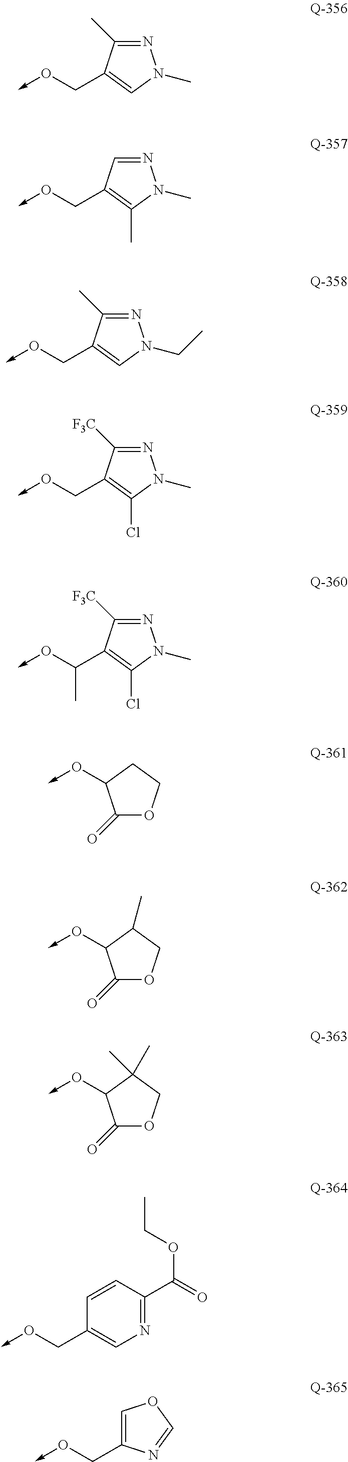

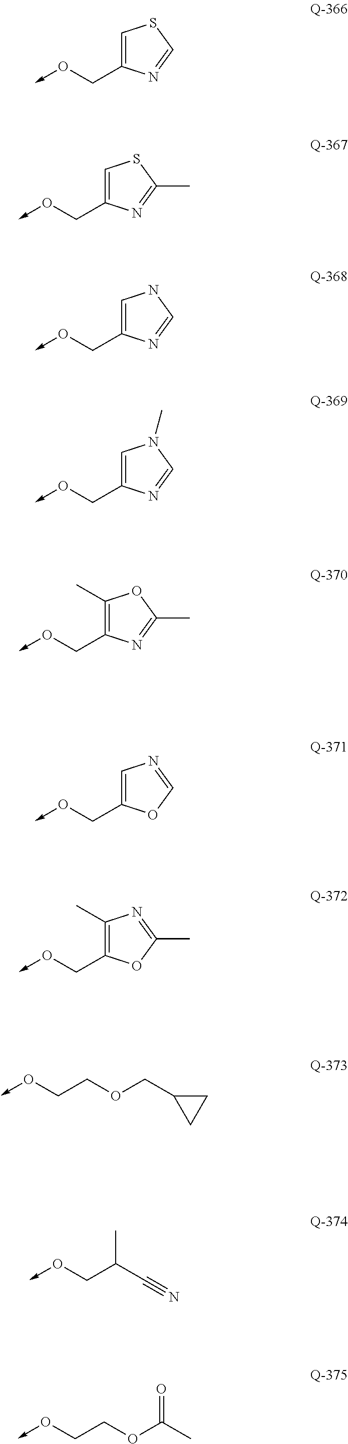

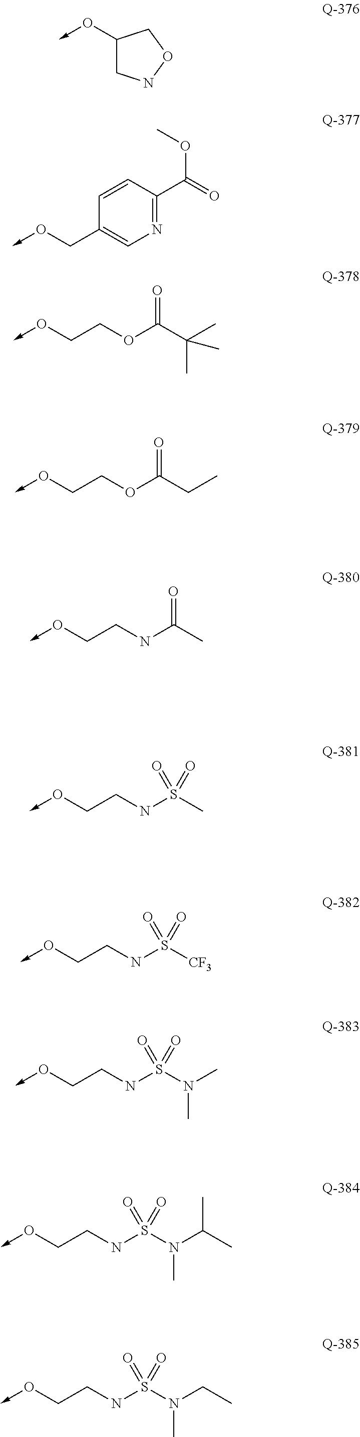

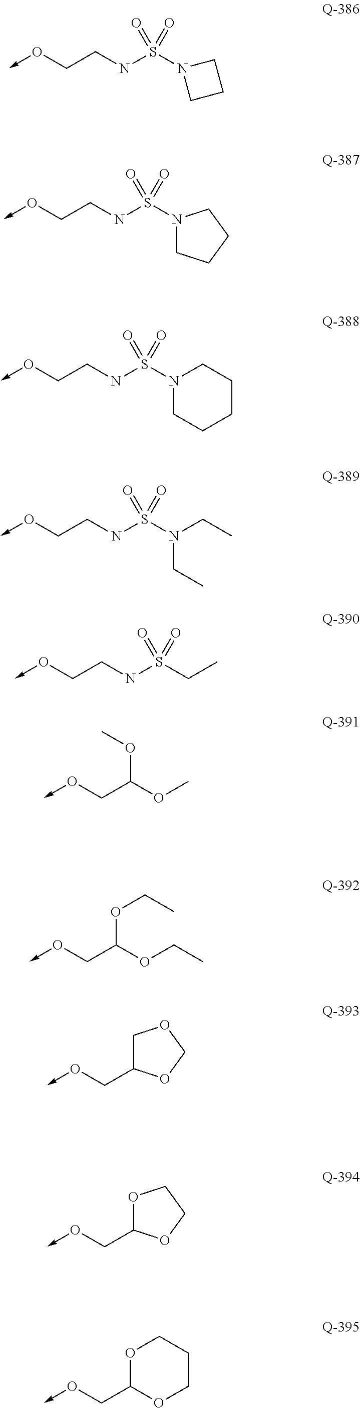

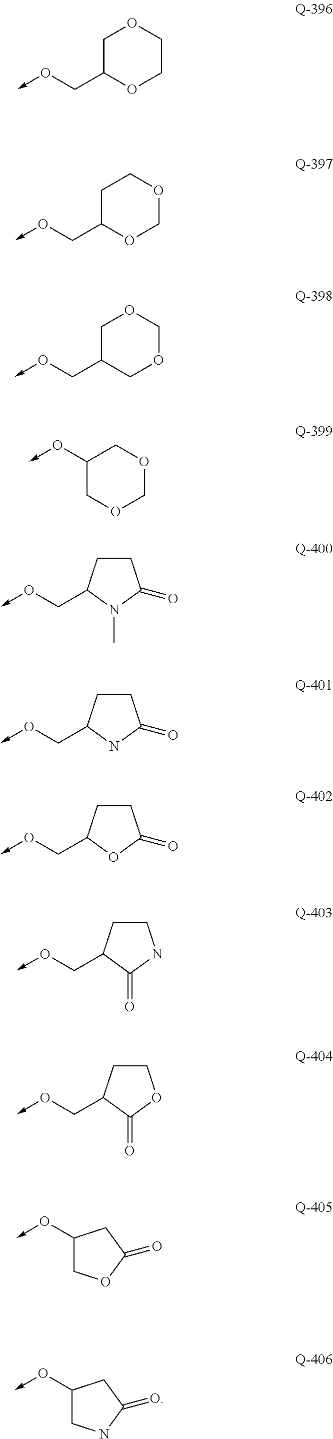

4. The compound of formula (I) as claimed in claim 3 and/or salt thereof, wherein
R¹ is hydrogen, fluorine, chlorine, bromine, iodine, cyano, methyl, ethyl, trifluoromethyl, difluoromethyl, methoxy, ethoxy, difluoromethoxy, trifluoromethoxy,
R² is fluorine,
R³ is hydrogen, fluorine, chlorine, bromine, methoxy,
R⁴ is fluorine, chlorine, bromine, cyano, NO₂, C(O)NH₂, C(S)NH₂, trifluoromethyl, ethynyl, propyn-1-yl,
R⁵, R⁶, R⁷ are independently hydrogen, fluorine, chlorine, bromine, iodine, cyano, methyl, ethyl, trifluoromethyl, difluoromethyl, methoxy, ethoxy, difluoromethoxy, trifluoromethoxy,
G is methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, 1,1-dimethylethyl-1-ene, 2,2-dimethylethyl-1-ene, 1-ethylethyl-1-ene, 2-ethylethyl-1-ene, 1-(prop-1-yl)ethyl-1-ene, 2-(prop-1-yl)ethyl-1-ene, 1-(prop-2-yl)ethyl-1-ene, 2-(prop-2-yl)ethyl-1-ene, n-pentylene, 1-methylbutyl-1-ene, 2-methylbutyl-1-ene, 3-methylbutyl-1-ene, 4-methylbutyl-1-ene, 1,1-dimethylpropyl-1-ene, 2,2-dimethylpropyl-1-ene, 3,3-dimethylpropyl-1-ene, 1,2-dimethylpropyl-1-ene, 1,3-dimethylpropyl-1-ene, 1-ethylpropyl-1-ene, n-hexylene,
X and Y are independently O (oxygen) or S (sulfur)
and
Q is one of the moieties Q-1 to Q-406.

5. The compound of formula (I) as claimed in claim 3 and/or salt thereof, wherein
R¹ is hydrogen, fluorine, chlorine, bromine, cyano, methyl, trifluoromethyl, methoxy, trifluoromethoxy,
R² is fluorine,
R³ is fluorine,
R⁴ is chlorine, bromine, cyano, NO₂, C(O)NH₂, C(S)NH₂,
R⁵, R⁶ and R⁷ are independently hydrogen, fluorine, chlorine, bromine, cyano, methyl, trifluoromethyl, methoxy, trifluoromethoxy,
G is methylene, (methyl)methylene, (ethyl)methylene, (dimethyl)methylene, ethylene, n-propylene, (1-methyl)ethyl-1-ene, (2-methyl)ethyl-1-ene, n-butylene, 1-methylpropyl-1-ene, 2-methylpropyl-1-ene, 3-methylpropyl-1-ene, n-pentylene, n-hexylene,
X and Y are independently O (oxygen) or S (sulfur)
and
Q is one of the moieties Q-1 to Q-406.

6. The compound of formula (I) as claimed in claim 3 and/or salt thereof, wherein
R¹ is hydrogen,
R² is fluorine,
R³ is fluorine,
R⁴ is chlorine, bromine, cyano, NO₂,
R⁵ is hydrogen, fluorine, chlorine, bromine,
R⁶ is hydrogen, fluorine, chlorine, bromine, cyano,
R⁷ is hydrogen,
G is methylene, (methyl)methylene,
X is O (oxygen) or S (sulfur),
Y is O (oxygen)
and
Q is one of the moieties Q-1 to Q-406.

7. The compound of formula (I) as claimed in claim 3 and/or salt thereof, wherein
R¹ is hydrogen,
R² is fluorine,
R³ is fluorine,
R⁴ is chlorine, bromine, cyano, NO₂,
R⁵ is hydrogen, fluorine,
R⁶ is hydrogen, fluorine, bromine, cyano,
R⁷ is hydrogen,
G is methylene, (methyl)methylene,
X is O (oxygen) or S (sulfur),
Y is O (oxygen)
and
Q is one of the moieties Q-1 to Q-35, Q-41 to Q-45, Q-58, Q-71 to Q-80, Q-89, Q-94, Q-95, Q-115, Q-120 to Q-123, Q-152 to Q-155, Q-166 to Q-170, Q-176 to Q-190, Q-261 to Q-348, Q-352 to Q-372, Q-377, Q-391-Q-399.

8. The compound of formula (I) as claimed in claim 3 and/or salt thereof, wherein
R¹ is hydrogen,
R² is fluorine,
R³ is fluorine,
R⁴ is chlorine, bromine, cyano, NO₂,
R⁵ is hydrogen, fluorine,
R⁶ is hydrogen, fluorine, bromine, cyano,
R⁷ is hydrogen,
G is methylene, (methyl)methylene,
X is O (oxygen) or S (sulfur),
Y is O (oxygen)
and
Q is one of the moieties Q-1, Q-2, Q-3, Q-4, Q-7, Q-8, Q-9, Q-17, Q-18, Q-23, Q-24, Q-26, Q-27, Q-41, Q-42, Q-43, Q-58, Q-71, Q-72, Q-89, Q-94, Q-115, Q-121, Q-176, Q-177, Q-179, Q-183, Q-272, Q-274, Q-275, Q-276, Q-277, Q-278, Q-281, Q-282, Q-283, Q-284, Q-286, Q-288, Q-291, Q-296, Q-301, Q-302, Q-303, Q-308, Q-309, Q-321, Q-327, Q-328, Q-329, Q-331, Q-335, Q-339, Q-356, Q-365, Q-366, Q-367, Q-371, Q-394.

9. A product comprising one or more compounds of formula (I) as defined in claim 1 and/or salts thereof, comprising a herbicide and/or plant growth regulator product, optionally adapted for one or more crops of one or more useful plants and/or ornamentals.

10. A herbicidal and/or plant growth-regulating composition, wherein the composition comprises one or more compounds of formula (I) as defined in claim 1 and/or salts thereof, and one or more further substances selected from groups (i) and/or (ii), comprising
(i) one or more further agrochemically active substances, optionally selected from the group consisting of insecticides, acaricides, nematicides, further herbicides, fungicides, safeners, fertilizers and/or further growth regulators,
(ii) one or more formulation auxiliaries customary in crop protection.

11. A method of controlling one or more harmful plants and/or for regulating the growth of one or more plants, comprising applying an effective amount
of one or more compounds of formula (I), as defined in claim 1 and/or salts thereof, or
a composition thereof,
to the plants, seeds of plants, soil in which or on which plants grow and/or an area under cultivation.
